(19) United States
(12) Patent Application Publication
(10) Pub. No.: US 2021/0293820 A1
(43) Pub. Date: Sep. 23, 2021

(54) METHODS OF ACTIVATING DYSFUNCTIONAL IMMUNE CELLS AND TREATMENT OF CANCER

(71) Applicants: Yeda Research and Development Co. Ltd., Rehovot (IL); Stichting Het Nederlands Kanker Instituut - Antoni Van Leeuwenhoek Ziekenhuis, Amsterdam (NL)

(72) Inventors: Ido AMIT, Rehovot (IL); Hanjie LI, Rehovot (IL); Amos TANAY, Rehovot (IL); Ido YOFE, Rehovot (IL); Yaniv EYAL-LUBLING, Rehovot (IL); Antonius Nicolaas Maria SCHUMACHER, Amsterdam (NL); Anne Magdalena VAN DER LEUN, Amsterdam (NL)

(73) Assignees: Yeda Research and Development Co. Ltd., Rehovot (IL); Stichting Het Nederlands Kanker Instituut - Antoni Van Leeuwenhoek Ziekenhuis, Amsterdam (NL)

(21) Appl. No.: 17/334,862

(22) Filed: May 31, 2021

Related U.S. Application Data
(63) Continuation of application No. PCT/IL2019/051312, filed on Nov. 28, 2019.

(30) Foreign Application Priority Data
Nov. 29, 2018 (IL) 263394

(51) Int. Cl. G01N 33/574; C12N 15/113; G01N 33/50

(52) U.S. Cl. CPC G01N 33/574; C12Q 2600/156; G01N 33/505; C12N 15/113

(57) ABSTRACT

Method of activating dysfunctional T cells, determining responsiveness of a subject having a tumor to an immune checkpoint inhibition are provided. Also provided is an agent capable of inhibiting a target gene or expression product thereof for use in treating a subject having a tumor. Additionally or alternatively provided is an immune checkpoint inhibitor and an agent capable of inhibiting a target gene or expression product thereof for use in treating a subject having a tumor.

### RELATED APPLICATIONS

This application is a Continuation patent application of PCT Application No. PCT/IL2019/051312 filed on Nov. 28, 2019, which claims the benefit of priority of Israel Patent Application No. 263394 filed on Nov. 29, 2018. The contents of the above applications are all incorporated by reference as if fully set forth herein in their entirety.

### SEQUENCE LISTING STATEMENT

The ASCII file, entitled 88026Sequence Listing.txt, created on May 31, 2021, comprising 283,926 bytes, submitted concurrently with the filing of this application is incorporated herein by reference.

### FIELD AND BACKGROUND OF THE INVENTION

The present invention, in some embodiments thereof, relates to methods of activating dysfunctional immune cells and treatment of cancer.

T cell checkpoint blockade therapies that aim to reactivate tumor-specific T cell responses have revolutionized cancer treatment, resulting in durable responses in patients with advanced disease (Ribas and Wolchok, 2018; Sharma and Allison, 2015). Nevertheless, many patients do not achieve long-term clinical benefit, and our understanding of the mechanisms underlying response or resistance to these therapies are still incomplete (Reading et al., 2018; Sharma et al., 2017; Śledzińska et al., 2015). Recent single cell RNA sequencing-based studies of tumor infiltrating immune cell populations in melanoma and other tumor types provide evidence for a highly heterogeneous make-up of immune cell infiltrates, and this heterogeneity is likely to form a determining factor in therapy outcome (Azizi et al., 2018; Guo et al., 2018; Lavin et al., 2017; Sade-Feldman et al., 2018; Savas et al., 2018; Tirosh et al., 2016; Zhang et al., 2018; Zheng et al., 2017). Continuous increases in sample size and quality, diversity of patients sampled, and analysis methodology are required to uncover the mechanisms that underlie successful immunotherapy response.

Within the heterogeneous tumor microenvironment, T cells make up a considerable part of the immune infiltrate. The intratumoral T cell compartment comprises effector, memory, and regulatory T cells. In addition, a subset of CD8 T cells that has acquired a state of ‘dysfunction’ or ‘exhaustion’ is frequently observed. Such dysfunctional T cells are characterized by a loss of classical CD8 T cell effector functions, such as cytotoxicity (Hashimoto et al., 2018; Pauken and Wherry, 2015; Wherry and Kurachi, 2015). In addition, the dysfunctional T cells in human tumors display a unique T cell cytokine secretion signature (Guo et al., 2018; Sade-Feldman et al., 2018; Savas et al., 2018; Thommen and Schumacher, 2018; Thommen et al., 2018). Whereas T cell exhaustion was previously associated with a loss of proliferative capacity, recent studies are providing evidence for a proliferative potential of T cells with high levels of PD-1 expression in human tumors (Guo et al., 2018; Savas et al., 2018; Thommen et al., 2018; Zhang et al., 2018). In addition to high levels of PD-1 expression, the dysfunctional T cell compartment is characterized by increased expression of inhibitory checkpoint molecules such as TIM-3 and LAGS (Thommen and Schumacher, 2018; Wherry and Kurachi, 2015). Furthermore, characterization of dysfunctional T cell populations in murine tumor and chronic viral infection models has demonstrated that dysfunctionality of T cells in these models is associated with the expression of transcriptional regulators such as Prdm1, Maf1, and Eomes (Chihara et al., 2018; Paley et al., 2012; Shin et al., 2009). To what extent these and other factors drive T cell dysfunction in human melanoma, and how their expression is induced, remain open and important questions.

The role and predictive potential of T cells with different levels of expression of exhaustion markers is presently a matter of debate. In murine models, T cells with high expression of markers of T cell exhaustion appear refractory to reinvigoration by PD-1 blockade (Blackburn et al., 2008; Im et al., 2016; Pauken et al., 2016; Philip et al., 2017; Schietinger et al., 2016). Nevertheless, the frequency of dysfunctional T cells expressing high levels of PD-1 has been shown to correlate with clinical response to anti-PD-1 therapy in NSCLC patients (Thommen et al., 2018). As a second issue, a significant complication in the analysis of T cell states in human tumors is that T cell infiltrates at tumor sites express a variable degree of tumor-reactivity (Scheper et al., in press; Simoni et al., 2018). For this reason, detailed characterization of dysfunctional T cells in a setting in which TCR clonality and the level of tumor-reactivity is known would be of value.

Hence, detailed characterization of dysfunctional T cells can lead to better understanding of their role in immune regulation and function and to identification of novel immune modulatory pathways and further optimization of strategies for T-cell activation, vital for establishing improved response across diverse human tumors.

Additional related background art:

### US Publication Number 20110105341

### WO2017191274

### SUMMARY OF THE INVENTION

According to an aspect of some embodiments of the present invention there is provided a method of activating dysfunctional CD8⁺/Lag3⁺/PD1⁺/Tim3⁺/CD103⁺/CD39⁺/CD137⁺/Klrg1⁻ T cells, the method comprising, contacting dysfunctional CD8⁺/Lag3⁺/PD1⁺/Tim3⁺/CD103⁺/CD39⁺/CD137⁺/Klrg1⁻ T cells with an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby activating the dysfunctional immune cells.

According to an aspect of some embodiments of the present invention there is provided a method of determining responsiveness of a subject having a tumor to an immune checkpoint inhibition, the method comprising determining in a tumor of a subject a level of dysfunctional CD8⁺/Lag3⁺/PD1⁺/Tim3⁺/CD103⁺/CD39⁺/CD137⁺/Klrg1⁻ T cells, wherein a level of the dysfunctional cells above a predetermined threshold is indicative of a response to an immune checkpoint inhibition.

According to an aspect of some embodiments of the present invention there is provided a method of treating a subject having a tumor, the method comprising:

(a) determining responsiveness of a subject to immune checkpoint inhibition as described herein; and
(i) wherein when the level of the dysfunctional cells is above the predetermined threshold, treating or selecting treatment for the subject with immune checkpoint inhibition; or
(ii) wherein when the level of the dysfunctional cells is below the predetermined threshold, subjecting the dysfunctional cells to ex vivo expansion and subsequently treating or selecting treatment for the subject with the immune checkpoint inhibition.

According to an aspect of some embodiments of the present invention there is provided a method of treating a subject having a tumor, the method comprising administering to the subject an immune checkpoint inhibitor and an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby treating the subject having the tumor.

According to an aspect of some embodiments of the present invention there is provided a method of treating a subject having a tumor, the method comprising administering to the subject an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby treating the subject having the tumor.

According to an aspect of some embodiments of the present invention there is provided an agent capable of inhibiting a target gene or expression product thereof selected from the group consisting of AKAP5, DGKH, PAG1, GALM, FUT8, WARS, CBLB, PIK3AP1, APOBEC3G, SLAMF7, SIRPG, GALNT1 for use in treating a subject having a tumor.

According to an aspect of some embodiments of the present invention there is provided an immune checkpoint inhibitor and an agent capable of inhibiting a target gene or expression product thereof selected from the group consisting of AKAP5, DGKH, PAG1, GALM, FUT8, WARS, CBLB, PIK3AP1, APOBEC3G, SLAMF7, SIRPG, GALNT1 for use in treating a subject having a tumor.

According to some embodiments of the invention, the administering the immune checkpoint inhibitor is following the administering the agent.

According to some embodiments of the invention, the activating is performed ex-vivo.

According to some embodiments of the invention, the activating is performed in-vivo.

According to some embodiments of the invention, the dysfunctional cells are in a proliferative cell state and/or are not in growth arrest.

According to some embodiments of the invention, the tumor is a solid tumor.

According to some embodiments of the invention, the solid tumor is a melanoma.

According to some embodiments of the invention, the dysfunctional T cells are tumor infiltrating cells.

According to some embodiments of the invention, the immune checkpoint is selected from the group consisting of cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD244 (2B4), CD160, GARP, OX40, CD137 (4-1BB), CD25, VISTA, BTLA, TNFR25, CD57, CCR2, CCRS, CCR6, CD39, CD73, CD4, CD18, CD49b, CD1d, CDS, CD21, TIMI, CD19, CD20, CD23, CD24, CD38, CD93, IgM, B220 (CD45R), CD317, CD11b, Ly6G, ICAM-1, FAP, PDGFR, Podoplanin, and TIGIT.

According to some embodiments of the invention, the determining the level of dysfunctional cells is performed by fluorescence activated cell sorting (FACS).

According to some embodiments of the invention, the determining the level of dysfunctional cells is performed by single cell transcriptome analysis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A, 1B, 1C, 1D, 1E and 1F Profiling immune infiltrates in human melanoma with scRNA-seq and scTCR-seq. A. Graphical overview of the experimental setting. Single immune cells were collected from human melanoma, and processed by MARS-seq for transcriptional profiling, and scTCR-seq for clonotype analysis of T cells. B. Two-dimensional (2D) projection of expression profiles of 47,772 immune cells partitioned into 324 metacells. Single cells are shown in dots. Metacells consisting of related cells are connected with edges and are positioned in proximity, broad immune cell subsets are annotated and marked by color code. C. 2D projection of sub-clustered T and NK cells. A total of 29,825 cells are represented in 218 metacells from the model shown in B, annotated in 9 groups and marked by color code. D. Expression (molecules/1,000 UMIs) of select genes across the T/NK metacell model. E. 2D projection of a selected set of marker genes over the metacell model. F. Distribution of the number of patients contributing to each metacell. Only patient with at least 2 cells in the metacell are considered as contributors.

FIGS. 1G, 1H, 1I, 1J, 1K, 1L, 1M, 1N 1O, 1P and 1Q G. FACS sorting strategy for T cells (CD45+/CD3+) and non-T cells (CD45+/CD3−) from tumor single cell suspension, as shown for three patients (bottom). H. Confusion matrix of all tumor immune infiltrates as shown in FIG. 1B. I-J. Metacell size distribution (C) and metacell ribosomal load compared to mean total UMI (D). K. Confusion matrix of T and NK cells. L. Distribution of FACS indices (measured by index-sorting) for CD4 and CD8 across different T cell types and NK cells for a representative patient. Values are logicle transformed (using the flowCore R package from Bioconductor). Colors represent cell types as in panel E. M. Scatter plot comparing mean absolute gene UMIs (log 2) of naive-like CD4 and naive-like CD8 T cells, based on FACS indices for CD4 and CD8. N. Naive gene enrichment as compared to non-naive T cells versus naive mean gene expression. O. Genes characterizing memory T cells, showing top 70 enriched genes, averaged across the 3 memory T metacells. P. Genes characterizing dysfunctional CD4 T cells, showing the top 70 enriched genes of the dysfunctional CD4 metacell. Q. Comparing cytotoxic T cells and NK cells gene enrichment, selecting the top 20 enriched genes within each group.

FIGS. 2A, 2B, 2C, 2D, 2E, 2F, 2G, 2H, 2I, 2J and 2K: Transcriptional gradients of tumor infiltrating T cells. A. Gene-gene correlation heatmap of top variable genes within CD8+ T metacells. B. Bar graph showing top 30 genes that are most correlated to LAG3 across CD8+ T metacells. The 30 “dysfunctional” genes are used to calculate the dysfunctional score. Scatter plots are depicting the dysfunctional score per metacell versus log enrichment of a selected set of dysfunctional genes. C. Similar to panel B, but showing top 30 genes correlated to FGFBP2, which define the cytotoxic score. The correlation of a selected set of cytotoxic genes with the cytotoxic score per metacell is shown. D. Cytotoxic score versus dysfunctional score on CD8+ metacells. E. Top transcription factors correlated with the dysfunctional score (right) and cytotoxic score (left). F. Shown are differences of dysfunctional and cytotoxic scores per metacell (X axis) versus the prediction (Y axis) of a linear model using TF expression alone (10-fold cross validation, lasso regularized). Inferred non-zero coefficients for TF variables are shown to the right. G. Bar graph showing the top 30 genes with the highest correlation to IL2RA (Treg score). H. Transcription factors with highest correlation to the Treg score. I. Linear model using transcription factors to predict the Treg score, similar to panel F. J. Gene enrichment in dysfunctional and Treg cells over naïve-like cells. A selected set of highly expressed genes (mean molecules per cell >=0.05) are shown, highlighting key genes either distinct or shared among the two groups. K. Treg-score versus dysfunctional-score on all metacells. Different groups of metacell are color-coded as in panel D.

FIGS. 2L, 2M, 2N, 2O, 2P, 2Q, 2R and 2S. L. Gene-gene correlation matrix for the top variable genes in CD8+ T cells, identical to FIG. 2A. Shown here with all gene names. M. Gene correlation to LAG3 on single cells (using spearman correlation on UMI counts, y-axis) and on metacells (using Pearson correlation on gene enrichment, x-axis). N-O. Sixty genes that most correlated with the dysfunctional score (C) and cytotoxic score (D) over CD8+ cells. Genes comprising these scores are colored, and were removed from the score when calculating their correlation to it. Number in parenthesis is the rank of the gene in the metacell correlation to the anchor gene defining the dysfunctional and cytotoxic scores (LAG3 or FGFBP2). P. Comparing cytotoxic to dysfunctional features on single cells. Showing the fraction of cytotoxic UMIs and dysfunctional UMIs, separating sub-panel per group of metacells (transitional, dysfunctional, cytotoxic). In each panel all CD8 cells are in grey and the group cells overlay them in color. Q. Stratifying dysfunctional T cells by the dysfunctional score, showing fraction of UMIs of genes from the dysfunctional score, TIGIT and ID3, in each decile. Difference between adjacent decile is tested for significance by chisq-test (*P<0.05; **P<0.01; ***P<1×10-3). R. Gene enrichment in Treg metacells of several representative genes comprising the Treg score versus Treg-score. S. Gene enrichment of Tfh and Treg cells over naïve-like cells. Showing data for strongly expressed genes (mean molecules per cell >=0.05) and labelling genes of interest and strongly enriched genes.

FIGS. 3A, 3B, 3C, 3D, 3E and 3F: Heterogeneity and transcriptional gradients of different myeloid populations. A. 2D projection of 16,142 non-T/NK cells partitioned into 100 metacells and 7 metacell groups annotated and marked by color code. B. Expression (molecules/1,000 UMIs) of marker genes in B cells, plasma cells, and different myeloid cells. C.

Top differentially expressed genes in macrophages (left), monocytes (center), and dendritic cells (right), as compared to the other two groups. D. Scatter plot comparing the monocyte, macrophage and DC scores per metacell as shown for myeloid metacells. E. Top transcription factors correlated with the monocyte score (upper) and macrophage score (lower). DC is excluded from this analysis due to the small number of DC metacells. F. Data illustrating the performance of a linear model using TFs to model the difference between monocyte and macrophage scores per metacell. Computed as in FIG. 2F.

FIGS. 3G, 3H, 3I and 3J. G. Confusion matrix for non-T/NK cells. H. Marker gene expression heat map for non-T/NK cells. Each column is a cell and each row is a gene. Cells are separated into different groups, colors as in FIG. 3A-F. I. Gene-gene correlation on gene enrichments in metacells for monocytes, macrophage, and dendritic cells metacells. J. Stratifying classical monocyte cells by the percentage of UMIs from the monocyte program, showing fraction of UMIs of the monocyte program genes, LYZ, and CEBPB in each quantile, difference between adjacent quantiles is tested for significance by Chisq-test (*P<0.05; **P<0.01; ***P<1×10-3).

FIGS. 4A, 4B, 4C and 4D: Inter-patient variation in the composition of dysfunctional CD8+ T cells and other immune cells. A. 2D projections of the composition of T and NK cells (top), and non-T/NK immune cells (bottom) in different patients. Showing six representative patients ordered by their fraction of dysfunctional CD8+ T cells within all T cells. B. Metacells (columns) are ordered by groups and clustered within each group. Showing from top to bottom the number of cells in the metacell, the top contributing patients to each metacell. Bar height is the fraction of cells, top patient in dark green, second patient in light green, the rest in white, patients' contribution to metacells, patients are ordered based on the frequency of dysfunctional CD8+ T cells, and metacell groups. Compositions of T cells and other immune cells are shown on the right (colors as in FIG. 1 for T/NK cells and FIG. 3 for non-T/NK cells). C. Patients are ordered by the fraction of dysfunctional CD8+ T cells within all T cells and binned into 3 groups (low, medium, and high dysfunctional). Disease stage, tumor location (LN: Lymph node; MSC: muscle; (S)C: (sub)cutaneous; p(S)C: primary (sub)cutaneous), treatment background (N: naïve; T: treated; IT: immunotherapy treated), percentage of CD3+ cells within total immune cells measured by FACS, and percentage of tumor immune infiltrates measured by histology are shown (Method), grey bars represent missing data. D. Frequency of different immune populations within the three groups of patients defined in panel (C) with each circle representing a patient and horizontal line the group median. Spearman correlation between the fraction of the group and the fraction of the dysfunctional group are shown on top, with stars marking significant p-value of a Mann-Whitney test between patients in the low and high groups (*P <0.05; **P <0.01; ***P <1×10-3).

FIGS. 4E, 4F, 4G and 4H. E. Type I IFN-induced gene expression intensity per patient. Showing the total UMIs fraction of type I IFN-induced genes across all patient cells, ordering patient by median percentage. F. Genes in monocytes and B cells that are most highly correlated with dysfunctional load across patients. Using patients with more than 50 cells in each group, ending up with 14 patients for the monocyte and 12 for B cells, showing genes total UMI count in parenthesis. G. T cell subtype compositions in treatment naive patients. Percentage of CD3+ cells in total CD45+ cells (analyzed by FACS) and percentage of immune infiltrates (analyzed by histology) are shown. H. Comparison of T cell subtype composition between metastases from the same patient. Two patients, p12 and p17, are shown.

FIGS. 5A, 5B, 5C, 5D, 5E, 5F, 5G, 5H, 5I, 5J, 5K and 5L: Clonal expansion and proliferation within the dysfunctional CD8+ T cell compartment. A. Graphical overview of the use of scTCR-seq to identify shared clones between two independent lesions from the same patient. B. Overview of TCR clonality for all patients, showing the size of each T cell clone (largest at the bottom, smallest at the top) in each tumor. Corresponding TCRs from two lesions of the same patient are marked. C. Patients' clonal composition, showing from top to bottom the number of distinct clones per patient, the number of T cells of which the TCR was retrieved, the distribution of clones by size for size one, size two and size >2 cells clones, and the T cells subtype composition of each group of clones, shown in pie charts (if the group has >=10 cells). Patients are ordered by the fraction of their size-one clones. D. Clonality of the TCR repertoire of the three groups of patients as defined in FIG. 4C: low, medium, and high dysfunctional. Showing the fraction of clones with >2 cells (left) and patient clonality score (right; defined by Pielou's evenness). Showing median as horizontal line, correlation and significance as in FIG. 4D. E. T cell subtype composition of cells with unique TCR (clone size=1) or shared TCR (clone size >2). F. Fraction of proliferating cells per metacell, calculated by defining a cell as proliferative using analysis of the bimodal distribution of cell cycle gene expression. Circle size reflects the fraction of proliferating cells in the metacell, using 2D projection as in FIG. 1C. G. percentage of proliferating cells in different immune cell types and subtypes, number of cells per metacell group are shown on the right. H-I. Fraction of proliferating cells in dysfunctional (H) and Treg (I) groups classified into bins according to their dysfunctional score (H) and Treg score (I). Scores defined as in FIGS. 2B and 2G. J. Gene enrichment over dysfunctional CD8 T cells stratified by the dysfunctional score load, showing highly varying genes, sorting genes by the decile they pick in and by their enrichment in that decile. K. Cell subtype composition of T cell clones of intermediate size (shared by 8-20 cells, left) or large size (shared by more than 20 cells, right). Clones are hierarchically clustered, matching patients are shown on bottom and clone ID on top. L. Pairwise clone sharing propensity by cell group. Showing the enrichment of the observed number of cell pairs sharing clones by their associated group over a control generated by random sampling cells by patients, preserving the patient cell composition, TCR detection probability, number of clones and clone sizes.

FIGS. 5M, 5N, 5O, 5P, 5Q, 5R, 5S and 5T. M. T cell subtype composition for T cells with different levels of TCR mRNA transcript (percentage of TRAC and TRBC2 genes UMIs); probability of detecting the TCR sequence in T cells with different levels of TCR mRNA transcript are shown as dot. N. Fraction of detected TCR per metacell versus the gene enrichment difference of CD8A plus CD8B and minus CD4. O. Correlation of genes from the cell cycle gene module on T and NK metacells. P. Frequency distribution of cell cycle score on all T cells. Cell cycle score is calculated as percentage of cell cycle gene UMIs out of total UMIs. Dashed line mark the threshold for marking a cell as proliferative. Q. Empiric cumulative distribution plots of cell cycle score per group. Note that the score is negated, and cells below the dashed line are the proliferative ones (fraction of proliferation in the legend) R. FACS analysis of PD-1 and Ki-67 expression in tumor infiltrating CD8+ T cells from three patients (p8, p28, p10). S. Cell cycle profile for PD-1 positive CD8 T cells from one representative tumor (p28), as measured by DNA staining with DAPI and distinguishing G0/G1 phase, S phase, and G2/M phase. T. Gene enrichment over Treg cells stratified by the Treg-score load, showing highly varying genes, sorting genes by the decile they pick in and by their enrichment in that decile.

FIGS. 6A, 6B and 6C: The relationship between cellular states and tumor reactivity of T cells. A. Graphical overview of the method to assess T cell reactivity. Ex vivo expanded TILs were co-cultured with autologous tumor cells to examine the presence of a tumor-specific TCR repertoire by measuring IFNg and TNFa secretion. B. tumor reactivity was measured as the percentage of T cells that secrete IFNg or TNFa after co-culture with autologous tumor material. Tumor reactivity of the tumor samples from 10 patients were assayed. C. The fraction of UMIs from genes of the dysfunctional program and cytotoxic program were calculated for each CD8+ T cell, and the distribution of these fractions across all CD8+ T cells are shown per patient. Patients are ordered by the median of the UMI percentage of dysfunctional gene program. T cell subtype composition within the CD8+ T cells are shown on top for each patient. Green and red circles mark reactive and non-reactive patients, respectively.

FIGS. 6D, 6E, 6F, 6G and 6H. D. Two-dimensional projection of expression profiles of 17,452 cells partitioned into 102 metacells. Single cells are shown in dots. Showing cells from: PBMCs of melanoma patients (p13, p17, p27), PBMCs from healthy donors, a healthy tissue that was misdiagnosed as tumor lesion (p22), and a residual tumor lesion that underwent regression after treatment (p24-1). E. Confusion matrix for all the cells as shown in panel A. C. Expression (molecules/1,000 transcripts) of marker genes, same genes as in FIG. 1D. F-H. Marker gene expression heat map for cells shown in panel A. Each column is a cell and each row is a gene. G. 2D projection of the composition of immune cells for PBMCs from p13, p17, and p27. Immune infiltrates from a healthy tissue what was misdiagnosed as tumor lesion from p22 and a residual tumor lesion that underwent regression after treatment from p24 are also shown.

FIG. 7 schematically depicts an embodiment for carrying out single cell transcriptome analysis.

FIG. 8 is a bar graph showing the percentage of CXCL13 positive cells within the GFP positive CD8 population, either without stimulation or upon stimulation with plate bound CD3 and soluble CD28 (CD3/CD28).

### DESCRIPTION OF SPECIFIC EMBODIMENTS OF THE INVENTION

The present invention, in some embodiments thereof, relates to methods of activating dysfunctional immune cells and treatment of cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Tumor immune cell compositions play a major role in response to immunotherapy but their heterogeneity and dynamics remain poorly characterized.

Whilst reducing embodiments of the invention to practice, the present inventors combined single cell RNA-sequencing, TCR-sequencing and T cell reactivity assays to trace immune cell dynamics in melanoma patients. Using this approach, they were able to demonstrate the presence of two effector T cell subsets, of which only one transitions into a dysfunctional T cell pool, as based both on a conserved gradient of expression of dysfunction-associated genes, and TCR sharing. Contrary to the prevailing paradigm that “exhausted” T cells are differentiating from the cytotoxic T cell compartment. Further, dysfunctional T cells are the major proliferating immune cell compartment, and that the strength of the T cell dysfunction signature is associated with tumor reactivity. Data from 25 melanoma patients confirm the universality of this observation and distinguish shared and divergent regulatory modules between dysfunctional CD8 and regulatory T cells. The present data demonstrate that CD8 T cells previously associated with a dysfunctional or exhausted state are in fact a highly proliferating and dynamically regulated population within the human tumor microenvironment. Such a T cell population can be used both as a diagnostic marker for the cytotoxic T cell potential of the cancer affected patient as well as a target for activation, in both in vivo and ex vivo settings. Thus, according to an aspect of the invention there is provided a method of determining responsiveness of a subject having a tumor to an immune checkpoint inhibition, the method comprising determining in a tumor of a subject a level of dysfunctional CD8⁺/Lag3⁺/PD1⁺/Tim3⁺/CD103⁺/CD39⁺/CD137⁺/Klrg1⁻ T cells, wherein a level of said dysfunctional cells above a predetermined threshold is indicative of a response to an immune checkpoint inhibition.

As used herein “subject” refers to a mammal, e.g., human, diagnosed with cancer. The terms “cancer” and “cancerous” refer to or describe the physiological condition in mammals that is typically characterized by unregulated malignant cell growth.

Examples of cancers that can be analyzed and treated according to some embodiments of the invention, include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcino sarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to a specific embodiment, the cancer is melanoma.

According to a specific embodiment, the cancer is a solid tumor.

According to a specific embodiment, the cancer is a primary tumor.

According to a specific embodiment, the cancer is metastatic.

According to a specific embodiment, the cancer is a secondary tumor.

As the significance of personalized treatment becomes more apparent, also known as “precision medicine”, it is important to determine the genetic makeup of subjects prior to initiation.

As used herein “determining responsiveness” refers to an ex vivo method for determining the likelihood of a subject to benefit from a treatment as described herein. The likelihood may be based on gene expression, immune assays, cell proliferation or combinations of same.

Responsiveness is determined when any of the above criteria shows at least a statistically significant change (dependent on the assay) as compared to a non-responsive control.

According to a specific embodiment, an increase in responsiveness as compared to a non-responsive control is of at least 10%, 20%, 30%, 40%, 50%, 60%, 70%, 80%, 90% or more.

As used herein “immune checkpoint inhibition” refers to cancer immunotherapy. The therapy targets immune checkpoints, key regulators of the immune system that stimulate or inhibit its actions, which tumors can use to protect themselves from attacks by the immune system. Checkpoint therapy can block inhibitory checkpoints, activate stimulatory functions, thereby restoring immune system function. Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274).

Examples of immune checkpoint inhibitors include, but are not limited to, of cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD244 (2B4), CD160, GARP, OX40, CD137 (4-1BB), CD25, VISTA, BTLA, TNFR25, CD57, CCR2, CCRS, CCR6, CD39, CD73, CD4, CD18, CD49b, CD1d, CDS, CD21, TIMI, CD19, CD20, CD23, CD24, CD38, CD93, IgM, B220 (CD45R), CD317, CD11b, Ly6G, ICAM-1, FAP, PDGFR, Podoplanin, and TIGIT.

Examples of clinically approved immune checkpoint inhibitors include, but are not limited to, Ipilimumab, (anti CTLA-4), Nivolimumab (anti PD-1) and Pembrolizumab (anti PD 1).

As mentioned, the method is effected by determining in a tumor of a subject the level of dysfunctional CD8⁺/Lag3⁺/PD1⁺/Tim3⁺/CD103⁺/CD39⁺/CD137⁺/Klrg1⁻ T cells.

As used herein “dysfunctional CD8⁺/Lag3⁺/PD1⁺/Tim3⁺/CD103⁺/CD39⁺/CD137⁺/Klrg1⁻ T cells” refers to T cells, also known as “exhausted T cells”, that are in a proliferative state, however, exhibit poor (ineffective) effector function, expression of inhibitory receptor molecules and a transcriptional state that is distinct from that of functional effector cells or naive T cells.

According to a specific embodiment, the dysfunctional T cells, as described herein, are characterized by a gene expression profile, as listed in Table 1 hereinbelow.

TABLE 1
Gene expression enrichment table (Dysfunctional over naive T cells)

| gene | Log2 enrichment | gene | Log2 enrichment | gene | Log2 enrichment |
|---|---|---|---|---|---|
| KIR2DL4 | 4.490732143 | HLA-DRB1 | 2.056594732 | SIRPG | 1.468527293 |
| CCL3 | 4.004577849 | TRGC2 | 2.044328432 | HOPX | 1.468016815 |
| CCL4L2 | 3.999896786 | GNLY | 2.0358099 | GABARAPL1 | 1.438919269 |
| VCAM1 | 3.969975947 | CD8B | 2.029955887 | TTN | 1.4327192 |
| SPRY2 | 3.931655941 | CCL5 | 1.999008669 | F2R | 1.424682579 |
| TNFRSF9 | 3.814912466 | KLRC3 | 1.946597734 | TRPS1 | 1.394265236 |
| CCL4L1 | 3.765283822 | HLA-DPB1 | 1.946126846 | PCNA | 1.385862832 |
| CCL4 | 3.584087737 | KLRK1 | 1.943475387 | CD2BP2 | 1.359807185 |
| GZMB | 3.414998534 | HLA-DRA | 1.940498471 | MCM5 | 1.356498879 |
| CXCL13 | 3.225302969 | LYST | 1.922323385 | DUSP4 | 1.352008488 |
| NKG7 | 3.224628478 | PDCD1 | 1.88892228 | ATXN1 | 1.340471952 |
| GPR56 | 3.214909407 | TOP2A | 1.876717438 | PLA2G16 | 1.335699176 |
| AKAP5 | 3.190543821 | HIST1H1B | 1.871529434 | TAPBPL | 1.32968705 |
| KLRC2 | 3.07986313 | TOX | 1.860832979 | FAM105B | 1.299457067 |
| IFNG | 3.023685758 | DGKH | 1.832450982 | HLA-DQB1 | 1.276384107 |
| ZBED2 | 3.006054003 | PIK3AP1 | 1.831395119 | SNX9 | 1.252655186 |
| MIR155HG | 3.005452717 | ITGAE | 1.787252658 | TUBA1B | 1.248830683 |
| RDH10 | 2.985007184 | XCL1 | 1.784476906 | AD000671.6 | 1.246266457 |
| LAG3 | 2.941694402 | CTSW | 1.781971159 | CD27 | 1.228474064 |
| KLRD1 | 2.887714855 | SNAP47 | 1.778086444 | ID2 | 1.219909107 |
| CD8A | 2.814011346 | UBE2F | 1.771708602 | PPP1R16B | 1.218086614 |
| FCRL3 | 2.801517444 | NUSAP1 | 1.763442642 | IFI27L2 | 1.217989994 |
| FAM3C | 2.73095743 | TOX2 | 1.762632921 | IL2RB | 1.209285565 |
| GFOD1 | 2.715219172 | XXbac- | 1.762277298 | GALNT1 | 1.205077078 |
| | | BPG254F23.6 | | | |
| TNIP3 | 2.629363508 | HLA-DPA1 | 1.756087635 | APOBEC3C | 1.201949055 |
| CRTAM | 2.495450012 | ZEB2 | 1.748952373 | MCM6 | 1.182082011 |
| XCL2 | 2.488569474 | CD74 | 1.729597749 | RNU2-2P | 1.167309262 |
| PTMS | 2.481079283 | NAB1 | 1.725000601 | TNFRSF1B | 1.16673391 |
| ID3 | 2.467155489 | WARS | 1.704170551 | IKZF3 | 1.156061281 |
| MK167 | 2.466407488 | CENPF | 1.674894093 | PTPN6 | 1.148259466 |
| HAVCR2 | 2.463106931 | HLA-DRB5 | 1.662792933 | GGA2 | 1.1440375 |
| ENTPD1 | 2.454383944 | CD38 | 1.654686237 | RFTN1 | 1.141330623 |
| HLA-DQA1 | 2.389376746 | METRNL | 1.638056742 | SYT11 | 1.121439198 |
| EOMES | 2.366789379 | APOBEC3G | 1.635277528 | HIST1H4C | 1.119372493 |
| GZMH | 2.351709126 | RIN3 | 1.599236078 | GBP2 | 1.112680616 |
| PRF1 | 2.334922721 | STMN1 | 1.592882744 | DUSP2 | 1.111771358 |
| FASLG | 2.3211962 | RAB27A | 1.557588099 | ABI3 | 1.111291789 |
| PHLDA1 | 2.250461525 | CTLA4 | 1.547774899 | KIF20B | 1.101567556 |
| HLA-DRB6 | 2.249952345 | CD63 | 1.522760313 | CD84 | 1.089153328 |
| CSF1 | 2.235526487 | NR4A2 | 1.501423197 | ARPC5L | 1.080873084 |
| TIGIT | 2.228280115 | AC092580.4 | 1.499468647 | ITM2A | 1.080354662 |
| HLA-DMA | 2.155555695 | CBLB | 1.498923491 | TRGC1 | 1.077011593 |
| CST7 | 2.14725879 | SLAMF7 | 1.482145187 | SH2D2A | 1.0757751 |
| NBL1 | 2.111557553 | TRAF5 | 1.480749707 | MPHOSPH9 | 1.07069683 |
| PSTPIP1 | 1.069986924 | CHST12 | 0.878623806 | NCOA1 | 0.765855226 |
| ZFP36L1 | 1.068194989 | SNORD3A | 0.87559877 | PSMB9 | 0.764075393 |
| CD7 | 1.06428792 | SH2D1A | 0.873117753 | TAP1 | 0.764063866 |
| SYTL3 | 1.060595686 | RAB1B | 0.867612725 | AKR1B1 | 0.763693165 |
| PLEK | 1.057743433 | PTPN22 | 0.865283024 | RANBP1 | 0.76355573 |
| MIAT | 1.049975924 | PTPN11 | 0.862481115 | TBCD | 0.759087868 |
| GZMA | 1.032440074 | RBPJ | 0.858278624 | C11orf48 | 0.759045305 |
| HMGN3 | 1.024445927 | SRI | 0.857465561 | OASL | 0.756166035 |
| LINC00152 | 1.023060248 | CDK2AP2 | 0.849223113 | ATP1B3 | 0.75608455 |
| GZMK | 1.019996804 | AGFG1 | 0.845833047 | HMGB1P5 | 0.749209003 |
| GBP5 | 1.019496738 | ##### | 0.845389729 | HMGN2 | 0.748154151 |
| MFSD6 | 1.01473735 | LSM2 | 0.842808972 | SPATS2L | 0.747833924 |
| HSPB11 | 1.009086603 | BATF | 0.839311624 | RNA5-8SP6 | 0.743125245 |
| RNF19A | 0.999305652 | FKBP1A | 0.83817168 | VIMP | 0.740307829 |
| ITGB7 | 0.995408172 | PSMD8 | 0.837041871 | SURF4 | 0.735657861 |
| SERPINB1 | 0.988131839 | USP24 | 0.833602695 | PSMB8 | 0.733526327 |
| APOBEC3D | 0.985730472 | COTL1 | 0.828665295 | TMPO | 0.732176424 |
| ANXA5 | 0.983363554 | CD81 | 0.826240194 | CCND2 | 0.730944468 |
| LITAF | 0.977883155 | SDF2L1 | 0.824265289 | RCC2 | 0.728775622 |
| IGF2R | 0.974011679 | SMC4 | 0.822965909 | CALR | 0.728577573 |
| TRIM8 | 0.964281324 | PMF1 | 0.81947984 | RAB7L1 | 0.724281302 |
| GSTP1 | 0.963111439 | TRIM69 | 0.818961728 | EZR | 0.723353885 |
| SHFM1 | 0.958160361 | PTPRA | 0.818853728 | KCTD20 | 0.723204854 |
| TXNDC17 | 0.956062973 | SLA | 0.818302708 | RHOC | 0.721258064 |
| PKM | 0.953726223 | C12orf75 | 0.816897669 | EXOSC9 | 0.717378397 |
| LSP1 | 0.941442562 | APMAP | 0.815963632 | CCDC69 | 0.717287609 |
| SRGN | 0.939304612 | PYHIN1 | 0.813585594 | SLC20A1 | 0.715469689 |
| IRF2 | 0.937052626 | GBP4 | 0.811721375 | C5orf56 | 0.715162048 |
| PTPN7 | 0.934842321 | TUBB | 0.811166985 | TMEM189 | 0.714866167 |
| GAPDHP40 | 0.9281729 | ZCRB1 | 0.800785097 | VOPP1 | 0.714408628 |
| GALM | 0.927456627 | MDM2 | 0.799550754 | PYCARD | 0.713406744 |
| SERPINB9 | 0.91962915 | PPM1M | 0.797606265 | CTSD | 0.710740997 |
| PTTG1 | 0.918339081 | C17orf49 | 0.796792332 | PSMA2 | 0.707531441 |
| MAPRE2 | 0.910691685 | DGKZ | 0.795608007 | UBE2A | 0.706959784 |
| TXNDC11 | 0.910594325 | CCR5 | 0.790428507 | RC3H1 | 0.705979072 |
| MAP2K2 | 0.90694099 | RNF115 | 0.78975068 | ENSA | 0.705264749 |
| ARL6IP1 | 0.904515172 | MBD2 | 0.789138893 | MIS18BP1 | 0.704956252 |
| PRKCH | 0.902286887 | TBC1D4 | 0.783033145 | ANKRD32 | 0.704818058 |
| HMGB2 | 0.89647923 | RGS2 | 0.782221627 | MEA1 | 0.697988454 |
| ACTN4 | 0.896103528 | NASP | 0.781024945 | HNRNPLL | 0.693861252 |
| CLEC2D | 0.894950814 | LASP1 | 0.777574929 | SLA2 | 0.690396657 |
| FAM53B | 0.893456661 | PCED1B | 0.773658204 | PGAM1 | 0.68951194 |
| ITGA1 | 0.884707243 | SPATA13 | 0.772467917 | CXCR6 | 0.68821601 |
| FUT8 | 0.882003415 | H2AFZ | 0.766115765 | DEK | 0.687235987 |
| CLIC1 | 0.687111406 | VPS26A | 0.630215471 | NUCKS1 | 0.575340857 |
| ZFP91 | 0.679084757 | SNRPG | 0.628362888 | YARS | 0.575246268 |
| HSBP1 | 0.678358777 | RNASEH2C | 0.625435284 | ST8SIA4 | 0.573175086 |
| LSMD1 | 0.677859686 | SUB1 | 0.624503735 | TYMP | 0.572377274 |
| IFI16 | 0.676971277 | AMICA1 | 0.622193744 | RARRES3 | 0.57074508 |
| SP140 | 0.67424705 | MCTS1 | 0.621753893 | SNRNP27 | 0.569661634 |
| CKAP2 | 0.673259099 | MCM3 | 0.621400261 | PSMB2 | 0.569646251 |
| POMP | 0.673255113 | ITGB1BP1 | 0.621337269 | ANAPC11 | 0.569282856 |
| PBDC1 | 0.670398514 | LBR | 0.620757543 | HMGB1 | 0.568539592 |
| ETF1 | 0.670164784 | SMC1A | 0.616654824 | ARPP19 | 0.567112735 |
| ATRAID | 0.670154265 | PSMB10 | 0.616478021 | CD3D | 0.566430265 |
| DNAJB14 | 0.670128866 | NPIPB5 | 0.615384681 | SREK1 | 0.563140105 |
| MAP4K1 | 0.668244511 | POLR2K | 0.614639793 | IWS1 | 0.560329292 |
| CSNK2B | 0.668083952 | SYNGR2 | 0.614434528 | CEBPZ | 0.560210911 |
| ADRM1 | 0.667439928 | ADNP | 0.613804593 | GTF2A2 | 0.557777631 |
| IL32 | 0.666871719 | VCP | 0.611263909 | HCST | 0.556700829 |
| PSME2 | 0.6649294 | SCARNA9 | 0.610456508 | PSMA5 | 0.55577973 |
| PFDN2 | 0.664674095 | ASXL2 | 0.608563436 | DNAJB11 | 0.554744341 |
| PFKP | 0.664566976 | RTF1 | 0.607273049 | SPCS2 | 0.552459222 |
| PDIA6 | 0.657820135 | PSMA4 | 0.606674353 | VTI1B | 0.552439747 |
| ORMDL3 | 0.656341159 | NAPA | 0.604978651 | TLN1 | 0.548061428 |
| HDGF | 0.653531895 | BST2 | 0.603729729 | FKBP2 | 0.547968703 |
| CD97 | 0.652232866 | MIR4435-1HG | 0.603130505 | PAG1 | 0.5453731 |
| U2AF1 | 0.651130901 | NLRC5 | 0.602865285 | UBE2J1 | 0.544816824 |
| RABAC1 | 0.64851274 | ZFP91-CNTF | 0.602069129 | RBBP7 | 0.544405216 |
| SNRPB | 0.647877047 | FSCN3 | 0.601349734 | NUDT21 | 0.54343484 |
| VAMPS | 0.647641837 | LGALS1 | 0.600819117 | GTF3C6 | 0.542229894 |
| RBM12 | 0.647419094 | ODC1 | 0.600312371 | PSMD4 | 0.542167091 |
| PLEKHF1 | 0.644817513 | ZC3H7A | 0.598006709 | RNF187 | 0.541650857 |
| HNRNPAB | 0.642530956 | LRBA | 0.592201609 | AKAP13 | 0.541364603 |
| ARPC2 | 0.642244765 | STAT3 | 0.590541518 | RUNX3 | 0.53748367 |
| FBXL18 | 0.641251253 | ZNF106 | 0.58785756 | PLSCR1 | 0.537136982 |
| UBB | 0.638477541 | ZBTB7A | 0.587063132 | CSK | 0.536484213 |
| CALM3 | 0.635962806 | CYTH4 | 0.586946581 | SMC5 | 0.536182949 |
| VMP1 | 0.634803097 | GBP1 | 0.58687048 | HERPUD2 | 0.533861732 |
| MARK3 | 0.634359636 | ABHD17A | 0.583400338 | EID1 | 0.52931039 |
| ZCCHC6 | 0.634356785 | AL353354.1 | 0.581014846 | SPEN | 0.528913357 |
| DCAF7 | 0.6335917 | MYO9B | 0.579371611 | SLAMF6 | 0.525054691 |
| ARHGAP9 | 0.632565876 | SNRPD1 | 0.579366551 | SCLT1 | 0.524314986 |
| CDK6 | 0.631432859 | SNRPF | 0.578176389 | THRAP3 | 0.524313535 |
| UBE2L6 | 0.631242868 | TRAC | 0.577482435 | CLTA | 0.524191224 |
| TRAF1 | 0.631188664 | HLA-A | 0.576666225 | FAM111A | 0.523669776 |
| SNF8 | 0.631157433 | CMIP | 0.576612356 | WDR1 | 0.523114952 |
| IPCEF1 | 0.631100818 | SPN | 0.576094192 | MOB3A | 0.52308913 |
| WSB1 | 0.52300756 | CLEC2B | 0.477936471 | Sep. 2 | 0.45124665 |
| SRSF1 | 0.522624848 | HSPB1 | 0.476589491 | M6PR | 0.447944245 |
| ADRBK1 | 0.521508413 | ITGB2 | 0.476249767 | HSP90B1 | 0.447660785 |
| USP16 | 0.521189482 | PARP4 | 0.475025059 | PPM1G | 0.447491837 |
| KRAS | 0.52043565 | HLA-B | 0.474420176 | HIST1H1C | 0.446833301 |
| DRAP1 | 0.520036912 | EIF3B | 0.473965281 | TCEB2 | 0.446819937 |
| MIEN1 | 0.519664844 | Sep. 7 | 0.47145212 | AC012379.1 | 0.446728033 |
| SRSF9 | 0.518037521 | CMTM6 | 0.47050531 | TXN | 0.446391075 |
| PSMD1 | 0.51526274 | WIPF2 | 0.470327016 | BZW1 | 0.444621266 |
| SZRD1 | 0.51102297 | FAM192A | 0.470262138 | CWC15 | 0.444598676 |
| RAB8B | 0.508415712 | ARGLU1 | 0.469914568 | LCP2 | 0.443536893 |
| FYN | 0.508311977 | SFPQ | 0.469496696 | YWHAH | 0.44254042 |
| HSPA4 | 0.508059077 | PRDM1 | 0.468670533 | PAFAH1B2 | 0.442334057 |
| MSI2 | 0.507299642 | PSMC3 | 0.468520404 | CTC-260F20.3 | 0.442050649 |
| OTUB1 | 0.506365953 | PDCD10 | 0.467881212 | EMC7 | 0.442034035 |
| CRIP1 | 0.505317652 | PPDPF | 0.467661485 | TMEM256 | 0.441689954 |
| ZBTB38 | 0.505214395 | FUS | 0.467117845 | AP2B1 | 0.440891599 |
| MSN | 0.504987534 | MCL1 | 0.465921176 | RN7SL2 | 0.440799897 |
| SEC61B | 0.504504344 | SMS | 0.46452993 | DNMT1 | 0.440549587 |
| MAT2A | 0.502032324 | DDX27 | 0.463779764 | PPP1CA | 0.440207464 |
| MYO1F | 0.501124601 | NAA38 | 0.46341298 | EIF4H | 0.440192051 |
| RNF213 | 0.498254058 | AIP | 0.46236438 | FBXO7 | 0.439614509 |
| ITGAL | 0.497577817 | PARP1 | 0.462079322 | RHOA | 0.43951829 |
| PITPNB | 0.497564735 | FAM49B | 0.461954973 | RAB10 | 0.438013842 |
| RAC1 | 0.49695249 | PJA2 | 0.461824295 | UBE2N | 0.437924428 |
| CD3G | 0.496558156 | LSM6 | 0.461728236 | CARD16 | 0.436697784 |
| TRBC2 | 0.495369298 | PSMB4 | 0.461393033 | PPP2R1A | 0.436240923 |
| ANAPC5 | 0.49464261 | SKA2 | 0.461231874 | TUBB4B | 0.435782507 |
| DCP2 | 0.491910991 | BAZ1B | 0.460867863 | C1orf43 | 0.435522735 |
| MYO1G | 0.491739093 | RNU4ATAC | 0.460744289 | AMZ2 | 0.433537674 |
| SLFN12L | 0.490332356 | BSG | 0.458957846 | TMEM109 | 0.433370415 |
| RAPGEF1 | 0.490235164 | YWHAE | 0.458886137 | C7orf73 | 0.433310991 |
| HCP5 | 0.490115253 | TOX4 | 0.457410102 | KPNA6 | 0.433043585 |
| MAPKAPK2 | 0.489798794 | HMGN1P38 | 0.456952704 | DYNLL1 | 0.432056016 |
| PSMB6 | 0.488702093 | BUB3 | 0.456352248 | PTMAP2 | 0.431326304 |
| PSMA3 | 0.487467225 | IRF9 | 0.456318123 | NHP2 | 0.431156727 |
| SUMO1 | 0.486799076 | SCAMP2 | 0.456281295 | SMC3 | 0.430895996 |
| CTSC | 0.486308027 | XIAP | 0.455729993 | SCAND1 | 0.430450539 |
| ARHGAP30 | 0.484803336 | CHTOP | 0.454948952 | CNPY3 | 0.429641645 |
| COMMD7 | 0.484774877 | EWSR1 | 0.454260707 | RASAL3 | 0.429593816 |
| EDF1 | 0.482387687 | AKT1 | 0.454159396 | SQSTM1 | 0.428940663 |
| ARAP2 | 0.480452518 | DTX3L | 0.452261577 | IFI30 | 0.428022 |
| MANF | 0.479342441 | PPP2CA | 0.451971075 | PPP1R11 | 0.427863648 |
| EIF4E2 | 0.479193576 | LPIN1 | 0.451910062 | SAP30BP | 0.42779976 |
| EFHD2 | 0.427201098 | LAMTOR5 | 0.400662398 | GNB2 | 0.380658167 |
| CFL1 | 0.426883263 | IVNS1ABP | 0.400622696 | HNRNPM | 0.38062134 |
| POLR2E | 0.426752831 | SUMO2 | 0.399914179 | ABCF1 | 0.379878832 |
| RNASEK- | 0.426545806 | SRP9 | 0.399214464 | PARVG | 0.37928924 |
| C17orf49 | | | | | |
| DNAJC2 | 0.426171753 | SH3KBP1 | 0.39890884 | SRP14 | 0.378813984 |
| RN7SL4P | 0.425644053 | CSNK2B- | 0.398870209 | HSPA5 | 0.377777129 |
| | | LY6G5B-1181 | | | |
| ARPC1B | 0.424960986 | PCMT1 | 0.398120494 | PSD4 | 0.377712772 |
| FAM120A | 0.424902009 | RBCK1 | 0.39744028 | RBBP4 | 0.377662235 |
| SEL1L3 | 0.423783016 | NEDD8 | 0.397388281 | NAP1L4 | 0.37702568 |
| DNAJC1 | 0.422738017 | POLR2B | 0.396052078 | PARP9 | 0.37670255 |
| PSMD7 | 0.421663153 | ZNF655 | 0.395970938 | IQGAP1 | 0.374341351 |
| EAPP | 0.42121925 | IRF1 | 0.395805162 | TAF9 | 0.373872967 |
| DENND2D | 0.420098458 | TMCO1 | 0.39574203 | NUCB1 | 0.373626613 |
| UFD1L | 0.419856801 | EIF2S1 | 0.395516038 | CTCF | 0.373036854 |
| HN1 | 0.419032439 | KRT222 | 0.395237153 | ZNF207 | 0.371685618 |
| CAPZA1 | 0.418881908 | NKTR | 0.394532855 | HNRNPD | 0.371603547 |
| CASP4 | 0.418567423 | CHST11 | 0.393997097 | TOP1 | 0.371527887 |
| PRKAR1A | 0.41823527 | SIT1 | 0.39332859 | SMG1 | 0.370580974 |
| CREB1 | 0.417255846 | DDB1 | 0.392285537 | TPP1 | 0.370156543 |
| HNRNPA2B1 | 0.416815297 | OGT | 0.391263918 | HLA-C | 0.369770436 |
| HIST2H2AC | 0.416492104 | CTD-3214H19.4 | 0.390529331 | NRBP1 | 0.368446511 |
| COPZ1 | 0.416103134 | FERMT3 | 0.390488576 | ITPKB | 0.367831308 |
| ATPSEP2 | 0.41596389 | RFC1 | 0.38973581 | TACC1 | 0.36763938 |
| GNAS | 0.414013788 | PTMAP5 | 0.38929649 | CNDP2 | 0.367258284 |
| KRCC1 | 0.412505358 | GPR171 | 0.389172996 | CEP57 | 0.367221849 |
| ANXA6 | 0.412421308 | SUZ12 | 0.388537252 | HNRNPR | 0.367037912 |
| NEDD9 | 0.411406663 | PSMA7 | 0.388426829 | LBH | 0.366320201 |
| CCT8 | 0.409780094 | KMT2A | 0.387590569 | AP2M1 | 0.366227081 |
| WAS | 0.409444959 | CELF2 | 0.387499581 | LMAN1 | 0.36613928 |
| PSMB1 | 0.40911407 | AC104698.1 | 0.387175508 | SASH3 | 0.363606007 |
| ZNRF1 | 0.40878235 | SH3GLB1 | 0.387068254 | PDIA3 | 0.362949724 |
| RAN | 0.408396333 | HERPUD1 | 0.386250247 | RER1 | 0.362883465 |
| HNRNPA3 | 0.408348607 | COPA | 0.386225463 | ARF6 | 0.361881914 |
| SH3BP1 | 0.40825723 | SEMA4D | 0.385957779 | MOB1A | 0.359900502 |
| ZYX | 0.406007345 | MAPK1IP1L | 0.385660385 | RAD23B | 0.359708748 |
| SF3B5 | 0.405995826 | RBM8A | 0.385042803 | CTDSP1 | 0.359580903 |
| DDX39B | 0.405975635 | SPTAN1 | 0.384407985 | CSNK1D | 0.357642414 |
| NSRP1 | 0.405914573 | SRSF4 | 0.384182413 | FAM32A | 0.357217257 |
| TOP2B | 0.405212862 | PUF60 | 0.383631406 | FRG1 | 0.357206223 |
| PSMA1 | 0.405143618 | TXNL4A | 0.382836719 | SRSF11 | 0.35679426 |
| WNK1 | 0.405019131 | SAMSN1 | 0.382763904 | AP2S1 | 0.356535413 |
| OAZ1 | 0.40404809 | SEC11A | 0.382379146 | ZNHIT1 | 0.356370827 |
| SNX17 | 0.403238879 | PFN1 | 0.381752567 | PVRIG | 0.355429545 |
| NOP58 | 0.402936725 | CBX5 | 0.380763987 | MAN1A2 | 0.355164867 |
| PTPRCAP | 0.355148611 | MGEA5 | 0.333922976 | TRIM38 | 0.315024393 |
| PNISR | 0.354506328 | PAIP2 | 0.333870435 | CDC37 | 0.31489579 |
| TROVE2 | 0.353935489 | TRA2B | 0.333580647 | Y_RNA | 0.313676892 |
| SAMD9L | 0.353479999 | PSME1 | 0.331975352 | LMAN2 | 0.313540404 |
| PPP1R12A | 0.353379543 | SUPT16H | 0.331935088 | DDOST | 0.313531649 |
| FUBP1 | 0.352943329 | RECQL | 0.331930959 | C17orf62 | 0.313125044 |
| ENY2 | 0.352909428 | LSM3 | 0.331671958 | GYG1 | 0.312429274 |
| HSP90AA1 | 0.352176901 | LY6E | 0.33100507 | PSMC5 | 0.309305294 |
| SEC11C | 0.352025897 | ARPC4 | 0.330703384 | UBE2L3 | 0.308614651 |
| DDX58 | 0.350661116 | NOL7 | 0.330591619 | PAFAH1B1 | 0.308524092 |
| CLIP1 | 0.350635164 | ERICH1 | 0.330085117 | NFATC3 | 0.307463021 |
| TPM4 | 0.350085933 | PRPF38B | 0.329795914 | PNN | 0.307409754 |
| BHLHE40 | 0.349443112 | CXCR3 | 0.329743227 | MAP1LC3B | 0.307354257 |
| LIMS1 | 0.349004992 | SMARCE1 | 0.329317082 | PPP1CC | 0.307100617 |
| RNF181 | 0.34860977 | SRSF7 | 0.329077923 | IL2RG | 0.306904292 |
| MIF | 0.348375436 | CCDC142 | 0.329067758 | SLU7 | 0.30678132 |
| AKNA | 0.348370278 | ADD1 | 0.328170078 | MESDC2 | 0.306778585 |
| CALM2 | 0.347603954 | SH3BGRL3 | 0.328111255 | H2AFY | 0.306680552 |
| PAPOLA | 0.345501816 | GBP3 | 0.327960408 | SSR1 | 0.306235552 |
| RBM25 | 0.345501816 | SRSF2 | 0.327671982 | FMNL1 | 0.306150476 |
| DERL1 | 0.345161879 | ANP32E | 0.327480116 | SET | 0.305859679 |
| HDDC2 | 0.34374274 | EIF1B | 0.326710808 | CAPRIN1 | 0.305338077 |
| ORAI1 | 0.343304253 | HP1BP3 | 0.326531451 | LRP10 | 0.304143062 |
| PARP14 | 0.342005772 | PREX1 | 0.32650236 | USP7 | 0.30396405 |
| SEPHS2 | 0.341607894 | ZFC3H1 | 0.326011132 | MAP4 | 0.303493242 |
| RPN2 | 0.341401807 | DPP8 | 0.325872182 | FLNA | 0.303342124 |
| CAP1 | 0.3413674 | HNRNPL | 0.325777187 | UBL5 | 0.303066469 |
| CNOT1 | 0.341344887 | CHD6 | 0.325607926 | ZBTB1 | 0.302783199 |
| TRAPPC1 | 0.341299511 | ATP6V0C | 0.324497907 | ARPC3 | 0.30172489 |
| TRMT112 | 0.340218056 | CNOT2 | 0.32423314 | USP1 | 0.301723181 |
| ZNF292 | 0.340070457 | UBE4A | 0.323747989 | SMEK1 | 0.301566892 |
| CLTC | 0.340014435 | SLC3A2 | 0.322451839 | CDK13 | 0.30147008 |
| GSDMD | 0.338752139 | PABPN1 | 0.322307337 | SNX20 | 0.301271992 |
| RPL22L1 | 0.337846256 | COPE | 0.322214156 | CHMP2A | 0.300546586 |
| USP8 | 0.337665839 | METAP2 | 0.319873793 | POLR2L | 0.300535232 |
| SRSF3 | 0.337275283 | C8orf59 | 0.319851017 | MXD4 | 0.30037844 |
| PSMD2 | 0.336938788 | BTN3A1 | 0.319330286 | RALA | 0.300077082 |
| DNTTIP2 | 0.335987061 | RNASEH2B | 0.319115312 | UBXN4 | 0.299633831 |
| ATG12 | 0.335700992 | SNRPC | 0.318807981 | HIST1H1E | 0.298074848 |
| SNW1 | 0.335395311 | ARF1 | 0.317498663 | LARP7 | 0.29800218 |
| ITGA4 | 0.335040272 | HMGN1 | 0.316946986 | RASGRP1 | 0.297676704 |
| VPS28 | 0.334938917 | TMEM167A | 0.316902327 | RPL7L1 | 0.297414316 |
| C4orf3 | 0.334735326 | AP000721.4 | 0.316775281 | PRR13 | 0.296511051 |
| COX6CP1 | 0.334336334 | RAB6A | 0.316761559 | GNAI2 | 0.296451464 |
| RNF10 | 0.295974057 | BUD31 | 0.279329791 | NOP56 | 0.259067319 |
| PSMC2 | 0.294932436 | HSPH1 | 0.278381267 | YWHAQ | 0.258993874 |
| SPCS1 | 0.294370243 | SF3A3 | 0.278015815 | CD2 | 0.258786707 |
| JAK3 | 0.294266263 | BIN1 | 0.277805095 | SNORA67 | 0.257871132 |
| CCNK | 0.294043399 | C1orf63 | 0.276125941 | OAS2 | 0.256911981 |
| PDCD5 | 0.293864364 | NELL2 | 0.275578883 | RUNX1 | 0.256720812 |
| NAA50 | 0.293817053 | TMX4 | 0.275066579 | SNRPE | 0.256720812 |
| SSRP1 | 0.293593209 | TAF7 | 0.274937844 | BRD7 | 0.255465458 |
| BFAR | 0.293074395 | PSMG2 | 0.274724538 | SELK | 0.254445951 |
| SNRPD3 | 0.292598834 | AP3D1 | 0.274542463 | CCNDBP1 | 0.253914486 |
| NONO | 0.292522266 | RALY | 0.274533875 | RNPS1 | 0.253098084 |
| XRN1 | 0.292478389 | PRKACB | 0.274514957 | HNRNPF | 0.252724266 |
| NFATC2IP | 0.292072562 | RPL17-C18orf32 | 0.273477907 | ZNFX1 | 0.252183535 |
| SNX5 | 0.291058161 | RAB7A | 0.272341752 | AC004057.1 | 0.251936232 |
| NCOR1 | 0.290949706 | ZFR | 0.271752577 | SRPR | 0.251152016 |
| SRP19 | 0.290887702 | VAPA | 0.271525811 | KRT10 | 0.251098482 |
| CYBA | 0.28991218 | RGS1 | 0.271485129 | CCT6A | 0.250971389 |
| CTD- | 0.289649887 | ARHGEF3 | 0.271352533 | ZFAND5 | 0.250478211 |
| 2545G14.7 | | | | | |
| MORF4L1 | 0.28911482 | FAM96B | 0.271204264 | TMSB4XP4 | 0.249450865 |
| MITD1 | 0.287983326 | LAMTOR1 | 0.270811576 | ADAR | 0.24913663 |
| FAM208A | 0.287957455 | PPHLN1 | 0.270433323 | CAPZB | 0.249125122 |
| DNAJC8 | 0.287953454 | PDAP1 | 0.269987173 | PTBP1 | 0.249094935 |
| RBX1 | 0.287645363 | EIF5 | 0.269630475 | PTMA | 0.248504371 |
| POLR2G | 0.286721018 | HDAC1 | 0.268944097 | USO1 | 0.248199818 |
| ARPC5 | 0.286228153 | BIRC6 | 0.268863507 | GNPTAB | 0.248144941 |
| SSR3 | 0.284877483 | UHMK1 | 0.268691446 | LRRFIP1 | 0.24657496 |
| DYNLT1 | 0.284514033 | TBL1XR1 | 0.266866944 | SON | 0.246066903 |
| CNOT6L | 0.28408865 | TERF2IP | 0.26601867 | PGK1 | 0.24589605 |
| ZNF644 | 0.283210449 | PTGER4 | 0.265880286 | U2SURP | 0.245462365 |
| PPP6R1 | 0.283125599 | H2AFV | 0.265808922 | TMEM14B | 0.244664934 |
| HLA-F | 0.282800185 | COPS6 | 0.265492185 | APOL6 | 0.244544444 |
| HERC1 | 0.282713116 | GABARAPL2 | 0.265489902 | INPP4B | 0.242637112 |
| GIGYF2 | 0.282589655 | WDR83O5 | 0.265138339 | CNPY2 | 0.242248848 |
| ARHGDIA | 0.282393661 | PRRC2C | 0.264780713 | DHX36 | 0.239562743 |
| CBX3 | 0.282319213 | UBLCP1 | 0.263869785 | ANP32B | 0.238914275 |
| TOR1AIP1 | 0.281985928 | GNG2 | 0.263681025 | TRAT1 | 0.238209091 |
| VASP | 0.281611172 | POLR2J | 0.263453997 | ATRX | 0.237307866 |
| KIF5B | 0.281340652 | PDCD6 | 0.26316521 | SUPT5H | 0.236939507 |
| NCL | 0.281217567 | MGAT1 | 0.263118886 | CAPNS1 | 0.236858284 |
| C9orf16 | 0.280993291 | SHKBP1 | 0.262055667 | UBC | 0.236650885 |
| MYL6 | 0.280984045 | USP48 | 0.262055667 | AP003419.11 | 0.236423788 |
| EVL | 0.280153233 | USF2 | 0.261995623 | ACTB | 0.235310537 |
| FDFT1 | 0.279736807 | PRPF40A | 0.261963889 | SYNE1 | 0.235166289 |
| TMSB4XP8 | 0.279448086 | UBE3A | 0.260057701 | LDHA | 0.234424436 |
| BTN3A3 | 0.233804839 | AIMP1 | 0.213964675 | RPF1 | 0.194116357 |
| TOB2 | 0.233471523 | THOC7 | 0.212902014 | ARF4 | 0.193968454 |
| ZNF24 | 0.233370869 | UBA2 | 0.21176386 | ACTG1 | 0.19379579 |
| BRD4 | 0.232745836 | ACTR2 | 0.211654569 | ATP2B4 | 0.193631382 |
| RBM23 | 0.232457296 | CERS2 | 0.210308533 | C14orf166 | 0.193220165 |
| NHP2L1 | 0.23165042 | HNRNPK | 0.209197551 | FOXN3 | 0.193219175 |
| IST1 | 0.231409723 | ZRANB2 | 0.209010695 | DAD1 | 0.192668226 |
| DDX46 | 0.231224953 | BTN3A2 | 0.208923563 | TRA2A | 0.192112123 |
| CD3E | 0.230619198 | RAB11A | 0.208557108 | SYNCRIP | 0.191714949 |
| DDX39A | 0.230151692 | C19orf53 | 0.207810394 | LNPEP | 0.191472767 |
| HDLBP | 0.230016398 | RANBP2 | 0.207785498 | ZNF706 | 0.191386583 |
| RN7SL3 | 0.228040813 | ATP11B | 0.207293014 | SEC61G | 0.190925601 |
| EMC10 | 0.227389742 | DDX60 | 0.206641679 | ALOX5AP | 0.190673778 |
| HNRNPC | 0.227360597 | TBCB | 0.206048918 | STAT2 | 0.19012373 |
| ENO1 | 0.227175129 | DOCK8 | 0.205820844 | EIF3J | 0.188656784 |
| ILF2 | 0.226397881 | TMED5 | 0.205775266 | PA2G4 | 0.188421922 |
| PNRC2 | 0.226119395 | TAPBP | 0.205754492 | PFN1P1 | 0.188395485 |
| NFATC2 | 0.225741436 | ANXA11 | 0.205731543 | OS9 | 0.188063177 |
| THEMIS | 0.225629989 | EIF31 | 0.205554041 | SIVA1 | 0.18761931 |
| MTPN | 0.224489135 | CELF1 | 0.205292436 | ARL6IP5 | 0.186848091 |
| SERF2 | 0.224018531 | EPS15 | 0.205179684 | ANKRD17 | 0.186810614 |
| BAZ2A | 0.222997155 | TMEM59 | 0.204796959 | SRM | 0.18608177 |
| N4BP1 | 0.222705807 | TMEM258 | 0.204583066 | SREK1IP1 | 0.185518442 |
| PTBP3 | 0.222700584 | MATR3 | 0.204052917 | ACIN1 | 0.185231681 |
| RNF4 | 0.222301958 | PRPF8 | 0.203874747 | Sep. 1 | 0.184502814 |
| UBA1 | 0.222161962 | PIK3CD | 0.203549859 | WAC | 0.18409035 |
| ABI1 | 0.221744062 | SRSF10 | 0.203056165 | ACTR3 | 0.184021861 |
| PSMB7 | 0.221368277 | DDX23 | 0.202669046 | DDX17 | 0.183822584 |
| CD164 | 0.220971634 | GGNBP2 | 0.20185104 | USP15 | 0.18369593 |
| DHX15 | 0.22075958 | KHDRBS1 | 0.200516121 | NLRP1 | 0.183278068 |
| FKBP3 | 0.220110666 | GTF2F1 | 0.200282478 | TM9SF2 | 0.182859671 |
| CCDC85B | 0.2199542 | LIMD2 | 0.199935027 | PCBP1 | 0.182135867 |
| CTSS | 0.218874591 | SDF4 | 0.199435264 | ITSN2 | 0.182074213 |
| DOCK2 | 0.218106684 | GOLGA7 | 0.199126123 | XPO1 | 0.181786421 |
| ZMAT2 | 0.217985152 | SMARCC1 | 0.197970972 | RSRC2 | 0.179799278 |
| YTHDF2 | 0.217912967 | GPRIN3 | 0.197393197 | C10orf118 | 0.179715115 |
| TRIP12 | 0.217437996 | TCERG1 | 0.196701631 | GDI2 | 0.178812199 |
| BRD2 | 0.216567496 | UBE2K | 0.196372563 | SERINC3 | 0.177933216 |
| RN7SL1 | 0.215898959 | CANX | 0.19617537 | ILF3 | 0.177851421 |
| ELK4 | 0.215318395 | FBXW5 | 0.196083325 | ATP6V1E1 | 0.177342173 |
| THOC2 | 0.215069339 | Z82188.1 | 0.195917756 | CPNE1 | 0.177065782 |
| CFLAR | 0.214841285 | FAM104A | 0.195162508 | ANP32A | 0.176346837 |
| B2M | 0.214661773 | SRRM1 | 0.194388972 | SLC9A3R1 | 0.17551176 |
| ROCK1 | 0.214016757 | UPF2 | 0.19416293 | TCEB1 | 0.175010869 |
| PCM1 | 0.174874062 | SHISA5 | 0.15525488 | EIF2AK1 | 0.136371144 |
| BCLAF1 | 0.17447155 | ZNF148 | 0.155175513 | BECN1 | 0.136219082 |
| SAMD3 | 0.17443151 | GNB1 | 0.154264741 | CHD1 | 0.13478989 |
| DPY30 | 0.173926779 | LCK | 0.154208513 | RNF145 | 0.13429457 |
| XBP1 | 0.173735013 | SAP18 | 0.153940385 | STAU1 | 0.133701333 |
| ZC3H11A | 0.172778159 | GPATCH8 | 0.15385323 | PSMF1 | 0.133652302 |
| CDC5L | 0.172317677 | XRCC5 | 0.153761571 | ERGIC2 | 0.133525033 |
| ARHGEF1 | 0.171496334 | SELT | 0.153112012 | ALKBH5 | 0.131229233 |
| HSP90AB3P | 0.171414548 | PPP2R5C | 0.152992205 | PTGES3 | 0.130681337 |
| TXNL1 | 0.170766389 | GSPT1 | 0.152713888 | NIN | 0.130475215 |
| NLRC3 | 0.170506145 | DPP7 | 0.151698739 | PPT1 | 0.128889289 |
| CUTA | 0.170441481 | XAF1 | 0.151099143 | EFCAB14 | 0.128827943 |
| RAB8A | 0.169056726 | CKLF | 0.151077862 | LSM7 | 0.128710202 |
| KRR1 | 0.168986823 | CGGBP1 | 0.150466817 | LUC7L3 | 0.128481534 |
| VAMP2 | 0.168541506 | COPB1 | 0.15008826 | KMT2E | 0.127380033 |
| BIRC2 | 0.168195348 | DNAJA1 | 0.14956126 | INPP5D | 0.126118571 |
| RSBN1L | 0.167782831 | GLG1 | 0.148860815 | BNIP2 | 0.125560722 |
| QARS | 0.16736974 | PUM2 | 0.148860815 | CAPN2 | 0.124935296 |
| PTPN18 | 0.166780326 | XRCC6 | 0.14875673 | RBMX | 0.124882747 |
| ANKRD11 | 0.166636475 | STAT1 | 0.147990083 | TMEM230 | 0.124873632 |
| SPCS3 | 0.166462847 | H1FX | 0.147344333 | EIF4G2 | 0.123710306 |
| CCT4 | 0.166443165 | SF3B2 | 0.147121332 | PPIA | 0.123672611 |
| RBM3 | 0.166118603 | SYNRG | 0.146848967 | FRYL | 0.123058119 |
| PPP2R5A | 0.165549791 | CCT2 | 0.14619569 | PHIP | 0.12287934 |
| PCSK7 | 0.16491811 | CTB-89H12.4 | 0.144560233 | VPS29 | 0.122248697 |
| H3F3B | 0.164878262 | MX1 | 0.14430441 | SKAP1 | 0.12219721 |
| UBE2I | 0.164549395 | DYNC1H1 | 0.14422707 | CAPZA2 | 0.121196651 |
| PCGF5 | 0.164332364 | EPSTI1 | 0.143836217 | BCAP31 | 0.121068535 |
| IFITM2 | 0.164321747 | TCP1 | 0.143818016 | UBE2D3 | 0.120028225 |
| UBE2G2 | 0.162561312 | ATP6V1F | 0.14361021 | ANKHD1- | 0.119476771 |
| | | | | EIF4EBP3 | |
| RAP1A | 0.162407141 | DDX6 | 0.14309102 | REEP5 | 0.118908143 |
| ARID4B | 0.162385343 | NEMF | 0.141753533 | FAM89B | 0.11871513 |
| KIAA0040 | 0.161827689 | HNRNPU | 0.141075409 | PPP1R18 | 0.1179741 |
| AUP1 | 0.161534545 | SRP72 | 0.140928238 | SMARCC2 | 0.117972262 |
| AC011737.2 | 0.161430153 | CCAR1 | 0.140723227 | IL10RA | 0.117305062 |
| C11orf58 | 0.16072967 | PSMD6 | 0.140355021 | SAMD9 | 0.117144669 |
| RNF7 | 0.159214337 | WAPAL | 0.140033439 | PHF11 | 0.116609696 |
| TPR | 0.158665391 | DENND1C | 0.139160723 | DDX24 | 0.116453895 |
| CAB39 | 0.158338768 | RASSF5 | 0.138758852 | TPM3 | 0.115821814 |
| TRIM28 | 0.15832187 | MAPK1 | 0.137547606 | PPP2R5E | 0.115389643 |
| MTDH | 0.157362483 | HCLS1 | 0.137458999 | DAXX | 0.114941562 |
| PPP1R7 | 0.156640269 | RTFDC1 | 0.137225966 | MDFIC | 0.114600493 |
| BLOC1S2 | 0.156366542 | MEAF6 | 0.136934254 | EFR3A | 0.114424092 |
| PRKCB | 0.155741682 | PTPN1 | 0.136380606 | CD46 | 0.11437728 |
| EMC4 | 0.113678151 | TRIP11 | 0.093867919 | EIF5B | 0.076214436 |
| EP300 | 0.112915733 | NMI | 0.09336339 | NOP10 | 0.075891175 |
| CALCOCO2 | 0.112093107 | BRK1 | 0.093310333 | TBCA | 0.074465066 |
| STIM2 | 0.111772267 | C19orf43 | 0.093003703 | GPBP1 | 0.074079371 |
| SNRNP70 | 0.11165773 | ORMDL1 | 0.092968295 | ADH5 | 0.073780496 |
| MED4 | 0.111439927 | RABEP1 | 0.092783849 | GIMAP6 | 0.073430914 |
| HNRNPA1P48 | 0.111330917 | PRKX | 0.090834248 | EEF1DP1 | 0.073329792 |
| NCKAP1L | 0.11111549 | IFI6 | 0.090196789 | MED13 | 0.071943166 |
| SF3B14 | 0.110994801 | TMEM50A | 0.089636598 | TMEM248 | 0.071180486 |
| DNAJC21 | 0.110097683 | EIF2AK2 | 0.088605622 | AFTPH | 0.071173791 |
| MDM4 | 0.109959679 | RNF114 | 0.088296598 | FNBP1 | 0.071166584 |
| MAPRE1 | 0.109270486 | FNTA | 0.087974514 | GNA13 | 0.071070746 |
| WIPF1 | 0.109143849 | VPS4B | 0.087723389 | EIF1AX | 0.07096605 |
| RBM17 | 0.108722567 | MT2A | 0.08734337 | SBNO1 | 0.069546306 |
| SF3B1 | 0.108199704 | EIF4EBP2 | 0.086615656 | ATP6V0E1 | 0.068309896 |
| SERBP1 | 0.107671208 | MYEOV2 | 0.086367168 | CCNL1 | 0.067967172 |
| IRF3 | 0.105660089 | DDT | 0.085375522 | IRF7 | 0.067892217 |
| GLIPR2 | 0.10454944 | ARF3 | 0.085361278 | C19orf66 | 0.067254319 |
| GOLGA4 | 0.104005677 | URI1 | 0.085266145 | ARHGAP25 | 0.066230345 |
| CCSER2 | 0.103900699 | TCF25 | 0.085025488 | OIP5-AS1 | 0.065719911 |
| SLTM | 0.103174595 | WHSC1L1 | 0.084751123 | SF3A1 | 0.064523225 |
| OPTN | 0.103113725 | RPL15P3 | 0.084747675 | CIRBP | 0.06420065 |
| ARID5B | 0.103033616 | RAD21 | 0.083483132 | HUWE1 | 0.063032567 |
| UBE2D2 | 0.101893175 | VPS35 | 0.082739981 | NUP210 | 0.062303008 |
| Sep. 15 | 0.101816114 | EIF3A | 0.082628283 | HNRNPDL | 0.061584612 |
| PTPRC | 0.101641671 | SDAD1 | 0.082564995 | CD96 | 0.061449634 |
| CDV3 | 0.1015913 | IFITM1 | 0.082457967 | TSPAN14 | 0.061250235 |
| ST3GAL1 | 0.101399461 | KDM5A | 0.082366496 | HNRNPH1 | 0.060777882 |
| POLR3GL | 0.100966671 | WASF2 | 0.081728166 | HSP90AB1 | 0.060697144 |
| MVP | 0.099995099 | RABGAP1L | 0.0811494 | DARS | 0.059772198 |
| PTP4A2 | 0.099486812 | HNRNPUL2 | 0.080390778 | TNIP1 | 0.058901653 |
| CACYBP | 0.098806281 | C6orf62 | 0.0801926 | SNRPD2 | 0.058833167 |
| IDS | 0.098802048 | UFC1 | 0.079703354 | TBC1D10C | 0.058247243 |
| PDCD2 | 0.096987995 | SNRNP200 | 0.079431356 | TRIM22 | 0.056920742 |
| RAD50 | 0.096987995 | TMBIM6 | 0.079302256 | ANAPC16 | 0.056796813 |
| ERH | 0.096921244 | RBM5 | 0.078442109 | RPS15AP38 | 0.056668456 |
| ALDOA | 0.096884203 | MACF1 | 0.078399207 | KIAA2026 | 0.055914864 |
| ICAM3 | 0.096290018 | CORO1A | 0.078364371 | PRDM2 | 0.055897144 |
| ATF7IP | 0.095087098 | FTL | 0.07800592 | STAT6 | 0.054299972 |
| PPIB | 0.094587842 | SRSF5 | 0.077751998 | PRPF4B | 0.053501209 |
| TMED2 | 0.09442747 | CCDC91 | 0.077179822 | TMED4 | 0.053141317 |
| SH3BGRL | 0.094425019 | SYAP1 | 0.076863988 | PPIG | 0.052967482 |
| DYNLRB1 | 0.094372436 | EPC1 | 0.076472624 | TTC1 | 0.052437342 |
| TUG1 | 0.094223659 | CCT5 | 0.07630883 | EIF3M | 0.05234172 |
| SNRPB2 | 0.051746365 | CSDE1 | 0.034518408 | CNOT7 | 0.007232642 |
| SETX | 0.051684093 | STK10 | 0.032534293 | PHF20L1 | 0.007133327 |
| DOK2 | 0.051516687 | PAK2 | 0.032446534 | CTB-36O1.7 | 0.006810488 |
| VPS13C | 0.051432629 | FBXW7 | 0.031338392 | STAT5B | 0.006048252 |
| LPXN | 0.050412229 | ZCCHC11 | 0.031181928 | ARID1A | 0.005722394 |
| CTR9 | 0.049923019 | DR1 | 0.031008994 | CTDNEP1 | 0.005369374 |
| RAP1B | 0.049635469 | SUMO3 | 0.030834926 | RASA2 | 0.003477636 |
| ACP1 | 0.049624672 | SLC38A1 | 0.030034926 | STAG2 | 0.00239101 |
| UTRN | 0.049331826 | UBQLN1 | 0.029670912 | NOLC1 | 0.002270043 |
| NFKB1 | 0.04869259 | SMARCA2 | 0.029174605 | CIR1 | 0.001659941 |
| CASP1 | 0.048249673 | YWHAB | 0.02823678 | ITGB1 | 0.001435134 |
| ELF1 | 0.047209266 | CNTRL | 0.027520812 | GPBP1L1 | 0.000720532 |
| ZBTB4 | 0.047102076 | YWHAZ | 0.027066103 | ZKSCAN1 | 2.88E−05 |
| HIST1H1D | 0.046458256 | PTMAP4 | 0.026925292 | PEBP1 | 2.51E−05 |
| G3BP2 | 0.046012174 | WDR26 | 0.026017021 | FYB | −0.000992234 |
| RBM39 | 0.045932293 | TANK | 0.025146574 | ARCN1 | −0.001635646 |
| PSMB3 | 0.04590686 | LINC00493 | 0.024446523 | KCNAB2 | −0.00262286 |
| PPA1 | 0.045804939 | USP14 | 0.024216482 | RHOF | −0.002980605 |
| YTHDC1 | 0.045614349 | TM9SF3 | 0.024149605 | ATF4 | −0.00351396 |
| USP47 | 0.045255138 | SP110 | 0.023359562 | SMARCA5 | −0.003546764 |
| SRRM2 | 0.044840424 | YWHAG | 0.022782721 | ZNF638 | −0.004154354 |
| DBNL | 0.044819155 | NME2 | 0.022768371 | NUFIP2 | −0.005339637 |
| MZT2B | 0.044380871 | TMSB4X | 0.022440831 | TALDO1 | −0.006359566 |
| TAX1BP1 | 0.043943706 | MYL12B | 0.021222141 | DYRK1A | −0.006699275 |
| WBP11 | 0.043130194 | KIAA1109 | 0.018470701 | HNRNPH3 | −0.007866518 |
| USP9X | 0.042509604 | IK | 0.01821637 | ESYT1 | −0.008376456 |
| RAD23A | 0.042195181 | AKAP9 | 0.017769389 | UBE2R2 | −0.008492178 |
| TMEM30A | 0.041495316 | C12orf57 | 0.016943589 | ZC3H13 | −0.008868228 |
| VAMP8 | 0.04123862 | CHD4 | 0.016203864 | SEPW1 | −0.009716326 |
| YBX1 | 0.040244484 | DENR | 0.015290422 | LAMTOR4 | −0.010671456 |
| ARID5A | 0.039713629 | N4BP2L2 | 0.014694262 | USP34 | −0.010952694 |
| STX16 | 0.039704509 | RBBP6 | 0.014474945 | SYNE2 | −0.011093572 |
| CSNK1G3 | 0.039407523 | UBE2Q1 | 0.01428704 | H3F3AP6 | −0.011256756 |
| KPNB1 | 0.039140251 | AL136419.6 | 0.013957554 | MED15 | −0.011359543 |
| HSPA8 | 0.038619362 | SCAF11 | 0.013487926 | ELOVL5 | −0.011434195 |
| TRAPPC5 | 0.038492262 | NMT1 | 0.013132997 | DDX5 | −0.012372733 |
| TIAL1 | 0.038020443 | HIF1A | 0.011916565 | TAPSAR1 | −0.013052188 |
| TARDBP | 0.037580733 | LAMP1 | 0.011407931 | SIPA1 | −0.013679828 |
| ZC3H15 | 0.037109931 | MAP3K2 | 0.011302887 | HELZ | −0.015104435 |
| PRDX1 | 0.036618152 | CDC42 | 0.011175877 | APBB1IP | −0.015965174 |
| TAF10 | 0.035943768 | TMED10 | 0.010971933 | HLA-E | −0.016019852 |
| TRAF3IP3 | 0.035894027 | IMP3 | 0.00851047 | TAOK1 | −0.016397656 |
| JTB | 0.034797702 | SAT1 | 0.00788386 | MAF | −0.017871328 |
| ASH1L | 0.034712466 | MAT2B | 0.007369696 | UFM1 | −0.018472271 |
| GMFG | −0.018611922 | FNBP4 | −0.047815699 | NIPBL | −0.07472685 |
| CLNS1A | −0.018982662 | SSB | −0.04839845 | MAP7D1 | −0.077572831 |
| SAR1A | −0.019645369 | CCNI | −0.048932299 | ERAP2 | −0.078795192 |
| RPS24P8 | −0.020454204 | EIF1 | −0.049284926 | PDS5A | −0.080299973 |
| UBR4 | −0.021089524 | LSM4 | −0.050322676 | SEC63 | −0.080906166 |
| RB1CC1 | −0.022352539 | SETD2 | −0.050693456 | CLDND1 | −0.082243716 |
| AZIN1 | −0.022753906 | CKLF-CMTM1 | −0.051508746 | RAB14 | −0.082581069 |
| SENP6 | −0.023703306 | MRFAP1L1 | −0.051720218 | HEBP2 | −0.082880568 |
| CBFB | −0.025707654 | GOLGB1 | −0.052028667 | HMOX2 | −0.083242015 |
| DDX18 | −0.025739079 | GYPC | −0.053195338 | BPTF | −0.083407765 |
| OAS3 | −0.026824239 | RIF1 | −0.053403877 | EHD1 | −0.083477219 |
| CSTB | −0.026942588 | G3BP1 | −0.054250625 | PCNP | −0.083724193 |
| CIB1 | −0.028053869 | ARL6IP4 | −0.054376759 | PLEKHA2 | −0.08510135 |
| HNRNPA1 | −0.031583297 | DDX3X | −0.058756581 | ATP6AP2 | −0.085196117 |
| FAM177A1 | −0.031831575 | SSU72 | −0.058764656 | RPS4Y1 | −0.086515586 |
| BOD1L1 | −0.031962267 | OSTC | −0.058809315 | ITK | −0.088061747 |
| PRRC2B | −0.032152358 | CEP350 | −0.058868809 | FXR1 | −0.088800254 |
| SKP1 | −0.033180062 | ZAP70 | −0.058913473 | HNRNPUL1 | −0.089452204 |
| GIT2 | −0.033502566 | TAOK3 | −0.059262314 | AC011933.1 | −0.089691379 |
| PBRM1 | −0.035937938 | TCEA1 | −0.060618094 | NUP62 | −0.092338021 |
| AL590762.1 | −0.036488169 | CMTM3 | −0.060903091 | COMMD6 | −0.092432268 |
| KAT2B | −0.037114519 | TAB2 | −0.06160049 | ANXA2 | −0.092660534 |
| CLSTN1 | −0.037480827 | ISG20 | −0.062190049 | FLOT2 | −0.093039292 |
| NR3C1 | −0.037796276 | TRAM1 | −0.062491603 | DOCK11 | −0.096053757 |
| ARID4A | −0.038197984 | FIP1L1 | −0.062655518 | PPP1R2 | −0.096715936 |
| MPHOSPH8 | −0.038379258 | CREM | −0.063961985 | BIRC3 | −0.097715444 |
| SP100 | −0.038386751 | RPL35AP21 | −0.064280532 | PDE3B | −0.098700294 |
| RPS27L | −0.039037286 | TMA7 | −0.064536047 | DNAJC3 | −0.099119174 |
| DIAPH1 | −0.039381517 | PPCS | −0.065291041 | MIER1 | −0.099766443 |
| RHOG | −0.039423649 | RNPEPL1 | −0.067284571 | DDX21 | −0.100409245 |
| ARFGEF1 | −0.039781033 | KDM2A | −0.067776136 | RPL24P2 | −0.100539591 |
| TSC22D4 | −0.04011824 | LARP1 | −0.068202656 | UBE2H | −0.104899558 |
| RALBP1 | −0.040649678 | MZT2A | −0.068483733 | CCT3 | −0.105718255 |
| DHX9 | −0.041538242 | MYL12A | −0.068886876 | FYTTD1 | −0.107162473 |
| TAF1D | −0.042427875 | MYH9 | −0.070052578 | EIF2S2 | −0.10889976 |
| RHOH | −0.042864968 | WDR82 | −0.070365753 | BTF3 | −0.110634243 |
| PRKDC | −0.04348065 | BRWD1 | −0.071004737 | LSM14A | −0.111120199 |
| SUGT1 | −0.043627832 | SNX6 | −0.07123632 | MRFAP1 | −0.111602959 |
| CNBP | −0.043667454 | NPM1 | −0.071366304 | APOL3 | −0.112867845 |
| RPL36AP21 | −0.044041986 | AKAP11 | −0.072138033 | SMCHD1 | −0.114690261 |
| CYFIP2 | −0.044536272 | REST | −0.07336251 | ARRB2 | −0.115280648 |
| PURA | −0.044600275 | RPA2 | −0.073433536 | PSIP1 | −0.115496776 |
| ABRACL | −0.044657647 | U2AF2 | −0.073520607 | LPIN2 | −0.117326436 |
| TGOLN2 | −0.047708734 | GATA3 | −0.074180401 | NUDT3 | −0.11737082 |
| P1P4K2A | −0.117485038 | IER2 | −0.161241913 | DHRS7 | −0.201766808 |
| EVI2B | −0.119966052 | AC005884.1 | −0.161551305 | RPS24 | −0.202048301 |
| RSF1 | −0.120717473 | STK17B | −0.163135954 | ACAP2 | −0.202059374 |
| PCBP2 | −0.121723289 | CHD2 | −0.164951502 | PCED1B-AS1 | −0.203464933 |
| CTBP1 | −0.123032756 | PNRC1 | −0.165664054 | RPL36AL | −0.203500955 |
| MKNK2 | −0.123315384 | JUN | −0.165706656 | KIAA1551 | −0.204904822 |
| YY1 | −0.124058353 | RPL21P28 | −0.16766389 | POLE3 | −0.205148804 |
| RBMS1 | −0.124059655 | CLK1 | −0.168186138 | CHD3 | −0.205226279 |
| STK17A | −0.124701155 | DAZAP2 | −0.168747449 | ARHGDIB | −0.207633796 |
| BDP1 | −0.126215933 | SYF2 | −0.169060104 | SNURF | −0.207838585 |
| GPR65 | −0.126254245 | SLFN5 | −0.169849998 | NDFIP1 | −0.209123998 |
| EPRS | −0.129668351 | KIF2A | −0.169858868 | SUN2 | −0.21062428 |
| ACAP1 | −0.130106898 | AP000936.1 | −0.169965813 | TMSB10 | −0.211177014 |
| ATXN7L3B | −0.130121758 | ERGIC3 | −0.170632513 | CUL3 | −0.212321451 |
| NT5C3A | −0.130234629 | O5T4 | −0.172067524 | GLRX | −0.21279021 |
| JAK1 | −0.130860951 | FAM204A | −0.174443988 | ETS1 | −0.214478946 |
| APRT | −0.131134682 | RTN4 | −0.174814215 | UBXN1 | −0.214648923 |
| GVINP1 | −0.13124866 | S100A11 | −0.175328651 | FGFR1OP2 | −0.214928598 |
| BAZ1A | −0.132555609 | UBTF | −0.175348794 | TLK1 | −0.214970248 |
| CDC42SE2 | −0.133260981 | KLF13 | −0.175647933 | XRN2 | −0.215541069 |
| RAB2A | −0.13632851 | CDC42SE1 | −0.17622252 | CTB-79E8.3 | −0.215736783 |
| EIF3K | −0.137332296 | PHACTR2 | −0.178594703 | RSAD2 | −0.215892212 |
| RWDD1 | −0.137802685 | FAM107B | −0.17909001 | CALM1 | −0.216514782 |
| MYCBP2 | −0.139537204 | DYNLL2 | −0.180588358 | DEF6 | −0.220283475 |
| POLR1D | −0.140099655 | PPP1R10 | −0.181689245 | PSAP | −0.224363567 |
| RNF149 | −0.141238412 | ZC3H6 | −0.181918678 | ARL4C | −0.2258361 |
| PTPN2 | −0.1421677 | SF1 | −0.182483832 | DNAJA2 | −0.227098909 |
| ARHGEF6 | −0.145417047 | RPS19 | −0.182583631 | TRABD | −0.227098909 |
| ZNF101 | −0.147203347 | GLIPR1 | −0.182601606 | CCND3 | −0.227682528 |
| NUP50 | −0.147414201 | ERBB2IP | −0.183273078 | TMF1 | −0.228156223 |
| RBM27 | −0.149439516 | SLK | −0.186022813 | STT3B | −0.228780885 |
| NFE2L2 | −0.149878184 | EIF3H | −0.1868365 | ZNF217 | −0.230778267 |
| ABHD14B | −0.150789741 | STK4 | −0.187068491 | BBX | −0.231499054 |
| CD53 | −0.151273603 | CMPK1 | −0.187627488 | RPL21P75 | −0.231591617 |
| PDE4DIP | −0.151745449 | CTD-2013N17.1 | −0.187766493 | CEP85L | −0.231901677 |
| RPL12 | −0.15183916 | THUMPD1 | −0.191619961 | UBALD2 | −0.233445601 |
| LIME1 | −0.153470846 | MLLT6 | −0.192655381 | TGFBR2 | −0.235113527 |
| RPL24P8 | −0.153945057 | PFDN5 | −0.193391155 | NUB1 | −0.235195426 |
| SSR2 | −0.154341857 | XPC | −0.19608081 | SIGIRR | −0.236247702 |
| JUND | −0.155077314 | MBNL1 | −0.197783986 | LAPTM5 | −0.23755316 |
| NSA2 | −0.155601632 | GIMAP4 | −0.198255484 | EIF3D | −0.239271246 |
| C19orf60 | −0.159516543 | KMT2C | −0.199005214 | ANKRD12 | −0.241653199 |
| RPS7P10 | −0.160485837 | CD47 | −0.199072365 | STK24 | −0.242474797 |
| AKIRIN1 | −0.161120635 | KTN1 | −0.200661105 | PHF15 | −0.242562114 |

TABLE 2
Gene expression enrichment table (Dysfunctional over cytotoxic T cells)

| gene | Log2 enrichment | gene | Log2 enrichment | gene | Log2 enrichment |
|---|---|---|---|---|---|
| TOX2 | 4.246364078 | TBC1D4 | 1.647325425 | AGFG1 | 1.076866057 |
| CXCL13 | 4.036278733 | TNIP3 | 1.645146769 | RANBP1 | 1.066980292 |
| ZBED2 | 3.176146138 | TOX | 1.599865108 | PTTG1 | 1.065344039 |
| DUSP4 | 3.165131762 | GALM | 1.583036233 | CRTAM | 1.05518417 |
| AKAP5 | 3.034198419 | LYST | 1.582198919 | ANXA5 | 1.053531721 |
| CSF1 | 3.01220214 | FUT8 | 1.577463535 | CBLB | 1.047953007 |
| TNFRSF9 | 2.782034838 | COTL1 | 1.573092823 | NASP | 1.046075219 |
| NR4A2 | 2.780719901 | STMN1 | 1.532278061 | TNFRSF1B | 1.045451402 |
| RDH10 | 2.76319932 | CD38 | 1.530849128 | TMPO | 1.028116806 |
| ID3 | 2.747769245 | RBPJ | 1.514824455 | HIST1H4C | 1.016315726 |
| TTN | 2.719796984 | XXbac- | 1.50438335 | GBP2 | 1.01151009 |
| | | BPG254F23.6 | | | |
| MIR155HG | 2.710836178 | CENPF | 1.502396387 | CCL4 | 1.010269474 |
| CCL3 | 2.685067084 | KIF20B | 1.499161911 | MBD2 | 1.001780582 |
| VCAM1 | 2.596824313 | AD000671.6 | 1.476665438 | PTPN11 | 1.000436748 |
| PDCD1 | 2.557412907 | GFOD1 | 1.448634439 | HMGB2 | 0.997510965 |
| CTLA4 | 2.556476899 | HLA-DPA1 | 1.383121077 | TNFAIP3 | 0.992321248 |
| KIR2DL4 | 2.543002006 | HLA-DRB1 | 1.331322029 | MFSD6 | 0.989933143 |
| SIRPG | 2.488019188 | MAF | 1.324824159 | SLA | 0.98736254 |
| SNX9 | 2.482502044 | TRAF5 | 1.293235526 | SMC1A | 0.987267398 |
| LAG3 | 2.478477582 | TUBA1B | 1.282131565 | FKBP1A | 0.986748255 |
| ENTPD1 | 2.472322061 | ZFP36L1 | 1.275419265 | CD2BP2 | 0.982720037 |
| SNAP47 | 2.427842609 | CCR5 | 1.272368513 | TRIM8 | 0.979740278 |
| CXCR6 | 2.373113741 | RGS2 | 1.269947688 | HMGN2 | 0.977658867 |
| MKI67 | 2.368854732 | LIMS1 | 1.264511425 | SEL1L3 | 0.973893962 |
| DGKH | 2.367918725 | EOMES | 1.261949587 | CD7 | 0.968202549 |
| HAVCR2 | 2.336536442 | PCNA | 1.256492775 | RAB27A | 0.965540958 |
| HLA-DQA1 | 2.307890337 | INPP4B | 1.255985082 | BIRC3 | 0.962794223 |
| IFNG | 2.285146668 | TAPBPL | 1.253710336 | CBX5 | 0.961792412 |
| CD27 | 2.263888577 | PKM | 1.240482322 | BATF | 0.956074735 |
| TOP2A | 2.242875364 | ANKRD32 | 1.221168454 | GGA2 | 0.955323838 |
| CCL4L2 | 2.223429546 | ATXN1 | 1.199378459 | STAT3 | 0.952182056 |
| TIGIT | 2.21696405 | HLA-DRB5 | 1.198549528 | RALA | 0.949090895 |
| PTMS | 2.149830649 | HIST1H1B | 1.18284669 | RNU2-2P | 0.935307315 |
| RGS1 | 2.044538264 | TRAF1 | 1.182491823 | GPR171 | 0.933986674 |
| AMICA1 | 1.992383484 | VOPP1 | 1.181603795 | GBP5 | 0.927116589 |
| HLA-DMA | 1.919395493 | HNRNPLL | 1.180543596 | IFI27L2 | 0.922544425 |
| FCRL3 | 1.834842207 | WARS | 1.170128203 | RGCC | 0.919755511 |
| ITGAE | 1.831629894 | TRAC | 1.14591268 | CALM3 | 0.915665164 |
| PHLDA1 | 1.818600144 | SIT1 | 1.116619677 | SPTAN1 | 0.914641526 |
| ITM2A | 1.784882554 | CD8A | 1.103531809 | SMC4 | 0.908938074 |
| FAM3C | 1.762646221 | TYMP | 1.099328056 | IRF2 | 0.902095396 |
| HLA-DRA | 1.708018888 | GZMK | 1.096456346 | ALOX5AP | 0.900858005 |
| CD74 | 1.661364154 | IKZF3 | 1.090838634 | GBP4 | 0.900259565 |
| PAG1 | 1.647865112 | PPP1R16B | 1.081329324 | CDK6 | 0.898863904 |
| DYNLL1 | 0.892535525 | COPA | 0.725293847 | HMGB1P5 | 0.625179453 |
| ASXL2 | 0.89061564 | GALNT1 | 0.723248126 | APOBEC3C | 0.625034645 |
| LINC00152 | 0.88998062 | MAP4K1 | 0.72132236 | ZNF106 | 0.624697318 |
| STAT1 | 0.889229438 | YWHAE | 0.720866763 | LSP1 | 0.621687448 |
| DUSP2 | 0.887379787 | IFI6 | 0.720528564 | CLTA | 0.620404225 |
| METRNL | 0.872057512 | TUBB | 0.708919567 | WAS | 0.619746318 |
| PSTPIP1 | 0.869113687 | PTMAP5 | 0.707794072 | JAK3 | 0.616593092 |
| CD84 | 0.851598986 | ANAPC11 | 0.706838636 | XRN1 | 0.614832454 |
| AC011737.2 | 0.84612485 | ZNF638 | 0.697508255 | PARP14 | 0.613951488 |
| LSM2 | 0.841079926 | DHX15 | 0.696811446 | SYNGR2 | 0.613487727 |
| SYT11 | 0.83484488 | DRAP1 | 0.695703247 | GNAS | 0.608154087 |
| TRIM69 | 0.823772426 | ARID5B | 0.694871249 | TLK1 | 0.60633379 |
| HLA-DPB1 | 0.822260522 | HN1 | 0.6919225 | SUMO1 | 0.606050335 |
| CTSB | 0.821634166 | BRD7 | 0.691260018 | ANP32B | 0.605368677 |
| SHFM1 | 0.812708345 | PTPRA | 0.689574108 | SURF4 | 0.605294588 |
| RFTN1 | 0.812699209 | DDX27 | 0.687807787 | M6PR | 0.602011588 |
| ORMDL3 | 0.809926529 | SERPINB1 | 0.687568148 | SH2D1A | 0.601321877 |
| RCC2 | 0.808021023 | FAM105B | 0.67910903 | STAU1 | 0.598405892 |
| EZR | 0.806057497 | PIK3AP1 | 0.675162927 | HMGB1 | 0.5975204 |
| SUZ12 | 0.800703687 | DENND2D | 0.674579507 | RNF187 | 0.597106882 |
| TRAT1 | 0.789682368 | FAM89B | 0.672659025 | SRI | 0.594954783 |
| PCED1B | 0.789199538 | MAPRE2 | 0.668657269 | BST2 | 0.594740624 |
| AC104698.1 | 0.785866589 | H1FX | 0.666948101 | SMG1 | 0.594442358 |
| VAMP5 | 0.782113691 | CD8B | 0.666526652 | IPCEF1 | 0.593965588 |
| PLEC | 0.782062326 | FKBP3 | 0.663361426 | CCL5 | 0.592797613 |
| WNK1 | 0.780557611 | TXNDC17 | 0.663007232 | SUPT16H | 0.591548379 |
| MCL1 | 0.780174304 | CNTRL | 0.660879058 | FYB | 0.586576058 |
| ZNF101 | 0.774681742 | PIM2 | 0.66016627 | C17orf49 | 0.583627795 |
| ANKRD11 | 0.767035756 | UBE2L6 | 0.660145229 | MESDC2 | 0.583350664 |
| HIST1H1C | 0.7615626 | GTF2F1 | 0.653671504 | PTBP1 | 0.582904981 |
| PARP1 | 0.758089061 | FAM53B | 0.652781348 | NAP1L4 | 0.576169578 |
| TRBC2 | 0.755886085 | KMT2A | 0.65266019 | PSME2 | 0.576017571 |
| EPSTI1 | 0.754279546 | PSMD8 | 0.652154347 | ARL6IP1 | 0.573107458 |
| MAP4 | 0.751917934 | YARS | 0.647966746 | PTPN6 | 0.571298578 |
| RIN3 | 0.744640669 | DEK | 0.6444439 | PTMAP2 | 0.570633916 |
| NUDT21 | 0.741564846 | CCND2 | 0.644431523 | SRGN | 0.567983421 |
| HSPB1 | 0.740244189 | LASP1 | 0.643401628 | MZT2A | 0.567448017 |
| AC092580.4 | 0.737905348 | OASL | 0.63913066 | CXCR3 | 0.566107583 |
| SH3KBP1 | 0.735969205 | RPL7L1 | 0.636564657 | CLEC2D | 0.563914801 |
| AKR1B1 | 0.733202642 | RNF213 | 0.635970285 | HDLBP | 0.563442063 |
| C11orf48 | 0.729379307 | UBE2A | 0.630286398 | PTPN7 | 0.56292947 |
| CD2 | 0.728623474 | ADNP | 0.629420545 | TERF2IP | 0.559488602 |
| UBE2F | 0.726326883 | ANP32E | 0.626293512 | PDCD6 | 0.555049355 |
| RNF19A | 0.726197745 | H2AFZ | 0.625509269 | PSMB8 | 0.552867191 |
| UBA1 | 0.552316856 | ZNFX1 | 0.505994847 | SEMA4D | 0.459732034 |
| WIPF2 | 0.552289321 | ARID5A | 0.504826272 | RAN | 0.458953036 |
| SSU72 | 0.550979254 | SFPQ | 0.504506933 | GSPT1 | 0.456527563 |
| DDX60 | 0.547347871 | ZC3H7A | 0.503555517 | RAB10 | 0.454998678 |
| PPP1CC | 0.546217626 | ID2 | 0.500933348 | MTDH | 0.454480028 |
| RNF145 | 0.545880799 | MIF | 0.500797543 | TAP1 | 0.450497073 |
| NFKBIA | 0.544910885 | EIF5 | 0.498063108 | GTF3C6 | 0.450428009 |
| YWHAH | 0.541900475 | ITSN2 | 0.49662149 | SSRP1 | 0.450325607 |
| LRP10 | 0.540474592 | GPRIN3 | 0.495574217 | MAPKAPK2 | 0.448743651 |
| TXNL4A | 0.53843111 | NKTR | 0.492593393 | MEA1 | 0.448270003 |
| APOBEC3D | 0.538325054 | ZCRB1 | 0.491155307 | DDX24 | 0.447681633 |
| ITPKB | 0.538088143 | IFI44 | 0.490139019 | LCP2 | 0.447077411 |
| ALKBH5 | 0.537460838 | TOP1 | 0.489234918 | ANP32A | 0.446485582 |
| MIR4435- | 0.536182252 | SMC5 | 0.488741083 | ERAP2 | 0.445328144 |
| 1HG | | | | | |
| MAT2A | 0.533279922 | XCL2 | 0.487939169 | PRRC2C | 0.444916869 |
| TBL1XR1 | 0.530811281 | HLA-A | 0.48747415 | NUCKS1 | 0.443929423 |
| PLA2G16 | 0.53078605 | MAP2K2 | 0.487269557 | SEPHS2 | 0.442695289 |
| SBNO1 | 0.529318114 | SH2D2A | 0.484328388 | CBX3 | 0.442092845 |
| WDR83OS | 0.528477459 | ALDOA | 0.483724564 | CNOT6L | 0.441119331 |
| ZBTB1 | 0.527631001 | PSMD4 | 0.482137763 | TAB2 | 0.440718127 |
| MIAT | 0.52731174 | PEBP1 | 0.481567473 | LEPROTL1 | 0.440526303 |
| SMARCA2 | 0.52731174 | MXD4 | 0.481331911 | PCGF5 | 0.438257207 |
| ZNF292 | 0.52727842 | C14orf166 | 0.479916125 | ST8SIA4 | 0.438107925 |
| ST3GAL1 | 0.527009433 | CD3E | 0.479272556 | AKT1 | 0.437045335 |
| BAZ1B | 0.526100401 | GATA3 | 0.478611636 | REST | 0.437008757 |
| LDHA | 0.525935727 | SET | 0.478334803 | RPS6KA3 | 0.436060204 |
| HNRNPR | 0.525837897 | IL2RG | 0.477566652 | BRD4 | 0.436013137 |
| CTCF | 0.525506973 | IGF2R | 0.475221768 | LIMD2 | 0.435831308 |
| PAFAH1B2 | 0.524899475 | SREK1 | 0.474282517 | RFC1 | 0.435349958 |
| CSK | 0.524822404 | IL2RB | 0.474189483 | CALR | 0.434341512 |
| NFKB1 | 0.521941702 | KCTD20 | 0.469830632 | SUPT5H | 0.43377704 |
| VPS28 | 0.521030752 | IFI16 | 0.469794872 | AC004057.1 | 0.433717104 |
| RAC1 | 0.520984458 | LSMD1 | 0.468353031 | TMEM189 | 0.43318335 |
| AC012379.1 | 0.519015002 | AP2B1 | 0.467857313 | PDIA6 | 0.43190232 |
| SP140 | 0.517637088 | CTD-3214H19.4 | 0.466203275 | ZCCHC6 | 0.431305922 |
| EEF1DP1 | 0.516673862 | SUB1 | 0.465826755 | CKLF | 0.43033687 |
| UPF2 | 0.514465181 | HNRNPAB | 0.465741153 | AC013394.2 | 0.430000007 |
| SAMSN1 | 0.513363791 | NR3C1 | 0.465347735 | WBP11 | 0.430000007 |
| FASLG | 0.511021853 | HNRNPL | 0.464851546 | LPIN2 | 0.429466396 |
| CTSD | 0.510736628 | PSMB2 | 0.463028468 | IFI44L | 0.429414423 |
| PSMB6 | 0.509220101 | VPS35 | 0.46253711 | DGKZ | 0.428966431 |
| NPIPB5 | 0.508696741 | YTHDF2 | 0.462162738 | TRMT112 | 0.428888094 |
| HMGN1 | 0.506734239 | ACTN4 | 0.461375457 | FIP1L1 | 0.428004484 |
| NONO | 0.506421759 | PARP4 | 0.460246044 | HNRNPD | 0.427346898 |
| SLA2 | 0.427153705 | SNORD3A | 0.38880082 | DDIT4 | 0.347258738 |
| PSMC3 | 0.427104125 | SMEK1 | 0.386458941 | CREM | 0.347182223 |
| HERC1 | 0.426850811 | N4BP1 | 0.385430883 | C5orf56 | 0.345862739 |
| NOP58 | 0.426430512 | HNRNPA2B1 | 0.384476991 | LMAN2 | 0.345631694 |
| PRPF40A | 0.426148731 | NCL | 0.38227436 | PTGER4 | 0.345380449 |
| OAS3 | 0.422944618 | KHDRBS1 | 0.37925823 | ILF3 | 0.344968283 |
| FAM102A | 0.422683019 | ARID4B | 0.377392964 | COPE | 0.344898207 |
| PSMB9 | 0.421897655 | NAPA | 0.376994218 | SMC3 | 0.344330832 |
| UBXN4 | 0.421384293 | HERPUD1 | 0.376848051 | TOP2B | 0.343981781 |
| SNRPD1 | 0.421333877 | CD96 | 0.376452428 | CCT6A | 0.343933428 |
| NCOA1 | 0.419805905 | RGS10 | 0.376320151 | GIGYF2 | 0.343369834 |
| PSMA4 | 0.41949318 | EDF1 | 0.376146695 | PPP6R1 | 0.343277162 |
| SEC11A | 0.418671841 | H2AFY | 0.375848572 | HSPH1 | 0.342468891 |
| RN7SL2 | 0.417518678 | RBM23 | 0.375273114 | LY6E | 0.3415914 |
| VCP | 0.416319554 | UBE2N | 0.37491543 | PSMB7 | 0.34085043 |
| AP2M1 | 0.416123896 | DNTTIP2 | 0.373643038 | EIF3A | 0.338959864 |
| NPM1 | 0.415517077 | ELK4 | 0.372690398 | ARHGEF1 | 0.338532396 |
| SNRPB | 0.415024907 | HP1BP3 | 0.372581398 | HMGN3 | 0.338135925 |
| PIK3IP1 | 0.413426752 | PSMB4 | 0.372264551 | UBE2D2 | 0.337475696 |
| ARHGEF6 | 0.412879256 | SRP14 | 0.371827485 | RPL21P28 | 0.337096099 |
| EXOSC9 | 0.409965134 | USP1 | 0.370320339 | GZMB | 0.336257247 |
| CDK2AP2 | 0.408688249 | ELF1 | 0.369428174 | PGK1 | 0.335674801 |
| APOL3 | 0.408582987 | RPL35AP21 | 0.367463081 | PPP1R12A | 0.33566646 |
| MYCBP2 | 0.408471656 | FYN | 0.36596036 | ZBTB38 | 0.335459702 |
| RTF1 | 0.408326763 | STAT5B | 0.363876856 | PSMA2 | 0.335097975 |
| NCOR1 | 0.407901837 | ARPP19 | 0.363259795 | AIMP1 | 0.334696429 |
| DYNC1H1 | 0.407208195 | MATR3 | 0.362859276 | CSNK2B | 0.334448261 |
| RAD21 | 0.406986052 | C1orf43 | 0.362415782 | PSMC2 | 0.334243167 |
| PTMA | 0.404079084 | XAF1 | 0.362255888 | C17orf62 | 0.334171842 |
| GBP1 | 0.402180733 | EP300 | 0.36184353 | FERMT3 | 0.333789907 |
| HDGF | 0.401213799 | RPL15P3 | 0.361781602 | PTPN22 | 0.333351638 |
| ABI3 | 0.400601137 | HERPUD2 | 0.361572713 | KMT2E | 0.331556622 |
| U2AF1 | 0.399775529 | RNA5-8SP6 | 0.36135302 | LAMTOR5 | 0.331413077 |
| MX1 | 0.399425923 | MGAT1 | 0.361348955 | DARS | 0.331120524 |
| ENO1 | 0.399206926 | EVL | 0.358935162 | RTFDC1 | 0.330398207 |
| PCM1 | 0.397477784 | COPZ1 | 0.358440955 | LARP1 | 0.330351499 |
| MAPK1IP1L | 0.393693949 | GOLGB1 | 0.358402342 | CEP57 | 0.330306238 |
| SAMD9L | 0.392185573 | DNAJA1 | 0.356645656 | PDS5A | 0.32834655 |
| PJA2 | 0.391372023 | CRIP1 | 0.355011285 | XCL1 | 0.328124586 |
| SPEN | 0.390920275 | ADRBK1 | 0.354381022 | RABAC1 | 0.327977864 |
| OS9 | 0.389486974 | KIF5B | 0.354360866 | CCDC142 | 0.326119983 |
| CHST12 | 0.389291896 | SPATA13 | 0.353321756 | WSB1 | 0.323681837 |
| UBB | 0.389255659 | DNMT1 | 0.351316443 | OAS2 | 0.322836397 |
| RARRES3 | 0.388984896 | ISG15 | 0.347423175 | ENSA | 0.322421755 |
| CCDC85B | 0.321817265 | PSMA7 | 0.293405399 | SMCHD1 | 0.270660024 |
| CEP350 | 0.321038972 | PPP1R2 | 0.292240975 | PNN | 0.268689234 |
| ACIN1 | 0.319889267 | THRAP3 | 0.291088641 | ARPC2 | 0.268632115 |
| EIF4H | 0.319766339 | CSDE1 | 0.290597862 | AC016739.2 | 0.268009878 |
| ANAPC5 | 0.319444633 | CALM2 | 0.290529487 | CD63 | 0.267848768 |
| DHX9 | 0.318002082 | USP7 | 0.289729785 | SERPINB9 | 0.266398255 |
| SNX20 | 0.317665744 | SYNCRIP | 0.288816582 | HNRNPC | 0.265560988 |
| CTC- | 0.317297433 | TCEB2 | 0.288589435 | RPL22L1 | 0.26541366 |
| 260F20.3 | | | | | |
| PA2G4 | 0.316779278 | TPR | 0.288155483 | CD81 | 0.264797055 |
| CAPZB | 0.316670336 | TMX4 | 0.287825226 | CMIP | 0.264361513 |
| GGNBP2 | 0.316097522 | PDCD10 | 0.286659347 | EIF2AK2 | 0.263414493 |
| ICAM3 | 0.313922978 | Sep-07 | 0.286620849 | CYLD | 0.26303073 |
| RPL12 | 0.31363482 | PPM1G | 0.285812947 | THOC2 | 0.262329327 |
| RBCK1 | 0.312418857 | SQSTM1 | 0.285732425 | RNASET2 | 0.261691037 |
| SNORA67 | 0.311505608 | WDR82 | 0.285732425 | CANX | 0.261386548 |
| AZIN1 | 0.311321402 | TMSB4XP8 | 0.284821132 | PHIP | 0.260957574 |
| DTX3L | 0.310760678 | NAP1L1 | 0.28424766 | NSA2 | 0.260944411 |
| CHST11 | 0.310653724 | CCAR1 | 0.28393594 | ARPC1B | 0.260876995 |
| METAP2 | 0.310138412 | CREB1 | 0.28364883 | PSMA1 | 0.259772317 |
| DCP2 | 0.309649927 | POLR2K | 0.283588479 | TMEM258 | 0.259693201 |
| CCDC69 | 0.309369794 | FAM208A | 0.283550471 | UBE3A | 0.259041429 |
| MT2A | 0.308521335 | APOBEC3G | 0.283130243 | PSIP1 | 0.258490499 |
| DNAJB11 | 0.308235661 | POLR2J | 0.281999753 | SPCS2 | 0.257179124 |
| ITGA4 | 0.307796461 | FKBP5 | 0.281857341 | YY1 | 0.256864316 |
| SCAMP2 | 0.307553847 | ATP1B3 | 0.281377957 | CCSER2 | 0.256687314 |
| Sep-02 | 0.307541262 | ETF1 | 0.280461324 | CDC37 | 0.256341834 |
| HNRNPM | 0.305402589 | CLTC | 0.280197081 | CCNI | 0.255907945 |
| HNRNPU | 0.304999955 | PTPRCAP | 0.280145096 | PDAP1 | 0.255814348 |
| SNRNP200 | 0.304798164 | PSMA5 | 0.279113555 | CTD- | 0.255697259 |
| | | | | 2013N17.1 | |
| NUP210 | 0.304773661 | WHSC1L1 | 0.27881603 | ARHGAP30 | 0.254195231 |
| DCAF7 | 0.304147848 | NEMF | 0.278323664 | SNRPD3 | 0.254068624 |
| GDI2 | 0.303519072 | TAF9 | 0.277210934 | SRSF2 | 0.252989823 |
| AL136419.6 | 0.30234308 | UBE2K | 0.275500744 | SRSF4 | 0.252707069 |
| BDP1 | 0.302095673 | F2R | 0.275236545 | C12orf57 | 0.25250224 |
| FBXL18 | 0.301974586 | MZT2B | 0.275090518 | NMT1 | 0.252477908 |
| ATP5EP2 | 0.301970891 | TMSB4XP4 | 0.275053377 | SCAF11 | 0.251831991 |
| SNW1 | 0.300364727 | IL32 | 0.273725665 | PRPF4B | 0.250652518 |
| NFATC2IP | 0.300340643 | XPO1 | 0.272893766 | SERBP1 | 0.25006245 |
| SRSF7 | 0.29733719 | OIP5-AS1 | 0.272265884 | HNRNPK | 0.249542019 |
| HSP90B1 | 0.297214204 | RHOH | 0.271636127 | ARF6 | 0.247882735 |
| XIAP | 0.296824219 | MAP1LC3B | 0.2712585 | AP000721.4 | 0.247593978 |
| C4orf3 | 0.295831448 | ADD1 | 0.271001628 | DDX23 | 0.247252697 |
| CLIP1 | 0.295263999 | ATP6V0C | 0.270724079 | PPDPF | 0.24689439 |
| PSMB1 | 0.293920472 | HSP90AB1 | 0.270676774 | C7orf73 | 0.246315677 |
| CAPRIN1 | 0.245082291 | KPNB1 | 0.221336781 | ETS1 | 0.198858062 |
| CKLF-CMTM1 | 0.244643357 | SF3B5 | 0.220717379 | KDM2A | 0.198796033 |
| RIF1 | 0.244447439 | TRA2B | 0.220274406 | TLN1 | 0.198517426 |
| CTD- | 0.244446222 | SLFN5 | 0.219228599 | PSMB3 | 0.197880758 |
| 2545G14.7 | | | | | |
| VMP1 | 0.24441297 | PSMF1 | 0.218976541 | SNRPG | 0.197785053 |
| UFD1L | 0.243238883 | ARID4A | 0.21831269 | ANAPC16 | 0.197450637 |
| ZFR | 0.242855148 | SON | 0.217010756 | URI1 | 0.197288136 |
| FNBP1 | 0.241822336 | SRP9 | 0.216907033 | FUBP1 | 0.19724479 |
| PRKAR1A | 0.240371938 | STK24 | 0.216847337 | GTF2A2 | 0.197039178 |
| RECQL | 0.240293844 | STX16 | 0.216154523 | CHD4 | 0.195350536 |
| LPIN1 | 0.240033468 | PHF11 | 0.214805415 | Sep-01 | 0.195344317 |
| LBR | 0.239829153 | TGOLN2 | 0.214450371 | UBC | 0.195006468 |
| PARP9 | 0.239612636 | RANBP2 | 0.214325817 | CSNK1G3 | 0.19452606 |
| HUWE1 | 0.239495452 | RSRC2 | 0.214126642 | BSG | 0.194377891 |
| IRF9 | 0.239050425 | NLRC5 | 0.214022143 | PSMG2 | 0.192109049 |
| RPN2 | 0.238720297 | HLA-B | 0.213657414 | RAB14 | 0.191786374 |
| XRCC6 | 0.238720297 | ATF7IP | 0.213607456 | PPIG | 0.18983423 |
| SH3BP1 | 0.238565059 | DDX46 | 0.213381969 | SF3B2 | 0.189822497 |
| PSME1 | 0.238009448 | NOP10 | 0.21317667 | ARHGAP9 | 0.189822053 |
| DNAJB1 | 0.23691789 | HSP90AA1 | 0.212163064 | HCP5 | 0.189790771 |
| SRSF9 | 0.236802449 | BRK1 | 0.211449744 | BIRC6 | 0.189777521 |
| CCDC91 | 0.236749183 | SKAP1 | 0.209534658 | RAD23B | 0.18977087 |
| PRKCH | 0.235954924 | NLRC3 | 0.20948355 | OAZ1 | 0.188666311 |
| TXN | 0.235781977 | WAPAL | 0.20899374 | SUMO2 | 0.188119097 |
| COX6CP1 | 0.235511827 | COL6A3 | 0.208835877 | RNASEH2B | 0.187853408 |
| PRDM1 | 0.235429816 | RHOA | 0.20862293 | PRRC2B | 0.187796977 |
| NIN | 0.234812893 | POMP | 0.207822339 | CAB39 | 0.187646531 |
| PAK2 | 0.233639959 | ZNF706 | 0.207325778 | UBE2I | 0.186741083 |
| DOCK2 | 0.232606256 | ATRX | 0.207043163 | SYNRG | 0.185455511 |
| ARPC5L | 0.23244805 | CEBPZ | 0.20674695 | HCLS1 | 0.18531104 |
| DNAJC21 | 0.232275074 | SMARCA5 | 0.206198364 | ARHGDIA | 0.185147333 |
| PSMA3 | 0.231877403 | EIF5A | 0.20573454 | LAPTM5 | 0.184260847 |
| DNAJB14 | 0.231688698 | EID1 | 0.205623418 | ZNF644 | 0.18377627 |
| TRAPPC1 | 0.231493538 | RNPS1 | 0.205158683 | ATP6V1F | 0.183510234 |
| ADAR | 0.230997889 | ILF2 | 0.204954056 | BRD2 | 0.18291514 |
| PPIB | 0.230206531 | RSAD2 | 0.204567358 | HSPA4 | 0.18284669 |
| C9orf16 | 0.22812413 | TMEM109 | 0.204320429 | BHLHE40 | 0.18258839 |
| CALCOCO2 | 0.227987155 | EIF4G2 | 0.204185762 | CST7 | 0.182179545 |
| NHP2L1 | 0.226409747 | CCNDBP1 | 0.203965267 | SNRPF | 0.180961279 |
| NOP56 | 0.224375458 | XRN2 | 0.203347 | LNPEP | 0.179912476 |
| KRAS | 0.224223879 | CPNE1 | 0.203168869 | TRIP12 | 0.179296361 |
| RPS15AP38 | 0.224135958 | CIB1 | 0.202520288 | POLR2G | 0.179178851 |
| ZNF207 | 0.2240937 | GNAI2 | 0.201481506 | CARD16 | 0.178933405 |
| STK17B | 0.223947314 | LPXN | 0.19935179 | SPCS1 | 0.178870849 |
| SNRPC | 0.17804751 | PTEN | 0.154100495 | YWHAB | 0.131702373 |
| H2AFV | 0.175565051 | RPL31P2 | 0.153698842 | YBX1 | 0.131604481 |
| CNOT2 | 0.175371862 | LGALS3 | 0.153500804 | TPP1 | 0.131256054 |
| BAZ2A | 0.175251856 | ATP11B | 0.153494544 | PTMAP4 | 0.130874761 |
| USF2 | 0.175222801 | JUNB | 0.152534927 | CWC15 | 0.130568513 |
| RBBP7 | 0.174895988 | MKNK2 | 0.15148958 | MYL6 | 0.129693682 |
| UBE2R2 | 0.174843825 | ARGLU1 | 0.15071639 | ZNHIT1 | 0.129346624 |
| CCT8 | 0.174798512 | NHP2 | 0.150580515 | LCK | 0.129141764 |
| DDX39B | 0.174691693 | SNRPD2 | 0.15050376 | CHMP2A | 0.128778014 |
| GYG1 | 0.17316829 | SREK1IP1 | 0.150390781 | JUN | 0.128437664 |
| ESYT1 | 0.17313407 | STK17A | 0.149798567 | KRT222 | 0.128037947 |
| NAA38 | 0.172809705 | USP16 | 0.149022108 | ATRAID | 0.127543843 |
| SETX | 0.172741608 | PLP2 | 0.148762582 | PAIP2 | 0.125890524 |
| RAB1B | 0.17239724 | LARP7 | 0.14843159 | RASGRP1 | 0.125587779 |
| KIAA1551 | 0.171165525 | TROVE2 | 0.147944068 | SLC20A1 | 0.12528062 |
| RBBP4 | 0.17115318 | CFL1 | 0.14671075 | ARPC3 | 0.125276898 |
| SUMO3 | 0.17099179 | AKAP13 | 0.146699473 | RABGAP1L | 0.125227581 |
| CD3D | 0.170772941 | RNASEK-C17orf49 | 0.146362474 | STAG2 | 0.124871845 |
| YTHDC1 | 0.170333693 | LMAN1 | 0.145482125 | PTP4A2 | 0.124852893 |
| EMC4 | 0.169995865 | H3F3B | 0.145242453 | MRFAP1L1 | 0.124839784 |
| SNURF | 0.169143954 | COPB1 | 0.143796989 | SENP6 | 0.123914076 |
| PREX1 | 0.169042132 | TAX1BP1 | 0.143758437 | RBMX | 0.12341348 |
| ATF4 | 0.168142279 | MGEA5 | 0.143303946 | RBM25 | 0.122238515 |
| TAF7 | 0.167423109 | ASH1L | 0.142027419 | GIMAP4 | 0.122151831 |
| HDAC1 | 0.167369176 | UBALD2 | 0.141979017 | HIST2H2AC | 0.121885978 |
| PNRC2 | 0.167091029 | SAT1 | 0.140903562 | PPA1 | 0.121140331 |
| GABARAPL2 | 0.163281449 | IRF1 | 0.140469736 | PDIA3 | 0.11982068 |
| SRRM1 | 0.16323734 | CNOT1 | 0.140207551 | TNIP1 | 0.119469707 |
| NOL7 | 0.163140553 | YWHAG | 0.139876894 | SNRNP27 | 0.119446741 |
| SF3B14 | 0.16270508 | AIP | 0.139697985 | UBXN1 | 0.118846431 |
| C1orf63 | 0.161985279 | TIAL1 | 0.138752115 | VAPA | 0.118790032 |
| HNRNPUL2 | 0.161759559 | ZC3H11A | 0.138637909 | BUB3 | 0.118161966 |
| AKAP9 | 0.161078317 | RPL23AP2 | 0.138563448 | FAM120A | 0.117470416 |
| HNRNPF | 0.16019408 | SAMD9 | 0.137944683 | DYNLT1 | 0.116841046 |
| XRCC5 | 0.158236841 | PPP2R5A | 0.137346946 | SH3BGRL | 0.116832336 |
| SEC61B | 0.158220213 | DENR | 0.137171709 | RAB8B | 0.116186801 |
| KIF2A | 0.157802549 | ANXA11 | 0.137050674 | PSMB10 | 0.116143887 |
| TTC39C | 0.15756368 | SLU7 | 0.136928537 | ANKHD1- | 0.115811629 |
| | | | | EIF4EBP3 | |
| RBM5 | 0.157321232 | SRPR | 0.135664451 | TAF1D | 0.115318077 |
| VAMP8 | 0.156665237 | RAP1A | 0.135208899 | SKA2 | 0.115100847 |
| EIF3H | 0.156490396 | RN7SL4P | 0.134993068 | TMBIM6 | 0.114113485 |
| CERS2 | 0.155944326 | IRF7 | 0.134732449 | GMFG | 0.113425345 |
| SMARCC1 | 0.155742355 | APOL6 | 0.132135102 | GSDMD | 0.112829575 |
| PNISR | 0.155010038 | SF3A1 | 0.132080128 | GYPC | 0.111854097 |
| U2SURP | 0.111752312 | EIF3D | 0.088230409 | UHMK1 | 0.061920074 |
| TAOK1 | 0.111731481 | CMTM6 | 0.08813014 | SMARCE1 | 0.061648343 |
| CLIC1 | 0.109008392 | HSPA5 | 0.086726058 | VPS13C | 0.061530023 |
| RBM17 | 0.10820711 | HNRNPH1 | 0.086550546 | SELT | 0.061067628 |
| FAM192A | 0.107643278 | RPL21P75 | 0.085610439 | KTN1 | 0.060395273 |
| PAPOLA | 0.107078708 | PIK3CD | 0.085350023 | EPC1 | 0.060162726 |
| CGGBP1 | 0.10699988 | RSBN1L | 0.085084039 | IL16 | 0.0600978 |
| USP15 | 0.10694309 | BUD31 | 0.082804038 | ZYX | 0.059050962 |
| HNRNPA1 | 0.106350615 | RPL24P2 | 0.082270005 | RSF1 | 0.058906012 |
| SRSF3 | 0.106207717 | IL10RA | 0.082006746 | KAT6A | 0.058774038 |
| UBL5 | 0.104509291 | VTI1B | 0.081948888 | EFCAB14 | 0.058553379 |
| BECN1 | 0.104132088 | MSN | 0.081776819 | ZMAT2 | 0.058127851 |
| UBE2G2 | 0.10297544 | ANXA6 | 0.080909697 | NFATC2 | 0.056180019 |
| MRFAP1 | 0.102643987 | YPEL5 | 0.080570055 | CD3G | 0.055677304 |
| SLAMF6 | 0.102035395 | DDOST | 0.080280182 | PPP1R11 | 0.054687617 |
| PPP1R7 | 0.102018688 | RAB8A | 0.079442326 | RPLP1 | 0.054314584 |
| RNF181 | 0.102018688 | LSM6 | 0.07942426 | RBBP6 | 0.054258246 |
| CACYBP | 0.101847654 | TMCO1 | 0.079149181 | EIF2AK1 | 0.053698257 |
| PCBP2 | 0.101712377 | CMTM3 | 0.079022289 | ARL6IP5 | 0.052616423 |
| ITGB7 | 0.101563234 | PCBP1 | 0.078942189 | SRSF10 | 0.051800456 |
| G3BP2 | 0.101272715 | RPS2P7 | 0.076023892 | GNB1 | 0.051687138 |
| DDX17 | 0.101182235 | LRRFIP1 | 0.075210787 | KLRK1 | 0.051637603 |
| PRPF8 | 0.099398324 | DOCK8 | 0.074887483 | C6orf48 | 0.051503409 |
| FUS | 0.098913773 | SP100 | 0.074547155 | ARAP2 | 0.050604495 |
| ARF1 | 0.097887288 | IQGAP1 | 0.074539539 | RPL41 | 0.049845478 |
| EPS15 | 0.097512464 | WDR26 | 0.074109181 | TNFSF10 | 0.04966911 |
| RALY | 0.096908382 | RASAL3 | 0.07400762 | SRSF5 | 0.048702513 |
| CAPZA1 | 0.09643566 | RSL1D1 | 0.073359865 | RPL10AP6 | 0.04744867 |
| C19orf43 | 0.09549839 | PPP2CA | 0.073269337 | NAA50 | 0.047380383 |
| GSTP1 | 0.095271207 | WASF2 | 0.072560237 | GNB2 | 0.047262147 |
| FDFT1 | 0.095198416 | CELF2 | 0.072292836 | SLC38A1 | 0.046596369 |
| SRSF11 | 0.094651272 | NUFIP2 | 0.07191049 | BTN3A2 | 0.045930013 |
| EVI2A | 0.094391459 | DYNLRB1 | 0.071684526 | SRSF1 | 0.045829939 |
| PSMC5 | 0.094377118 | PPP1CA | 0.070796133 | USP47 | 0.045063464 |
| RASSF5 | 0.092957125 | SP110 | 0.070088833 | SPCS3 | 0.043946366 |
| PTGES3 | 0.092711391 | CXCR4 | 0.069976709 | AMZ2 | 0.043566006 |
| COPS6 | 0.092691196 | CTSS | 0.067209039 | HSPA8 | 0.042906926 |
| TBCB | 0.09179731 | STT3B | 0.067014606 | BCLAF1 | 0.042813492 |
| DBNL | 0.091765099 | C8orf59 | 0.065169336 | HNRNPUL1 | 0.042746834 |
| WDR1 | 0.090856872 | EIF3I | 0.064765418 | FAM96B | 0.041332565 |
| TRIM22 | 0.090279448 | PNRC1 | 0.063621961 | PDCD5 | 0.040926508 |
| PVRIG | 0.090265018 | TARDBP | 0.062815268 | ZC3H13 | 0.039334246 |
| RPL24P8 | 0.090263179 | RPL39 | 0.062274182 | AC005884.1 | 0.038868837 |
| SASH3 | 0.088683297 | ENY2 | 0.06209453 | FRG1 | 0.038842744 |
| NDFIP1 | 0.038761753 | MAT2B | 0.010954865 | CFLAR | −0.012715678 |
| NEDD8 | 0.038397505 | CTB-89H12.4 | 0.010821529 | RPS24P8 | −0.013369414 |
| AC007969.5 | 0.038374511 | SLTM | 0.010196189 | KIAA2026 | −0.013943406 |
| SRRM2 | 0.038338342 | PRDX1 | 0.0099881 | ARPC4 | −0.014334415 |
| TCEB1 | 0.037743683 | PTPN2 | 0.009827899 | TANK | −0.014408258 |
| ORMDL1 | 0.037712921 | SNRPB2 | 0.009776683 | PUM2 | −0.014451429 |
| TMEM14B | 0.036594785 | ITK | 0.009737801 | PDCD4 | −0.014874019 |
| ZCCHC11 | 0.036555997 | CHD2 | 0.009713873 | TRAPPC5 | −0.0150988 |
| SRP72 | 0.034402353 | MOB3A | 0.008932209 | EWSR1 | −0.0155502 |
| Z82188.1 | 0.033307447 | HLA-F | 0.0087219 | SERP1 | −0.016642758 |
| IRF3 | 0.032198616 | COMMD6 | 0.008021766 | CCT4 | −0.016670327 |
| POLR2L | 0.031960801 | NIPBL | 0.0068219 | VAMP2 | −0.016700464 |
| TGFBR2 | 0.031403734 | Sep-06 | 0.006185718 | ABCF1 | −0.017223053 |
| PPIA | 0.030597894 | RPS7P10 | 0.005722805 | EIF1 | −0.018428428 |
| NSRP1 | 0.03052581 | USP8 | 0.00441441 | ATG12 | −0.019200245 |
| ZNF655 | 0.030413444 | KRR1 | 0.004334469 | SH3GLB1 | −0.019653456 |
| CLSTN1 | 0.030190966 | BCL11B | 0.002194462 | TMEM50A | −0.019935272 |
| MAP3K2 | 0.029283421 | JTB | 0.001819719 | NMI | −0.020082922 |
| CTDSP1 | 0.029167881 | SRP19 | 0.001293363 | ARL6IP4 | −0.020218201 |
| PAFAH1B1 | 0.028939588 | PFDN5 | 0.001144198 | RPL13AP25 | −0.020649975 |
| DEF6 | 0.028419796 | PHF20L1 | 0.000236008 | REEP5 | −0.02133552 |
| S100A11 | 0.02826972 | RPS27L | −0.000823517 | C19orf66 | −0.021790298 |
| BTF3 | 0.02761011 | SF3B1 | −0.002144295 | ACAP1 | −0.022344379 |
| UBE2D3 | 0.026701524 | UGP2 | −0.002216546 | CIRBP | −0.023031622 |
| CCNL1 | 0.02566998 | RBX1 | −0.002424582 | TUBA4A | −0.023967648 |
| RN7SL3 | 0.024793287 | BPTF | −0.002601679 | ATP6V1E1 | −0.023983981 |
| SNHG5 | 0.024140916 | PSMD7 | −0.003503329 | ARID1A | −0.025101483 |
| RPL15P2 | 0.023340972 | GZMA | −0.004158427 | GOLGA4 | −0.025180935 |
| OGT | 0.023287502 | B2M | −0.005672531 | PRPF38B | −0.026004767 |
| MYH9 | 0.022992128 | LSM3 | −0.006267521 | RPL37P2 | −0.026203044 |
| BTG2 | 0.022874684 | ZBTB4 | −0.006454513 | GOLGA7 | −0.027209403 |
| ST13 | 0.021546424 | APBB1IP | −0.006743845 | BTN3A1 | −0.02807256 |
| CELF1 | 0.02077087 | TMEM59 | −0.006986916 | CDV3 | −0.030008369 |
| CCT5 | 0.020540796 | GIMAP6 | −0.007148931 | SERINC3 | −0.031067606 |
| SKP1 | 0.019809653 | POLR3GL | −0.00785893 | EIF2S2 | −0.031307582 |
| FMNL1 | 0.019678021 | CSNK1D | −0.008289983 | SZRD1 | −0.031977594 |
| MYEOV2 | 0.018856706 | EIF5B | −0.00886603 | HNRNPA3 | −0.032544112 |
| FAIM3 | 0.015992601 | RAB11A | −0.008979241 | PPHLN1 | −0.033783729 |
| DAD1 | 0.015359033 | CCNK | −0.009655606 | RBM8A | −0.034320058 |
| CNOT7 | 0.014633937 | SCARNA9 | −0.009820924 | SF1 | −0.034563195 |
| ATP6V0E1 | 0.013665649 | MPHOSPH8 | −0.010046952 | BCAP31 | −0.035443903 |
| MEAF6 | 0.013032566 | CDC5L | −0.010726421 | ANKRD12 | −0.036038968 |
| C19orf53 | 0.012942939 | CD6 | −0.012116002 | DHX36 | −0.036141879 |
| RAD23A | 0.010954954 | SNX6 | −0.012275943 | ATP2B4 | −0.036281233 |
| SETD2 | −0.037398696 | SEC62 | −0.063851504 | OSBPL8 | −0.093209559 |
| C6orf62 | −0.037737369 | RPS2 | −0.06411251 | RPS3AP47 | −0.093319792 |
| JAK1 | −0.03900138 | ROCK1 | −0.064393921 | SSR1 | −0.093883225 |
| HLA-E | −0.039391871 | ISG20 | −0.065598687 | MANF | −0.096102028 |
| MDM4 | −0.040533527 | IMP3 | −0.065663171 | CSTB | −0.09635792 |
| SVIP | −0.040643888 | TMED2 | −0.067690089 | WAC | −0.096455975 |
| SMAP2 | −0.041022065 | RPL30P4 | −0.067858328 | PCNP | −0.096898523 |
| ERGIC3 | −0.041499903 | KRCC1 | −0.069024246 | THEMIS | −0.098481079 |
| VIM | −0.041539044 | AC079250.1 | −0.070048536 | RN7SL1 | −0.098981497 |
| CAP1 | −0.045378062 | LAMP1 | −0.070340814 | HIST1H1E | −0.10076121 |
| PRF1 | −0.045987136 | RNF7 | −0.070781572 | RPL24 | −0.100968457 |
| RPSAP58 | −0.047146354 | EIF3L | −0.071313518 | SH3BGRL3 | −0.100970387 |
| PTPRC | −0.047580328 | RNPEPL1 | −0.071319784 | ACTB | −0.101772165 |
| PSD4 | −0.047705386 | DERL1 | −0.071603176 | PRKX | −0.102479135 |
| SYTL3 | −0.047945019 | CREBBP | −0.071781376 | OST4 | −0.103730141 |
| SAP18 | −0.049073141 | EEF1D | −0.072467016 | CTD- | −0.103874802 |
| | | | | 2666L21.3 | |
| OPTN | −0.049167769 | CDC42SE2 | −0.073300911 | EML4 | −0.104978591 |
| FAM111A | −0.04960079 | TMEM66 | −0.073502701 | KDM5A | −0.105916999 |
| EIF3J | −0.049707972 | ACTR3 | −0.074027482 | BNIP2 | −0.106129617 |
| HLA-C | −0.050251124 | RAB7A | −0.074289553 | FGFR1OP2 | −0.106315442 |
| FTL | −0.052870009 | CD164 | −0.074994296 | PYHIN1 | −0.106573223 |
| TRGC2 | −0.053667903 | CCT3 | −0.075978703 | ZFP36 | −0.106966454 |
| ERP29 | −0.055401631 | AKNA | −0.077014819 | STK10 | −0.107110288 |
| AKIRIN1 | −0.055745135 | CORO1A | −0.077634737 | EEF2 | −0.109152482 |
| RER1 | −0.056573664 | RPL23 | −0.078822704 | RPL17- | −0.109212835 |
| | | | | C18orf32 | |
| RPL4P2 | −0.057271821 | ACP1 | −0.07955762 | BAZ1A | −0.109883026 |
| WIPF1 | −0.057594992 | ZNF24 | −0.080383433 | TACC1 | −0.110006478 |
| TMOD3 | −0.057672672 | MOB1A | −0.08131844 | CDK13 | −0.111159584 |
| SEC11C | −0.057967924 | GNB2L1 | −0.082179675 | TMED4 | −0.111663182 |
| TTC3 | −0.057967924 | PPP1CB | −0.082201217 | EIF4A2 | −0.112194726 |
| C10orf118 | −0.058340379 | CAPNS1 | −0.082581904 | RABEP1 | −0.112444545 |
| NACA3P | −0.059857311 | SERF2 | −0.082724931 | TSPAN14 | −0.113960615 |
| C11orf58 | −0.060021127 | CHD3 | −0.085463003 | RPL18 | −0.114110165 |
| DDX18 | −0.06004019 | C19orf60 | −0.085920636 | CTC- | −0.115901455 |
| | | | | 260E6.2 | |
| TAF10 | −0.060638964 | CDKN1B | −0.086624861 | MYL12B | −0.117179701 |
| FBXO7 | −0.060966509 | ZBTB7A | −0.087163451 | LSM14A | −0.117766662 |
| EIF3M | −0.061260343 | SSR3 | −0.087529358 | CNN2 | −0.117829629 |
| TMSB4X | −0.062351588 | UBE2Q1 | −0.08947315 | TRAM1 | −0.11825899 |
| RHOF | −0.0627426 | PBXIP1 | −0.090850945 | RPS24 | −0.118581805 |
| FNTA | −0.062872363 | RPL15 | −0.091486361 | SMEK2 | −0.119655545 |
| SLFN12L | −0.063585539 | ACTR2 | −0.091831451 | RBM3 | −0.119754358 |
| TAPBP | −0.063723974 | RPS12P23 | −0.09190132 | TPM3 | −0.119777113 |
| CUTA | −0.063755192 | HNRNPDL | −0.092136116 | DDX21 | −0.11980006 |
| IDS | −0.063851504 | CASP1 | −0.092402279 | RPS9 | −0.121540668 |
| IVNS1ABP | −0.12158007 | NACA | −0.151596608 | TCF25 | −0.180945814 |
| SNX3 | −0.121609463 | CTDNEP1 | −0.154618858 | ABI1 | −0.181662943 |
| CLEC2B | −0.122920895 | CD37 | −0.156561252 | ACTG1 | −0.184532237 |
| RPL36AP21 | −0.123360187 | BIRC2 | −0.157231303 | Sep-15 | −0.185678711 |
| FOXN3 | −0.123467066 | PRDM2 | −0.158660987 | MAN1A2 | −0.185930556 |
| CASP4 | −0.123819949 | ABRACL | −0.158882369 | PTBP3 | −0.186273888 |
| SYNE2 | −0.124035393 | GPATCH8 | −0.159646408 | UBLCP1 | −0.186682452 |
| SEC61G | −0.124207171 | FNBP4 | −0.159676682 | RPL26 | −0.186769584 |
| GPSM3 | −0.124379551 | EIF1AX | −0.1602536 | DDX5 | −0.186988399 |
| BIN1 | −0.125227436 | ZC3H15 | −0.160256906 | TAOK3 | −0.187478157 |
| UFC1 | −0.125291809 | SYAP1 | −0.162041364 | STK4 | −0.188592106 |
| MORF4L1 | −0.126135937 | TNFAIP8 | −0.162208153 | NUDT3 | −0.188805524 |
| JUND | −0.128028965 | CLK1 | −0.162698219 | RPL13AP5 | −0.189380987 |
| C17orf76- | −0.12893012 | MACF1 | −0.162754529 | RPS15 | −0.189440725 |
| AS1 | | | | | |
| AC107983.4 | −0.129538226 | PSAP | −0.163122426 | LUC7L3 | −0.191458984 |
| MAPRE1 | −0.129858004 | EIF3K | −0.163251894 | TMC8 | −0.191966763 |
| YWHAQ | −0.130110034 | EHD1 | −0.163703865 | CAPN2 | −0.192708785 |
| NKG7 | −0.130438624 | EIF2S3 | −0.163902962 | CMPK1 | −0.194677042 |
| PRR13 | −0.130796742 | CIR1 | −0.164026009 | RPS11 | −0.194980043 |
| LAMTOR4 | −0.134628045 | PABPN1 | −0.164547602 | GCC2 | −0.195022324 |
| FAM107B | −0.134697894 | AC009245.3 | −0.164684305 | VPS26A | −0.196000279 |
| RPL13P12 | −0.134722144 | N4BP2L2 | −0.165695528 | RPL28 | −0.197016778 |
| DR1 | −0.136890795 | EIF3E | −0.166240992 | SELK | −0.198198615 |
| BZW1 | −0.13702138 | DNAJC1 | −0.166742608 | RPS28 | −0.198586267 |
| CTSC | −0.13825913 | RHOG | −0.166747616 | VPS4B | −0.198877451 |
| POLR1D | −0.138563442 | PHF3 | −0.166988373 | AC002994.1 | −0.200994224 |
| FXR1 | −0.138800171 | NUB1 | −0.170624903 | CTD- | −0.201224969 |
| | | | | 2031P19.4 | |
| Y_RNA | −0.138927143 | TMA7 | −0.170806026 | RPS4XP11 | −0.201545131 |
| IK | −0.139414778 | GPBP1 | −0.171083144 | IFITM1 | −0.202443669 |
| TM9SF3 | −0.141108094 | RPS19 | −0.17168262 | CNBP | −0.202575159 |
| BOD1L1 | −0.141212614 | GLG1 | −0.172331088 | DAZAP2 | −0.202681623 |
| EVI2B | −0.143209796 | MIER1 | −0.173609885 | OSTC | −0.203288822 |
| FBL | −0.14362595 | ERBB2IP | −0.173722743 | RB1CC1 | −0.204047876 |
| PTPN1 | −0.145750995 | RPS5 | −0.173780158 | APMAP | −0.20537768 |
| IFIT3 | −0.146804479 | PFN1 | −0.174140254 | TMEM30A | −0.205666002 |
| DNAJC8 | −0.14740075 | GIMAP7 | −0.17428042 | ARCN1 | −0.20811445 |
| SHISA5 | −0.147668798 | RBM39 | −0.175638122 | RPL36 | −0.210730161 |
| TMEM248 | −0.148161155 | ARHGAP15 | −0.177722986 | RPS7P1 | −0.211644539 |
| RPS20P10 | −0.148595464 | CARD8 | −0.17834958 | UBE2L3 | −0.213178433 |
| UTRN | −0.149091904 | Sep-09 | −0.178488895 | BBX | −0.214405098 |
| HSPA1A | −0.14913323 | ABHD14B | −0.178733578 | RPS4XP7 | −0.215281803 |
| IL27RA | −0.150107847 | RPL8 | −0.178814725 | NME2 | −0.215675151 |
| AP000936.1 | −0.150443504 | ZFC3H1 | −0.180140888 | SNX5 | −0.218087705 |
| RPL36AL | −0.15119346 | MED15 | −0.180195744 | CTB-79E8.3 | −0.218518674 |
| FXYD5 | −0.219351533 | CTB-36O1.7 | −0.263001729 | CYTIP | −0.310302171 |
| RPS2P5 | −0.220331891 | MIR3654 | −0.263542682 | RPL34P31 | −0.310721325 |
| SSR2 | −0.221608698 | SSB | −0.264922036 | C9orf78 | −0.31189415 |
| GTF3A | −0.221685178 | RASA2 | −0.265325831 | RPL7A | −0.31275119 |
| PRKACB | −0.222712831 | DOCK11 | −0.265485377 | RTN4 | −0.314300532 |
| CCDC88C | −0.223180899 | SPOCK2 | −0.265601897 | AUP1 | −0.314466466 |
| RPLP0 | −0.223279724 | EIF3F | −0.266478943 | UXT | −0.314975237 |
| USP34 | −0.224805723 | IRF2BP2 | −0.266962959 | RPL24P4 | −0.315978041 |
| RPS17L | −0.225225183 | TRIM38 | −0.27114843 | FBXW7 | −0.317509859 |
| KIAA1109 | −0.225335468 | ARPC5 | −0.272953339 | RPS8 | −0.318182689 |
| SEPW1 | −0.226941559 | TMED5 | −0.273058482 | PPP2R5C | −0.319603403 |
| TMEM230 | −0.227105375 | CD97 | −0.273305799 | RPS29 | −0.321786177 |
| KAT2B | −0.227525436 | LGALS8 | −0.274578987 | GAS5 | −0.324742248 |
| TRAF3IP3 | −0.228006404 | TBC1D10C | −0.27472127 | TCP1 | −0.324829087 |
| UBA52 | −0.228416064 | KMT2C | −0.275546829 | POLE3 | −0.324890498 |
| MBNL1 | −0.229118933 | RPS20P14 | −0.276016714 | TMED10 | −0.325736583 |
| PPP1R18 | −0.234681037 | RPL4 | −0.276710928 | LITAF | −0.325938927 |
| EIF3G | −0.235069861 | EPRS | −0.28030505 | RPS7 | −0.327699603 |
| EEFIA1 | −0.235303198 | SAR1A | −0.280414393 | SERINC1 | −0.328704591 |
| LIME1 | −0.238075392 | RPL7AP6 | −0.281465502 | RPS6 | −0.332389009 |
| RPL13 | −0.240291423 | RPS16 | −0.281628935 | PRR5L | −0.332558036 |
| IER2 | −0.240612338 | TMSB10 | −0.281749382 | RPL11 | −0.333188387 |
| ARHGEF3 | −0.241111029 | SYF2 | −0.282363097 | GIMAP1 | −0.334896758 |
| KCNAB2 | −0.243722341 | YIPF4 | −0.283038248 | RPL32 | −0.337468933 |
| MTPN | −0.245460018 | HSPA1B | −0.283655145 | ZC3HAV1 | −0.337631997 |
| TRAPPC10 | −0.246811714 | HIST1H1D | −0.283785417 | SLC9A3R1 | −0.338404542 |
| SLC2A3 | −0.24821468 | CYFIP2 | −0.286467763 | RPS4Y1 | −0.338906161 |
| RPS13 | −0.248799118 | EMB | −0.287425109 | RPL37P23 | −0.33994822 |
| DIAPH1 | −0.250676191 | DUSP1 | −0.288644193 | RWDD1 | −0.340369336 |
| FAM204A | −0.250740522 | IKZF1 | −0.290300258 | CYBA | −0.342016405 |
| SSBP4 | −0.250893389 | RPL29 | −0.293134432 | FTH1P8 | −0.342133345 |
| TAPSAR1 | −0.251065027 | RPS4X | −0.294921021 | GIMAP2 | −0.343693458 |
| RCSD1 | −0.251309619 | CCT2 | −0.295357204 | CTSW | −0.344433828 |
| ATXN7L3B | −0.251958976 | HMHA1 | −0.295389994 | TPM4 | −0.345947599 |
| ITM2B | −0.254045123 | LTB | −0.29664062 | GIMAP5 | −0.347025418 |
| ARHGDIB | −0.255130822 | H3F3AP6 | −0.296956226 | RPL6 | −0.347056726 |
| ZAP70 | −0.255963101 | CTBP1 | −0.297066795 | CTD- | −0.348848562 |
| | | | | 2248H3.1 | |
| GLTSCR2 | −0.256536249 | RPL27 | −0.29842396 | HNRNPH3 | −0.34960096 |
| ARFGEF1 | −0.256882983 | YWHAZ | −0.299657535 | TMC6 | −0.350286646 |
| APRT | −0.257193446 | ATP6V1G1 | −0.300452772 | TNRC6B | −0.350799523 |
| FAU | −0.257302852 | SEC31A | −0.301193633 | ABHD17A | −0.352364961 |
| INPP5D | −0.258894485 | DNAJC3 | −0.30470924 | PIK3R1 | −0.352367914 |
| RPL19 | −0.261886679 | PPCS | −0.307776947 | RPL35 | −0.35346904 |
| FAM49B | −0.262974567 | ORAI1 | −0.3095392 | ACAP2 | −0.355022173 |
| TBCA | −0.356257272 | PABPC1 | −0.399541338 | GLIPR1 | −0.496231063 |
| TAGLN2 | −0.357358971 | ITGB2 | −0.403724103 | RUNX3 | −0.496579207 |
| CDC42 | −0.358108273 | AC007387.2 | −0.403786531 | C16orf54 | −0.498090567 |
| CD44 | −0.358946364 | PPP2R5E | −0.40413355 | EEF1A1P5 | −0.50000952 |
| RPS27A | −0.359326551 | AC073610.5 | −0.404957188 | RPS3AP6 | −0.500033907 |
| BTG1 | −0.359572701 | ARIH2 | −0.405972077 | EIF4EBP2 | −0.500722325 |
| CTB-63M22.1 | −0.360443861 | RPL22 | −0.406320842 | UCHL5 | −0.502398902 |
| HECA | −0.361548025 | ERH | −0.4130443 | RPL5P34 | −0.510281152 |
| RPS7P11 | −0.361744555 | THUMPD1 | −0.414779474 | STIM2 | −0.512925218 |
| RPL35P5 | −0.363854577 | RPL5P17 | −0.416500556 | RPL34P18 | −0.514295577 |
| RPL37 | −0.364068248 | AB019441.29 | −0.419315073 | SC22CB- | −0.519881101 |
| | | | | 1E7.1 | |
| CHMP4B | −0.364343787 | CHURC1 | −0.422231738 | CTD- | −0.520635164 |
| | | | | 3035D6.1 | |
| DCAF5 | −0.366730799 | UBE2B | −0.424956806 | RPL10P3 | −0.525284451 |
| PTPN18 | −0.368280268 | RPS4XP6 | −0.425443121 | RPS20 | −0.533255911 |
| PFNIPI | −0.368744698 | C12orf75 | −0.433479927 | EIF4B | −0.533440201 |
| TC2N | −0.37003223 | RPL31 | −0.43429294 | ZRANB2 | −0.533743912 |
| DNAJA2 | −0.370569027 | GLRX | −0.439300585 | RPL6P27 | −0.538367849 |
| RPS23P8 | −0.371150898 | RPL37A | −0.442077183 | PCSK7 | −0.540501279 |
| SEC63 | −0.371616662 | LAT | −0.443458511 | VASP | −0.540620986 |
| ZFAS1 | −0.372587831 | OFD1 | −0.444224074 | CALM1 | −0.542949477 |
| RPSA | −0.373012718 | OTUB1 | −0.445001729 | RPS3 | −0.544583766 |
| PIN1 | −0.374535165 | DICER1 | −0.448035883 | RPL36A- | −0.545292364 |
| | | | | HNRNPH2 | |
| RPL18AP3 | −0.375433413 | CTD-2192J16.15 | −0.449594972 | DIP2A | −0.547308309 |
| HOPX | −0.376528944 | RSL24D1 | −0.450922135 | MYO1G | −0.550959474 |
| RPL27A | −0.377772021 | SNORA52 | −0.45589142 | SELPLG | −0.55168156 |
| THOC7 | −0.378018027 | TTC14 | −0.456906815 | MIR142 | −0.558066774 |
| ITGAL | −0.380932577 | RPL23A | −0.459197855 | RPL31P7 | −0.55816466 |
| MLLT6 | −0.382420422 | STK39 | −0.466777449 | AL161626.1 | −0.558179008 |
| RPLP2 | −0.382553276 | SLAMF7 | −0.468207641 | KLRD1 | −0.560502179 |
| NFATC3 | −0.384049221 | ZNF33A | −0.469326351 | RPL30 | −0.561155983 |
| ANXA2 | −0.386048512 | RPS12 | −0.470744295 | GPR56 | −0.56133914 |
| FGD3 | −0.387623409 | RPL34P34 | −0.472622763 | SPN | −0.563471577 |
| AES | −0.387639784 | CD53 | −0.475938556 | MYO1F | −0.564436468 |
| RPS17 | −0.389423746 | RNU4ATAC | −0.476978087 | TAGAP | −0.565442062 |
| UFM1 | −0.390415053 | RPS23 | −0.477394261 | ARRB2 | −0.566035484 |
| RBL2 | −0.391717547 | SP140L | −0.477783169 | AD000092.3 | −0.566809964 |
| HCST | −0.392143307 | RPS21 | −0.479033914 | FLNA | −0.569143069 |
| RPL38 | −0.393335811 | ARHGAP25 | −0.480066448 | RPL9P3 | −0.574133513 |
| RAB7L1 | −0.394660391 | RPS3AP26 | −0.484455302 | RPL7AP30 | −0.574350599 |
| TPT1 | −0.395874419 | RPA2 | −0.484906287 | XBP1 | −0.574782928 |
| SCFD1 | −0.396394599 | RNF125 | −0.485039157 | RBMS1 | −0.57619459 |
| PCED1B-AS1 | −0.397901124 | RPL5 | −0.487037702 | AL590762.1 | −0.5764221 |
| PIP4K2A | −0.398286821 | EIF4E3 | −0.488849707 | PRKCB | −0.578155694 |
| GNPTAB | −0.399422527 | FAUP1 | −0.493343397 | RPS25 | −0.580554197 |
| SNORD100 | −0.58220023 | IFITM2 | −0.670686661 | ADAM8 | −0.825269248 |
| MYL12A | −0.582771606 | FOS | −0.673433452 | SIGIRR | −0.833731267 |
| SIPA1 | −0.584897652 | PLEKHB2 | −0.681668338 | PLEKHA2 | −0.839042314 |
| SUN2 | −0.585136068 | SNHG8 | −0.695961478 | S100A4 | −0.841020512 |
| GRK6 | −0.589466476 | FAM129A | −0.698234346 | TMEM123 | −0.842897864 |
| RPL14 | −0.59008004 | AC022431.1 | −0.69857368 | RPL10 | −0.845234718 |
| LGALS1 | −0.595719441 | SNHG6 | −0.698822917 | ITGB1 | −0.845683317 |
| FLI1 | −0.596180878 | SNRK | −0.704268205 | AHNAK | −0.859507184 |
| GVINP1 | −0.596331935 | RPL5P1 | −0.70937821 | FUT11 | −0.864078312 |
| GNG2 | −0.597687614 | CD247 | −0.709958047 | SYVN1 | −0.86793939 |
| RPS10 | −0.600311759 | EEF1B2 | −0.71040053 | ATM | −0.877577565 |
| FOXP1 | −0.600402865 | MAPK1 | −0.712218527 | STAT4 | −0.888586443 |
| RGS19 | −0.605813217 | RPL15P18 | −0.714258581 | PIM1 | −0.90799456 |
| RPL14P1 | −0.60602406 | EPB41 | −0.714472359 | GPR65 | −0.909871415 |
| GRAP2 | −0.610420938 | CD69 | −0.717470849 | LPCAT1 | −0.909977135 |
| RASSF1 | −0.612688241 | PRMT2 | −0.723038476 | AIM1 | −0.919403438 |
| TSC22D3 | −0.615274117 | S100A6 | −0.727843176 | PICALM | −0.923473932 |
| DHRS7 | −0.623628873 | SAMHD1 | −0.730009225 | CTB-13H5.1 | −0.932207045 |
| SNORA21 | −0.62691957 | CD52 | −0.732758071 | CRBN | −0.935918926 |
| RPL18A | −0.628322526 | CYTH1 | −0.734132177 | SAMD3 | −0.940268415 |
| RAB2A | −0.630585899 | KLF6 | −0.734514718 | RPS26P3 | −0.955180916 |
| RPL5P4 | −0.631338004 | RPL4P4 | −0.735719058 | RPS18P12 | −0.976116547 |
| AC004453.8 | −0.637981458 | CSNK1G2 | −0.736540976 | MIR4426 | −0.986216635 |
| CD48 | −0.638514194 | ZEB2 | −0.744273126 | RORA | −1.011381106 |
| RPS12P26 | −0.639841255 | MYADM | −0.74630447 | SNORD54 | −1.023561261 |
| RPL23P8 | −0.640779211 | CD5 | −0.747964528 | TGFB1 | −1.032744394 |
| KLF13 | −0.641312841 | GZMH | −0.748507537 | S1PR4 | −1.04680028 |
| PTPRE | −0.645341521 | SNORD33 | −0.757431514 | ARL4C | −1.047573013 |
| RAP1B | −0.65206146 | RPL3P4 | −0.758710036 | ADD3 | −1.074733464 |
| PARP8 | −0.652583813 | RPS3AP25 | −0.766671093 | RPS3A | −1.091806291 |
| CAST | −0.654455736 | TRGC1 | −0.768882262 | RASA3 | −1.117467173 |
| IQGAP2 | −0.655475282 | SYNE1 | −0.776726743 | CISH | −1.121199368 |
| GLIPR2 | −0.65740651 | SYTL1 | −0.788414043 | GLUL | −1.123797764 |
| IFITM3 | −0.660585759 | TPST2 | −0.789248047 | KLRG1 | −1.126206038 |
| DOK2 | −0.661249412 | ST6GAL1 | −0.79169706 | TRDC | −1.175148522 |
| RPL13A | −0.663177113 | ZFP36L2 | −0.794711311 | FLT3LG | −1.195045669 |
| CTC- | −0.665145234 | SYTL2 | −0.799462016 | FAM101B | −1.195185566 |
| 575D19.1 | | | | | |
| RPL17 | −0.665440982 | MBP | −0.799765267 | RPL3P2 | −1.211121384 |
| CD47 | −0.666468952 | CDC25B | −0.800933715 | TES | −1.219027083 |
| KLRC3 | −0.666528125 | RPL39P3 | −0.802602245 | BIN2 | −1.22399095 |
| RPS15AP24 | −0.666945763 | CCND3 | −0.815121492 | EMP3 | −1.238574249 |
| MIR4461 | −0.667423432 | CDC42SE1 | −0.819824071 | DSTN | −1.256907828 |
| RPS11P5 | −0.667506025 | RPS3AP5 | −0.822432246 | TBX21 | −1.257847021 |
| RPS27 | −0.670207707 | ZNF91 | −0.824138188 | RPL9 | −1.257929751 |
| SCARNA17 | −1.258055987 | FGFBP2 | −3.625303303 | | |
| EFHD2 | −1.284676374 | CX3CR1 | −3.89599549 | | |
| ANXA1 | −1.319255793 | | | | |
| RPL3 | −1.321233896 | | | | |
| S100A10 | −1.333249522 | | | | |
| TXNIP | −1.336561642 | | | | |
| LYAR | −1.34307284 | | | | |
| ZNF683 | −1.344891044 | | | | |
| STK38 | −1.373902943 | | | | |
| RAP2B | −1.390194315 | | | | |
| CDC42EP3 | −1.412009563 | | | | |
| RPS4XP22 | −1.415835472 | | | | |
| LINC00861 | −1.477545987 | | | | |
| TCF7 | −1.547463756 | | | | |
| RPS26P13 | −1.594276603 | | | | |
| KLRB1 | −1.61268254 | | | | |
| PLEK | −1.653288825 | | | | |
| PTGER2 | −1.680635716 | | | | |
| SELL | −1.681257967 | | | | |
| FCRL6 | −1.700521674 | | | | |
| MGAT4A | −1.712793728 | | | | |
| MATK | −1.723951247 | | | | |
| LCP1 | −1.736741652 | | | | |
| FAM65B | −1.952662791 | | | | |
| PXN | −1.960906257 | | | | |
| SPON2 | −1.973436319 | | | | |
| C1orf21 | −2.000006077 | | | | |
| FGR | −2.160079299 | | | | |
| SSR4 | −2.230095632 | | | | |
| KIR2DL1 | −2.255399813 | | | | |
| SORL1 | −2.273390624 | | | | |
| FCGR3A | −2.359501383 | | | | |
| MYBL1 | −2.465472748 | | | | |
| KLF2 | −2.480976663 | | | | |
| GNLY | −2.511289185 | | | | |
| C1orf162 | −2.636492352 | | | | |
| IL7R | −2.702324738 | | | | |
| TGFBR3 | −2.726648453 | | | | |
| CD300A | −2.828201035 | | | | |
| S1PR1 | −2.895840659 | | | | |
| PLAC8 | −2.953691051 | | | | |
| S1PR5 | −3.362079875 | | | | |
| TYROBP | −3.428687971 | | | | |
| KLRF1 | −3.573438845 | | | | |

According to a specific embodiment, the cells are tumor infiltrating lymphocytes (TILs).

Methods of isolating TILs are well known in the art. Typically, this is done by dissociating the tumor tissue in the presence of a protease following by a centrifugation on a discontinuous Percoll gradient (e.g., GE Healthcare). Isolated cells are then used in various assays of T cell function, expression, cell cycle or a combination of same. Other exemplary methods of isolating TILs are further described hereinbelow.

According to a specific embodiment, following isolation, the preparation is essentially free of tumor cells e.g., less than 20%, 10% or 5% tumor cells are in the preparation.

Methods of determining gene expression profiles can be performed at the RNA or protein level.

Below is a more detailed description of methods that can be used to analyze expression of a plurality of genes on the single cell level.

Methods of Analyzing and/or Quantifying RNA

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Single Cell Transcriptome Analysis

This method relies on sequencing the transcriptome of a single cell. In one embodiment a high-throughput method is used, where the RNAs from different cells are tagged individually, allowing a single library to be created while retaining the cell identity of each read. The method can be carried out a number of ways—see for example US Patent Application No. 20100203597 and US Patent Application No. 20180100201, the contents of which are incorporated herein by reference.

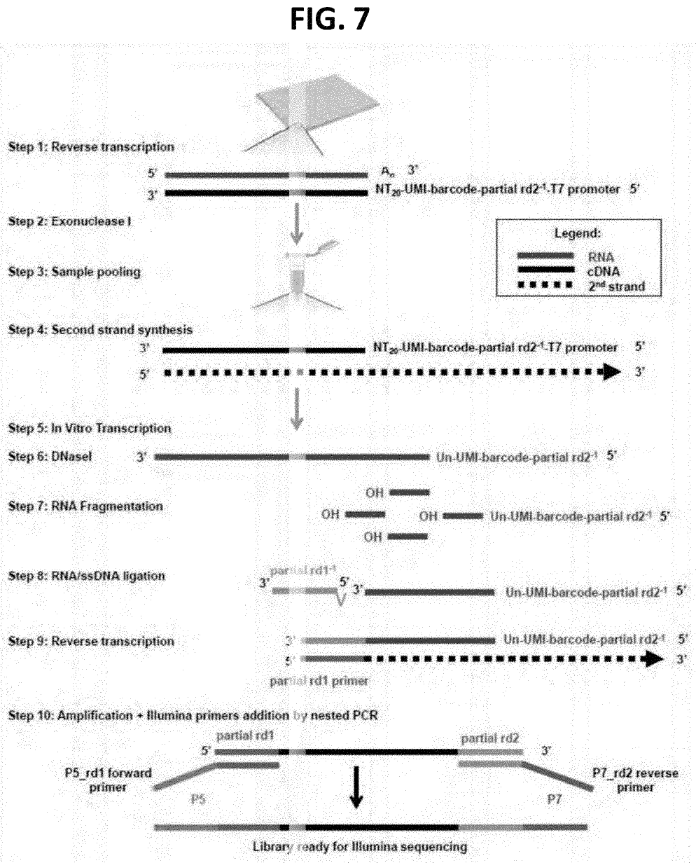

One particular method for carrying out single cell transcriptome analysis is summarized in FIG. 7 and detailed herein below.

Cells are typically aliquoted into wells such that only one cell is present per well. Cells are treated with an agent that disrupts the cell and nuclear membrane making the RNA of the cell accessible to sequencing reactions.

According to one embodiment, the RNA is amplified using the following in vitro transcription amplification protocol:

(Step 1) contacting the RNA of a single cell with an oligonucleotide comprising a polydT sequence at its terminal 3′ end, a T7 RNA polymerase promoter sequence at its terminal 5′ end and a barcode sequence positioned between the polydT sequence and the RNA polymerase promoter sequence under conditions that allow synthesis of a single stranded DNA molecule from the RNA, wherein the barcode sequence comprises a cell barcode and a molecular identifier;

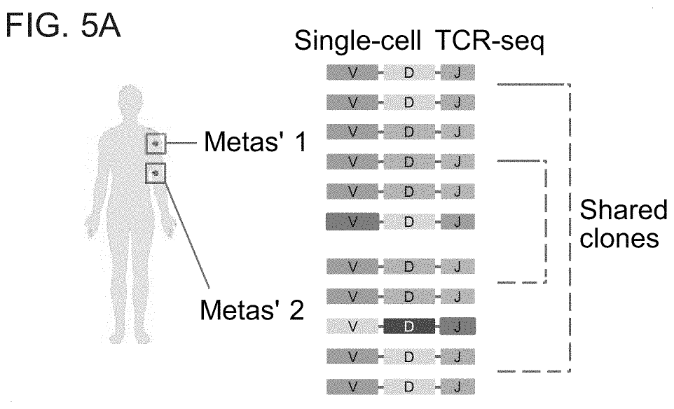

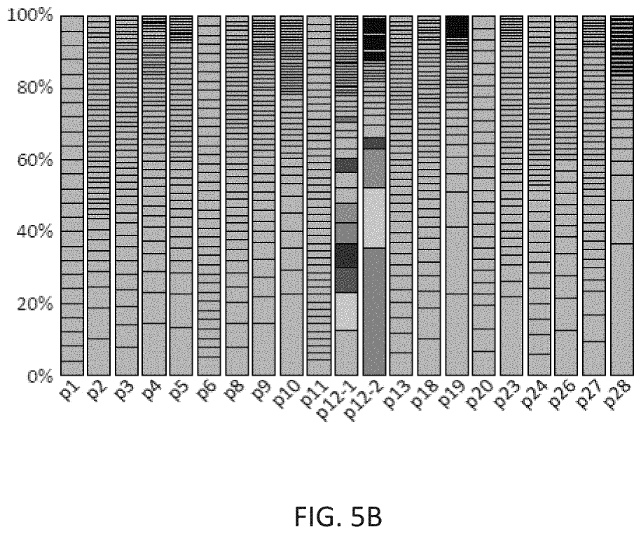

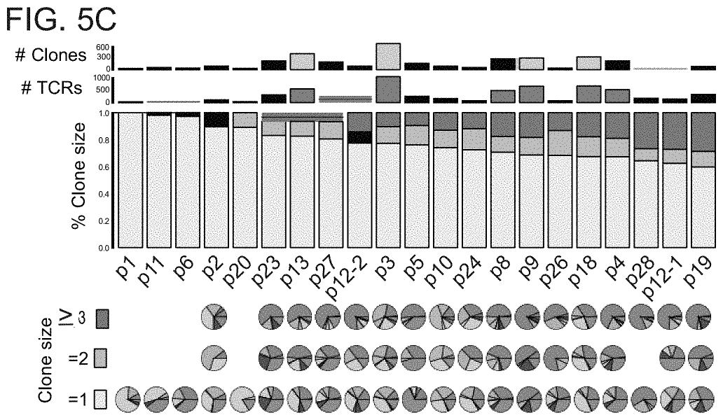

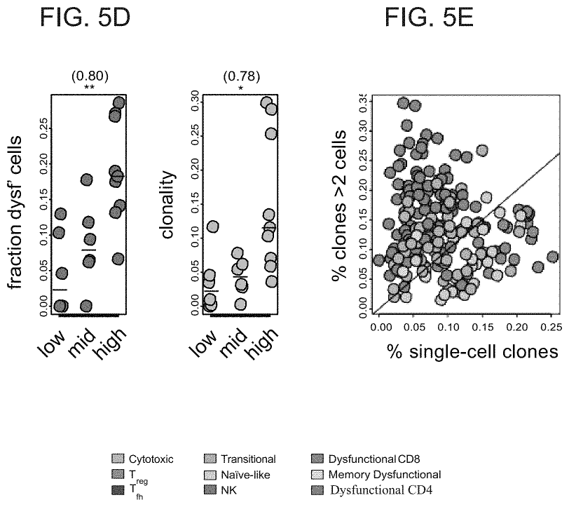

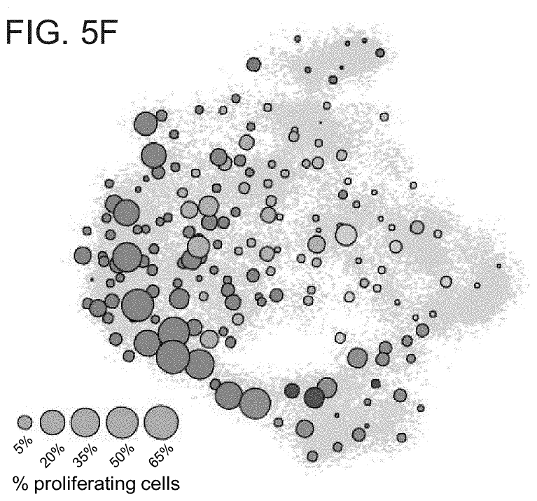

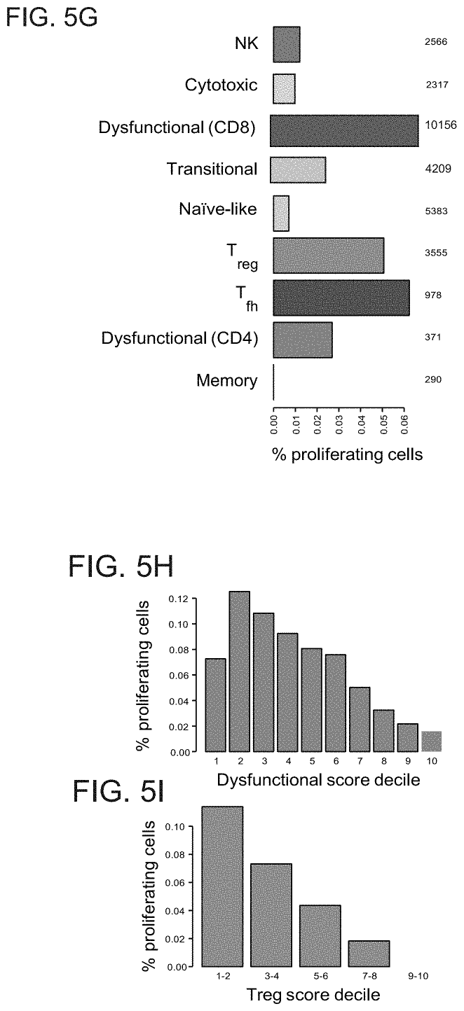

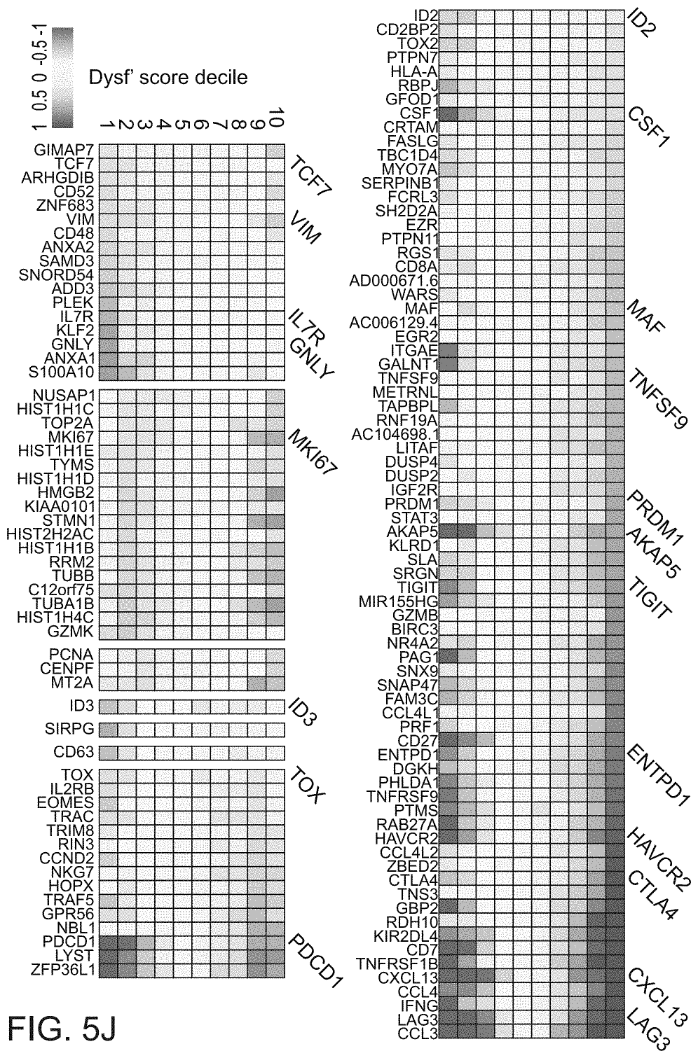

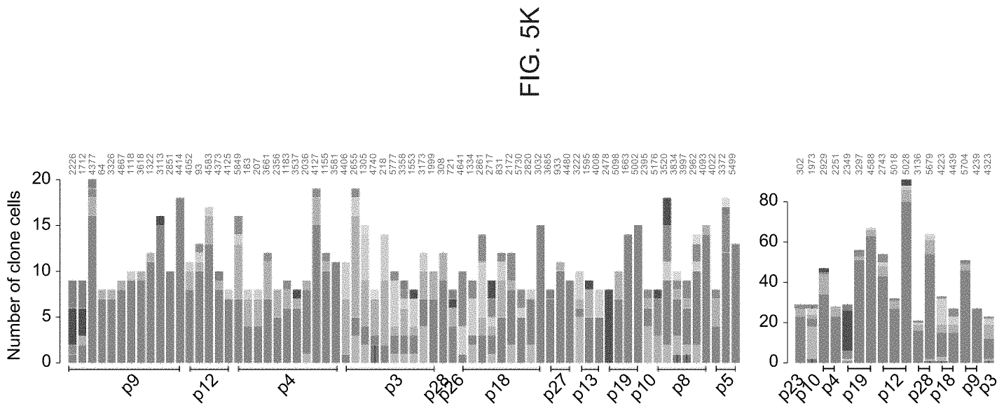

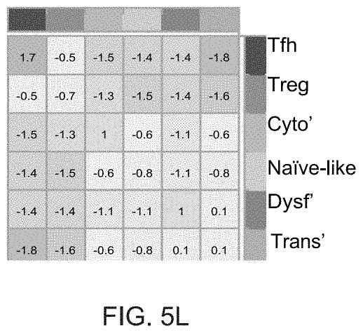

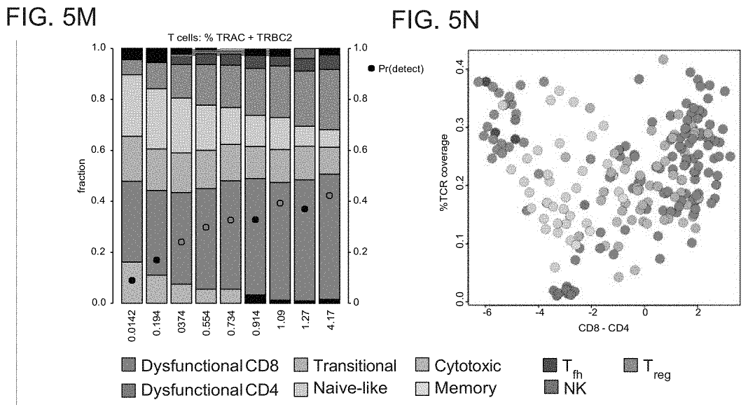

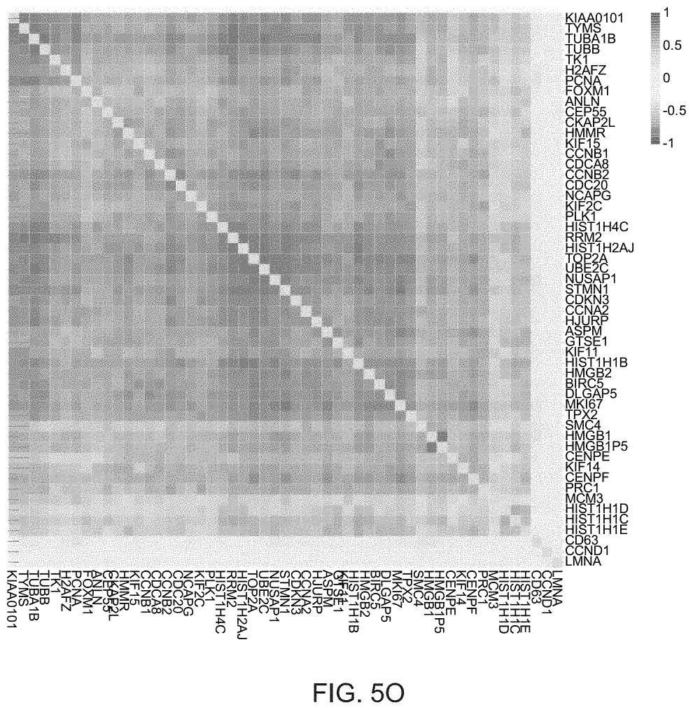

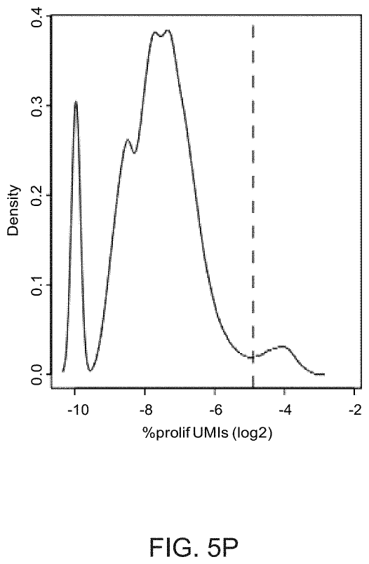

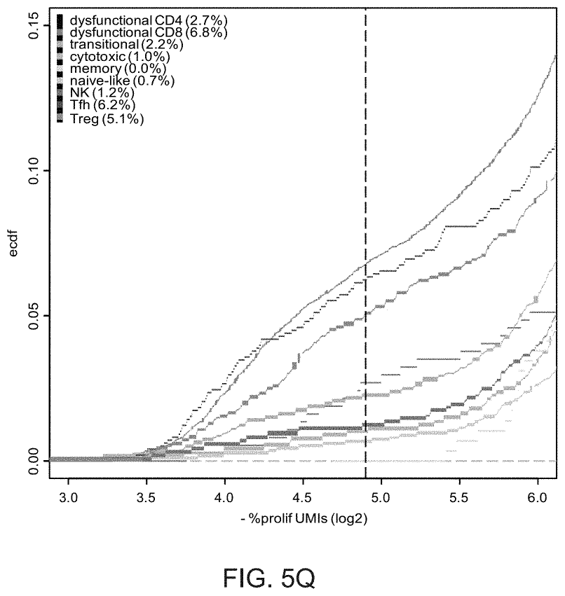

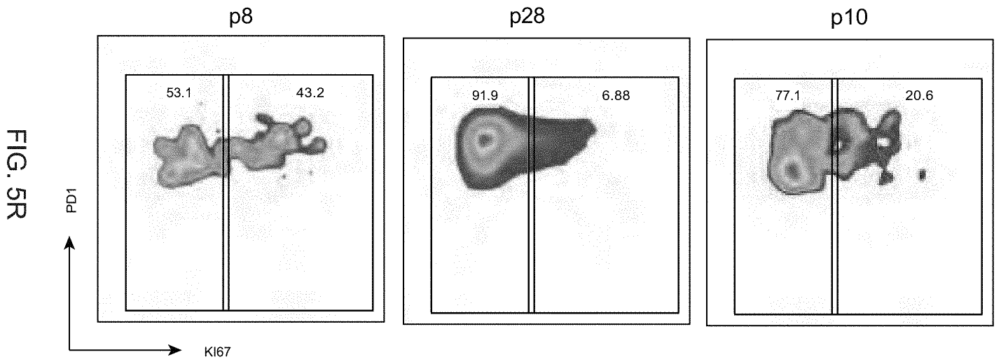

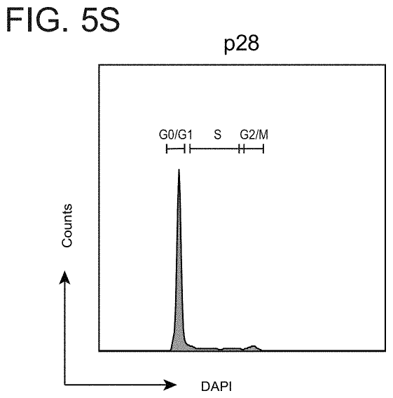

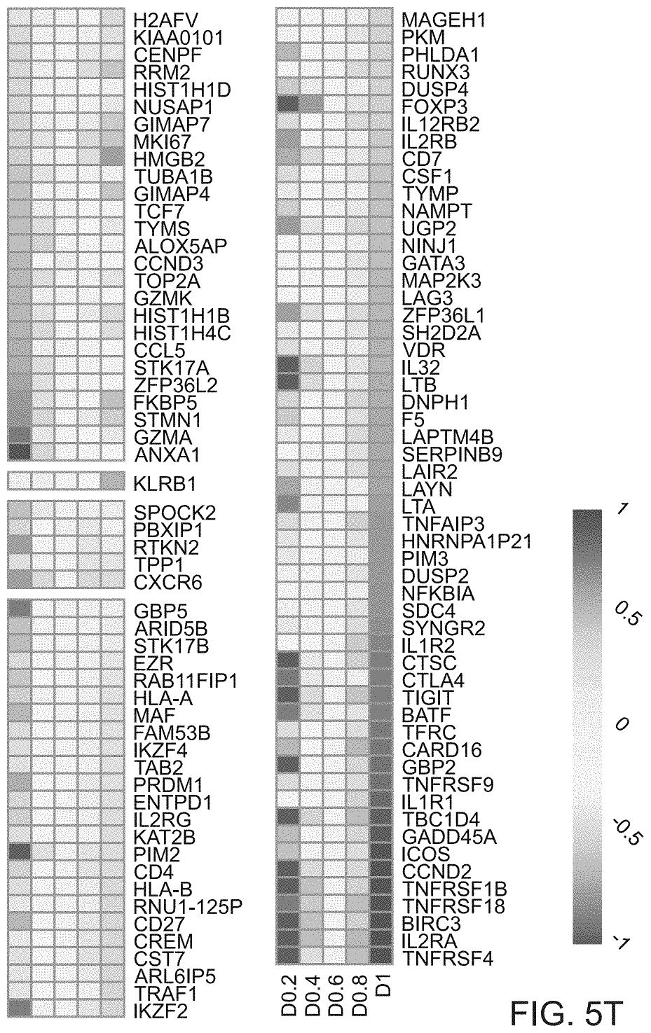

The polydT oligonucleotide of this embodiment may optionally comprise an adapter sequence required for sequencing—see for example FIG. 5.

RNA polymerase promoter sequences are known in the art and include for example T7 RNA polymerase promoter sequence—e.g.

(SEQ ID NO: 63)
SCGATTGAGGCCGGTAATACGACTCACTATAGGGGC.

Preferably the polydT sequence comprises at least 5 nucleotides. According to another embodiment the polydT sequence is between about 5 to 50 nucleotides, more preferably between about 5-25 nucleotides, and even more preferably between about 12 to 14 nucleotides.

The barcode sequence is useful during multiplex reactions when a number of samples are pooled in a single reaction. The barcode sequence may be used to identify a particular molecule, sample or library. The barcode sequence is attached 5′ end of polydT sequence and 3′ of the T7 RNA polymerase sequence. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides.

In one embodiment, the barcode sequence is used to identify a cell type, or a cell source (e.g. a patient).

Molecular identifiers are useful to correct for amplification bias, which reduces quantitative accuracy of the method. The molecular identifier comprises between 4-20 bases. The molecular identifier is of a length such that each RNA molecule of the sample is catalogued (labeled) with a molecular identifier having a unique sequence.

Following annealing of a primer (e.g. polydT primer) to the RNA sample, an RNA-DNA hybrid may be synthesized by reverse transcription using an RNA-dependent DNA polymerase. Suitable RNA-dependent DNA polymerases for use in the methods and compositions of the invention include reverse transcriptases (RTs). RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules. However, in some instances, it is preferable to employ a RT which lacks or has substantially reduced RNase H activity.

RTs devoid of RNase H activity are known in the art, including those comprising a mutation of the wild type reverse transcriptase where the mutation eliminates the RNase H activity. Examples of RTs having reduced RNase H activity are described in US20100203597. In these cases, the addition of an RNase H from other sources, such as that isolated from E. coli, can be employed for the formation of the single stranded cDNA. Combinations of RTs are also contemplated, including combinations of different non-mutant RTs, combinations of different mutant RTs, and combinations of one or more non-mutant RT with one or more mutant RT.

Examples of suitable enzymes include, but are not limited to AffinityScript from Agilent or Superscript III from Invitrogen. Preferably the reverse transcriptase is devoid of terminal Deoxynucleotidyl Transferase (TdT) activity.

Additional components required in a reverse transcription reaction include dNTPS (dATP, dCTP, dGTP and dTTP) and optionally a reducing agent such as Dithiothreitol (DTT) and MnCl₂.

The polydT oligonucleotide may be attached to a solid support (e.g. beads) so that the cDNA which is synthesized may be purified.

Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

(Step 2): Once cDNA is generated, the cDNA may be pooled from cDNA generated from other single cells (using the same method as described herein above).

The sample may optionally be treated with an enzyme to remove excess primers, such as exonuclease I. Other options of purifying the single stranded DNA are also contemplated including for example the use of paramagnetic microparticles. This may be carried out following or prior to sample pooling.

(Step 3): Second strand synthesis.

Second strand synthesis of cDNA may be effected by incubating the sample in the presence of nucleotide triphosphates and a DNA polymerase. Commercial kits are available for this step which include additional enzymes such as RNAse H (to remove the RNA strand) and buffers. This reaction may optionally be performed in the presence of a DNA ligase. Following second strand synthesis, the product may be purified using methods known in the art including for example the use of paramagnetic microparticles.

(Step 4): Following synthesis of the second strand of the cDNA, RNA may be synthesized by incubating with a corresponding RNA polymerase. Commercially available kits may be used such as the T7 High Yield RNA polymerase IVT kit (New England Biolabs).

(Step 5): Prior to fragmentation of the amplified RNA, the DNA may be removed using a DNAse enzyme. The RNA may be purified as well prior to fragmentation. Fragmentation of the RNA may be carried out as known in the art. Fragmentation kits are commercially available such as the Ambion fragmentation kit.

(Step 6): The amplified and fragmented RNA is now labeled on its 3′ end. For this a ligase reaction is performed which essentially ligates single stranded DNA (ssDNA) to the RNA. Other methods of labeling the amplified and fragmented RNA are described in US Application No. 20170137806, the contents of which are incorporated herein by reference. The single stranded DNA has a free phosphate at its 5′end and optionally a blocking moiety at its 3′end in order to prevent head to tail ligation. Examples of blocking moieties include C3 spacer or a biotin moiety. Typically, the ssDNA is between 10-50 nucleotides in length and more preferably between 15 and 25 nucleotides.

(Step 7): Reverse transcription is then performed using a primer that is complementary to the primer used in the preceding step. The library may then be completed and amplified through a nested PCR reaction as illustrated in FIG. 5.

(Step 8): Amplification

Once the adapter polynucleotide of the present invention is ligated to the single stranded DNA (i.e. further to extension of the single stranded DNA), amplification reactions may be performed.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the adapter-extended DNA described herein together with any additional amplification reaction reagents under conditions which allow amplification of the target sequence.

The terms “forward primer” and “forward amplification primer” are used herein interchangeably, and refer to a primer that hybridizes (or anneals) to the target (template strand).

The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphosphate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favor solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England

Biolabs).

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Amplification products obtained using primers of the present invention may be detected using agarose gel electrophoresis and visualization by ethidium bromide staining and exposure to ultraviolet (UV) light or by sequence analysis of the amplification product.

According to one embodiment, the amplification and quantification of the amplification product may be effected in real-time (qRT-PCR).

(Step 9): Sequencing

Methods for sequence determination are generally known to the person skilled in the art. Preferred sequencing methods are next generation sequencing methods or parallel high throughput sequencing methods e.g. Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

It will be appreciated that methods which rely on microfluidics can also be used to carry out single cell transcriptome analysis.

Thus, a combination of molecular barcoding and emulsion-based microfluidics to isolate, lyse, barcode, and prepare nucleic acids from individual cells in high-throughput may be used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode about 10,000-100,000 cells. In this regard there can be a single-cell sequencing library which may comprise: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells: and preparing and sequencing a single composite RNA-Seq library, as described herein above. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2, all the contents and disclosure of each of which are herein incorporated by reference in their entirety.

Methods of Detecting Expression and/or Activity of Proteins

Expression and/or activity level of proteins expressed in the cells of the cultures of some embodiments of the invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to a specific embodiment, the gene expression is determined by transcriptome analysis.

According to a specific embodiment, the gene expression is determined by a single cell transcriptome analysis as described above.

According to a specific embodiment, the cells are determined by the level of at least one (e.g., 2, 3, 4, 5, 6, 7 or 8) but not more than 10 markers (e.g., 1-10, 2-10, 3-10, 4-10, 5-10, 6-10, 7-10, 8-10, 1-5, 2-5, 3-5, 4-5, 1-8, 2-8, 3-8, 4-8, 5-8), e.g., CD8⁺/Lag3⁺/PD1⁺/Tim3⁺/CD103⁺/CD39⁺/CD137⁺/Klrg1⁻. Positive (+) and negative (−) expression are determined as known in the art.

According to a specific embodiment, the marker is PD-1.

According to a specific embodiment, the markers are PD-1 and CD103.

According to a specific embodiment, the cells are in a proliferative state (G₂/M).

According to a specific embodiment, the cells occupy all stages of the cell cycle (G0/G1, S, and M), in stark contrast to cell cycle arrest (FIG. 5M-T).

Methods of determining cellular proliferation and cell cycle are well known in the art. Some are detailed hereinbelow and in the Examples section which follows.

Cell cycle analysis by DNA content measurement is a method that most frequently employs flow cytometry to distinguish cells in different phases of the cell cycle. Before analysis, the cells are usually permeabilised and treated with a fluorescent dye that stains DNA quantitatively, such as propidium iodide (PI) or 4,6-diamidino-2-phenylindole (DAPI). The fluorescence intensity of the stained cells correlates with the amount of DNA they contain. As the DNA content doubles during the S phase, the DNA content (and thereby intensity of fluorescence) of cells in the G₀ phase and G₁ phase (before S), in the S phase, and in the G₂ phase and M phase (after S) identifies the cell cycle phase position in the major phases (G₀/G₁ versus S versus G₂/M phase) of the cell cycle. The cellular DNA content of individual cells is often plotted as their frequency histogram to provide information about relative frequency (percentage) of cells in the major phases of the cell cycle.

Multiparameter analysis of the cell cycle includes, in addition to measurement of cellular DNA content, other cell cycle related constituents/features. The concurrent measurement of cellular DNA and RNA content, or DNA susceptibility to denaturation at low pH using the metachromatic dye acridine orange, reveals the G_{1Q}, G_{1A}, and G_{1B} cell cycle compartments and also makes it possible to discriminate between S, G₂ and mitotic cells. The cells in G_{1Q} are quiescent, temporarily withdrawn from the cell cycle (also identifiable as G₀), the G_{1A} are in the growth phase while G_{1B} are the cells just prior entering S, with their growth (RNA and protein content, size) similar to that of the cells initiating DNA replication. Similar cell cycle compartments are also recognized by multiparameter analysis that includes measurement of expression of cyclin D1, cyclin E, cyclin A and cyclin B1, each in relation to DNA content. Concurrent measurement of DNA content and of incorporation of DNA precursor 5-bromo-T-deoxyuridine (BrdU) by flow cytometry is an especially useful assay.

According to a specific embodiment, the cell proliferative state can also be determined by proliferation markers such as KI67 or MCM-2.

As used herein, expression of inhibitory receptor molecules include, but are not limited to, PD-1, LAG-3, TIM3, TIGIT, CD103, CD39, CD137.

Methods of determining a dysfunctional phenotype are well known in the art. These include, secretion of cytokines that are in direct correlation with cytotoxic T cell function. Examples include, but are not limited to, interleukin 2 (IL-2), tumor necrosis factor (TNF), and interferon gamma (IFNgamma, IFNg). Oftentimes, a dysfunctional phenotype is determined in the presence of the tumor cells or antigens thereof, as further described hereinbelow.

Products for determining T cell exhaustion are commercially available. Examples include, but are not limited to, the kits available from R&D systems, Cell Signaling Technology, Ultivue and the like.

As mentioned, a level of the dysfunctional cells above a predetermined threshold is indicative of a response to an immune checkpoint inhibition.

Without wishing to be bound by theory it is suggested that the higher the level of the dysfunctional cells the better is the prognosis. It is suggested that the subject has many T cells expressing TCRs that recognize the tumor.

According to another aspect of the invention, there is provided a method of treating a subject having a tumor, the method comprising:

(a) determining responsiveness of a subject to immune checkpoint inhibition according as described above; and
(i) wherein when the level of the dysfunctional cells is above the predetermined threshold, treating or selecting treatment for the subject with immune checkpoint inhibition; or
(ii) wherein when the level of the dysfunctional cells is below the predetermined threshold, subjecting the dysfunctional cells to ex vivo expansion and subsequently treating or selecting treatment for the subject with the immune checkpoint inhibition.

Hence, the level of dysfunctional cells dictates immediate treatment with immune checkpoint inhibitors or whether the cells need to be subjected to ex vivo expansion followed by the treatment with the immune checkpoint inhibition.

Thus, once an inadequate level of dysfunctional cells is observed e.g., below 20% (e.g., below 15%, 10%), the cells are subjected to ex vivo expansion and optionally stimulation (e.g., with neo-antigens, tissue fragments, antigen presenting cells loaded with the antigens, etc.).

Methods of ex vivo expanding TILs are well known in the art and are basically based on any protocol for adoptive cell therapy (ACT), e.g., Dudley et al. J Immunother. 2003; 26(4): 332-342, which is hereby incorporated by reference in its entirety.

Briefly and not meant to be limiting, a tumor sample is dissected free if surrounding normal tissue and necrotic areas. The sample is cut into fragments and each being placed in a well (e.g., 24 well plate) in the presence of a culture medium and IL-2. Each fragment is inspected every once in a while using low-power inverted microscope to monitor extrusion and proliferation of lymphocytes. The culture medium is typically changed 1 week following initiation of culturing.

Another approach is to use cultures derived from single-cell digests (see e.g., Riddell et al. Science 1992 257:238-241 which is hereby incorporated by reference in its entirety). Briefly and not meant to be limiting, each solid tumor specimen is dissected of surrounding tissue and necrotic areas. The specimen is fragmented and subjected to enzymatic (e.g., collagenase, hyaluronidase and DNAse) treatment in a culture medium under agitation. The single cell slurry is passed through sterile wire mesh to remove undigested tissue chunks. The digested single cells are washed in buffer and the viable cells are purified on a Ficoll gradient and the cells are resuspended for plating, typically in a 24 well plate. The plates are placed in a humidified incubator in the presence of IL-2. The cells are passaged as needed.

Another approach uses tumor infiltrating lymphocyte (TIL) cultures derived from physically disaggregation of tumor samples. Such an approach may use a dedicated machinery e.g., Medimachine (Becton Dickenson) including mini homogenizers. Fragments of tumor are prepared by dissection of biopsy specimens free from normal and necrotic tissue. Several fragments at a time are physically disaggregated by a 30-second Medimachine treatment, which disaggregated the tumor chunks using mechanical shear provided by a rotating disk that forces the tumor chunks across a small grater inside the medicon. The resulting slurry of single cells and small cell aggregates is washed, and resuspended in a culture medium. The cell suspension is layered onto a two-step gradient with a lower step of 100% Ficoll, and a middle step of 75% Ficoll and 25% CM. After 20 minutes' centrifugation at 2000 rpm (about 1100 g), the interfaces are collected. The lower interface containing the lymphocyte-enriched fraction is processed separately from the upper interface containing the tumor-enriched cells. Each fraction is washed. The lower, TIL-enriched fraction is plated in 24-well plates, and individual TIL cultures are generated exactly as for the single-cell suspensions derived by enzymatic degradation.

Regardless of the specific protocol used, the cultures can be further treated with an anti-CD3 antibody (and IL-2) optionally in the presence of irradiated, allogeneic feeder cells at a 200:1 ratio of feeder cells to responding TILs. —

TIL activity and specificity are determined by analysis of cytokine secretion as described above. Basically, the cells are washed to remove IL-2 prior to the assay. The cells are plated with stimulator cells (e.g., with the autologous tumor or antigen loaded-APCs).

Following culturing supernatants are collected and analyzed for TFNgamma secretion (e.g., by ELISA).

It will be appreciated that stimulation with anti CD3 and/or anti CD28 can be part of the expansion protocol. The protocol may include the use of specific antigens e.g., neo-antigens, where available, to promote the proliferation of tumor specific clones. The antigens can be loaded on antigen presenting cells or provided in a soluble form. The T cell activation needs two signals, One, activation of TCR complex. Another, co-stimulation of CD28 by CD80 or CD86. This stimulation is normally of two kinds, antigen dependent (in the presence of an antigen and co-stimulation from antigen presenting cells or anti-CD28 antibodies etc) and antigen independent (in the absence of an antigen but a mitogen [PHA or Con A] or anti-CD3 and anti-CD28 antibodies). Antigen dependent stimulation expands only antigen specific T cells whereas antigen independent stimulation expands all T cells in the sample culture.

The main principle behind stimulation by anti-CD3 and anti-CD28 antibodies is: the anti-CD3 will bind to CD3 and activate TCR complex without antigenic peptide from the antigen presenting cells; the anti-CD28 will bind to CD28 and stimulates the T cells without CD80 or CD86 from antigen presenting cells.

Once enough cells are at hand they are administered to the subject. According to a specific embodiment, the cells (in vitro expanded TILs) are autologous to the subject.

According to a specific embodiment, the cells (in vitro expanded TILs) are allogeneic to the subject.

According to a specific embodiment, the effective amount of cells is as typically used in adoptive cell therapy, e.g., 1-100*10⁶ cell.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. As used herein the condition refers to cancer.

According to a specific embodiment, the cells are non-genetically modified cells.

According to a specific embodiment, the cells are genetically modified to confer a function, e.g., T cell receptor specificity (e.g., CAR-T cells), immortalization and the like.

According to an aspect of the invention, there is provided a method of activating dysfunctional CD8⁺/Lag3⁺/PD1⁺/Tim3⁺/CD103⁺/CD39⁺/CD137⁺/Klrg1⁻ T cells, the method comprising, contacting dysfunctional CD8⁺/Lag3⁺/PD1⁺/Tim3⁺/CD103⁺/CD39⁺/CD137⁺/Klrg1⁻ T cells with an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby activating the dysfunctional immune cells.

According to an embodiment of the invention, the method is performed ex vivo.

According to an embodiment of the invention, the method is performed in vivo.

According to an aspect of the invention, there is provided a method of treating a subject having a tumor, the method comprising administering to the subject an immune checkpoint inhibitor and an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby treating the subject having the tumor.

According to an aspect of the invention, there is provided a method of treating a subject having a tumor, the method comprising administering to the subject an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby treating the subject having the tumor.

According to an aspect of the invention, there is provided an agent capable of inhibiting a target gene or expression product thereof selected from the group consisting of AKAP5, DGKH, PAG1, GALM, FUT8, WARS, CBLB, PIK3AP1, APOBEC3G, SLAMF7, SIRPG, GALNT1 for use in treating a subject having a tumor.

According to an aspect of the invention, there is provided an immune checkpoint inhibitor and an agent capable of inhibiting a target gene or expression product thereof selected from the group consisting of AKAP5, DGKH, PAG1, GALM, FUT8, WARS, CBLB, PIK3AP1, APOBEC3G, SLAMF7, SIRPG, GALNT1 for use in treating a subject having a tumor.

Any agent known in the art or generated by methods which are well known in the art to target these genes can be used according to the present teachings e.g., a CAR-T (e.g., Gogishvili Blood. 2017 Dec. 28; 130(26):2838-2847. doi: 10.1182/blood-2017-04-778423. Epub 2017 Oct. 31., which is hereby incorporated by reference in its entirety). Others are listed throughout the application.

As used herein “target gene” refers to the nucleic acid sequence encoding the gene, an mRNA product thereof or a polypeptide product thereof.

An “agent” refers to a chemical entity e.g., small molecule, peptide, a nucleic acid molecule.

As used herein “AKAP5” refers to the gene encoding A-kinase anchor protein 5. Aliases: AKAP5, AKAP75, AKAP79, H21, A-kinase anchoring protein 5. An exemplary mRNA sequence for human is provided in GenBank Accession Number NM_004857 (SEQ ID NO: 1). Exemplary protein sequences for human are provided in GenBank Accession Number NP_004848 (SEQ ID NO: 2) and NM_004848.3 (SEQ ID NO: 3). The protein is expressed in T lymphocytes and is known to interfere with IL-2 transcription.

As used herein “DGKH” refers to the gene encoding Diacylglycerol Kinase Eta, DGKeta or EC 2.7.1.107. This gene encodes a member of the diacylglycerol kinase (DGK) enzyme family. Members of this family are involved in regulating intracellular concentrations of diacylglycerol and phosphatidic acid. Protein products include, NP_178009 (SEQ ID NO: 4).

As used herein “PAG1” refers to the gene encoding phosphoprotein associated with glycosphingolipid-enriched microdomains 1. Aiases: CBP, PAG, phosphoprotein membrane anchor with glycosphingolipid microdomains 1. The protein encoded by this gene is a type III transmembrane adaptor protein that binds to the tyrosine kinase csk protein. It is involved in the regulation of T cell activation. An exemplary mRNA sequence for human is provided in GenBank Accession Number NM_018440 (SEQ ID NO: 5). An exemplary protein sequence for human is provided in GenBank Accession Number NP_060910 (SEQ ID NO: 6).

As used herein “GALM” refers to the gene encoding the enzyme that converts alpha-aldose to the beta-anomer. Aliases: BLOCK25, GLAT, HEL-S-63p, IBD1, galactose mutarotase (aldose 1-epimerase), galactose mutarotase. An exemplary mRNA sequence for human is provided in GenBank Accession Number NM_138801 (SEQ ID NO: 7). An exemplary protein sequence for human is provided in GenBank Accession Number NP_620156 (SEQ ID NO: 8).

As used herein “FUT8” refers to the gene encoding Alpha-(1,6)-fucosyltransferase. Aliases: FUT8, fucosyltransferase 8, CDGF. An exemplary mRNA sequence for human is provided in GenBank Accession Number NM_004480 (SEQ ID NO: 9), NM_178154 (SEQ ID NO: 10), NM_178155 (SEQ ID NO: 11), NM_178156 (SEQ ID NO: 12), NM_178157 (SEQ ID NO: 13). An exemplary protein sequence for human is provided in GenBank Accession Number NP_004471 (SEQ ID NO: 14), NP_835368 (SEQ ID NO: 15), NP_835369 (SEQ ID NO: 16).

As used herein “WARS” refers to the gene encoding the enzyme Tryptophanyl-tRNA synthetase. Aliases: Tryptophanyl-tRNA synthetase, cytoplasmic, GAMMA-2, IFI53, IFP53, tryptophanyl-tRNA synthetase, HMN9. An exemplary mRNA sequence for human is provided in GenBank Accession Number NM_004184 (SEQ ID NO: 17), NM_173701 (SEQ ID NO: 18), NM_213645 (SEQ ID NO: 19), NM_213646 (SEQ ID NO: 20). An exemplary protein sequence for human is provided in GenBank Accession Number NP_004175 (SEQ ID NO: 21), NP_776049 (SEQ ID NO: 22), NP_998810 (SEQ ID NO: 23), NP_998811 (SEQ ID NO: 24).

As used herein “CBLB” refers to the gene encoding the E3 ubiquitin-protein ligase, CBL-B. Aliases: Cbl-b, RNF56, Nbla00127, Cbl proto-oncogene B. An exemplary mRNA sequence for human is provided in GenBank Accession Number NM_170662 (SEQ ID NO: 25), NM_001321786 (SEQ ID NO: 26), NM_NM_001321788 (SEQ ID NO: 27), NM_NM_001321789 (SEQ ID NO: 28), NM_NM_00132190 (SEQ ID NO: 29). An exemplary protein sequence for human is provided in GenBank Accession Number NP_001308715 (SEQ ID NO: 30), NP_001308717 (SEQ ID NO: 31), NP_001308718 (SEQ ID NO: 32), NP_001308719 (SEQ ID NO: 33), NP_001308720 (SEQ ID NO: 34).

As used herein “PIK3AP1” refers to the gene encoding the enzyme Phosphoinositide 3-kinase adapter protein 1. Aliases: BCAP, phosphoinositide-3-kinase adaptor protein 1. An exemplary mRNA sequence for human is provided in GenBank Accession Number NM_152309 (SEQ ID NO: 35). An exemplary protein sequence for human is provided in GenBank Accession Number NP_689522 (SEQ ID NO: 36).

As used herein “APOBEC3G” refers to the gene encoding the enzyme, apolipoprotein B mRNA editing enzyme. Aliases: A3G, ARCD, ARP-9, ARP9, CEM-15, CEM15, MDS019, bK150C2.7, dJ494G10.1, apolipoprotein B mRNA editing enzyme catalytic subunit 3G. An exemplary mRNA sequence for human is provided in GenBank Accession Number NM_021822 (SEQ ID NO: 37). An exemplary protein sequence for human is provided in GenBank Accession Number NP_068594 (SEQ ID NO: 38), NP_001336365 (SEQ ID NO: 39), NP_001336366 (SEQ ID NO: 40), NP_001336367 (SEQ ID NO: 41).

As used herein “SLAM7” refers to the gene encoding the SLAM family member 7. Aliases: 19A, CD319, CRACC, CS1, SLAM family member 7. An exemplary mRNA sequence for human is provided in GenBank Accession Number NM_001282588 (SEQ ID NO: 42), NM_001282589 (SEQ ID NO: 43), NM_001282590 (SEQ ID NO: 44), NM_001282591 (SEQ ID NO: 45), NM_001282592 (SEQ ID NO: 46). An exemplary protein sequence for human is provided in GenBank Accession Number NP_001269517 (SEQ ID NO: 47), NP_001269518 (SEQ ID NO: 48), NP_001269519 (SEQ ID NO: 49), NP_001269520 (SEQ ID NO: 50), NP_001269521 (SEQ ID NO: 51).

As used herein “SIRPG” refers to the gene encoding Signal-regulatory protein gamma. Aliases: CD172g, SIRP-B2, SIRPB2, SIRPgamma, bA77C3.1, signal regulatory protein gamma. An exemplary mRNA sequence for human is provided in GenBank Accession Number NM_001039508 (SEQ ID NO: 52), NM_018556 (SEQ ID NO: 53), NM_080816 (SEQ ID NO: 54). An exemplary protein sequence for human is provided in GenBank Accession Number NP_001039508 (SEQ ID NO: 52), NP_018556 (SEQ ID NO: 55), NP_080816 (SEQ ID NO: 56).

As used herein “GALNT1” refers to the gene encoding the enzyme Polypeptide N-acetylgalactosaminyltransferase 1. Aliases: GALNAC-T1, polypeptide N-acetylgalactosaminyltransferase 1. An exemplary mRNA sequence for human is provided in GenBank Accession Number NM_001160401 (SEQ ID NO: 57), NM_013814 (SEQ ID NO: 58), NM_001361200 (SEQ ID NO: 59). An exemplary protein sequence for human is provided in GenBank Accession Number NP_001153876 (SEQ ID NO: 60), NP_038842 (SEQ ID NO: 61), NP_001348129 (SEQ ID NO: 62).

As used herein “an agent capable of down-regulating a target gene” refers to down-regulation of expression (mRNA or protein) or down-regulation of activity, e.g., enzymatic function.

As used herein the phrase “downregulates expression” refers to downregulating the expression of a protein (e.g. the protein product of the target gene, e.g., AKAP5) at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

For the same culture conditions the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating the target gene, e.g., AKAP5 expression are described in details hereinbelow.

Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding the target protein.

According to specific embodiments, the downregulating agent directly interacts with the target gene or expression product thereof.

According to specific embodiments, the agent directly binds the target gene of expression product thereof.

According to specific embodiments, the agent indirectly binds the target gene of expression product thereof (e.g. binds an effector of the target gene or expression product thereof).

According to specific embodiments the downregulating agent is an RNA silencing agent or a genome editing agent.

Thus, downregulation of the target gene or expression product thereof can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi: 10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the target gene mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of a miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-0,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation can be effected using an antisense polynucleotide capable of specifically hybridizing with an mRNA transcript encoding the expression product of the target gene.

Design of antisense molecules which can be used to efficiently downregulate an mRNA of the target gene must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Nucleic acid agents can also operate at the DNA level as summarized infra.

Downregulation of the target gene can also be achieved by inactivating the gene via introducing targeted mutations involving loss-of function alterations (e.g. point mutations, deletions and insertions) in the gene structure.

As used herein, the phrase “loss-of-function alterations” refers to any mutation in the DNA sequence of a gene, which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the mutation may be in a homozygous form or in a heterozygous form.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8, 124,369; 8, 129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fok1 domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], To12 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas To12 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of the target gene or expression product thereof may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Down-regulation at the polypeptide level is also contemplated herein.

According to specific embodiments the agent capable of downregulating a target gene product is an antibody or antibody fragment capable of specifically binding the protein. Preferably, the antibody specifically binds at least one epitope of the target protein. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

As the target protein is localized intracellularly, an antibody or antibody fragment capable of specifically binding the target protein is typically an intracellular antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Another agent which can be used along with some embodiments of the invention to downregulate the target protein is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another agent capable of downregulating the target protein would be any molecule which binds to and/or cleaves the target protein. Such molecules can be a small molecule, antagonists, or inhibitory peptide.

Treatment can be combined with any anti cancer treatment known in the art, including, but not limited to, chemotherapeutic agents, radiotherapeutic agents, hormonal therapy, immune modulators, engineered immune cell therapy (e.g., CAR-T) and other treatment regimens (e.g., surgery, cell transplantation e.g. hematopoietic stem cell transplantation) which are well known in the art.

The chemotherapeutic agent of the present invention can be, but not limited to, cytarabine (cytosine arabinoside, Ara-C, Cytosar-U), asprin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2 •difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including Nmethylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide;

progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

In some embodiments the chemotherapeutic agent of the present invention is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, Cytosar-U), melphalan, busulfan (Myleran®), thiotepa, bleomycin, platinum (cisplatin), cyclophosphamide, Cytoxan®)., daunorubicin, doxorubicin, idarubicin, mitoxantrone, 5-azacytidine, cladribine, fludarabine, hydroxyurea, 6-mercaptopurine, methotrexate, 6-thioguanine, or any combination thereof.

According to a specific embodiment, the treatment is combined with an immune checkpoint inhibitor, such as described above.

According to a specific embodiment, administering the immune checkpoint inhibitor is following administering the agent, as described herein, or the cells (e.g., TILs). It is suggested that treatment with the agent or the cells will prime treatment with the checkpoint inhibitors.

The agent/cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent/cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (agent/cells) effective to prevent, alleviate or ameliorate symptoms of a disorder (e.g., cancer. melanoma) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide agent/cells levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Tumor Dissociation

Fresh tumor tissue was dissociated by manual mincing followed by an incubation of 20 minutes at 37° C. in RPMI with collagenase IV (Sigma-Aldrich C5138) and pulmozyme (Roche), alternated with 3 rounds of dissociation with a gentlemacs dissociator (Miltenyi, 130-093-235, 130-096-334). After dissociation, cell suspensions were filtered with a 100 um filter, washed in RPMI 1640 medium with penicillin, streptomycin and human serum. Cell suspensions were frozen down in 90% FBS and 10% DMSO.

PBMC Isolation

PBMCs were isolated from blood using a Ficoll gradient. PBMCs were frozen in 90% FBS and 10% DMSO directly after collection.

Single Cell Sorting of Tumor and Blood Materials

Tumor cell suspensions and PBMCs were thawed in RPMI with human serum, penicillin and streptomycin, and 250 U/ml benzonase. Cells were stained in PBS with 0.5% BSA and 2 mM EDTA containing fluorochrome-conjugated antibodies. Cells were stained with propidium iodide (PI) immediately prior to sorting with a FACSaria Fusion. Forward and side scatter settings were used to select for immune cells and exclude doublets, viable cells were identified based on low PI staining, and immune cells were sorted based on CD45 expression. For each tumor, multiple plates were sorted with CD3+ and CD3− immune cells. Immune cells were single cell sorted using index sorting into 384 wells plates containing 2 μL of lysis solution with barcoded poly(T) reverse-transcription (RT) primers. Four empty wells were kept in each 384-well plate as a no-cell control. Plates were briefly centrifuged, snap frozen on dry ice, and stored at −80 degree. Antibodies used for sorting are CD3-FITC and CD45-APC or CD45-BV510, and for index sorting combinations of CD4-BV421, CD4-BV510, CD8-AF700, CD8-Pacific Orange, PD1-PE, CD103-BV711, PDL1-APC, CD11b-BV650, CD56-PE, TIM3-BV421, LAG3-AF700, OX40-BV711, CD137-APC. For PBMC sorting, CD45-FITC, CD3-FITC, CD8-AF700, PD1-PE, CD103-BV711, CCR7-PECF594, and CD45RA-PECY5.5 were used.

Single Cell Libraries Preparation

Single cell libraries were prepared with Massively Parallel Single-Cell RNA-seq Library (MARS-seq) (Jaitin et al., 2014). In brief, mRNA from tumor or immune cells sorted into cell capture plates are barcoded and converted into cDNA and pooled using an automated pipeline. The pooled sample is then linearly amplified by T7 in vitro transcription, and the resulting RNA is fragmented and converted into a sequencing-ready library by tagging the samples with pool barcodes and illumina sequences during ligation, reverse transcription, and PCR. Each pool of cells is tested for library quality and concentration is assessed.

Single Cell TCR-Seq (scTCR-Seq) Libraries Preparation

After linear amplification by T7 RNA polymerase-mediated in vitro transcription in MARS-seq library process, half of the resulting RNA material was reversed transcribed with a primer set that are specific to different Vβ segments of human T cell receptor β chain (Han et al., 2014). The resulting complementary DNA was then amplified with a set of nested primers (Han et al., 2014) and partial rd-2 primer. The PCR product was then cleaned up and used for a final amplification step with P7-rd1 and P5-rd2 primers. The TCR libraries were then sequenced in Illumina Miseq, with 165 bp of read1 sequence and 15 bp of read2 sequence. Primer sequences are listed Table 3 below.

TABLE 3

| TCRb | | | |
|---|---|---|---|
| primer | | | |
| set 1 | sequence | | |
| TRBV2 | CTGAAATATTCGATGATCAATTCTCAG | 64 | |
| TRBV3 | TCATTATAAATGAAACAGTTCCAAATCG | 65 | |
| TRBV4 | AGTGTGCCAAGTCGCTTCTCAC | 66 | |
| TRBV5-1 | GAGACACAGAGAAACAAAGGAAACTTC | 67 | |
| TRBV5-4,8 | AGAGGAAACTYCCCTCCTAGATT | 68 | C/T |
| 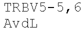 | 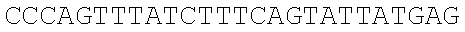 | 69 | |
| TRBV6- | AAGGAGAAGTCCCSAATGGCTACAA | 70 | C/G |
| 1,5,6 | | | |
| TRBV6-2,3 | AGGGTACAACTGCCAAAGGAGAGGT | 71 | |
| TRBV6-4 | GGCAAAGGAGAAGTCCCTGATGGTT | 72 | |
| TRBV6-8 | CTGACAAAGAAGTCCCCAATGGCTAC | 73 | |
| TRBV6-9 | CACTGACAAAGGAGAAGTCCCCGAT | 74 | |
| TRBV7-2,7 | AGACAAATCAGGGCTGCCCAGTGA | 75 | |
| TRBV7-3 | GACTCAGGGCTGCCCAACGAT | 76 | |
| 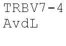 | 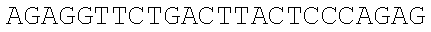 | 77 | |
| 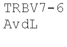 | 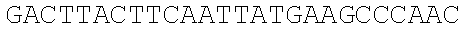 | 78 | |
| TRBV7-8 | CCAGAATGAAGCTCAACTAGACAA | 79 | |
| TRBV7-9 | GACTTACTTCCAGAATGAAGCTCAACT | 80 | |
| TRBV9 | GAGCAAAAGGAAACATTCTTGAACGATT | 81 | |
| TRBV10-1,3 | GGCTRATCCATTACTCATATGGTGTT | 82 | A/G |
| TRBV10-2 | GATAAAGGAGAAGTCCCCGATGGCT | 83 | |
| TRBV11 | GATTCACAGTTGCCTAAGGATCGAT | 84 | |
| TRBV12-3,4 | GATTCAGGGATGCCCGAGGATCG | 85 | |
| TRBV12-5 | GATTCGGGGATGCCGAAGGATCG | 86 | |
| TRBV13 | GCAGAGCGATAAAGGAAGCATCCCT | 87 | |
| TRBV14 | TCCGGTATGCCCAACAATCGATTCT | 88 | |
| TRBV15 | GATTTTAACAATGAAGCAGACACCCCT | 89 | |
| TRBV16 | GATGAAACAGGTATGCCCAAGGAAAG | 90 | |
| TRBV18 | TATCATAGATGAGTCAGGAATGCCAAAG | 91 | |
| TRBV19 | GACTTTCAGAAAGGAGATATAGCTGAA | 92 | |
| TRBV20 | CAAGGCCACATACGAGCAAGGCGTC | 93 | |
| TRBV24 | CAAAGATATAAACAAAGGAGAGATCTCT | 94 | |
| TRBV25 | AGAGAAGGGAGATCTTTCCTCTGAGT | 95 | |
| TRBV27 | GACTGATAAGGGAGATGTTCCTGAAG | 96 | |
| TRBV28 | GGCTGATCTATTTCTCATATGATGTTAA | 97 | |
| TRBV29 | GCCACATATGAGAGTGGATTTGTCATT | 98 | |
| TRBV30 | GGTGCCCCAGAATCTCTCAGCCT | 99 | |

scTCR-Seq Protocol Validation

Five different human T cell clones with known TCR sequences were stained with CD3-APC, CD3-FITC, CD3-PE, CD3-PerCP, and CD3-AF700 and subsequently mixed in a 1:1 ratio, and four single cell 384 well plates were sorted while recording index values, and processed with scTCR-seq method. The obtained TCRβ sequences were then compared with the known reference sequence per cell to determine the sensitivity and specificity. The present method was able to identify the TCRβ in 32% (490/1520) of cells sorted, with the correct TCRβ assigned in 97% (442/455) of cells with clear FACS index data to serve as a reference.

Ex Vivo T Cell Expansion

T cells were expanded from tumor fragments or tumor suspension by culturing in RPMI with human serum, penicillin and streptomycin and 6000 IU/ml IL2 for 14 days. For further expansion, T cells were cultured in RPMI with human serum, penicillin and streptomycin with 3000 IU/ml IL2, 30 ng/ml aCD3 antibody, and 1:200 irradiated PBMCs (40Gy) for an additional 14 days.

Cell Cycle Staining

Tumor single cell suspensions were stained with Live/dead fixable near-IR dead cell stain kit, CD45-APC, CX3CR1-PE, PD1-PeCy7, CD8-AF700 and CD3-FITC. After fixation with the FOXP3/transcription factor staining buffer set and additional fixation with 70% ethanol, cells were permeabilized and stained with KI67-PerCPCy5.5. Prior to read-out, 1 ug/ml DAPI was added.

Tumor Reactivity Assays

Ex vivo expanded T cells and tumor suspensions were incubated in RPMI with human serum, penicillin and streptomycin, and benzonase for 45-60 min. T cells were labeled with cell trace violet according to manufacturer's protocol. T cells and tumor cells were overnight co-cultured in a 1:1 ratio in RPMI with human serum, penicillin and streptomycin. 60 minutes after starting the co-culture, golgiplug (BD Biosciences) was added to the medium. Cell suspension were extracellularly stained with CD3-APC, CD8-AF700, and IRDye in PBS with 0.5% BSA and 2 mM EDTA. Cells were fixated and permeabilized in 1% PFA and 0.1% Triton. Intracellular staining for IFNg-PE, TNFa-AF499, and CD137-BV650 was done in 1% PFA, 0.1% Triton, and 1% BSA.

Histology Analysis

Paraffin sections were cut at 3 um from FFPE tumor material. Slides were stained with Hematoxylin and Bluing Reagent and manually scored for the percentage of immune infiltrate.

Quantification and Statistical Analysis

Low-Level MARS-Seq Processing

All scRNA-seq libraries (pooled at equimolar concentration) were sequenced using Illumina NextSeq 500 at a median sequencing depth of ˜40,000 reads per cell. Sequences were mapped to human genome (hg19), demultiplexed, and filtered as previously described (Jaitin et al., 2014) with the following adaptations. Mapping of reads was done using HISAT (version 0.1.6); reads with multiple mapping positions were excluded. Reads were associated with genes if they were mapped to an exon, using the UCSC genome browser for reference. Exons of different genes that shared genomic position on the same strand were considered a single gene with a concatenated gene symbol. The level of spurious UMIs in the data was estimated using statistics on empty MARS-seq wells, and excluded rare cases with estimated noise >5% (median estimated noise over all experiment was 2%).

scTCR-Seq Raw Data Processing and Analysis

TCR sequences were generated from 384 well plates, including a barcode and UMI, similar to MARS-seq. Initial filtering was performed on each plate independently. Barcode sequencing errors were corrected by grouping reads with similar barcodes (hamming distance <=2) and filtering reads with similar UMIs but different barcodes. Typical sequencing coverage for TCR molecules was high after extracting positions 80-130 base pairs in the sequence for the hyper variable region, and used filtering of low coverage UMIs since these were observed to be strongly enriched for errors and contamination (i.e. as demonstrated previously in MARS-seq). These filtering stages provided us with a filtered table of candidate TCR sequences per barcode.

The remaining pre-processing was similar to Tracer (Stubbington et al., 2016). The fasta files from the first step were used as the input of IgBlast (ftp://ftp(dot)ncbi(dot)nlm(dot)nih(dot)gov/blast/executables/igblast/release/1.7.0/). Reads were then grouped according to the TCR sequence that represented them best, provided mapping was to the correct gene segments and the E-values for the reported V and J alignments were below 5e-3. A csv file with statistics from IgBlast was generated for all the productive reads (not shown).

Metacell Modelling

The MetaCell package (Baran et al. Submitted, see software resources) was used with the following specific parameters (complete script reproducing all analysis from raw data will be available). Specific mitochondrial, Immunoglobulins, high abundance lincRNA, and genes linked with poorly supported transcriptional models were removed (annotated with the prefix “RP-”). Cells with less than 500 UMIs or total fraction of mitochondrial gene expression exceeding 60% were filtered. Gene features were selected using the parameter Tvm=0.2 and minimal total umi >200. Gene features associated with the cell cycle, type I Interferon (IFN) response, and general stress using a clustering approach were used. To this end first all genes with correlation coefficient at least 0.1 for one of the anchor genes MKI67, HIST1H1D, PCNA, SMC4, MCM3 (cell cycle), ISG15, OAS1, WARS, IFIT1 (type I IFN) and TXN, HSP90AB1, HSPA1A, FOS, HIF1A (stress) were identified. The present inventors then hierarchically clustered the correlation matrix between these genes (filtering genes with low coverage and compute correlation using a down-sampled UMI matrix).

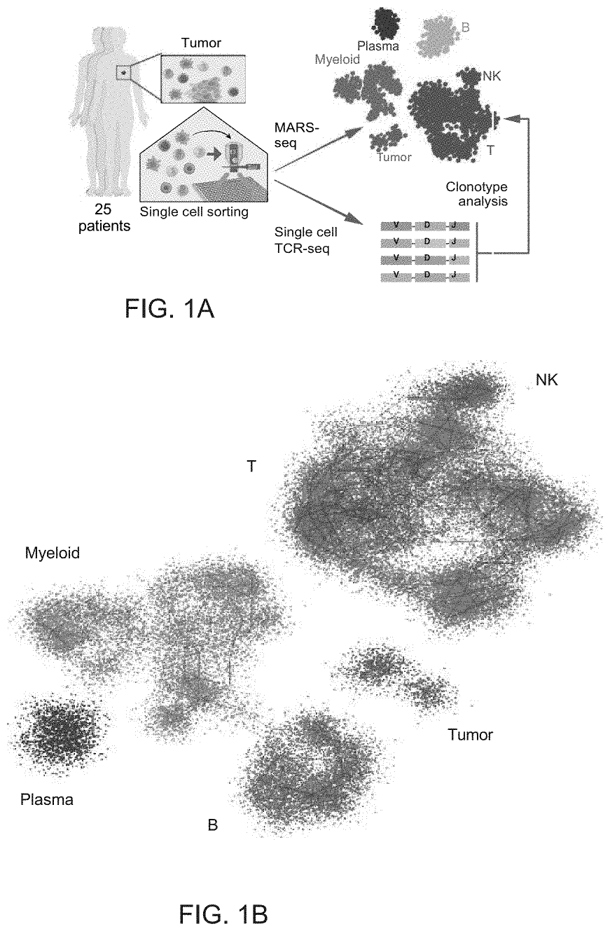

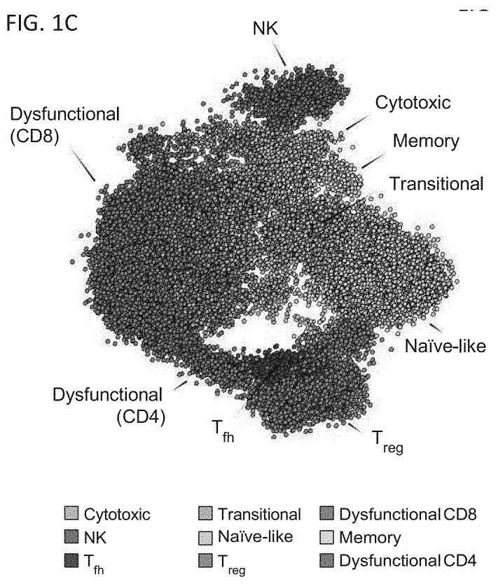

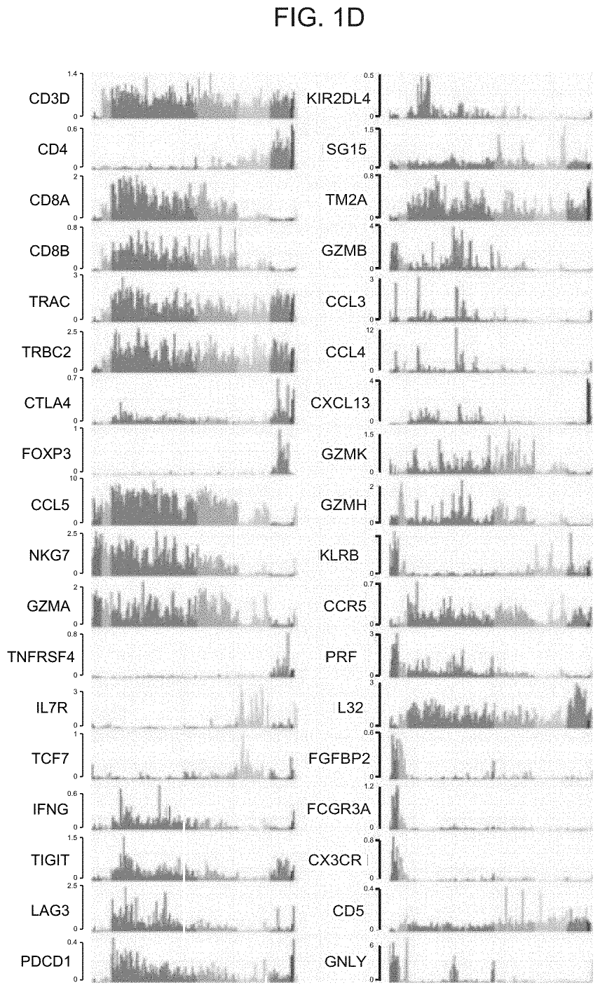

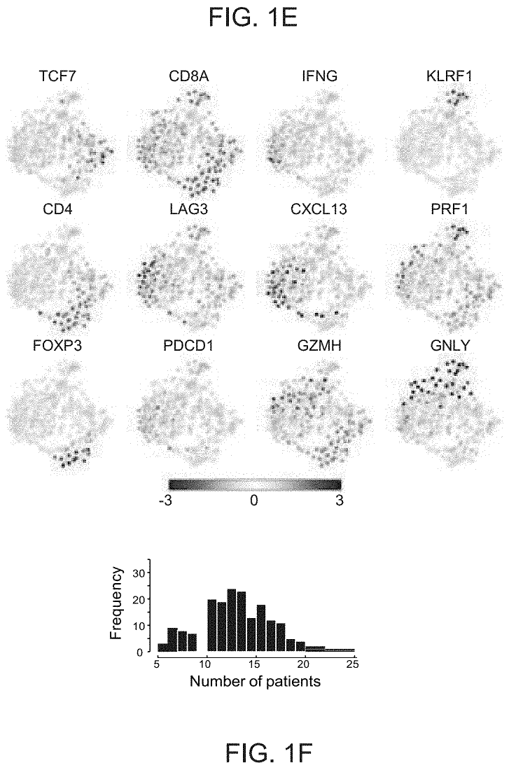

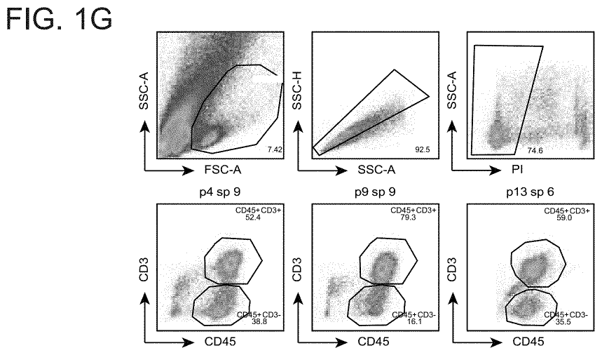

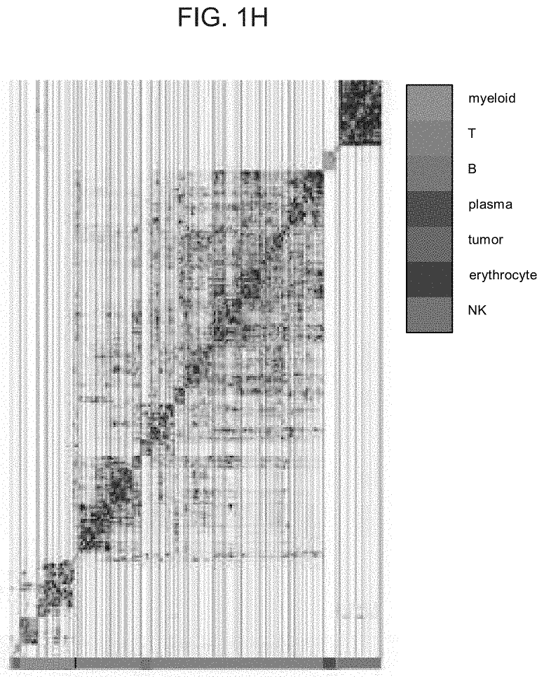

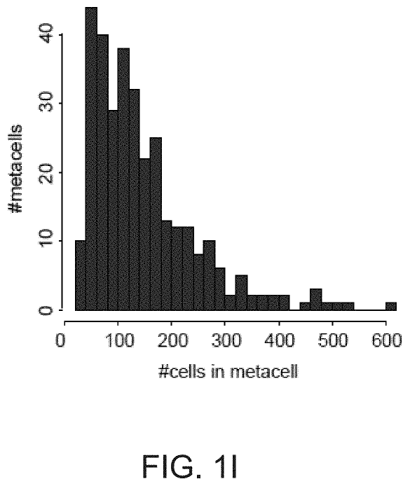

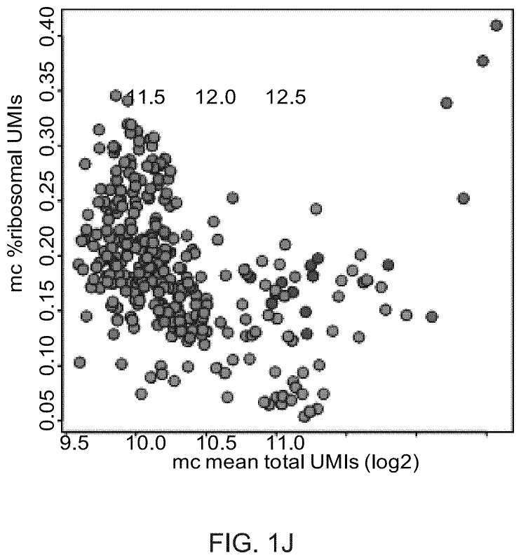

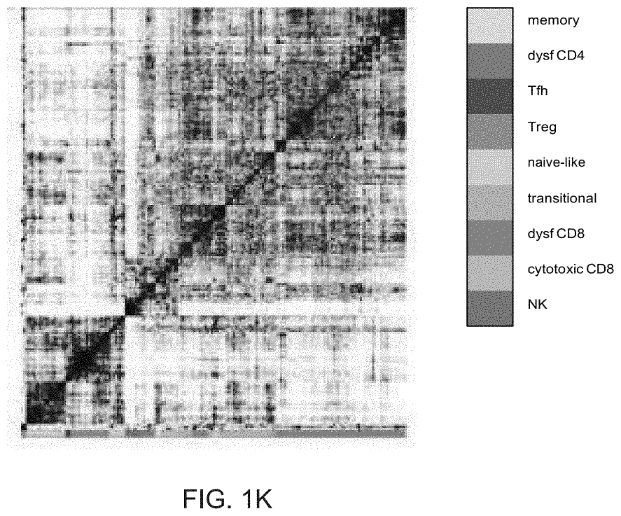

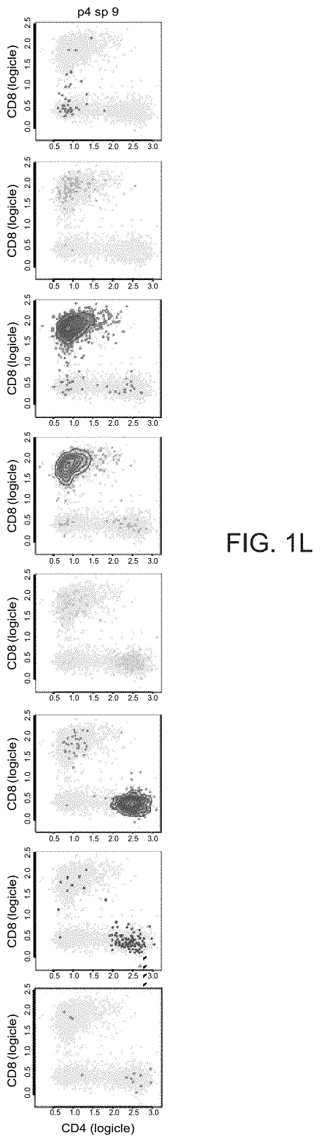

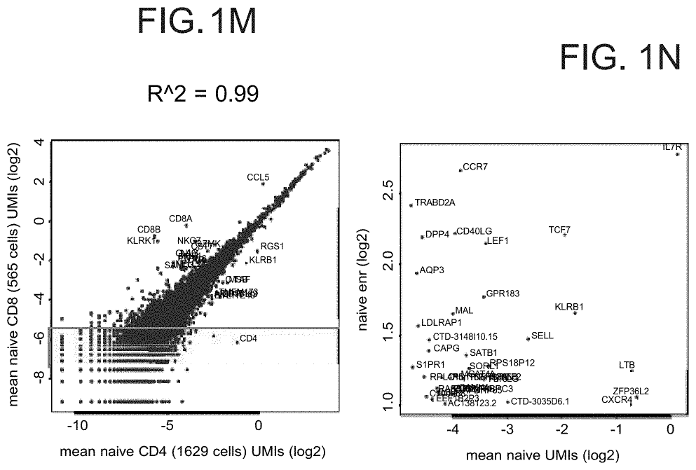

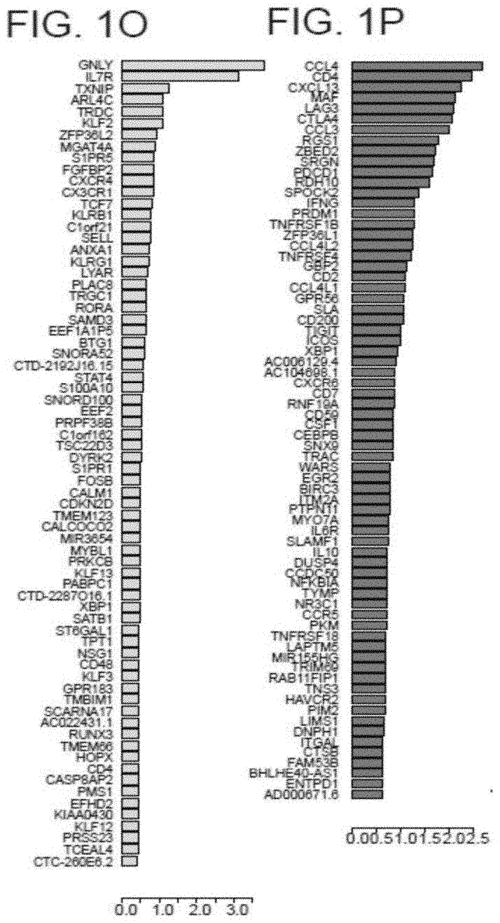

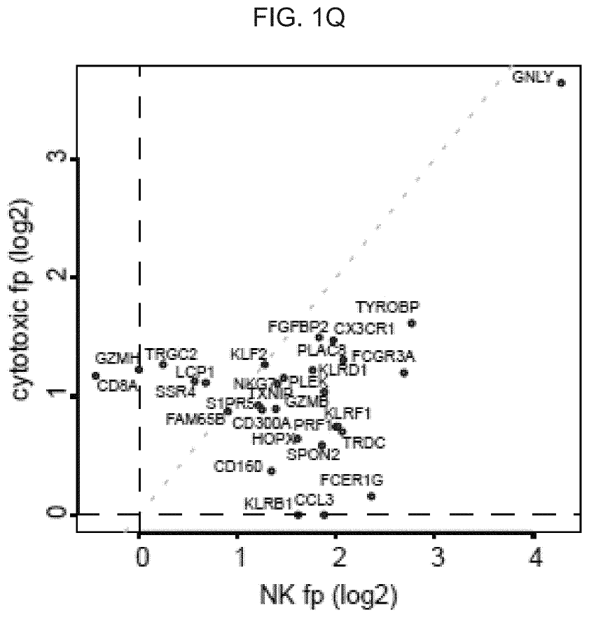

For the MC model in FIG. 1-5, cells from 25 patients with validated pathological profile (two patients had 2 metastases) were used. It is of note that no patients were filtered before the MC creation. On some panels, a minimum of 100 T/NK cells per patient was enforced. The gene selection strategy discussed above retained a total of 1,675 gene features for the computation of the Metacell balanced similarity graph. The present inventors used K=200, 500 bootstrap iterations and otherwise standard parameters. No outlier filtering was applied, but the MC splitting phase was performed.

Annotation of the MC model was done using the MC confusion matrix and analysis of marker genes. A single erythrocyte MC of 14 cells was removed, and classified the remaining MCs as T/NK or other, using straightforward analysis of known cell type markers (e.g. CD3D, CD3G, CD4, CD8A-B, and more). Detailed annotation within the T/NK MC model was performed using hierarchical clustering of the T/NK confusion matrix (FIG. 1K) and supervised analysis of enriched genes as shown in the text.

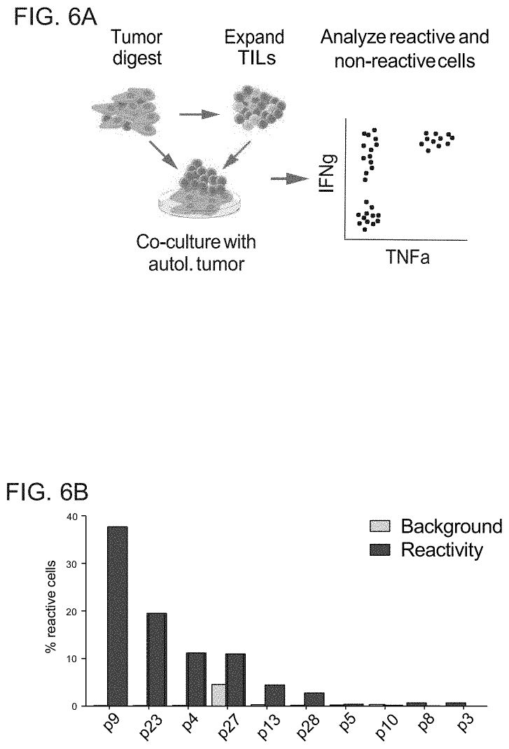

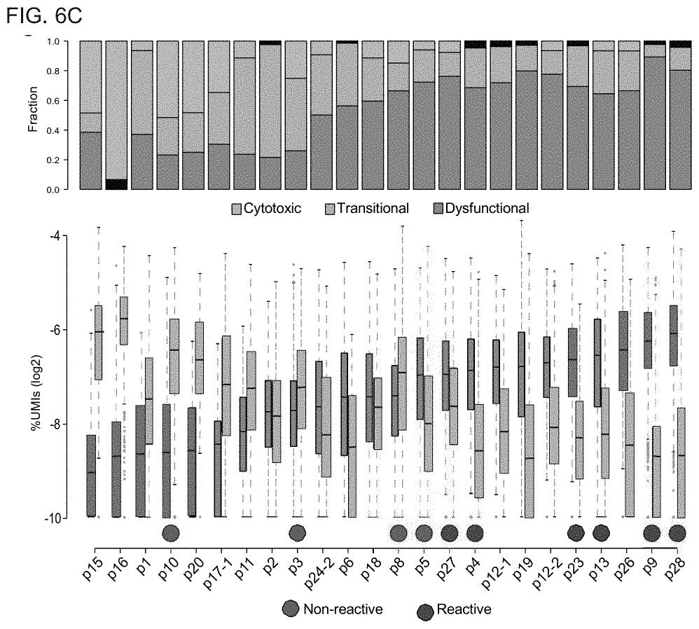

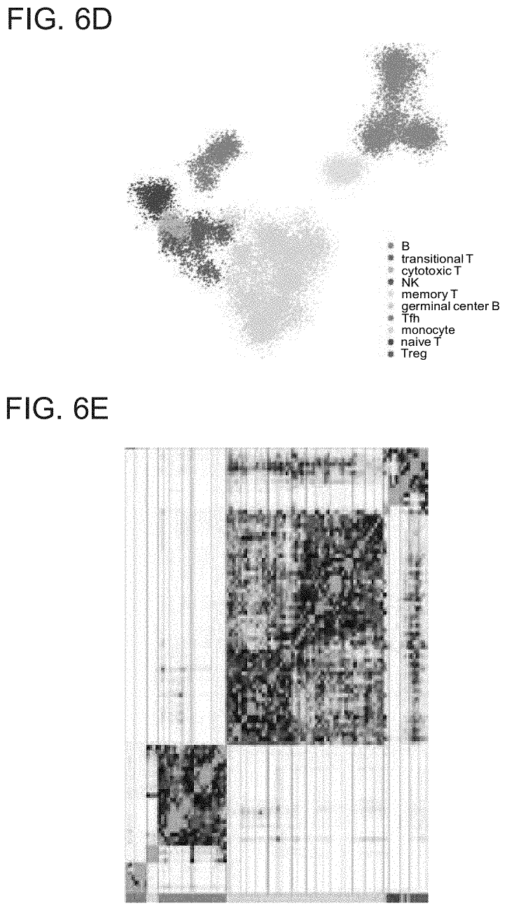

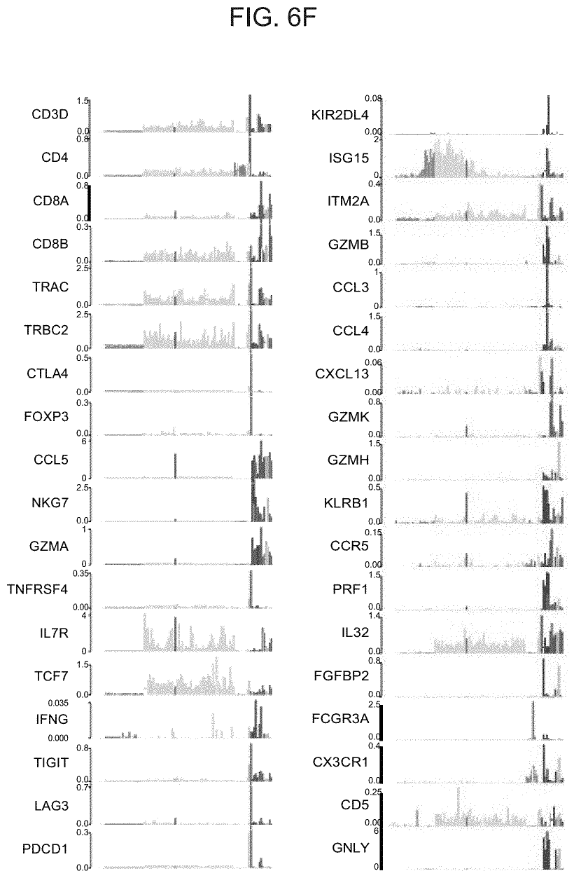

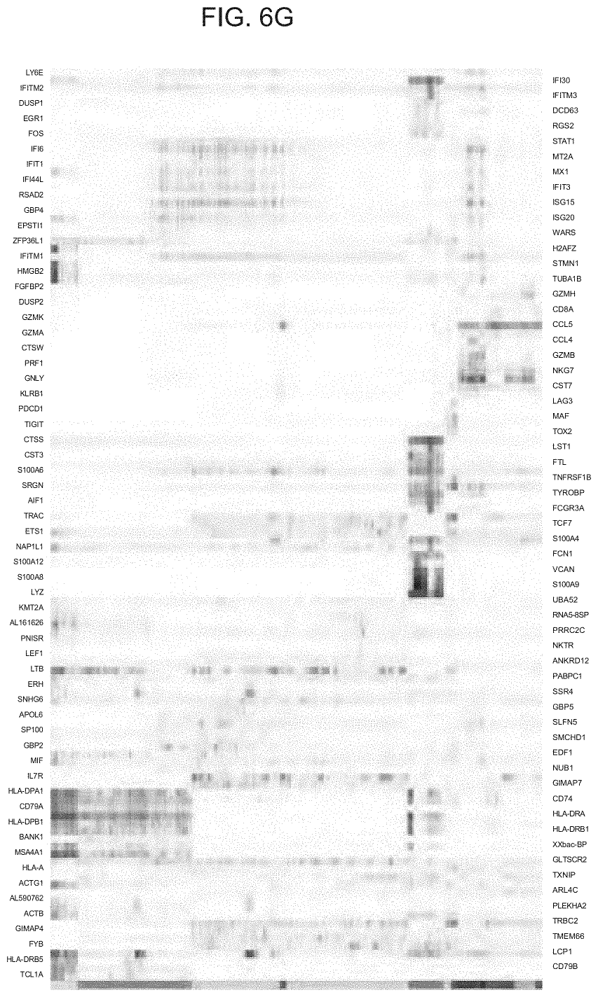

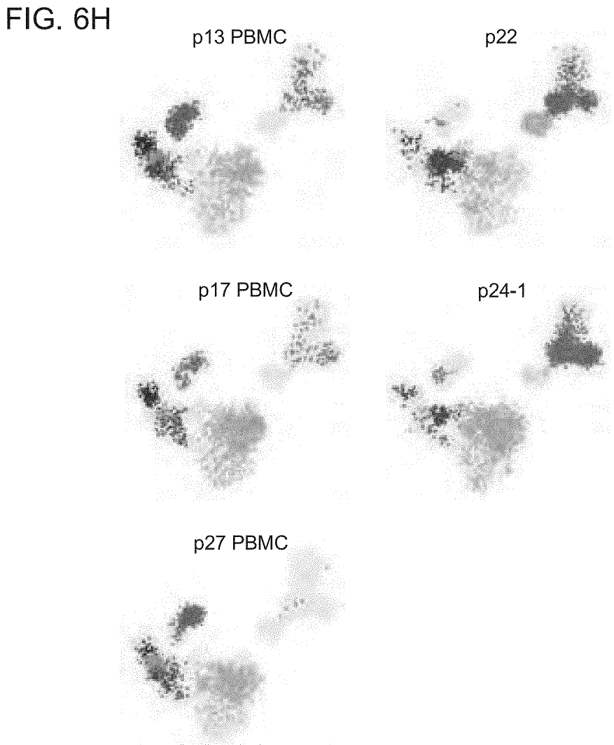

Analysis of PBMC data (FIGS. 6D-H) was performed using similar parameters, with K=200, and analysis of marker genes shown to be enriched in the infiltrates model.

Defining Differentiation Gene Modules and Gradients

To account for the complex gene expression in dysfunctional CD8, cytotoxic CD8, T regulatory, macrophage, and monocytes, a metacell analysis was combined with an approach aiming at identifying quantitative gene signatures. Given any list of signature genes for a certain differentiation fate, the signature's scores for each metacell were identified by averaging the metacell log enrichment scores (lfp values) of the genes in the set. Note that using this approach the contribution of highly expressed genes to the score was limited, and relied on the regularization of the metacell computation of gene enrichment scores to restrict the noise levels inflicted over the differentiation scores.

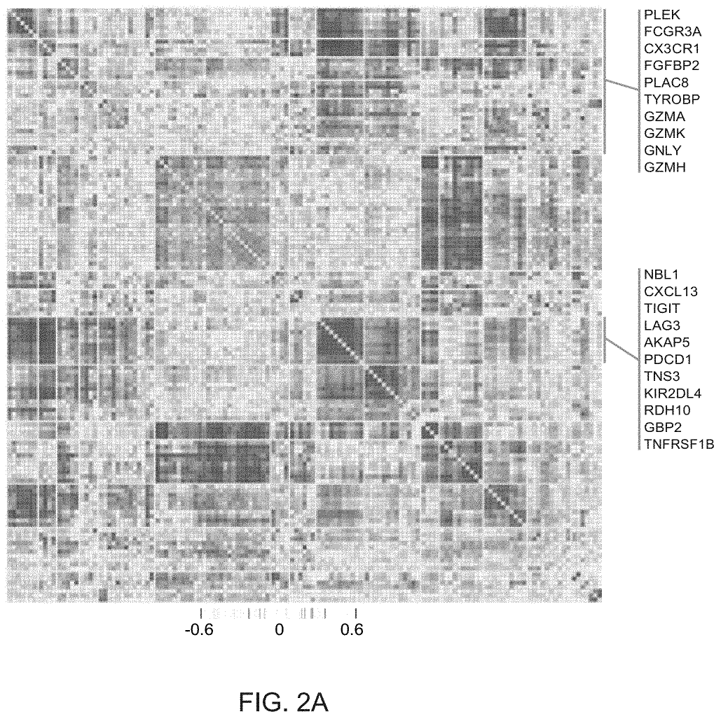

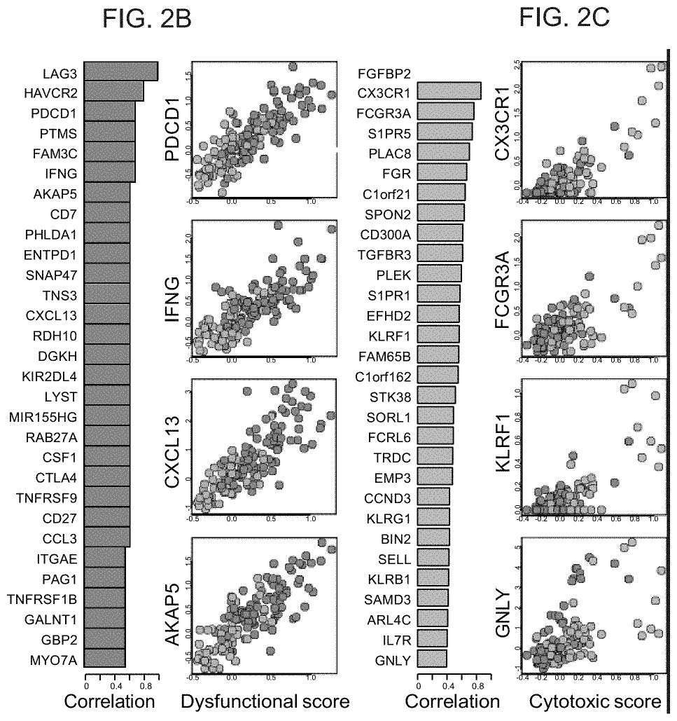

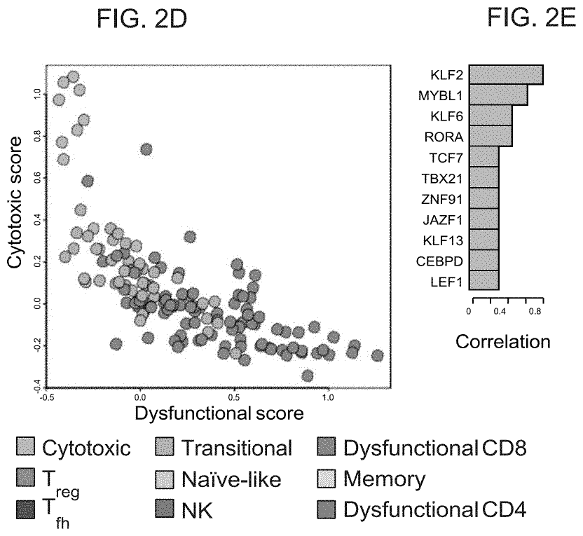

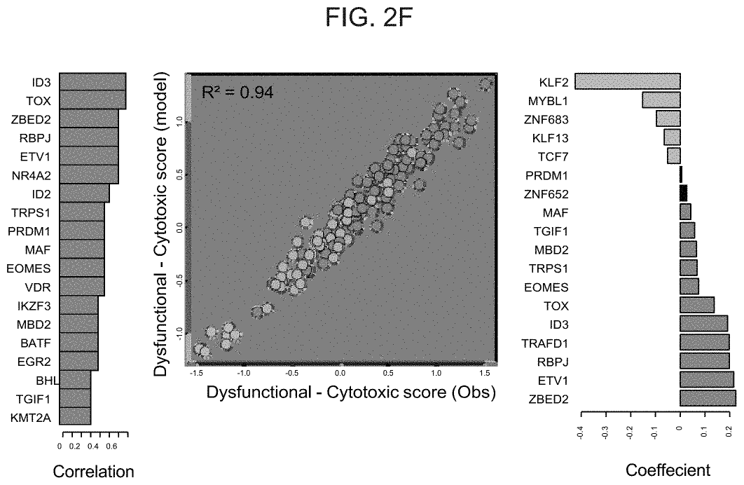

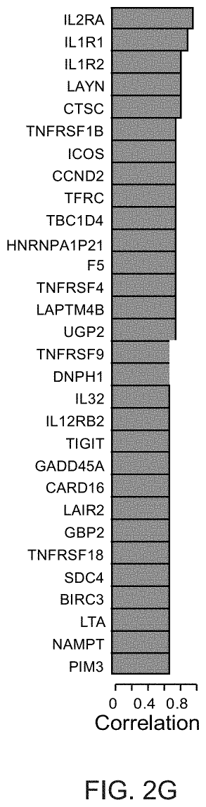

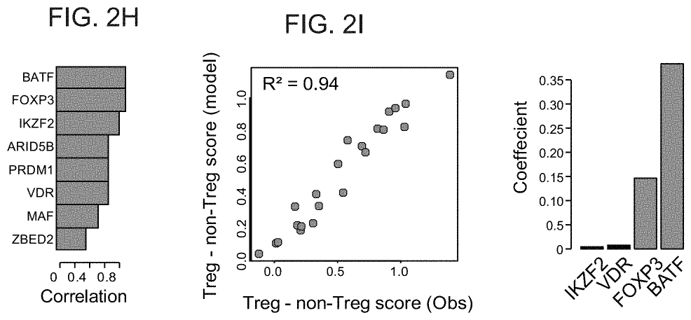

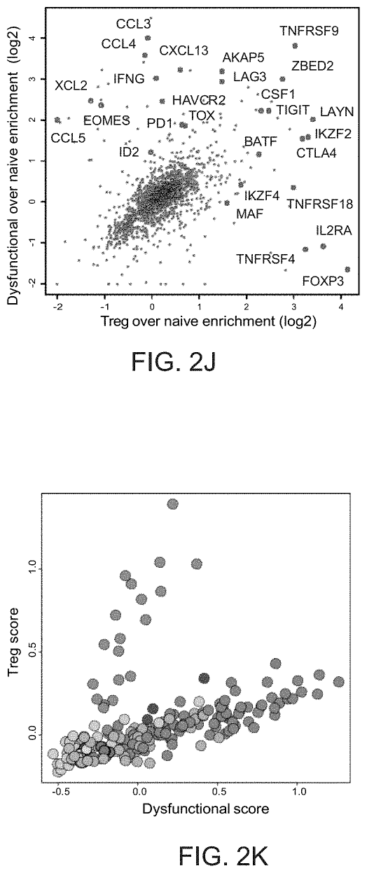

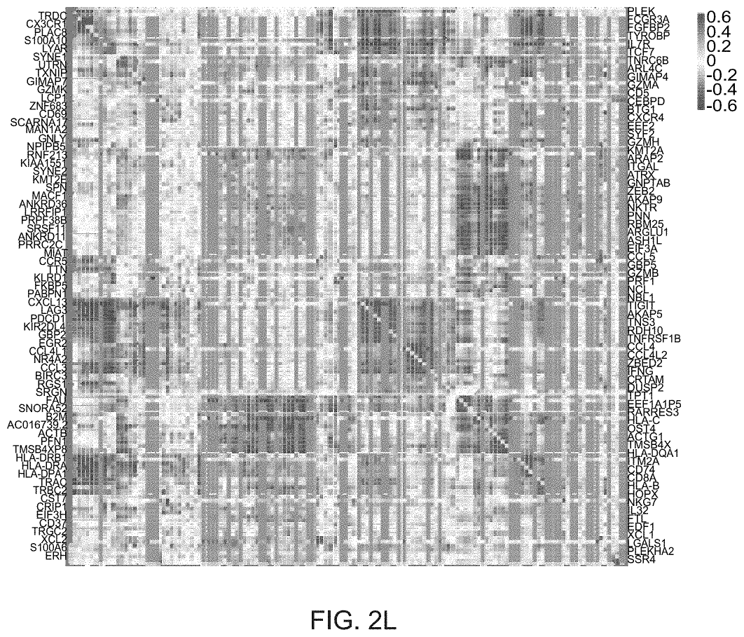

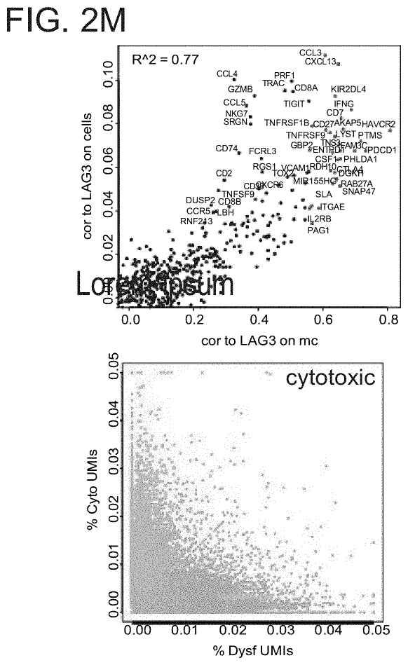

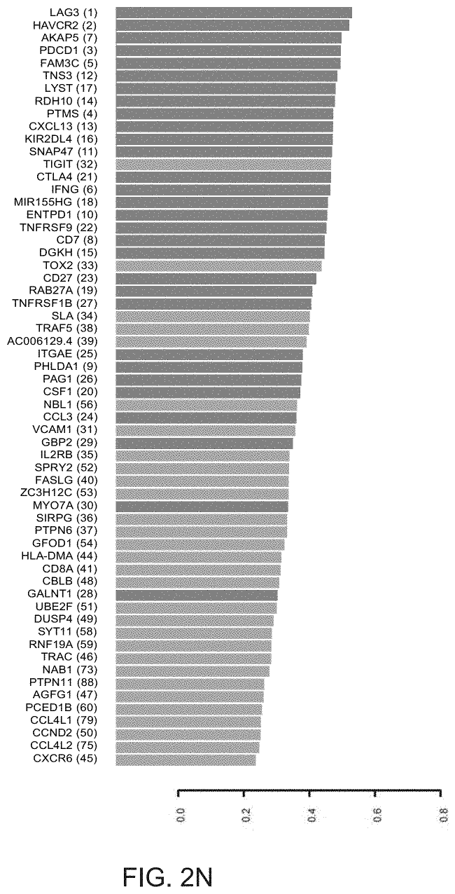

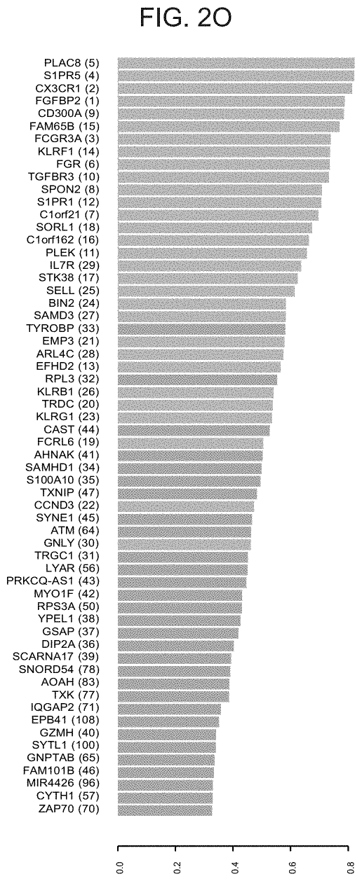

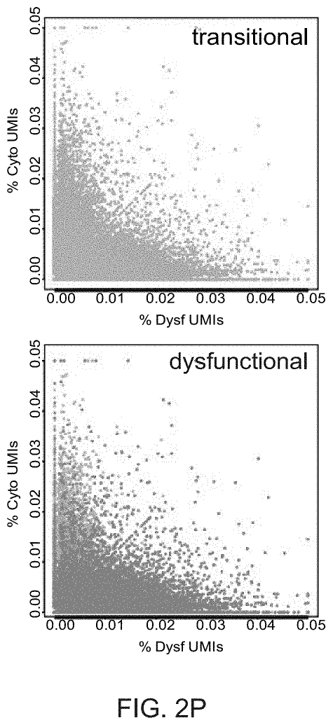

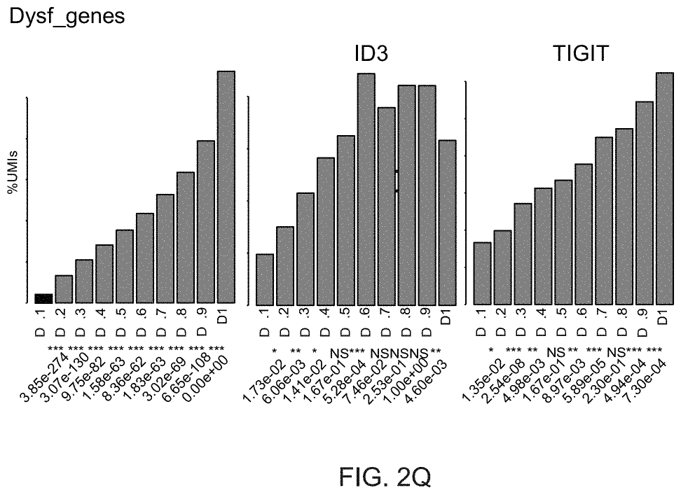

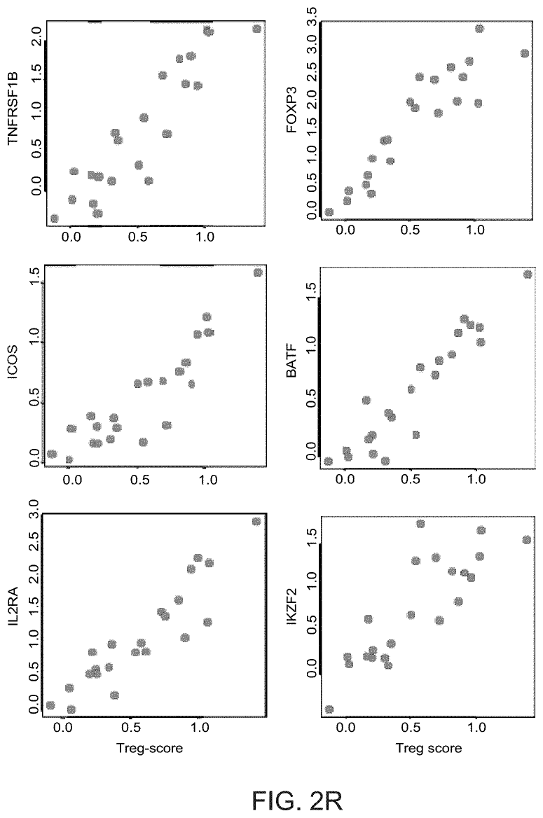

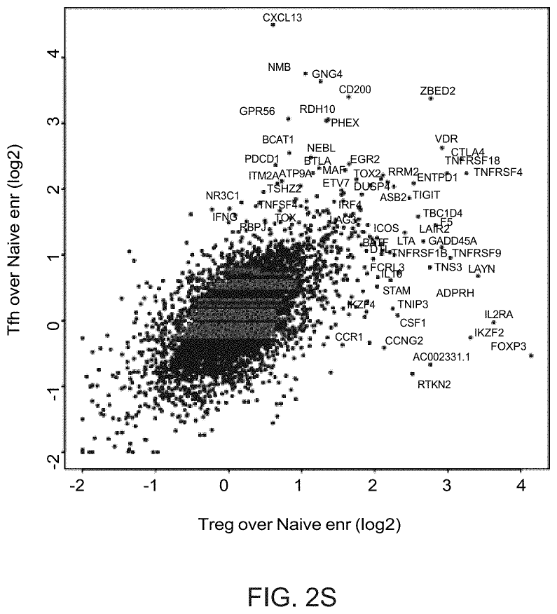

To define signatures gene sets for CD8 dysfunctional, CD8 cytotoxic, and regulatory T-cells, groups of 30 non-TF genes that were maximally correlated to selected anchor genes (LAG3, FGFBP2 and IL2RA) were identified, using linear correlation over metacells' log enrichment scores. Genes associated with cell cycle, type I IFN and stress were filtered from these lists. Anchors were validated by testing correlation of the signature scores computed from their derived gene sets to all genes while excluding the gene itself from the score (FIGS. 2N-O), establishing consistency and robustness (genes remained top ranking even when omitting them from the score) to anchor selection. The anchor approach for T-cells was preferred over the alternative approach of finding genes with maximal enrichment for a selected metacell, given the high complexity and multiple regulatory processes affecting the T-cells transcriptional space. Selecting and validating a consistent gene set starting from a well-defined anchor ensured that our score is based on the pathway of interest, and imitated some classical concepts from biclustering analysis.

For defining myeloid signature gene sets the simpler approach of selecting the genes with highest enrichment in metacells annotated as monocytes, DC, or macrophage (excluding one metacell annotated as non-classical monocytes) was used. To this end the log enrichment scores of each gene was averaged over the metacells in the respective groups, but excluded genes for which only one metacell was enriched over the other metacells in the group by 8-fold or more.

To validate the significance of the transcriptional gradients that the present inventors observed when computing the various signature scores, the present inventors relied on the fact that metacells group cells into disjoint sets, and tested differential expression of genes that were not part of the signature gene set between bins of cells that were grouped into metacells with increasing ranges of signature scores (e.g. FIG. 2Q). To study potential transcriptional regulatory networks in T-cells and myeloid cells w a list of annotated transcription factors genes was used (Lambert et al., 2018) with some specific additions (ID2, ID3 and TOX). As mentioned above, TFs were not considered when defining the signature scores, so modelling was not over-fitted a-priori. For modelling the difference between the dysfunctional and cytotoxic signature scores in CD8+ metacells, candidate regulatory TFs were identified as those enriched at least two fold over the background in at least one CD8+ metacell and inferred a simple linear model aiming to predict the difference in signature scores using the enrichment scores of a subset of the TF candidates. This was done in the framework of a lasso-regularized cross validation scheme with the R package glmnet. A similar approach was applied to predict the T-reg score in T-reg metacells and the difference between monocyte and macrophages scores in myeloid metacells.

Analysis of Clonal-Sharing

scTCR-seq data can provide a clonal identifier for the T-cells in which it is observed. Cells sharing an identifier were therefore considered sharing a clonal origin. Cells missing an identifier were considered as not observed (rather than not clonal). A correlation was observed between the intensity of TCR expression (as estimated by MARS-seq) and the rate of scTCR-seq detection, which may suggest systematic bias in the analysis of clonality rates, enriching for signals in T-cell populations showing high levels of TCR (e.g. dysfunctional, Tfh, and Treg cells, FIG. 5M-N). To control for some of these effects, as well as for the variable efficiency of TCR detection in different patients, the present inventors used a resampling strategy to generate randomized TCR-seq information and control for clonality statistics. Resampling was performed by selecting at random a new cell for each observed TCR sequence, but forcing the cell to maintain the original patient source, and the original bin (one of 9 bins) of overall TCR-seq expression. This was repeated for 200 iterations and gave rise to a robust control dataset that preserve patient/clone size compositions and the overall relationship between TCR intensity and probability of TCR detection. Statistics on the metacell types of pairs of cells belonging to the same clone was thereby controlled for using comparison to the randomized data (FIG. 5L). NK cells (51 cells) with retrieved TCR were removed from this analysis as they represent misclassified T cells. Cells within the memory and dysfunctional CD4 metacells were also removed because their low frequency prevented the robust statistical analysis of the clonality signatures.

Data and Software Availability

The MetaCell R package and its open-source code are available from www(dot)bitbucket(dot)org/tanaylab/metacell/src/default/

### Example 1

### Transcriptional States of Immune Cells in Human Melanoma

In order to better understand the heterogeneity of immune cells within and across melanoma patients, a protocol for single cell transcriptomic and protein index characterization of immune (CD45+) cells, and in particular T (CD3+) cells in melanoma tumors was designed (FIGS. 1A and 1G). Design of the study was focused on maintaining the in situ RNA composition of tumor infiltrating immune cells, by immediate dissociation of tumor material for MARS-seq analysis (Jaitin et al., 2014). Data was collected on a total of 47,772 QC positive tumor infiltrating cells from 25 melanoma patients, including patients with stage 3 and stage 4 melanoma with a diverse treatment history, and 9 treatment-naïve, stage 3, in-transit melanomas (Table 4).

TABLE 4
Clinical metadata for sequenced patients. Related to FIG. 1A. Multiple
metastases from the same patient are marked with a -1 or -2 in the
patient ID. PBMCs are noted as -PB. Tumor location (primary (P),
(sub)cutaneous, lymphnode (LN), and musculus (MSC)), disease
stage, and immune checkpoint therapy prior to and at the time of
resection are shown.

| | Tumor | Disease | | On- | |
|---|---|---|---|---|---|
| Patient_ID | location | stage | Prior treatment | treatment | Notes |
| p1 | LN | 4 | | | |
| p10 | MSC | 4 | aCTLA4, aPD1 | | |
| p11 | (S)C | 3 | | | |
| p12-1 | (S)C | 3 | | | |
| p12-2 | (S)C | 3 | | | |
| p13 | (S)C | 3 | | | |
| p13-PB | PBMC | 3 | | | |
| p15 | (S)C | 3 | | | |
| p16 | (S)C | 3 | | | |
| p17-1 | (S)C | 3 | | | |
| p17-2 | (S)C | 3 | | | |
| p17-PB | PBMC | 3 | | | |
| p18 | (S)C | 3 | | | |
| p19 | (S)C | 3 | | | |
| p2 | LN | 4 | aPD1, aCTLA4 | | |
| p20 | (S)C | 3 | aPD1 | | |
| p21 | (S)C | 3 | | | |
| p22 | LN | 3 | | | No tumor |
| | | | | | cells left |
| p23 | LN | 3 | | | |
| p24-1 | LN | 3 | | | Healthy tissue |
| p24-2 | LN | 3 | | | |
| p25 | P | 3 | | | |
| p26 | P | 2 | | | |
| p27 | (S)C | 4 | aPD1 | aPD1 | |
| p27-PB | PBMC | 4 | | | |
| p28 | (S)C | 4 | aPD1, aCTLA4 | | |
| p3 | (S)C | 4 | | | |
| p4 | (S)C | 4 | aPD1 | | |
| p5 | LN | 4 | aCTLA4, aPD1 | | |
| p6 | (S)C | 4 | aPD1 | aPD1 | |
| p8 | (S)C | 4 | aPDL1, | | |
| | | | aCTLA4, aPD1 | | |
| p9 | (S)C | 4 | aCTLA4, aPD1 | aPD1 | |

Recent work demonstrates that the infiltrating T cell compartment in human tumors is composed of a mixture of tumor reactive and bystander T cells, including T cells reactive against human herpesviruses (Scheper et al.; Simoni et al., 2018). For this reason, it was important to characterize both the transcriptional state of T cells and their TCR, allowing one to determine which T cell states are clonally linked and which are potentially tumor-specific. To this end, a variant of MARS-seq was developed that provides information on both the TCR and the transcriptome of individual T cells (Methods). The MetaCell algorithm was used to identify homogeneous and robust groups of cells (“meta-cells”; Methods) from scRNA-seq data, resulting in a detailed map of 324 metacells organized into seven broad lineages, including T cells (characterized by expression of CD3), NK cells (KLRD1), dendritic cells (CD1C), macrophages (C1Q), monocytes (VCAN), B cells (CD19), and plasma cells (Ig) (FIGS. 1B and 1H-J; and data not shown).

### Example 2

### T Cells Form a Gradient of Transcriptional States within Tumors

Both T and NK cells were characterized by a diverse group of transcriptional states that annotated broadly using analysis of the metacell similarity matrix (similarity between 218 metacells, FIG. 1K) and its 2D projection (FIG. 1C). This mapping revealed naïve-like T cells, but also CD4 and CD8 T cell pools with different degrees of differentiation. T cells in the naïve-like subset were metabolically inactive cells that showed weak transcriptional activity (FIG. 1D-E). FACS-based index analysis allowed subdivision of some of the naïve subset into CD4+ and CD8+ (FIG. 1L), but apart from high level expression of the IL7R, CCR7, and the transcription factor TCF7, limited additional transcriptional activity was observed relative to other T cell pools (FIGS. 1E and M-L). In contrast, the non-naïve T cell metacells showed remarkable transcriptional heterogeneity. Another small group of cells that was made up of CD4 and CD8 T cells was putatively annotated as memory T cell population (FIG. 1O). The CD8 subset was initially subdivided into a transitional CD8 T (GZMK⁻) pool, a cytotoxic T effector (GZMH⁻) pool and a large cluster of dysfunctional CD8 T cells, marked by high expression of immune checkpoint molecules such as PD-1 and LAGS. The borders between these different CD8 classes were diffuse, even though metacell resampling analysis supported the robustness of the model. This observation implied that transcriptional gradients contribute to T cell heterogeneity as further discussed below. The CD4 T cell subset was dominated by FOXP3 expressing regulatory T cells (Treg) but also included a distinct subset with characteristics of follicular helper T cells (marked by CXCL13), and another smaller group of cells expressing various immune checkpoint molecules, similar to the ones observed in CD8 T cells (FIG. 1P). Finally, although CD3 negative, NK cells expressed many gene modules that were also observed in cytotoxic T cells (FIG. 1Q).

Importantly, despite the high diversity in T cell types and states, data from multiple patients contributed to the definition of all Metacells (FIG. 1F). This observation demonstrates that a robust universal regulatory process controls the T cell states that occur within human tumors. As such, the transcriptional T cell states that are observed pan-patient can be used for in-depth analysis of the possible gene regulatory mechanisms that give rise to the diversity of T cell states in melanoma immune infiltrates.

### Example 3

### Dysfunctional CD8 Cells Span a Regulated Differentiation Spectrum

Analysis of CD8+ metacells resulted in the de novo identification of a rich set of co-regulated gene modules (FIGS. 2A and 2L), including programs associated with basic cellular functions (ACTB and MHC-I genes), naïve T cell regulation (TCF7), and specific groups of genes linked with effector functions (GZMH, GNLY, FGFBP2, and CX3CR1) or the dysfunctional state (TIGIT, PD-1, and LAG3). Based on these data, two transcriptional scores were computed, one quantifying the activity of a dysfunctional gene module (correlated with LAG3) and the other assessing overall intensity of the expression of a cytotoxic gene module (correlated with FGFBP2) (FIGS. 2B-C and 2M-O). Then a quantitative comparison of the distribution of the two programs was performed over all CD8+ metacells and observed a spectrum of transcriptional intensities, demonstrating the presence of a transitional CD8+θstate that forms a continuum with the dysfunctional T cell state. Weaker support for a continuum between the transitional and cytotoxic effector states was observed (FIGS. 2D and 2P). The observation of this continuum from the transitional towards the dysfunctional state argues against models assuming three distinct and static molecular states (i.e. clusters of transitional, cytotoxic, and dysfunctional cells), or a differentiation regime that leads from the cytotoxic program towards a dysfunctional (“exhausted”) state. In addition, the molecular data suggested that the two trajectories that lead towards the cytotoxic or to the dysfunctional state are highly regulated. It was found that many transcription factors (TFs), partly not yet associated with a dysfunctional phenotype in human T cells, correlated with the dysfunctional program. These included the Notch signaling TF RBPJ as well as ZBED2, ETV1, MAF, PRDM1, and EOMES (FIG. 2E). In addition, distinct transcription factors that correlated specifically with the cytotoxic program (KLF2 and TBX21/T-bet) were also observed (FIG. 2E). Using the expression of these TFs, a simple linear regulatory model was inferred, predicting the contrasting dysfunctional and cytotoxic programs with high accuracy (R²=0.93) using lasso-regularized cross validation on 139 metacells (FIG. 2F). We note that metacells define disjoint and robust groups of single cells and provide statistical support for putative transcriptional gradients, which robustly control for smoothing artifacts or low-depth data (FIG. 2Q).

A similar analysis of CD4 Treg metacells identified a co-regulated gene module that includes IL2RA, ICOS and GITR (FIGS. 2G-H and 2R). Furthermore, analysis of this gene module and TFs that were correlated with it (BTAF, FOXP3, and IKZF2) across Treg metacells suggested that Tregs within tumors can be organized along a gradient of intensifying expression of characteristic Treg genes and specific TFs (FIG. 2I). Interestingly, some of the gene modules activated in Tregs overlapped with those characteristic of the dysfunctional program in CD8 cells, and a number of TFs, including PRDM1, VDR, MAF and ZBED2, were correlated with both programs. To further examine transcriptional overlap between dysfunctional CD8 T cells and regulatory T cells, the gene expression correlation was compared to the dysfunctional and Treg program (FIG. 2J). Genes that were correlated with both the dysfunctional and Treg gradient included regulatory molecules and many co-inhibitory and co-stimulatory receptors (e.g. TNFRSF9/CD137, CSF-1 and TIGIT). In contrast, FOXP3, IL2RA (CD25), and IKZF4 were only associated with regulatory T cells, while PD-1, CXCL13, IFNG, and EOMES were preferentially correlated with the dysfunctional and not the Treg program. Visualization of the dysfunctional and Treg scores for metacells confirmed the overlap between these two differentiation programs (FIG. 2K). Interestingly, CD4 Tfh metacells shared genes with the dysfunctional CD8 program that were distinctively missing in Tregs—including CXCL13 (FIG. 2S). In conclusion a dominant group of dysfunctional CD8 T cells was observed that is characterized by gradual rather than discrete activation of immune checkpoint gene expression, and that also partially shares regulatory mechanisms with regulatory CD4 T and Tfh cell populations. In contrast, the observed CD8 cytotoxic cell pool stands out as a more distinct population.

### Example 4

### Monocyte Differentiation and Bifurcation is Observed within Tumors

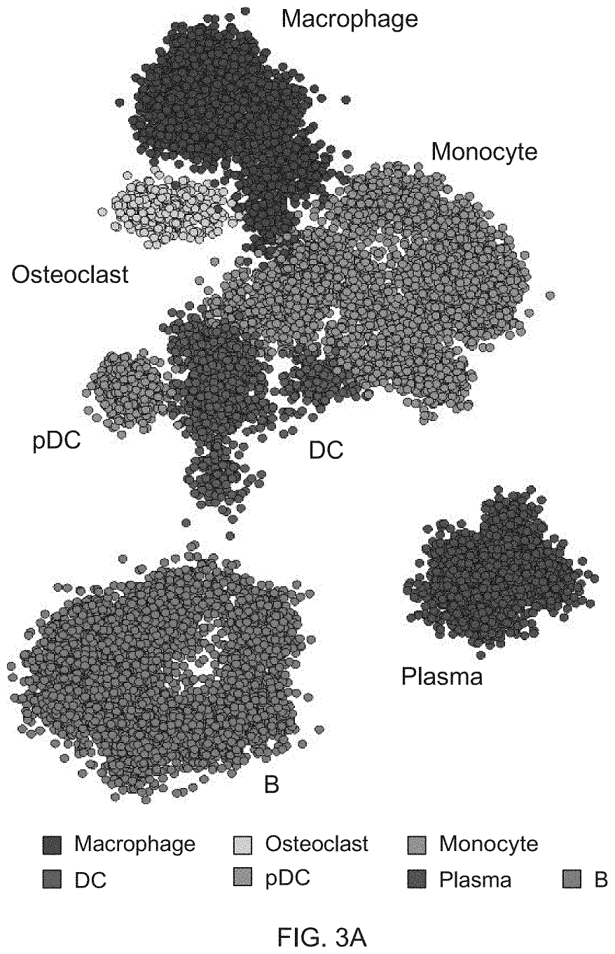

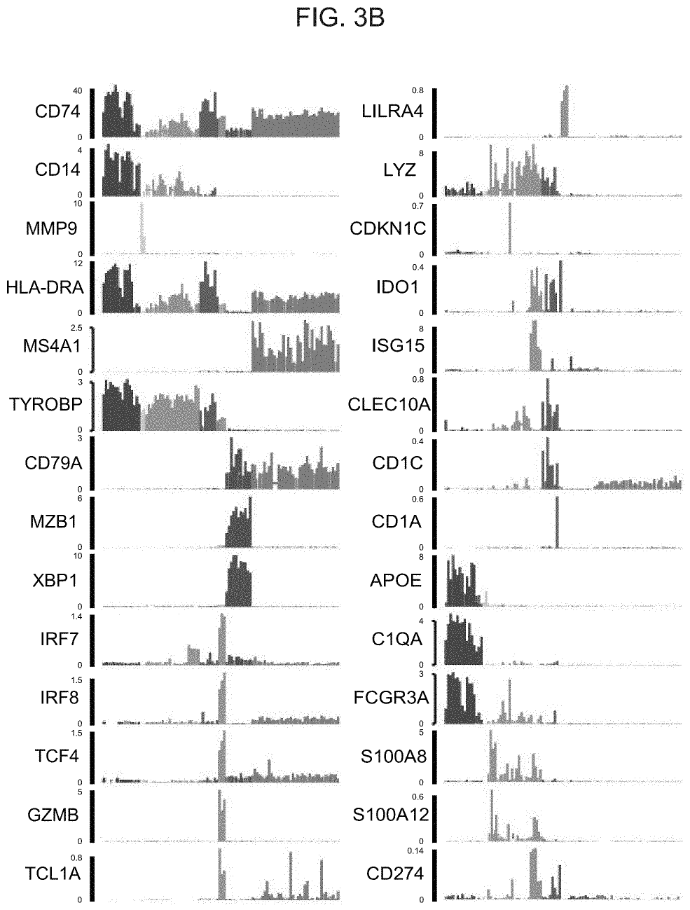

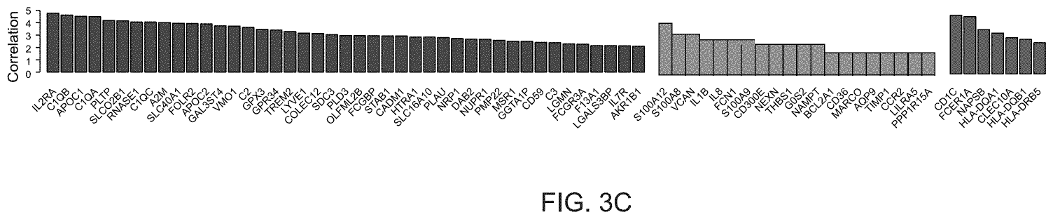

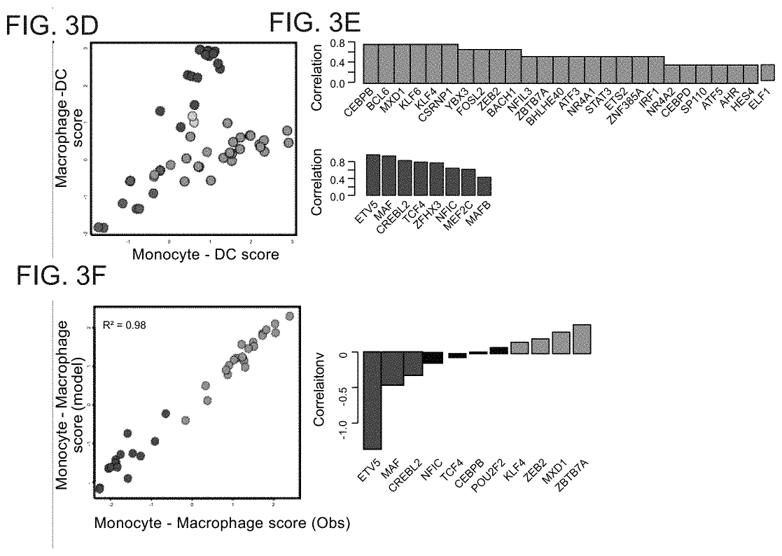

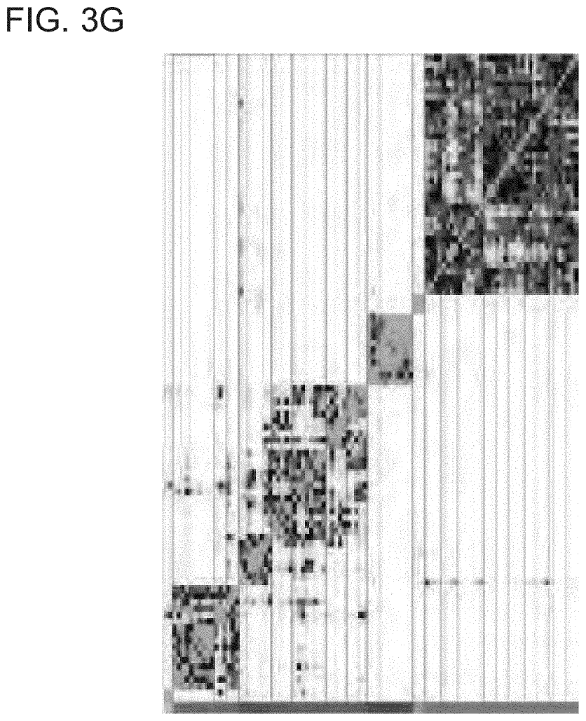

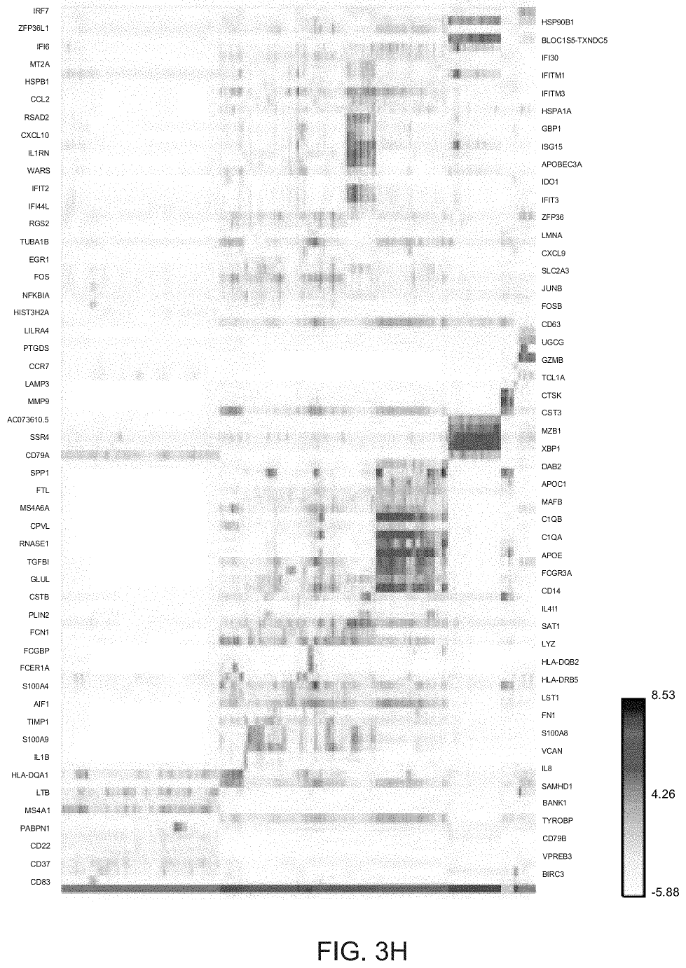

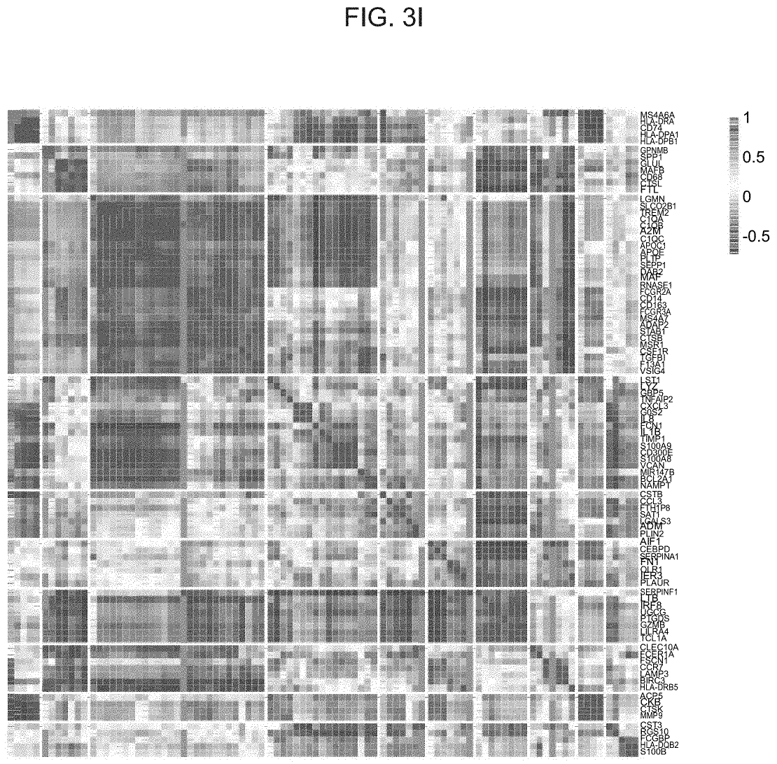

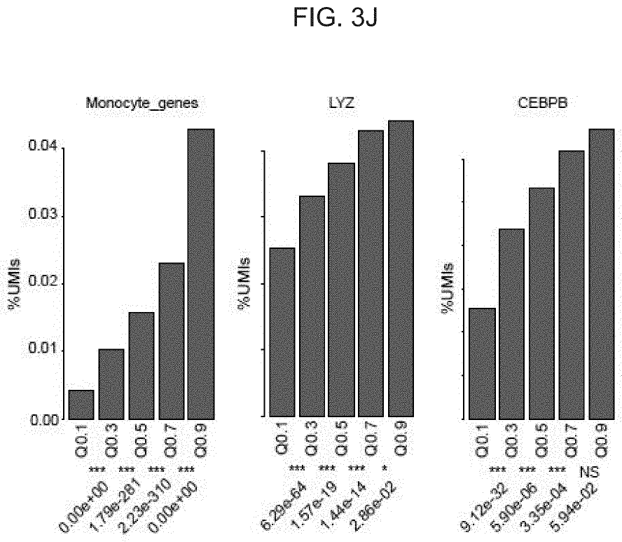

An in-depth analysis of the metacell map was performed consisting of 16,412 QC positive CD45+ cells, across all patients, following in silico removal of T/NK cells (FIG. 3A). Within this model diverse myeloid cell types were observed, including macrophages (C1Q), monocytes (VCAN), dendritic cells (DC; enriched for CLEC10A and CD1C), plasmacytoid dendritic cells (LILRA4), and also a small group of osteoclast-like (MMP9) cells (FIGS. 3B and 3E-F; and data not shown).

In addition, metacells defining B and plasma cells were observed. To characterize potential myeloid differentiation trajectories, the transcriptional signatures for monocyte, macrophage, and DC metacells were identified (FIGS. 3C and 3F-G). These signatures were then used to compute scores for each metacell in the myeloid model, resulting in a bifurcation like structure that connects specific transcriptional states that define the three programs through metacells expressing these pathways with transitional intensity (FIGS. 3D and 3H). A model was derived that accurately predicts monocyte and macrophage metacell expression signatures from TF expression alone (R²=0.99, cross validated lasso regularized linear model) (FIG. 3E-F). These data, and in particular the existence of monocytes in different stages of differentiation within tumors, suggest an ongoing development of monocytes within tumors, rather than the sole infiltration of mature myeloid types.

The presence of various myeloid cell populations has previously been suggested as a potential modifier of T cell activity in tumors (Binnewies et al., 2018; Lavin et al., 2017; Merad et al., 2013; Salmon et al., 2016; Tcyganov et al., 2018).

### Example 5

### Inter-Patient Variation of Dysfunctional CD8+ T Cells

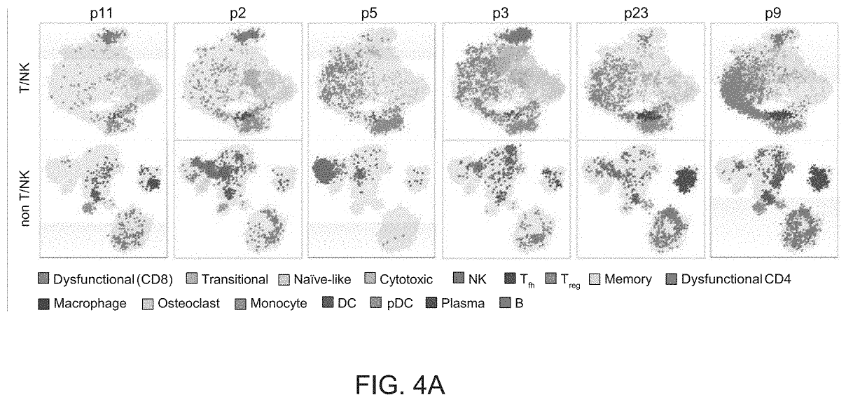

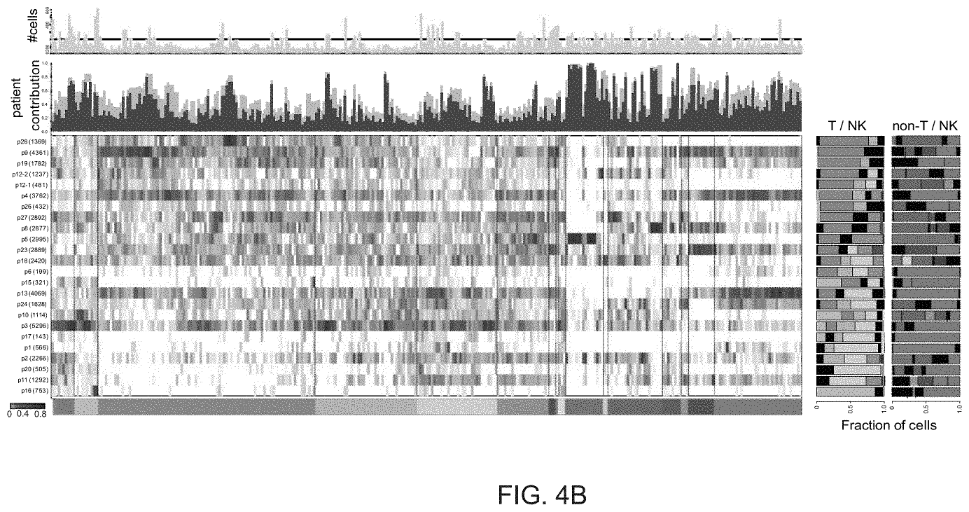

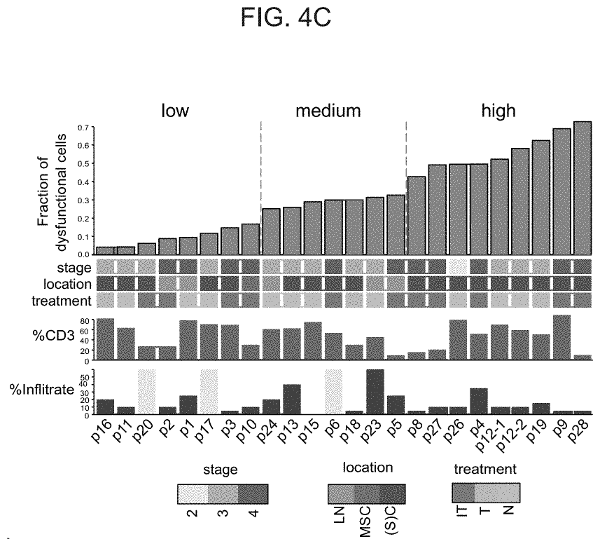

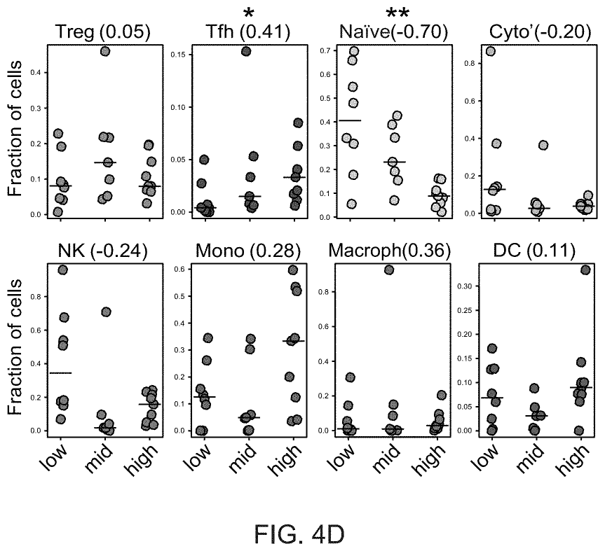

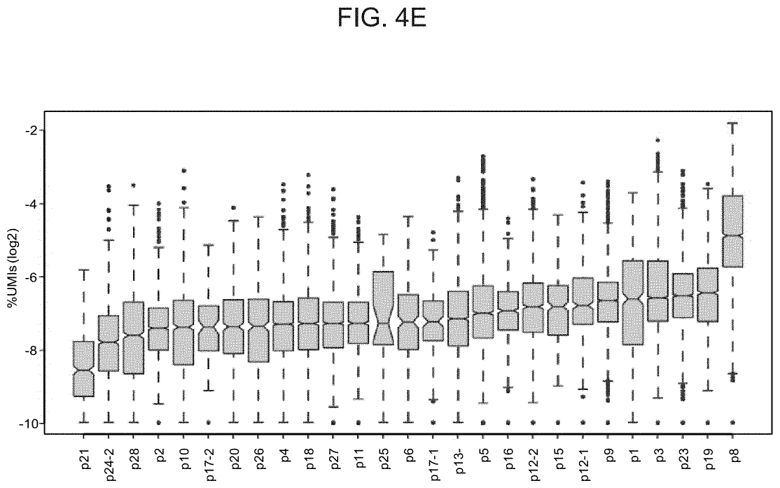

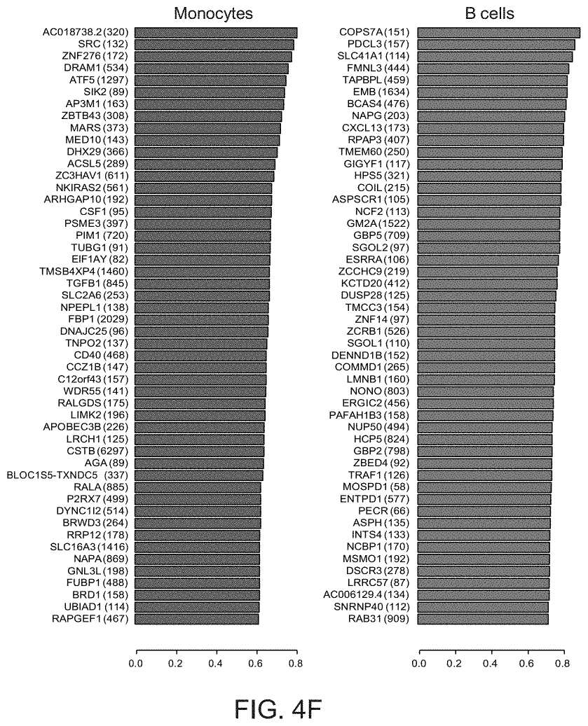

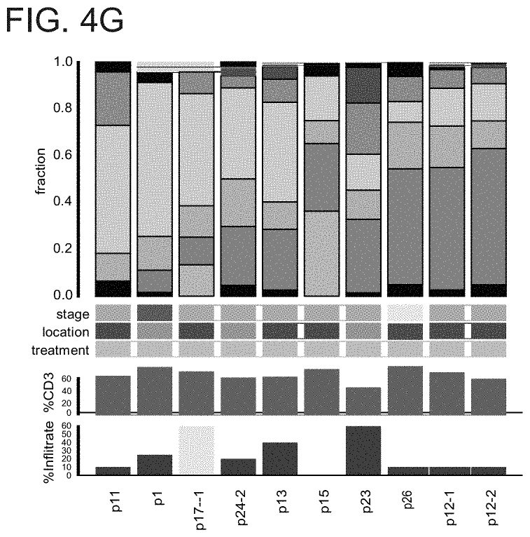

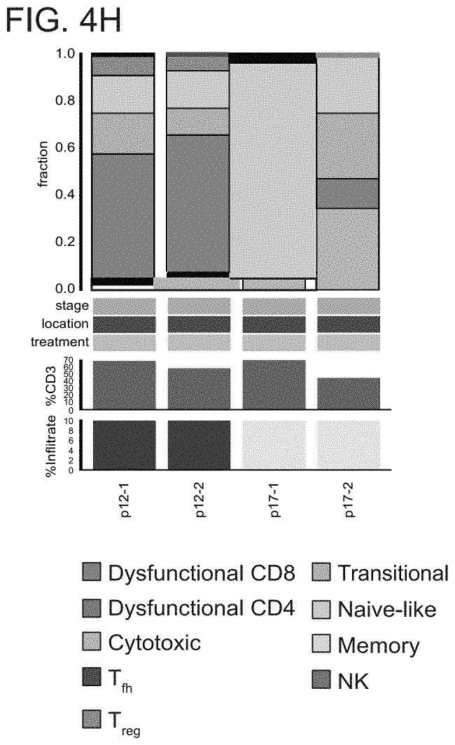

To map conserved and patient specific patterns in infiltrating immune cells, the immune composition of each patient was systematically analyzed (FIG. 4A-B). Globally, the majority of the observed T cell transcriptional states were shared across many patients, with relatively few cases of individual patients contributing more than half of the cells in a metacell. A more heterogeneous distribution was observed for myeloid cells, with many metacells composed by cells from a small number of patients. These patient-specific myeloid cell states included several macrophage and monocyte metacells highly enriched for type-I interferon signaling (FIGS. 4B and 4E). While the metacell model showed that the transcriptional states of T cells were conserved between patients, the frequency of these states in different patients was remarkably diverse. In particular, it was observed that the CD8 dysfunctional state constituted a highly variable fraction (ranging from 3.6% to 72.1%, median of 28.9%) of the tumor-infiltrating T cells (FIG. 4C). Analysis of possible correlations between dysfunctional CD8 T cell load and gene expression signatures in monocytes or in B cells showed no significant results, indicating that such correlations, if existing, are insufficiently strong to allow detection in our patient cohort (FIG. 4F). In contrast, we did observe the fraction of dysfunctional T cells to be negatively correlated to the fraction of naïve T cells (p<0.001; FIG. 4D), and positively correlated to the fraction of Tfh cells (p<0.05; FIG. 4D). The latter observation might indicate the presence of tertiary lymphoid structures with high levels of dysfunctional cells in a subset of tumors, as previously described in lung (Thommen et al., 2018) and breast cancer (Buisseret et al., 2017; Gu-Trantien et al., 2017). To start examining the possible connections between this differential representation of T cell states and treatment history or site of metastasis, heterogeneity was compared in T cell states either within a set of stage 3 and 4 tumors with different treatment history or within a set of 9 treatment-naïve, stage 3, in-transit melanomas. Notably, substantial heterogeneity was observed even when restricting analysis to treatment-naïve patients at the same anatomical site (FIG. 4G). Furthermore, analysis of 2 independent lesions for 2 patients revealed a similar composition (FIG. 4H), suggesting composition variability is not driven by technical biases. In summary, our data define a universal spectrum of dysfunctional T cell states within melanoma patients but show that the abundance of cells within this spectrum is remarkably different between patients. The current cohort does not support the existence of a strong link between myeloid cell compositions and dysfunctional or other T cell states, but does suggest that the load of CD8 dysfunctional T cells is an intrinsic, and possibly key feature of melanoma tumors.

### Example 6

### Dysfunctional CD8 Cells have Proliferative Capacity and Form Large Clones within Tumors

To understand the clonal structure of different T cell states, a modified version of the MARS-seq protocol was generated that allows coupled analysis of single cell transcriptomes and TCR sequences (FIG. 1A; Methods). With this strategy, it was possible to recover TCRβ sequence from 6,306 T cells, consisting of 3,492 unique TCRβ sequences. As expected, TCR clonotype composition was highly variable across patients, and the only case where same clones were identified from different biopsies was for the two independent metastases from patient p12, revealing remarkably similar clone compositions in the two metastatic lesions (FIG. 5A-B; not shown). Some T cell infiltrates showed a diverse TCR repertoire with minimal clonal expansion, while others were strongly dominated by a small number of T cell clones (FIG. 5B-C). Interestingly, larger clones showed a non-uniform distribution of functional states (FIG. 5C), with an enrichment for dysfunctional states and depletion of naïve states. Conversely, stratification of patients by their inferred dysfunctional T cell score showed clonality to be positively correlated with the fraction of dysfunctional cells (FIG. 5D). Projection of clonal composition data on the T cell metacells (FIG. 5E), and quantification of the fraction of cells linked with a singleton or with a clone >2 cells, highlighted dysfunctional metacells as being strongly enriched for larger clones (see additional control for TCR expression intensity in Figure M-N).

To link clonal composition with potential proliferation dynamics in tumors, we computed a proliferation score for each cell, by pooling the expression of cell cycle genes (FIG. 5O; not shown), and then used the resulting bimodal distribution to classify the proliferative state of individual cells (FIG. 5P; Methods). Notably, to avoid interference of parallel gene modules with analysis of cell state, proliferation genes were excluded during metacell derivation in the model described in FIG. 1. In contrast to expectations, a large fraction of T cells was observed to be cycling (in total 6.8%), with the fraction of proliferating cells showing a highly non-uniform distribution of T cell states (FIGS. 5F and 5Q). The highest fraction of proliferative cells was observed in dysfunctional T cells, which were nearly 10 fold more likely to proliferate than naïve-like T cells (FIGS. 5G and 5Q). FACS analysis of Ki-67, a nuclear protein marking cellular proliferation, validated the proliferative capacity of dysfunctional T cells (FIG. 5R). Furthermore, dysfunctional T cells were observed to occupy all stages of the cell cycle (G0/G1, S, and M), arguing against the possibility of cell cycle arrest (FIG. 5S). Tregs and Tfh CD4+ T cells also showed higher fractions of cells expressing proliferation-associated genes as compared to naïve or cytotoxic T cells, or as compared to NK cells. Analysis of the fraction of cells that show a proliferation signature along the dysfunctional and Treg transcriptional gradients indicated that proliferation was most profound in the earlier stages of both postulated differentiation trajectories (FIGS. 5H-J and 5T). In summary, both the proliferation dynamics and distribution of clone sizes support the model that dysfunctional T cells in human melanoma form a highly proliferative and dynamic compartment. In addition, regulatory T cells also appear to proliferate prior to full activation of their regulatory program (FIGS. 5I and 5T), but this presumed proliferation is not associated with the accumulation of large Treg clones in the tumors.

### Example 7

### Clonal Linkage of Naïve, Transitional and Dysfunctional T Cells

The clonal identifiers obtained by TCR analysis provide a unique data set to infer the lineage structure of T cells in tumors. As shown in FIG. 5K, the composition of clones generally shows high functional coherence, with the individual cells within a clone being allocated to the same functional class or related classes. In very rare cases, TCR clones were observed in sister cells that mapped to both CD4 and CD8 T metacells. Examination of index sorting data suggested that these cases originated from rare metacell mis-assignment of individual cells. To systematically analyze all available information on intra-clonal structure, including data from small clones, all pairs of cells sampled from the same TCR clone were identified and then estimated lineage relationship between cell types using pooled statistics of paired types, compared to shuffled controls (Methods). As expected, that data show a very clear separation between CD4+ cells and CD8+ cells. More interestingly, a strong clustering of transitional CD8+ and dysfunctional cells was observed, whereas cytotoxic cells formed a distinct group (FIG. 5L).

In contrast, CD8+ effector cytotoxic cells appear to originate from external inputs. To further examine this, additional 15,744 (11,872 QC positive) cells including PBMCs were pooled from 3 melanoma patients and 2 healthy donors (FIG. 6D-E). This analysis demonstrated a complete lack of dysfunctional cells within these samples but ample evidence for naïve-like, transitional CD8+ and cytotoxic T cells (FIG. 6F-H). This is consistent with a model of ongoing differentiation and proliferation of dysfunctional T cells at tumor sites, and suggests that their development may be distinct (in time and space) from the dynamics of cytotoxic T cell differentiation.

### Example 8

### High Levels of Dysfunctionality in CD8 T Cells is Associated with Tumor Reactivity

To understand the relationship between the presence of T cells with defined differentiation states and tumor reactivity of the intratumoral T cell pool, T cells from 10 patients were expanded ex vivo, and reactivity of expanded T cells towards autologous single cell tumor digest was tested. (FIG. 6A). Tumor reactivity, as measured by IFNg production, was highly variable between TILs derived from different patients. In six out of ten patients, 2%-38% of the T cells showed tumor reactivity, and for the remaining patients no reactivity above background could be detected (below 1%) (FIG. 6B). Of those 4 samples, tumor cells from one patient (p8) were HLA class I negative, thereby preventing proper analysis of CD8+ T cell reactivity. Interestingly, when ordering patients by the distribution of dysfunctional scores within CD8+ T cells, we observed that T cell pools with detectable reactivity against autologous tumor cells generally displayed a more prominent CD8+ dysfunctional state (FIG. 6C). When combined with the data on transcriptional gradients that characterize dysfunctional states, the clonal composition of the dysfunctional T cell pool and the evidence for ongoing proliferation of this subset, these data support a model in which an ongoing intratumoral T cell differentiation is induced by antigen-driven interactions with surrounding tumor cells.

### Example 9

### AKAP5 and ID3 Overexpression Primes Tumor Infiltrating T Cells Towards a Dysfunctional State

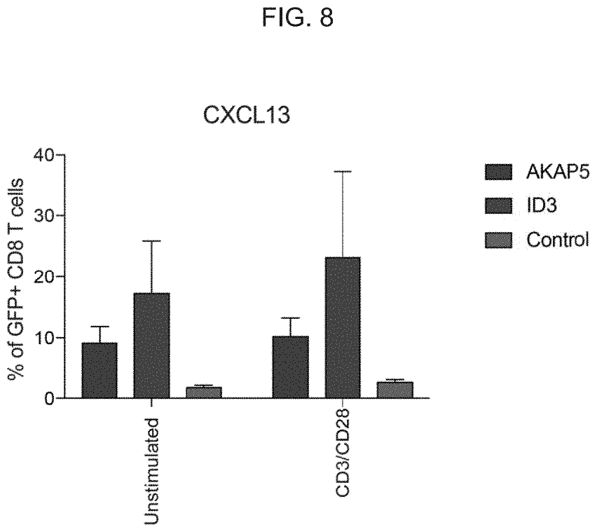

In order to evaluate the involvement of AKAP5 and ID3 in the priming of a dysfunctional phenotype in CD8 T cells an over expression model of AKAP5 and ID3 was used in T cells expanded from the tumor or heathy donor peripheral blood mononuclear cells (PBMC). AKAP5, ID3, and GFP control were overexpressed in T cells isolated from both the tumor and PBMC and the dysfunctional status was measured using FACS for intra cellular staining of CXCL13 production in CD8 T cells, in unstimulated conditions, or after TCR triggering by CD28 and CD3 for 12 hours (FIG. 8). Whereas CD8 and CD4 T cells from healthy blood and CD4 T cells expanded from the tumor did not produce CXCL13 upon overexpression of both AKAP5 and ID3 (data not shown), overexpression of either of these factors in tumor expanded CD8 T cells induced production of CXCL13, irrespective of TCR triggering.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

(other references are recited throughout the application)
Apetoh, L., Smyth, M. J., Drake, C. G., Abastado, J.-P., Apte, R. N., Ayyoub, M., Blay, J.-Y., Bonneville, M., Butterfield, L. H., Caignard, A., et al. (2015). Consensus nomenclature for CD8+ T cell phenotypes in cancer. Oncoimmunology 4, e998538.
Azizi, E., Carr, A. J., Plitas, G., Cornish, A. E., Konopacki, C., Prabhakaran, S., Nainys, J., Wu, K., Kiseliovas, V., Setty, M., et al. (2018). Single-Cell Map of Diverse Immune Phenotypes in the Breast Tumor Microenvironment. Cell 174, 1-16.
Binnewies, M., Roberts, E. W., Kersten, K., Chan, V., Fearon, D. F., Merad, M., Coussens, L. M., Gabrilovich, D. I., Ostrand-Rosenberg, S., Hedrick, C. C., et al. (2018). Understanding the tumor immune microenvironment (TIME) for effective therapy. Nat. Med. 24, 541-550.
Blackburn, S. D., Shin, H., Freeman, G. J., and Wherry, E. J. (2008). Selective expansion of a subset of exhausted CD8 T cells by alphaPD-L1 blockade. Proc. Natl. Acad. Sci. U.S.A 105, 15016-15021.
Borst, J., Ahrends, T., Bqbala, N., Melief, C. J. M., and Kastenmüller, W. (2018). CD4+ T cell help in cancer immunology and immunotherapy. Nat. Rev. Immunol. 1.
Buisseret, L., Garaud, S., de Wind, A., Van den Eynden, G., Boisson, A., Solinas, C., Gu-Trantien, C., Naveaux, C., Lodewyckx, J.-N., Duvillier, H., et al. (2017). Tumor-infiltrating lymphocyte composition, organization and PD-1/PD-L1 expression are linked in breast cancer. Oncoimmunology 6, e1257452.
Chihara, N., Madi, A., Kondo, T., Zhang, H., Acharya, N., Singer, M., Nyman, J., Marjanovic, N. D., Kowalczyk, M. S., Wang, C., et al. (2018). Induction and transcriptional regulation of the co-inhibitory gene module in T cells. Nature 558, 454-459.
Gu-Trantien, C., Migliori, E., Buisseret, L., Wind, A. de, Brohée, S., Garaud, S., Noel, G., Chi, V. L. D., Lodewyckx, J.-N., Naveaux, C., et al. (2017). CXCL13-producing TFH cells link immune suppression and adaptive memory in human breast cancer. JCI Insight 2.
Guo, X., Zhang, Y., Zheng, L., Zheng, C., and Song, J. (2018). Global characterization of T cells in non-small cell lung cancer by single-cell sequencing. Nat. Med. 1-18.
Han, A., Glanville, J., Hansmann, L., and Davis, M. M. (2014). Linking T-cell receptor sequence to functional phenotype at the single-cell level. Nat. Biotechnol. 32, 684-692.
Hashimoto, M., Kamphorst, A. O., Im, S. J., Kissick, H. T., Pillai, R. N., Ramalingam, S. S., Araki, K., and Ahmed, R. (2018). CD8 T Cell Exhaustion in Chronic Infection and Cancer: Opportunities for Interventions. Annu. Rev. Med 69, 301-318.
Im, S. J., Hashimoto, M., Gerner, M. Y., Lee, J., Kissick, H. T., Burger, M. C., Shan, Q., Hale, J. S., Lee, J., Nasti, T. H., et al. (2016). Defining CD8+ T cells that provide the proliferative burst after PD-1 therapy. Nature 537, 417-421.
Jaitin, D. A., Kenigsberg, E., Keren-Shaul, H., Elefant, N., Paul, F., Zaretsky, I., Mildner, A., Cohen, N., Jung, S., Tanay, A., et al. (2014). Massively parallel single-cell RNA-seq for marker-free decomposition of tissues into cell types. Science 343, 776-779.
Lambert, S. A., Jolma, A., Campitelli, L. F., Das, P. K., Yin, Y., Albu, M., Chen, X., Taipale, J., Hughes, T. R., and Weirauch, M. T. (2018). The Human Transcription Factors. Cell 172, 650-665.
Lavin, Y., Kobayashi, S., Leader, A., Amir, E. D., Elefant, N., Bigenwald, C., Remark, R., Sweeney, R., Becker, C. D., Levine, J. H., et al. (2017). Innate Immune Landscape in Early Lung Adenocarcinoma by Paired Single-Cell Analyses. Cell/69, 750-765.e17.
Merad, M., Sathe, P., Helft, J., Miller, J., and Mortha, A. (2013). The Dendritic Cell Lineage: Ontogeny and Function of Dendritic Cells and Their Subsets in the Steady State and the Inflamed Setting. Annu. Rev. Immunol 31, 563-604.
Paley, M. A., Kroy, D. C., Odorizzi, P. M., Johnnidis, J. B., Dolfi, D. V., Barnett, B. E., Bikoff, E. K., Robertson, E. J., Lauer, G. M., Reiner, S. L., et al. (2012). Progenitor and terminal subsets of CD8+ T cells cooperate to contain chronic viral infection. Science (80-). 338, 1220-1225.
Pauken, K. E., and Wherry, E. J. (2015). Overcoming T cell exhaustion in infection and cancer. Trends Immunol. 36, 265-276.
Pauken, K. E., Sammons, M. A., Odorizzi, P. M., Manne, S., Godec, J., Khan, O., Drake, A. M., Chen, Z., Sen, D. R., Kurachi, M., et al. (2016). Epigenetic stability of exhausted T cells limits durability of reinvigoration by PD-1 blockade. Science 354, 1160-1165.
Petrovas, C., Price, D. A., Mattapallil, J., Ambrozak, D. R., Geldmacher, C., Cecchinato, V., Vaccari, M., Tryniszewska, E., Gostick, E., Roederer, M., et al. (2007). SIV-specific CD8+ T cells express high levels of PD1 and cytokines but have impaired proliferative capacity in acute and chronic SIVmac251 infection. Blood 110, 928-936.
Reading, J. L., Galvez-Cancino, F., Swanton, C., Lladser, A., Peggs, K. S., and Quezada, S. A. (2018). The function and dysfunction of memory CD8+ T cells in tumor immunity. Immunol. Rev. 283, 194-212.
Ribas, A., and Wolchok, J. D. (2018). Cancer immunotherapy using checkpoint blockade. Science 359, 1350-1355.
Salmon, H., Idoyaga, J., Rahman, A., Leboeuf, M., Remark, R., Jordan, S., Casanova-Acebes, M., Khudoynazarova, M., Agudo, J., Tung, N., et al. (2016). Expansion and Activation of CD103(+) Dendritic Cell Progenitors at the Tumor Site Enhances Tumor Responses to Therapeutic PD-L1 and BRAF Inhibition. Immunity 44, 924-938.
Savas, P., Virassamy, B., Ye, C., Salim, A., Mintoff, C. P., Caramia, F., Salgado, R., Byrne, D. J., Teo, Z. L., Dushyanthen, S., et al. (2018). Single-cell profiling of breast cancer T cells reveals a tissue-resident memory subset associated with improved prognosis. Nat. Med. 24, 986-993.
Scheper, W., Kelderman, S., Fanchi, L. F., Linnemann, C., Bendle, G., Rooij, M. A. J. de, Hirt, C., Mezzadra, R., Slagter, M., Dijkstra, K., et al. Low and variable tumor-reactivity of the intratumoral TCR repertoire in human cancers. Nat. Med. In Press.
Sharma, P., and Allison, J. P. (2015). The future of immune checkpoint therapy. Science 348, 56-61.
Sharma, P., Hu-Lieskovan, S., Wargo, J. A., and Ribas, A. (2017). Primary, Adaptive, and Acquired Resistance to Cancer Immunotherapy. Cell 168, 707-723.
Shin, H., Blackburn, S. D., Blattman, J. N., and Wherry, E. J. (2007). Viral antigen and extensive division maintain virus-specific CD8 T cells during chronic infection. J. Exp. Med. 204, 941-949.
Shin, H., Blackburn, S. D., Intlekofer, A. M., Kao, C., Angelosanto, J. M., Reiner, S. L., and Wherry, E. J. (2009). A role for the transcriptional repressor Blimp-1 in CD8(+) T cell exhaustion during chronic viral infection. Immunity. 31, 309-320.
Simoni, Y., Becht, E., Fehlings, M., Loh, C. Y., Koo, S. L., Teng, K. W. W., Yeong, J. P. S., Nahar, R., Zhang, T., Kared, H., et al. (2018). Bystander CD8+ T cells are abundant and phenotypically distinct in human tumour infiltrates. Nature 557, 575-579.
Śledzińska, A., Menger, L., Bergerhoff, K., Peggs, K. S., and Quezada, S. A. (2015). Negative immune checkpoints on T lymphocytes and their relevance to cancer immunotherapy. Mol. Oncol. 9, 1936-1965.
Stubbington, M. J. T., Lönnberg, T., Proserpio, V., Clare, S., Speak, A. O., Dougan, G., and Teichmann, S. A. (2016). T cell fate and clonality inference from single-cell transcriptomes. Nat. Methods 13, 329-332.
Tcyganov, E., Mastio, J., Chen, E., and Gabrilovich, D. I. (2018). Plasticity of myeloid-derived suppressor cells in cancer. Curr. Opin. Immunol. 51, 76-82.
Thommen, D. S., and Schumacher, T. N. (2018). T Cell Dysfunction in Cancer. Cancer Cell 33, 547-562.
Thommen, D. S., Koelzer, V. H., Herzig, P., Roller, A., Trefny, M., Dimeloe, S., Kiialainen, A., Hanhart, J., Schill, C., Hess, C., et al. (2018). A transcriptionally and functionally distinct PD-1+ CD8+ T cell pool with predictive potential in non-small-cell lung cancer treated with PD-1 blockade. Nat. Med. 24, 994-1004.
Tirosh, I., Izar, B., Prakadan, S. M., Wadsworth, M. H., Treacy, D., Trombetta, J. J., Rotem, A., Rodman, C., Lian, C., Murphy, G., et al. (2016). Dissecting the multicellular ecosystem of metastatic melanoma by single-cell RNA-seq. Science 352, 189-196.
Wherry, E. J., and Kurachi, M. (2015). Molecular and cellular insights into T cell exhaustion. Nat. Rev. Immunol. 15, 486-499.
Zheng, C., Zheng, L., Yoo, J. K., Guo, H., Zhang, Y., Guo, X., Kang, B., Hu, R., Huang, J. Y., Zhang, Q., et al. (2017). Landscape of Infiltrating T Cells in Liver Cancer Revealed by Single-Cell Sequencing. Cell 169, 1342-1356.e16.

What is claimed is:

1. A method of activating dysfunctional CD8⁺/Lag3⁺/PD1⁺/Tim3⁺/CD103⁺/CD39⁺/CD137⁺/Klrg1⁻ T cells, the method comprising, contacting dysfunctional CD8⁺/Lag3⁺/PD1⁺/Tim3⁺/CD103⁺/CD39⁺/CD137⁺/Klrg1⁻ T cells with an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby activating the dysfunctional immune cells.

2. A method of treating a subject having a tumor, the method comprising:
(a) determining responsiveness of the subject to immune checkpoint inhibition by: determining in the tumor of the subject a level of dysfunctional CD8⁺/Lag3⁺/PD1⁺/Tim3⁺/CD103⁺/CD39⁺/CD137⁺/Klrg1⁻ T cells, wherein a level of said dysfunctional cells above a predetermined threshold is indicative of a response to the immune checkpoint inhibition; and
(i) wherein when said level of said dysfunctional cells is above said predetermined threshold, treating or selecting treatment for the subject with immune checkpoint inhibition; or
(ii) wherein when said level of said dysfunctional cells is below said predetermined threshold, subjecting said dysfunctional cells to ex vivo expansion and subsequently treating or selecting treatment for the subject with said immune checkpoint inhibition.

3. A method of treating a subject having a tumor, the method comprising administering to the subject an immune checkpoint inhibitor and an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby treating the subject having the tumor.

4. A method of treating a subject having a tumor, the method comprising administering to the subject an agent capable of down-regulating a target gene selected from the group consisting of AKAP5, DGKH, PAG1, GALM, FUT8, WARS, CBLB, PIK3AP1, APOBEC3G, SLAMF7, SIRPG, GALNT1 or an expression product thereof, thereby treating the subject having the tumor.

5. The method of claim 3, wherein said administering said immune checkpoint inhibitor is following said administering said agent.

6. The method of claim 1, wherein said activating is performed ex-vivo.

7. The method of claim 1, wherein said activating is performed in-vivo.

8. The method of claim 1, wherein said dysfunctional cells are in a proliferative cell state and/or are not in growth arrest.

9. The method of claim 2, wherein said tumor is a solid tumor.

10. The method of claim 9, wherein said solid tumor is a melanoma.

11. The method of claim 1, wherein said dysfunctional T cells are tumor infiltrating cells.

12. The method of claim 2, wherein said immune checkpoint is selected from the group consisting of cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD244 (2B4), CD160, GARP, OX40, CD137 (4-1BB), CD25, VISTA, BTLA, TNFR25, CD57, CCR2, CCRS, CCR6, CD39, CD73, CD4, CD18, CD49b, CD1d, CDS, CD21, TIMI, CD19, CD20, CD23, CD24, CD38, CD93, IgM, B220 (CD45R), CD317, CD11b, Ly6G, ICAM-1, FAP, PDGFR, Podoplanin, and TIGIT.

13. The method of claim 2, wherein said determining said level of dysfunctional cells is performed by fluorescence activated cell sorting (FACS).

14. The method of claim 2, wherein said determining said level of dysfunctional cells is performed by single cell transcriptome analysis.

15. The method of claim 2, wherein said dysfunctional cells are in a proliferative cell state and/or are not in growth arrest.


SEQUENCE LISTING (99 sequences)

SEQ ID NO: 1 - DNA, 6487 bp, Homo sapiens
gcagagggcgccttgctccgggtgcgaccgcggctccctggaggcctgggcggcgggccccgcgagcgcgccgccacctcgggagcagtgttggccaaggaccccgacgccctacgttgatctttaaataaagcaaaaatttatccaaagaagggctttcatcaagaaattaaagagagaaattacaagccatagagaaactgggcatttctatactagagaaaccacctaaaacaactgtatggagtaagatgaaaggtatgaatatgcctggaagaaattttacctagtgtgacatttttgctcatctttttgtcacaaaaggctgctaaggaagagagaatacagttttctagagaataagagtgcagtgtaaaaatggaaaccacaatttcagaaattcatgtagaaaacaaggatgagaagagatcagcagaaggtagtcctggggctgaaaggcagaaggaaaaggcatccatgctttgcttcaagagaagaaagaaagcagccaaagcactgaagcccaaagctggctctgaagctgctgatgtggcaaggaagtgtccacaagaagcaggagcttctgatcagccagagcccacacggggggcctgggcctcactcaaacgtcttgtaacacgcaggaaaaggtcagagtcttcaaagcagcaaaagccattggagggtgaaatgcaacctgcaataaatgctgaggatgctgatctttctaagaaaaaggcaaaatctagacttaagattccctgcataaaattcccaagagggccaaaaaggagtaatcattccaaaattatagaagactcagactgcagcatcaaagtccaggaagaagctgaaattttggatatacaaacacagaccccattgaatgatcaggcaacaaaggctaagtcaacccaggatctaagtgaaggcatctcacggaaagatggtgatgaggtctgtgaatcaaatgtgagcaatagcacaacttctggagagaaagtgatttcagtagaacttggattagataatgggcattctgctattcaaacgggaactctaatccttgaagaaattgaaacgatcaaggaaaaacaagatgttcaaccccagcaagcaagcccacttgaaacttcagaaacagaccatcagcagccagtactttctgatgttcctcctttacctgcaattccagatcaacaaattgtggaagaagccagtaacagtaccctagaaagtgcaccaaatggaaaagactatgaaagtacagagattgtagctgaagaaactaagccaaaagatactgaattgagccaagaatcagattttaaagaaaatgggatcactgaagagaaatccaaatcagaagaaagcaaaagaatggagccaattgctattattattacagacactgaaatcagtgaatttgatgttacaaaatctaaaaatgtccctaagcaattcttaatttcagctgaaaatgagcaagtaggggtttttgctaatgataatggttttgaggatagaacttcagaacaatatgaaacactcttaattgaaacagcctcttctctagtcaagaatgctattcagttgtcaatagaacagctggttaatgaaatggcctctgatgataataaaataaacaatcttctacagtgacttactctccagagtcacggcagaaaaaacaagcttaatgaagaattcttttatacatttgtggcatttcttactcagtaacaaatgagagatttatcatctctagaacttaaaaggtacaattccagcgcagaagtgctaaagattaagtcaagtttataaaactctaccactgaaatgcagtcacttctggattggaggaagtcagcacagattgcagtgtaaaatgacataacatacagggagttgctaaaatggcatatggagattggagtgctttgggggaggaaggtgtatttattgagcacttatggtatgccataagctttttaatgacctctgatataacaaagtctggtttccttttgataattcagctctgtgtataggctaacatcacattaagtttttaaaaacttattttttacagtgcacatatatgtttaaaatggtgaataaggtgagctacttttgtgaatgtgatttgtaattgttacattcctaagcagcaggcttaactcaaaaaatacattgcattttcccaatttcagcagatagtgtgcagaatatgcatattgatattaaacgtgagtggtttcctagtcttaagtctcttataagcgctatttggccaggggcagtggctcacgcctataatcccagcactttgggaggccaaggcgggtggatcagctaaggtcaggagttcgagaccagcctggccaatgtggtgaaaccccatctctactgaaaatacaaaaattagctgggcgtggtggtgcacgcctgtaatcccagctacttgggaggctgaggcaggagaattgcttgaacccgagaggtggaggttgcagggagccaagattgcaccactgcactccagtctgggcaacagagcgagactccttctcaaaaaaaaaaaaaaaaaaaaagagaaccattcacaaatccttcaatcaggcaaattgttactaagtgcaaaagtgtagaaacagggagagtgggtttttcccccgcttttctttctgcctcaacagatctgtatatatccatttctgcatggatttatctgtgaaaagttaaaaaatgaaacagcaaaaacaaaacaaggggaatattattcttcatccaggagaagggaaaattcccaagtaaattatcacagataatataggattcatcttgcagaattacttctttaagagccatattcatcaacttctctccaaagaggggagagtagattcaaagccagcctggtagtaatggtcctggaaaatcctaataatctagtgcaaatatttattctcggccaaaaatgagccatgaatgtgggtgaatagaaaagtgaaaagttattgctaaagcacattataatttaaatgtcagtattccttcatgtctgtgtgttgagaccagtaaaaagcattttacatgaatattgaaagttaactttttcatcactattttcttttcttttttttttttttttgagatggagtctcactctgtcgcccaggctggagtgcagtggcgcgatcttggctcactgcaagctccacctcctgggttcacaccattcttctgcctcagcctcccaagtagctgggactacaggcgcccactacaacgctcggctaattttttgtatttttagtagagatggggtttcaccgtgttagccaggatggtcttgatctcctgacctcatgatcctcccgcctcggcctcctaaagtgctgggattacaggcatgagccaccgcacccggccttcatcactattttcaatccagatatactttacaaaaatgagaagggcttatttgcatttttatttacatattgaaagggaagcatctgatctcatcaatgattgccatattttttgaaaaagtaatcctatttcaatctttccttgtgtgaaatggccataataccagagtgcacttccctacctcacaggttaggattcaaagtgtgtattcccccattgtgtaaattgagtgattgtgttactacttgaaacagatgaagtaataaagatgaagtatgagatgtaaggtatttgttcaagagctccacttggaggaaataatgcacattagatgattttaaatgatctgggtgaattcttgcattgtgtatggattgcatatagaaagtatttgctactttgtcctacagaatcatcttcatttctttctgtaaatacatattgagacctctaacaaaagaaactaatctgacttggaaattgaaccaaatcatggatattattagcaagattctttgctgaagtgcaaaaatatttcccccagattttcctaccttcatgcttataatccggggaggagggtggaaatcctgagcaagatgaaaagtgtcattcaaaacacatgacccaagaagtcaatttcttttaattgtacaacttttaaacattaaaaacaaaaaagacgagtcatcttctctgaaaacactctttaactcactatatgtggtgctaatggacttggacagctaccagctatcatatttaaaagtgaaccattttaaaaacctgactaaggttggtgcattttacagtgtattgaagaatcctgccctcatttactgcaaggatgcctccaagccaatacacttgcattgtaaatgtttagtaatctcatgaacaagcaaaaagtatagtacttagcagttattggaaatattttgggggatttttaaaaaaacagataaatggtaaatatgtgcaatgaattttttggttaagaaagtaaaattttatctaatactaggtttttacttttttcactgtggtttgatatatatttttggttagaatgtactgattgtttttatgataagatgtatattttacttgcattcattcttaagaggtttctgtttttctattgagaatatctgttgaattaagtaggatttcagttagaaaaaggaattccgaaattaactggtctcaatctgattatatgtgctacatggccaaatcctattacagtgaattctaaggtgattcaaattgttttattgttatagaaatatgcagaaatcagatggcttcagagtcataaaatatttttcttgtaatagtatttaagcaatttgaccttatatgcaaacgttaagtgaatataaataagttctgtttcaattcatgtccatttaacaacattaaaataattattaggaacaccacatgaattttctttacatttgctgtggtataaattctgcttttaatgaatgtttatttatttattagccaaaattcagtttctctttgaacccagaattttgcaaagctgggggttggggagggggattattagctacttgttaactttttcagcatattatcattcctcttttaagactcaagtattattcactttgcttcttacaacccagagaatgtgaaaattctcctctattttatgtagcactattcacttaacattttttcaaagcactttataggatataaaaatagtacccggtaacatttaagtaaaggtagcagaaaacaaaagaactttcaaacagaaattatgttccctgccacctcctccacctcaaagaataataagcatgtggcataaatggagttgttcagaaaagctgtctgcataattttccttcaaggtaatgtatttgacagcatgaagtttgtgatgatcttacttggtctactactcagcggagtcttagaatctcttttatttacttctgagagattgttatttcaccagtatatgtgaacaggcttgctaaaggcattaaagatatggatcatggaggcaccaaatttggcttctgtatgcaatccagcattaacttcatccatacatttctaacttcatccataaatttctaacttcaagttaccttattttttatagttaagaatgtttttcgaggtttacaacagaatttaaaaaataattgttaggctttagaatcccactgtttgaaaagatccttttaatgtcaactttattgaaatatgtagtctactttctaatttcatctgataatatagcaggatttgatttttctgcctgccatttgtgcatttaaaaaaaaatcctcatttaagatttagagttctttaggttcaaattcctatgatgttttattgggtaacatgaactctcttatacataattcaaaatttcaatccatggtatattttatggtacattttgaaaagaaaatttgtaatgctccttagttacaaaaattagaaggaagctacataaaattcaggtgtcctaattaaattcaatttgattcaacacacgtattaggcataactcatacctaacactatttgataagggagacaggctccctgccatccaagaactcaacctagggagcagtgacaacagaaaccgggttgggttaaggcaaagagccatggataatgctgcaacatgagtggcacagaggggagtacacagagagagcaattaatttggtgtggggaaggtggtgatctgaatacagcagcagtttgaaagtgttccgtttttaaataaacagtatgcttattttgatttgtgactaggtgtagaaaaaagcctgcatagtgttacatatttatagtagttctttgtaaaaatgttaagtggatgagctattatgaaagtaaaagtttcattttttctcattaaaattagtactaaattactatggatcagatgataatattaaatagtactctgtagaaaaaaggtttcaagttgaattaatttatgtactgattattaatacagaataaatgttatattgactca

SEQ ID NO: 2 - protein, 427 aa, Homo sapiens
METTISEIHVENKDEKRSAEGSPGAERQKEKASMLCFKRRKKAAKALKPKAGSEAADVARKCPQEAGASDQPEPTRGAWASLKRLVTRRKRSESSKQQKPLEGEMQPAINAEDADLSKKKAKSRLKIPCIKFPRGPKRSNHSKIIEDSDCSIKVQEEAEILDIQTQTPLNDQATKAKSTQDLSEGISRKDGDEVCESNVSNSTTSGEKVISVELGLDNGHSAIQTGTLILEEIETIKEKQDVQPQQASPLETSETDHQQPVLSDVPPLPAIPDQQIVEEASNSTLESAPNGKDYESTEIVAEETKPKDTELSQESDFKENGITEEKSKSEESKRMEPIAIIITDTEISEFDVTKSKNVPKQFLISAENEQVGVFANDNGFEDRTSEQYETLLIETASSLVKNAIQLSIEQLVNEMASDDNKINNLLQ

SEQ ID NO: 3 - DNA, 1651 bp, Homo sapiens
cagtctgagcccagagagccgcggggaccatggagccggtgccgctgcaggacttcgtgcgcgccttggaccccgcctccctcccgcgcgtgctgcgggtctgctcgggggtctacttcgagggctccatctatgagatctctgggaatgagtgctgcctctccacgggggacctgatcaaggtcacccaggtccgcctccagaaggtggtctgtgagaacccgaagaccagccagaccatggagctcgcccccaacttccagggctacttcacccccctcaacaccccacagagctatgaaaccctggaggagctggtctctgccacaactcagagctccaagcagctgcccacttgcttcatgtcgacccacaggattgtcacagagggcagggtggtgactgaggaccagctcctcatgcttgaggctgtggtgatgcacctcgggatccgctctgcccgctgtgtcctgggcatggagggtcagcaggtcatcctgcacctgcccctatcccagaaggggcccttctggacatgggagcctagtgcccctcgaactctgctccaggtcctacaggatccagccctgaaagacctcgtcctcacctgccccaccctgccctggcattccctgatcctgcggccccagtatgagatccaagccatcatgcacatcttctcaagtcttaggattgcagcaacacgctcggctgcccaaacccaaggcgaagaccttgccagagttcatcaaggatggctccagtacgtacagcaagattcctgcccacaggaagggccacaggcccgctaagccccaaaggcaggatctagatgatgatgaacatgattatgaagaaatacttgagcaatttcagaaaaccatctaagtgctggaggaaccacgcttcctaactgctgcttctcagggaatccgacaccagccaaccattttaagcctctaaaagacctcgggcaagtctcacagaaactgagctgcagacggggagtagctttgtggaaactgatttgatggacactgcaccagcttccttcaggttctagattcttgctacttagggcgggctggtttggacctaacatctcgcacgtgactccctcagcctcagagccttgggatgcagagcagctggcagggttcctctcaatcctgcaaccccagctgtcccaccggtggatgcagaggggaatccgaggccatcaaccttggtgacagcagcgcagtgccaatgctgatcacactgcatgggagattttgttaacgtctgccacccccactctcacccccaagctctaagcccccgggaggcctggactgtcttcctcatctctgtagcaccaagcctgatagatctgtatatggtaaacaggggtttaaccacatgtggttaacatggattaatgtgggaacttggcttcaagaacacaaccttaggaccttgggccccaaaagctggtggtgaaatgaggaggagccaatttaagaagacccttatggagacctgaggctgcagaaactggtaggtttcatcaggtggttaaagtcgtcaaagttgtaagtgactaaccaagattatttcattttaaaaccatagaataaaaatgacacctgagcttctctatgaatgagaa

SEQ ID NO: 4 - protein, 468 aa, Arabidopsis thaliana
MGSRYASHQLSNGLFVSGRPEQPKEKPPTMSSVAMPYTGGDIKKSGELGKMFDIPTDGTKSRKSGPITGGSSRSGAQSGPVPNATGRMSGNLASAGSNSMKKTNSGPLSKHGEPLKKSSGPQSGGVTRQNSGPIPILPTTGLITSGPITSGPLNSSGAPRKISGPLDYSGSMKTHMPSVVHNQAVTTLAPEDDFSCMKSFPKPVLWLVILIFVMGFLAGGFILGAVHNAILLIVVAVLFTVVAALFIWNISCERRGITDFIARYPDADLRTAKNGQYVKVTGVVTCGNVPLESSFHRVPRCVYTSTCLYEYRGWGSKPANASHRRFTWGLRSAERHVVDFYISDFQSGLRALVKTGNGAKVTPLVDDSVVIDFKPGNEQASPDFVRWLGKKNLTNDDRIMRLKEGYIKEGSTVSVIGVVQRNDNVLMIVPTTEPLAAGWQWSKCTFPASLEGIVLRCEDSSNVDAIPV

SEQ ID NO: 5 - DNA, 10744 bp, Homo sapiens
attctccgcggcgcggcggcgagtgctggctgcggtaccctcccgctcgcttgtccgtctgcgcgcccgcgatgtgaccagccgactgggggcgggctggcggctctgcctgcggcagcgcatcggtttttctcctggcaggtccctaattgagccgtgattcccttggagccagagacacccaccaggggctcggagcgccgcgctccggggctggaggcagctgcagcgctcgggtccgcgcggccgggtgcagtagcctcagtcccagccgccgcctcgctgagtcgtgtgccctggcgaatgcccggcgcccggcacagctgagccaaggcgactggtgcagagcggcggcgcctggtcctgcacttgccgctgctgccgggctgtgccggggcggcggcgctgcgcgccggagtttgcagaggctgcctccgagcgccgtccgccaggaagactccctgcctcctgagtcccaaaaacacgagatttttctcctatttcagcagttggccacaacttcattgctgggaaaaggccaagaaagaagagagaactctcaccacaacatttcaaagggaatacattgccagaacttggaatactctactggacaaacatttcctccaaggacacagctctctgcctccatgtcaccacctttgaaggactgactgattccctcggctggtgccagtgcctgctcctgccatggggcccgcggggagcctgctgggcagcggacagatgcagatcaccctgtggggaagtctggctgctgtcgccattttcttcgtcatcaccttcctcatcttcctgtgctctagttgtgacagggaaaagaagccgcgacagcatagtggggaccatgagaacctgatgaacgtgccttcagacaaggagatgttcagccgttcagttactagcctggcaacagatgctcctgccagcagtgagcagaatggggcactcaccaatggggacattctttcagaggacagtactctgacctgcatgcagcattacgaggaagtccagacatcggcctcggatctgctggattcccaggacagcacagggaaaccaaaatgtcatcagagtcgggagctgcccagaatccctcccgagagcgcagtggataccatgctcacggcgagaagtgtggacggggaccaggggctggggatggaagggccctatgaagtgctcaaggacagctcctcccaagaaaacatggtggaggactgcttgtatgaaactgtgaaagagatcaaggaggtggctgcagctgcacacctggagaaaggccacagtggcaaggcaaaatctacttctgcctcgaaagagctcccagggccccagactgaaggcaaagctgagtttgctgaatatgcctcggtggacagaaacaaaaaatgtcgtcaaagtgttaatgtagagagtatccttggaaattcatgtgatccagaagaggaggccccaccacctgtccctgttaagcttctggacgagaatgaaaaccttcaggagaaggaagggggagaggcggaagagagtgccacagacacgaccagtgaaactaacaagagatttagctcattgtcatacaagtctcgggaagaagaccccactctcacagaagaagagatctcagctatgtactcatcagtaaataaacctggacagttagtgaataaatcggggcagtcgcttacagttccggagtccacctacacctccattcaaggggacccacagaggtcaccctcctcctgtaatgatctctatgctactgttaaagacttcgaaaaaactccaaacagcacacttccaccagcagggaggcccagcgaggagccagagcctgattatgaagcgatacagactctcaacagagaggaagaaaaggccaccctggggaccaatggccaccacggtctcgtcccaaaggagaacgactacgagagcataagtgacttgcagcaaggcagagatattaccaggctctagcaacccagaagacaaccctgggtagcctgtgatcagtgtctggagacgtttcttctgtggaagagaagaagtgacacaaacctatacttcatatgctgctttagtcacctgaagatggttggagaggccctgtcgactgttctcccagttgttcagtttctgagacagagaggtacggactaggctgcacctgagtgtgcccctgcctgccagatggacaggtacacccaagcacatctccctgctgcaccctcaccacccacaaaagatcccagctgtcagtggtctcatctcattagtgaggaaagccaagctgtatggaaaagctgcactcaccaaggaccacaatgcccccggcctaaagtactgccattcagaaaagcaggtttttcttcctctctttccttttctctgtctgctactgactttaaggctttttcccccttgaaatgtccagattcctgtggttcatcccaaggaaattttcacacaaagcttggcctttgccctcaatataggtgttttaggatggtgacaaaccatggctgctgctttctgcccagctcgccagtcctccccaaagagttgcgcatcagcacctggggatctggaccctgcgggtgaagggatggggagggacgtccctggagtctcttctgtctttgttccttcttattttggcattcgatatcagcagcctctccccaaagtacttgaagtcagttttagatgctttattttatttttctagtcaaaaacgtgtttcccccagtgtttgaaaactcgtccgaatcttttcagtattttccatgagtattgtggtacttctagacttgtttaagcccagaactcattccttcaaaacagagagccttaatctttatgttgggacacagaccacatatttggacggcagccatgcatccatcgctgaagggctgtggacatgaatgtgtatttcccatggtctccgctgcccacaccaacagtgtggcatctcataagttaactgctaccctaaggtaatctaagattaaaatgtaaacatttatttttgttatgtaagtttataagatgttttatgttcaatgcctaatttctcaaaagtgccagaaaaaaatgtatattagctattttgattttatgtacaatgatttatactctccttttgaaaagataccataaagcacataagctagatcactacaaggagctgttatcttttttctaatcaagtgtttaaaacactgatggtttttaaagactcacctttttaaatggtacttggagctcctgattcaaattacctagaccccctagagaaataaatggaatatacataaataatcattttcagtggtttatggtgggcaatattgcaatatttgaaatggtaaaaatggaaagaagaacaaaatatgatgagaggtggctgtgaattataaacctcataaaagtgtcataattccattaaggtttaattatattttttcagaaaacagtgatgaattctgtagtccagtgcttgccaatgcaaattgcctattggaatcttcttcctatattttacaaacatcagtggctgaaatagctcagagtaagagctcagcctggtttgaatttaatcatctctttagatcttataaggccagcattaggaaacttgttcacttttcattttcaaaggagcctagttgaagtgctattatgagtgtgggctatggaaagacagcttttcctacactgataaagaaaaaaaatgaggaaattatttcatccccttgtgacatctgtgactttttggatttaataatcttgctgtttttcctctttatgacaaagaatataattgggaggatgaagtgtcttaaaaattgtagagaccagctcactggaatgtttttccatccctgtattcatggcttgactttgtgactgctctacactgcatgtctgacattgcagagtgagctatgttgaggtaaactggttggttgtcattattttgcaatcagcctggtctctcccatgaagatgtcgtgtgcataagcacaatcatcactgattagaagatcacagcagaatacccttggattagagagaagttcgtaccttgcatttctctgaattctagtctctcataagcactgctttgctggatgattttcactgctttgtgttaatgactttgagcgatctctcacatgatggggttctttagtacatggtaacagccatgtcatcttacacacctagcattgtgaatgctgtagtgacatcctttataggcaccttacagctcaaaacttttgtttcatttcatgccttacttatcaaaaaggcaggaaagtaggtatgatctctaaagtaaaaaaaaaaaaaaaaaaaaaactttttatagaaagctcataaataatcatgtcattttgcaattttgttaccaaaatttcccccaagagttttcaaatattagttctgcaatgtggctatgaaatatgcactgaaatataccttttaatttgagaaccagtggttagaataagctgtgatataaagtattttcagtgtacttttaaaggaactataaggccctccagcataaacgctaaaagaatagatggtagcacaggccatgagggctgggggagagaagcagagtgaaccttagaaagatggctcagctatttggagcactggatattttactgaagttatttactgaggcaccatcactgttttgactgtacagtatagtttttcataaatttcatcacatttactttgttcagaatctgggcttgaatctttgagttggacaaaagcctatggtttcttttaaaaagtttcatcttgagctaatgctacagtttaaataaaatgtatgaaaggtagttatcaaaaacaatttgtatatttaaaattctattttgacatccattttttacagcgaattatagcaggttatgtgaaatttaaaaataaattttaggaaatttttttatcttgtaagtaagaattataaatctatctcagttaaaagtaggatctttgtttttattcagatattttgaaagtttagaagcattttgtatgctactttggtatttcttaagtatagtcagcaggaaagggtaacctgttttatgcaaaatttttttaatttgtcaaatgtttttagtgtgtctactttctgaaatagtctcaatatcctgtctgtatcctaggcagataactacacaagaacaaaacgctatatagaaaagaaaatatttgaggaaatcttaaattccataagaaagtaattcttgcaagaatgttggctacatatttttttttctgtcctgaataaattacatcattaggtctgttgaagcttttgaagctaccactattctttctgggataaaggctaattactgatttatctgccctcaaaatcccagagttaattctaattgagtactaaattaacagtaagtagttaacacagagaactaaaatttaaccgcattatatcactgtatatatttctcatgatttttgctaattagagcatttacttaaccagtaataaactacacacacacacacacacacacacacgtatacgtgtgtgtgtgtatatacttggctctgacacaactctggtgcttaattaggatatgttctattatgtattttgaatgtttatcctgctagtgtgatgaacttttgttggaaaagcagaattagaaggaacagtttttcaatcagtttcatttggtaattacaggaaaaccttgagttgctctaatttaatttttccaattaatacatttagactttaaattgcatccgtaattctcttggctgaaaaacagtggtattttaagacagttttgttatacatgtgacttacttttgactcagcctgagaggaggaaatgtaaagagataagagatttggcccaaagcagcaagagactagtaaccttgagccaggacgaagccaagacaacgtgactcccaagtagcaggagcagaggtcggtcacctgtggcattctgtttcattcccttttaaaattatgagactttataggcagtgtaaaacatcagcaggcagcgatgtgataggatgtgcgaaaaagctggtctgataccatggctataattacagagctttagttcaattaggattttgtaaatgagcaaatgacctttttttccagtgccccttgtaatagttaatatgagtccatgcaatcttgtgatgccattctcctgaaggtgttgatttctggtacccagccagcttagctttggctgctggaagcacagtaggtactgatgattacttcctggaaatcttgacaggcttattgtactgtgtaatggggaaaagttaaagtataatgtttggtgttaataaatgactgatgtgaaaataggaacatgtgtctttaatatcaaatatggctttatttgcagtgaaatctaaattccttattctacagtagtattttcttgccctgtgttgtatattttcctaaatacatttttcctgacagtggctttaggccagttgaacacacttagactgaaagtgtttaacttaaagagatcagtccagaaaccatgataaattagaatctttatctggaattaccaaattaaaatttaaaatcatggtccagaagagaacaaacattcatggttttttttatcctactgctcatttttaggtctgtgtttacatctagtctctactatttgtgaagtatgtcattatttttttcaagttcttctttgacttaactgaaaaataatattccaaattcttaatgtaacatgaaaagagaaatacaatttttgcttaaaagacattttttaaaaagcgttcaattcagtaatcatttctgttaatacaggaagtttttttggcttgccagttatatactgtgggtattttttaaaatgtgctacccttgggttgtctcatatcaccttgcgtaatcatgtattacaaaggttgataattgtcatttctcagatgtctgtatgttacattggcagcagtaaaatgttttaatgttgttaccttttaaataattgtgttgtattaacatgcctcatattttctggggaaacttgaaatatatctaaacaaaaaaggtctgttataaataggaattggcatttcattgttaatgttttttcctcataaaaatagttacaccaaaagtgacatattgtctattacatgggcccaatcagtattaaagtactccccttactcaaaaatattaaaaaataattcatgacacttagtagcattttcattgattgtttcaaaagatcttcataatggtcaaattgtccaattcacaaaagagtgagaaagttgttttcagtactgggaatttttaaaccttagttttaagaccaaaaatatctcttattagcttgaacattttgtgattacttttccctccccggatctagtccttttaaggagtaagtctaaagaatgaggccatgaagtcactatttcctcccacctcagtttgtctcctggtacttagctggcctatcgctttgtgtggcaatacccctgggtcgcttccccactcagccttctgtatctgctgttcacaggacatgccattatgtctttccagtacggatcacagtgctgccatatcacagaccctgccagccggcacagaaccatgcctcgtcacagctctgcccaactcacaggctgccaagatgaggccacaggtccacctagggcggcagatgctggtgctatttgtcacgacttttgccaaaacatgtacactgttgctcacatctttcgtaagaatggttgacactaaggggccatggaaaagcacttttaaaaaattatgtggtggaatcaaactcacagaggcagaaatgttagtgccgtgctctaaccaagtaagcttagtgttccccacagtgatctgtatctggcctgagtctcctcaagcagcaacccccactgtccagcatgtggaaggtagatccttatgacaacaccagacccatagttaccaggaaagaaaaaggaagctgagtgttgtgaaggggaagaaatcctttttataagaagtaatcatttttaggccgggtgcagtggctcatgcctgtaatcccagcactttgggaggctgagacaggtggatcacttgaggtcaggagtttgagacctgcctggccaacatgatgaaaccctgtctctactaaaaatgcaaaaaaaattagccaggcatggtggcatgtgcctgtagtcccaactactcaggaggctgaggcaggagaatcacttgaacctgggaggcagaggttgcaatgagctgagatcgcaacactgcactccagcctgggcgacagagcaagactccatctcaaaaacaaaaacaaaaaaaaaacctttttaaagtaaattgagtgtaaaatgttttcaccatgaagccattgcaccctctcccccaggaaacagtgcctaaggatgatgtcaagttacagccagtttgctctgatcccccaggacctaagaaaactgtgagtgcttcagcatgcaaaggtagagctaccagataaggagaataacttggccatttatagcaaactgcctgactttggagtgagaaaccaagcaccttctaggtcctaggataggtgaattggaggtgcagctgctgaagttcccattttctacatacttcaggagccagggtgtttcatttggttttgtaaaactttgctgtaagtcatcacagttaattctttgaatggacattttagagtgtaactttttttcaaaattagcagaacagatatctctgctcattctctttatacatttagttttttaaagttcccctagtatacctgttaggcactctaaataggacacagttaaagctaaaagaaggaccagttgctccctagagcctacagttcagaactatcttgttgtcatgtcctggtttggtttggtttgtttttaactcaacaagttggaaaccatgagtttaaagcaaactatttccttttcttaagcttaaagagaattttttttttaatctttagctcctcatttaggtagtgaaggcttccctgaaagacagcagtgcccttctctggaagaagaggctgggaccaaaagcctgagtctatgggttgaggattttgagcattcctttgacaagcttctatgtatttttaatataagactcataaatgaacctgatgtggtgttcatgacccatcccttaagcatgcttgctttttttaactttactttttgagtatagtacttgtcttgtttttcctggagccatgctaggatttaattcctatgtgccactacccgcccatgtcctggaagatggccatgcctgcagcaatgctcatgtcttgaggagaagcaatgtgaaaagcatcaggaaagctgaaccttacgttcaagtcagcaaacatttgtgagcattctagtatgcttgcgaccttgacactttttaaaaattatctttttatttgcttcgagtatcaaccgtatagttgactctagtggggcgaagggagcagagatgagagcaaaattcagacttgctgcccctgtgtgtctcagttctcatgtctctctgagaaatgtgttggcccaattccctatctgccactctgagtctcttgagtaaatgtgtgtaaatgagaaaaattatctcaaagatttaacttattatttagatattttcttctttttaaactcaggaataaacctggtttgtagataatataatatggctactagagctataattgagtaggtgactagaagcattttttcaaatatatttcttgtaaagacatctactttgttttaactaggaaaagaaactcctcatattaaaataaacatttccataagcactttctagaagagtgtagcatccatctaccatccatcccagaatgtttgtatcttgctcatagtatttcaaaaatttttatgggtgaaaaaaatgcacttttttatttattggaaaagtgtagagttattgaattaaattctcaagtgggacaatttagtgtaaagggaaacatgggttttggagacaagagcacagggctgtggtccctgttccagcattgactttactatgtgacctcgagcaaatgatttaacttctctgtgcctcagtttctccatatacaaaatggggataatgatactaaccattgcctacctcatagagatgttatggggacaaacgaaataatagagataagcacgaatgctttcaactcttcggaagaaaggtgctatataaattgaagttgtgtttttaatgatccaattattaattatgtttagggtttaaataacaatactgttagtcatgttaagaataagttttgttttgatgcatatttccacttctcttctgtggccagatcttgatcattcaaccagtaatggtacctgaggaactgaaatgggtatttgttttcgtatgtttctgccagtagtatttcaattgaatatccagaaaagactggagtttaacttgtaatatcttatagtttacatgtaataaaacttattcaagctatatataaaagtatgattacatgtaaatagcaggtatgttgtaattaaaggcacttatcaaattgtctttgttcttttttaattgtaataaaagtcagttttacataaa

SEQ ID NO: 6 - protein, 432 aa, Homo sapiens
MGPAGSLLGSGQMQITLWGSLAAVAIFFVITFLIFLCSSCDREKKPRQHSGDHENLMNVPSDKEMFSRSVTSLATDAPASSEQNGALTNGDILSEDSTLTCMQHYEEVQTSASDLLDSQDSTGKPKCHQSRELPRIPPESAVDTMLTARSVDGDQGLGMEGPYEVLKDSSSQENMVEDCLYETVKEIKEVAAAAHLEKGHSGKAKSTSASKELPGPQTEGKAEFAEYASVDRNKKCRQSVNVESILGNSCDPEEEAPPPVPVKLLDENENLQEKEGGEAEESATDTTSETNKRFSSLSYKSREEDPTLTEEEISAMYSSVNKPGQLVNKSGQSLTVPESTYTSIQGDPQRSPSSCNDLYATVKDFEKTPNSTLPPAGRPSEEPEPDYEAIQTLNREEEKATLGTNGHHGLVPKENDYESISDLQQGRDITRL

SEQ ID NO: 7 - DNA, 2277 bp, Homo sapiens
gctggagtttgaagagcgggcagtggctgcacacgccaaactttccctatggcttcggtgaccagggccgtgtttggagagctgccctcgggaggagggacagtggagaagttccagctgcagtcagacctcttgagagtggacatcatctcctggggctgcacgatcacagccctagaggtcaaagacaggcaggggagagcctcggacgtggtgcttggcttcgccgagttggaaggatacctccaaaagcagccatactttggagcagttattgggagggtggccaaccgaatcgccaaaggaaccttcaaggtggatgggaaggagtatcacctggccattaacaaggaacccaacagtctgcatggaggagtcagagggtttgataaagtgctctggacccctcgggtgctgtcaaatggcgtccagttctcgcgcatcagtccagatggtgaagaaggctaccccggagagttaaaagtctgggtgacatacaccctggatggcggagagctcatagtcaactacagagcacaagccagtcaggccacaccagtcaacctgaccaaccattcttacttcaacctggcaggccaggcttccccaaatataaatgaccatgaagtcaccatagaagcggatacttatttgcctgtggatgaaaccctgattcctacaggagaagttgccccagtgcaaggcactgcattcgacctgagaaagccagtggagcttggaaaacacctgcaggacttccatctcaatggttttgaccacaatttctgtctgaagggatctaaagaaaagcatttttgtgcaagggtgcatcatgctgcaagcgggcgggtactagaagtatacaccacccagcccggggtccagttttacacgggcaacttcctggatggcacattaaagggcaagaatggagctgtctatcccaagcactccggtttctgcctggagactcagaactggcctgatgcagtcaatcagccccgcttccctcctgtgctgctgaggcctggtgaggagtatgaccacaccacctggttcaagttttctgtggcttaaggaagtgtgaagatatgatccagtccagggctaggctcagccacctgtctcctgtccagaaaaaaggtgaagattaagaagctttcagaatgattctatggattaaaatcatacaaatggtggctgttctgagaatcagtctgggtattgatttccttttccagtgactggctccaggccatgtctaatgaccagctcgattccctgtgcagttcagagggcaagtgaacccaaccaacaatgtcgtcatctaagccctgaccctagccagggactcccatgctgctgttggctccatctctccacactgcctctttcttttcaactttttgcccttcctttctttaaagctattctcacattgcttttatttcctcctccttcacctccaaccactgtcagcagcactctggagttttcaaatgtcacattagcctcaccctgcatgctaggagatggacctgtctctatacagcagtagatgattgataagtgaggaaactgaggcttacagaggttgaagaccaacaagctaataaataaaaagttgaggccgggcacggtggctcacgcctgtaatcccagcactttggaaggccgaggcaggcggatcacgaggtcaggagatcgagaccatcctggctaacacggtgaaaccccgtctctacataaaaaattagctgggtgtggtagcacgcacctgtagtcccagctacttgggaggctgaagcaggagaatcgcttgaacctgggaggtggaggttgcagtgagccgagatcatgccactgcactgggcaacagagcaaaactcgatctcaaaaaaaaaaaaaaaaaaaaggaaaaaaaagcagctctctctctcgggagagggaggggacttccgagaggaaaaggcctgcaagagcctttgggttaactgagaaagcacagcccggccaacgcttgggaagaaagacaggcaagcactggaaacaggagctattagggtgaggagagagccttgcctttcacagcacagtgaggatgacattgcaccagggacagagattgtatgtgacaaggctggggctccttctttcctagacaatcaaagtatcagtgatgggttttggttggtgattttgaaaaaaaaaaaaaaaagtatgttatactagtgtatgctgtactgaattgaaataaaaccaaaatctgcatcttca

SEQ ID NO: 8 - protein, 342 aa, Homo sapiens
MASVTRAVFGELPSGGGTVEKFQLQSDLLRVDIISWGCTITALEVKDRQGRASDVVLGFAELEGYLQKQPYFGAVIGRVANRIAKGTFKVDGKEYHLAINKEPNSLHGGVRGFDKVLWTPRVLSNGVQFSRISPDGEEGYPGELKVWVTYTLDGGELIVNYRAQASQATPVNLTNHSYFNLAGQASPNINDHEVTIEADTYLPVDETLIPTGEVAPVQGTAFDLRKPVELGKHLQDFHLNGFDHNFCLKGSKEKHFCARVHHAASGRVLEVYTTQPGVQFYTGNFLDGTLKGKNGAVYPKHSGFCLETQNWPDAVNQPRFPPVLLRPGEEYDHTTWFKFSVA

SEQ ID NO: 9 - DNA, 4749 bp, Homo sapiens
tttcagttggaaggaggtagggaaaataagtgctttaattcctaggattttggaagtaaaaatggaacaccagacaaaaatggaatttttaggcaaatgtaaagctttgtacttgggtatctcaaagtattatacagtgtaaactatcatgattattaaccaactagaagtagttcttttttttaaattttatttttaattttagacatgagggtcttgctatgttgcccaggctgaagtggctactgacaggcacgatgtggcactgcagcctggaactcttgggcttaagtgatccttcctgcttcttctatcttaaaaaaaaaaagttacgcttgcttgttaaaatatttttaaaatgctgtgtttaaactaagaagtaaatatccaccccacctctgacttctaaaaattatctaaagtatgactggtaagatttttacctttaatcccacataagtctaaaatttgtgttcatttccaacaaccaaagtgtgtcacttttataccagaattttgtcctgcttatacgtttagtacagaaatctcatgggagagagcatccatgcatttacaaattgttattgaattattttattgaatgatgacacccaaactgagctagaacataattctggctctgctagtacatcttctgtgtgatcttggacaagtcactctactttcctttcaattttcttttctcacagggagataatcataaaaacgactgtaaagtacagcacttcatagagtgctttttgtttaaagagctgacaataaatacgagtctcaaggtctaggaaagcctccctcacaacctgagctgcttgaggacaagggattttcttttgaatcagcagtaccttatttgtgtatctgtgatagagttcctggtacataagaaggtctcaataaatatgtgaatttatgaatattaggcagattgcaaccttgacaggccactgcctcttaaatctcctttctgtgatcttttaatatttaacatctaaaaggccgccgctacttgctttgggataagtatccccggtatgtactttaaaatgcccaagcctagagaaatgattcttgtcttaagggcaccatttcgctctcccaccgtaaagcgccccaggcttgggatctgggtcccaaggctacagggaagagtttggaacgggaagctcatcttccggccctctgattggccggctcgcactccactcacgcggcgcgcagctctgattggcctcggcggcacccctcgtcccgcgactactttgtgtgctggggcggcgcgctccggtcctcccgctcagctggcggtctgggctgctctggggcagcccttcggtccactgctctgcatcgcgggcgccgggaattttccgagtccgagcgggttgctgcttttgctcagaggacatccatgaccctaatggtctttttgttcaagataaagtgattttttgcctttgttgattaactggacaaattcaggataccagaaggccctattgatcaggggccagctataggaagagtacgcgttttagaagagcagcttgttaaggccaaagaacagattgaaaattacaagaaacagaccagaaatggtctggggaaggatcatgaaatcctgaggaggaggattgaaaatggagctaaagagctctggtttttcctacagagtgaattgaagaaattaaagaacttagaaggaaatgaactccaaagacatgcagatgaatttcttttggatttaggacatcatgaaaggtctataatgacggatctatactacctcagtcagacagatggagcaggtgattggcgggaaaaagaggccaaagatctgacagaactggttcagcggagaataacatatcttcagaatcccaaggactgcagcaaagccaaaaagctggtgtgtaatatcaacaaaggctgtggctatggctgtcagctccatcatgtggtctactgcttcatgattgcatatggcacccagcgaacactcatcttggaatctcagaattggcgctatgctactggtggatgggagactgtatttaggcctgtaagtgagacatgcacagacagatctggcatctccactggacactggtcaggtgaagtgaaggacaaaaatgttcaagtggtcgagcttcccattgtagacagtcttcatccccgtcctccatatttacccttggctgtaccagaagacctcgcagatcgacttgtacgagtgcatggtgaccctgcagtgtggtgggtgtctcagtttgtcaaatacttgatccgcccacagccttggctagaaaaagaaatagaagaagccaccaagaagcttggcttcaaacatccagttattggagtccatgtcagacgcacagacaaagtgggaacagaagctgccttccatcccattgaagagtacatggtgcatgttgaagaacattttcagcttcttgcacgcagaatgcaagtggacaaaaaaagagtgtatttggccacagatgacccttctttattaaaggaggcaaaaacaaagtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataaagctcagatggaagagataaacgaccaaactcagttcgaccaaactcagttcaaaccatttcagccaaactgtagatgaagagggctctgatctaacaaaataaggttatatgagtagatactctcagcaccaagagcagctgggaactgacataggcttcaattggtggaattcctctttaacaagggctgcaatgccctcatacccatgcacagtacaataatgtactcacatataacatgcaaacaggttgttttctactttgcccctttcagtatgtccccataagacaaacactgccatattgtgtaatttaagtgacacagacattttgtgtgagacttaaaacatggtgcctatatctgagagacctgtgtgaactattgagaagatcggaacagctccttactctgaggaagttgattcttatttgatggtggtattgtgaccactgaattcactccagtcaacagattcagaatgagaatggacgtttggtttttttttgtttttgtttttgttttttcctttataaggttgtctgtttttttttttttaaataattgcatcagttcattgacctcatcattaataagtgaagaatacatcagaaaataaaatattcactctccattagaaaattttgtaaaacaatgccatgaacaaattctttagtactcaatgtttctggacattctctttgataacaaaaaataaattttaaaaaggaattttgtaaagtttctagaattttatatcattggatgatatgttgatcagccttatgtggaagaactgtgataaaaagaggagctttttagtttttcagcttatttactttgttttttttcctgtttctgatatagtaactaattcttaactcagagacattggtccattttaatactgaaaaccaattttcattggtacacattacaaaattgctaagaacactgtttgggagctttcattctcatattttggacattgttttaattgagtgaaataatcataactccttgctcccagagaagctatcacctccatttctaaaaccatttcaggtttgtttggtagtctttcttaatgtatttattatcgctttttgtgagtagggtcctgttttatccaggaaccaatttctgccctagcctatcatgccctgcttttgagagtaccaggtatctttgggattgggaagctggctgtttcagaagtatatgtctcatagtgtgcagaacttggtgaccaagtgagaagcgggaccaatgggagactcacaatggactgagtcttgggattatcttttccaaattcttccatgttagaatcacttcagaaaataagactttgatgctttgtctctgagcatcatttttctcctcataaaaacacttccttattgtatgtagcctgcctcctacagaggcctggtaggtgttactgcattctaaaagaaaaatgtcatctctgtaggagcgactatcaggcctagtgtgaaatattagggatcctaggcagaagagctattagtcctggccttcatatcttcaccaaatgaaaatactgtatataaaatttcaccaccaaacttaactaaattctttttctcaagtcaagcctcccaaagaaaaaagaaattaacttcctacagtgtcagcaagcaattttccatttagttttggtacaaataaaagtcatttgaaacaaaaaaaaaaaaaaa

SEQ ID NO: 10 - DNA, 3291 bp, Homo sapiens
ggccgacccgagcagccggttccctcctctccaggccccctccccatcccacccccgccgcctggccccagccgacccgtcccttcgtctccccgcggaatggggccggcactgctcagggtcgcgcgccctggacccagctcgctctcggtctcgcgctgtcagcgactgcccggctcgcgccgcctcgcgctctgcctcagtcagtggcgccgaaggctccgttaagcggcggcggcggttcctgtttccgtttcttcctctccgttcggtcgggagtagcatcctccactcagccacccttcccactcccccatcgtggggcagctgcggctgagggctgtggctttggcagctgcgacggggagcggcggagaccgcctctgctcccgcctggggttgctgcttttgctcagaggacatccatgaccctaatggtctttttgttcaagataaagtgattttttgcctttgttgattaactggacaaattcagcatgtagagcgcatgaagtacaggacaataaagcttcctacacatatcaccaggaggatctctttgaaagattcactgcaggactaccagagagaataatttgtctgaagcatcatgtgttgaaacaacagaagtctattcacctgtgcactaactagaaacagagttacaatgttttcaattctttgagctccaggactccagggaagtgagttgaaaatctgaaaatgcggccatggactggttcctggcgttggattatgctcattctttttgcctgggggaccttgctgttttatataggtggtcacttggtacgagataatgaccatcctgatcactctagccgagaactgtccaagattctggcaaagcttgaacgcttaaaacaacagaatgaagacttgaggcgaatggccgaatctctccggataccagaaggccctattgatcaggggccagctataggaagagtacgcgttttagaagagcagcttgttaaggccaaagaacagattgaaaattacaagaaacagaccagaaatggtctggggaaggatcatgaaatcctgaggaggaggattgaaaatggagctaaagagctctggtttttcctacagagtgaattgaagaaattaaagaacttagaaggaaatgaactccaaagacatgcagatgaatttcttttggatttaggacatcatgaaaggtctataatgacggatctatactacctcagtcagacagatggagcaggtgattggcgggaaaaagaggccaaagatctgacagaactggttcagcggagaataacatatcttcagaatcccaaggactgcagcaaagccaaaaagctggtgtgtaatatcaacaaaggctgtggctatggctgtcagctccatcatgtggtctactgcttcatgattgcatatggcacccagcgaacactcatcttggaatctcagaattggcgctatgctactggtggatgggagactgtatttaggcctgtaagtgagacatgcacagacagatctggcatctccactggacactggtcaggtgaagtgaaggacaaaaatgttcaagtggtcgagcttcccattgtagacagtcttcatccccgtcctccatatttacccttggctgtaccagaagacctcgcagatcgacttgtacgagtgcatggtgaccctgcagtgtggtgggtgtctcagtttgtcaaatacttgatccgcccacagccttggctagaaaaagaaatagaagaagccaccaagaagcttggcttcaaacatccagttattggagtccatgtcagacgcacagacaaagtgggaacagaagctgccttccatcccattgaagagtacatggtgcatgttgaagaacattttcagcttcttgcacgcagaatgcaagtggacaaaaaaagagtgtatttggccacagatgacccttctttattaaaggaggcaaaaacaaagtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataaagctcagatggaagagataaacgaccaaactcagttcgaccaaactcagttcaaaccatttcagccaaactgtagatgaagagggctctgatctaacaaaataaggttatatgagtagatactctcagcaccaagagcagctgggaactgacataggcttcaattggtggaattcctctttaacaagggctgcaatgccctcatacccatgcacagtacaataatgtactcacatataacatgcaaacaggttgttttctactttgcccctttcagtatgtccccataagacaaacactgccatattgtgtaatttaagtgacacagacattttgtgtgagacttaaaacatggtgcctatatctgagagacctgtgtgaactattgagaagatcggaacagctccttactctgaggaagttgattcttatttgatggtggtattgtgaccactgaattcactccagtcaacagattcagaatgagaatggacgtttggtttttttttgtttttgtttttgttttttcctttataaggttgtctgtttttttttttttaaataattgcatcagttcattgacctcatcattaataagtgaagaatacatcagaaaataaaatattcactctccattagaaaattttgtaaaacaatgccatgaacaaattctttagtactcaatgtttctggacattctctttgataacaaaaaataaattttaaaaaggaattttgtaaagtttctagaattttatatcattggatgatatgttgatcagccttatgtggaagaactgtgataaaaagaggagctttttagtttttcagcttaaaaaaa

SEQ ID NO: 11 - DNA, 5179 bp, Homo sapiens
tttcagttggaaggaggtagggaaaataagtgctttaattcctaggattttggaagtaaaaatggaacaccagacaaaaatggaatttttaggcaaatgtaaagctttgtacttgggtatctcaaagtattatacagtgtaaactatcatgattattaaccaactagaagtagttcttttttttaaattttatttttaattttagacatgagggtcttgctatgttgcccaggctgaagtggctactgacaggcacgatgtggcactgcagcctggaactcttgggcttaagtgatccttcctgcttcttctatcttaaaaaaaaaaagttacgcttgcttgttaaaatatttttaaaatgctgtgtttaaactaagaagtaaatatccaccccacctctgacttctaaaaattatctaaagtatgactggtaagatttttacctttaatcccacataagtctaaaatttgtgttcatttccaacaaccaaagtgtgtcacttttataccagaattttgtcctgcttatacgtttagtacagaaatctcatgggagagagcatccatgcatttacaaattgttattgaattattttattgaatgatgacacccaaactgagctagaacataattctggctctgctagtacatcttctgtgtgatcttggacaagtcactctactttcctttcaattttcttttctcacagggagataatcataaaaacgactgtaaagtacagcacttcatagagtgctttttgtttaaagagctgacaataaatacgagtctcaaggtctaggaaagcctccctcacaacctgagctgcttgaggacaagggattttcttttgaatcagcagtaccttatttgtgtatctgtgatagagttcctggtacataagaaggtctcaataaatatgtgaatttatgaatattaggcagattgcaaccttgacaggccactgcctcttaaatctcctttctgtgatcttttaatatttaacatctaaaaggccgccgctacttgctttgggataagtatccccggtatgtactttaaaatgcccaagcctagagaaatgattcttgtcttaagggcaccatttcgctctcccaccgtaaagcgccccaggcttgggatctgggtcccaaggctacagggaagagtttggaacgggaagctcatcttccggccctctgattggccggctcgcactccactcacgcggcgcgcagctctgattggcctcggcggcacccctcgtcccgcgactactttgtgtgctggggcggcgcgctccggtcctcccgctcagctggcggtctgggctgctctggggcagcccttcggtccactgctctgcatcgcgggcgccgggaattttccgagtccgagcgggttgctgcttttgctcagaggacatccatgaccctaatggtctttttgttcaagataaagtgattttttgcctttgttgattaactggacaaattcagcatgtagagcgcatgaagtacaggacaataaagcttcctacacatatcaccaggaggatctctttgaaagattcactgcaggactaccagagagaataatttgtctgaagcatcatgtgttgaaacaacagaagtctattcacctgtgcactaactagaaacagagttacaatgttttcaattctttgagctccaggactccagggaagtgagttgaaaatctgaaaatgcggccatggactggttcctggcgttggattatgctcattctttttgcctgggggaccttgctgttttatataggtggtcacttggtacgagataatgaccatcctgatcactctagccgagaactgtccaagattctggcaaagcttgaacgcttaaaacaacagaatgaagacttgaggcgaatggccgaatctctccggataccagaaggccctattgatcaggggccagctataggaagagtacgcgttttagaagagcagcttgttaaggccaaagaacagattgaaaattacaagaaacagaccagaaatggtctggggaaggatcatgaaatcctgaggaggaggattgaaaatggagctaaagagctctggtttttcctacagagtgaattgaagaaattaaagaacttagaaggaaatgaactccaaagacatgcagatgaatttcttttggatttaggacatcatgaaaggtctataatgacggatctatactacctcagtcagacagatggagcaggtgattggcgggaaaaagaggccaaagatctgacagaactggttcagcggagaataacatatcttcagaatcccaaggactgcagcaaagccaaaaagctggtgtgtaatatcaacaaaggctgtggctatggctgtcagctccatcatgtggtctactgcttcatgattgcatatggcacccagcgaacactcatcttggaatctcagaattggcgctatgctactggtggatgggagactgtatttaggcctgtaagtgagacatgcacagacagatctggcatctccactggacactggtcaggtgaagtgaaggacaaaaatgttcaagtggtcgagcttcccattgtagacagtcttcatccccgtcctccatatttacccttggctgtaccagaagacctcgcagatcgacttgtacgagtgcatggtgaccctgcagtgtggtgggtgtctcagtttgtcaaatacttgatccgcccacagccttggctagaaaaagaaatagaagaagccaccaagaagcttggcttcaaacatccagttattggagtccatgtcagacgcacagacaaagtgggaacagaagctgccttccatcccattgaagagtacatggtgcatgttgaagaacattttcagcttcttgcacgcagaatgcaagtggacaaaaaaagagtgtatttggccacagatgacccttctttattaaaggaggcaaaaacaaagtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataaagctcagatggaagagataaacgaccaaactcagttcgaccaaactcagttcaaaccatttcagccaaactgtagatgaagagggctctgatctaacaaaataaggttatatgagtagatactctcagcaccaagagcagctgggaactgacataggcttcaattggtggaattcctctttaacaagggctgcaatgccctcatacccatgcacagtacaataatgtactcacatataacatgcaaacaggttgttttctactttgcccctttcagtatgtccccataagacaaacactgccatattgtgtaatttaagtgacacagacattttgtgtgagacttaaaacatggtgcctatatctgagagacctgtgtgaactattgagaagatcggaacagctccttactctgaggaagttgattcttatttgatggtggtattgtgaccactgaattcactccagtcaacagattcagaatgagaatggacgtttggtttttttttgtttttgtttttgttttttcctttataaggttgtctgtttttttttttttaaataattgcatcagttcattgacctcatcattaataagtgaagaatacatcagaaaataaaatattcactctccattagaaaattttgtaaaacaatgccatgaacaaattctttagtactcaatgtttctggacattctctttgataacaaaaaataaattttaaaaaggaattttgtaaagtttctagaattttatatcattggatgatatgttgatcagccttatgtggaagaactgtgataaaaagaggagctttttagtttttcagcttatttactttgttttttttcctgtttctgatatagtaactaattcttaactcagagacattggtccattttaatactgaaaaccaattttcattggtacacattacaaaattgctaagaacactgtttgggagctttcattctcatattttggacattgttttaattgagtgaaataatcataactccttgctcccagagaagctatcacctccatttctaaaaccatttcaggtttgtttggtagtctttcttaatgtatttattatcgctttttgtgagtagggtcctgttttatccaggaaccaatttctgccctagcctatcatgccctgcttttgagagtaccaggtatctttgggattgggaagctggctgtttcagaagtatatgtctcatagtgtgcagaacttggtgaccaagtgagaagcgggaccaatgggagactcacaatggactgagtcttgggattatcttttccaaattcttccatgttagaatcacttcagaaaataagactttgatgctttgtctctgagcatcatttttctcctcataaaaacacttccttattgtatgtagcctgcctcctacagaggcctggtaggtgttactgcattctaaaagaaaaatgtcatctctgtaggagcgactatcaggcctagtgtgaaatattagggatcctaggcagaagagctattagtcctggccttcatatcttcaccaaatgaaaatactgtatataaaatttcaccaccaaacttaactaaattctttttctcaagtcaagcctcccaaagaaaaaagaaattaacttcctacagtgtcagcaagcaattttccatttagttttggtacaaataaaagtcatttgaaacaaaaaaaaaaaaaaa

SEQ ID NO: 12 - DNA, 5081 bp, Homo sapiens
tttcagttggaaggaggtagggaaaataagtgctttaattcctaggattttggaagtaaaaatggaacaccagacaaaaatggaatttttaggcaaatgtaaagctttgtacttgggtatctcaaagtattatacagtgtaaactatcatgattattaaccaactagaagtagttcttttttttaaattttatttttaattttagacatgagggtcttgctatgttgcccaggctgaagtggctactgacaggcacgatgtggcactgcagcctggaactcttgggcttaagtgatccttcctgcttcttctatcttaaaaaaaaaaagttacgcttgcttgttaaaatatttttaaaatgctgtgtttaaactaagaagtaaatatccaccccacctctgacttctaaaaattatctaaagtatgactggtaagatttttacctttaatcccacataagtctaaaatttgtgttcatttccaacaaccaaagtgtgtcacttttataccagaattttgtcctgcttatacgtttagtacagaaatctcatgggagagagcatccatgcatttacaaattgttattgaattattttattgaatgatgacacccaaactgagctagaacataattctggctctgctagtacatcttctgtgtgatcttggacaagtcactctactttcctttcaattttcttttctcacagggagataatcataaaaacgactgtaaagtacagcacttcatagagtgctttttgtttaaagagctgacaataaatacgagtctcaaggtctaggaaagcctccctcacaacctgagctgcttgaggacaagggattttcttttgaatcagcagtaccttatttgtgtatctgtgatagagttcctggtacataagaaggtctcaataaatatgtgaatttatgaatattaggcagattgcaaccttgacaggccactgcctcttaaatctcctttctgtgatcttttaatatttaacatctaaaaggccgccgctacttgctttgggataagtatccccggtatgtactttaaaatgcccaagcctagagaaatgattcttgtcttaagggcaccatttcgctctcccaccgtaaagcgccccaggcttgggatctgggtcccaaggctacagggaagagtttggaacgggaagctcatcttccggccctctgattggccggctcgcactccactcacgcggcgcgcagctctgattggcctcggcggcacccctcgtcccgcgactactttgtgtgctggggcggcgcgctccggtcctcccgctcagctggcggtctgggctgctctggggcagcccttcggtccactgctctgcatcgcgggcgccgggaattttccgagtccgagcggcatgtagagcgcatgaagtacaggacaataaagcttcctacacatatcaccaggaggatctctttgaaagattcactgcaggactaccagagagaataatttgtctgaagcatcatgtgttgaaacaacagaagtctattcacctgtgcactaactagaaacagagttacaatgttttcaattctttgagctccaggactccagggaagtgagttgaaaatctgaaaatgcggccatggactggttcctggcgttggattatgctcattctttttgcctgggggaccttgctgttttatataggtggtcacttggtacgagataatgaccatcctgatcactctagccgagaactgtccaagattctggcaaagcttgaacgcttaaaacaacagaatgaagacttgaggcgaatggccgaatctctccggataccagaaggccctattgatcaggggccagctataggaagagtacgcgttttagaagagcagcttgttaaggccaaagaacagattgaaaattacaagaaacagaccagaaatggtctggggaaggatcatgaaatcctgaggaggaggattgaaaatggagctaaagagctctggtttttcctacagagtgaattgaagaaattaaagaacttagaaggaaatgaactccaaagacatgcagatgaatttcttttggatttaggacatcatgaaaggtctataatgacggatctatactacctcagtcagacagatggagcaggtgattggcgggaaaaagaggccaaagatctgacagaactggttcagcggagaataacatatcttcagaatcccaaggactgcagcaaagccaaaaagctggtgtgtaatatcaacaaaggctgtggctatggctgtcagctccatcatgtggtctactgcttcatgattgcatatggcacccagcgaacactcatcttggaatctcagaattggcgctatgctactggtggatgggagactgtatttaggcctgtaagtgagacatgcacagacagatctggcatctccactggacactggtcaggtgaagtgaaggacaaaaatgttcaagtggtcgagcttcccattgtagacagtcttcatccccgtcctccatatttacccttggctgtaccagaagacctcgcagatcgacttgtacgagtgcatggtgaccctgcagtgtggtgggtgtctcagtttgtcaaatacttgatccgcccacagccttggctagaaaaagaaatagaagaagccaccaagaagcttggcttcaaacatccagttattggagtccatgtcagacgcacagacaaagtgggaacagaagctgccttccatcccattgaagagtacatggtgcatgttgaagaacattttcagcttcttgcacgcagaatgcaagtggacaaaaaaagagtgtatttggccacagatgacccttctttattaaaggaggcaaaaacaaagtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataaagctcagatggaagagataaacgaccaaactcagttcgaccaaactcagttcaaaccatttcagccaaactgtagatgaagagggctctgatctaacaaaataaggttatatgagtagatactctcagcaccaagagcagctgggaactgacataggcttcaattggtggaattcctctttaacaagggctgcaatgccctcatacccatgcacagtacaataatgtactcacatataacatgcaaacaggttgttttctactttgcccctttcagtatgtccccataagacaaacactgccatattgtgtaatttaagtgacacagacattttgtgtgagacttaaaacatggtgcctatatctgagagacctgtgtgaactattgagaagatcggaacagctccttactctgaggaagttgattcttatttgatggtggtattgtgaccactgaattcactccagtcaacagattcagaatgagaatggacgtttggtttttttttgtttttgtttttgttttttcctttataaggttgtctgtttttttttttttaaataattgcatcagttcattgacctcatcattaataagtgaagaatacatcagaaaataaaatattcactctccattagaaaattttgtaaaacaatgccatgaacaaattctttagtactcaatgtttctggacattctctttgataacaaaaaataaattttaaaaaggaattttgtaaagtttctagaattttatatcattggatgatatgttgatcagccttatgtggaagaactgtgataaaaagaggagctttttagtttttcagcttatttactttgttttttttcctgtttctgatatagtaactaattcttaactcagagacattggtccattttaatactgaaaaccaattttcattggtacacattacaaaattgctaagaacactgtttgggagctttcattctcatattttggacattgttttaattgagtgaaataatcataactccttgctcccagagaagctatcacctccatttctaaaaccatttcaggtttgtttggtagtctttcttaatgtatttattatcgctttttgtgagtagggtcctgttttatccaggaaccaatttctgccctagcctatcatgccctgcttttgagagtaccaggtatctttgggattgggaagctggctgtttcagaagtatatgtctcatagtgtgcagaacttggtgaccaagtgagaagcgggaccaatgggagactcacaatggactgagtcttgggattatcttttccaaattcttccatgttagaatcacttcagaaaataagactttgatgctttgtctctgagcatcatttttctcctcataaaaacacttccttattgtatgtagcctgcctcctacagaggcctggtaggtgttactgcattctaaaagaaaaatgtcatctctgtaggagcgactatcaggcctagtgtgaaatattagggatcctaggcagaagagctattagtcctggccttcatatcttcaccaaatgaaaatactgtatataaaatttcaccaccaaacttaactaaattctttttctcaagtcaagcctcccaaagaaaaaagaaattaacttcctacagtgtcagcaagcaattttccatttagttttggtacaaataaaagtcatttgaaacaaaaaaaaaaaaaaa

SEQ ID NO: 13 - DNA, 1396 bp, Homo sapiens
ctgtatttaggcctgtaagtgagacatgcacagacagatctggcatctccactggacactggtcaggtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataaagctcagatggaagagataaacgaccaaactcagttcgaccaaactcagttcaaaccatttcagccaaactgtagatgaagagggctctgatctaacaaaataaggttatatgagtagatactctcagcaccaagagcagctgggaactgacataggcttcaattggtggaattcctctttaacaagggctgcaatgccctcatacccatgcacagtacaataatgtactcacatataacatgcaaacaggttgttttctactttgcccctttcagtatgtccccataagacaaacactgccatattgtgtaatttaagtgacacagacattttgtgtgagacttaaaacatggtgcctatatctgagagacctgtgtgaactattgagaagatcggaacagctccttactctgaggaagttgattcttatttgatggtggtattgtgaccactgaattcactccagtcaacagattcagaatgagaatggacgtttggtttttttttgtttttgtttttgttttttcctttataaggttgtctgtttttttttttttaaataattgcatcagttcattgacctcatcattaataagtgaagaatacatcagaaaataaaatattcactctccattagaaaattttgtaaaacaatgccatgaacaaattctttagtactcaatgtttctggacattctctttgataacaaaaaataaattttaaaaaggaattttgtaaagtttctagaattttatatcattggatgatatgttgatcagccttatgtggaagaactgtgataaaaagaggagctttttagtttttcagcttaaaaaaaaaaaaaaaaaaaaaaaaaaaa

SEQ ID NO: 14 - protein, 412 aa, Homo sapiens
MTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 15 - protein, 575 aa, Homo sapiens
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 16 - protein, 575 aa, Homo sapiens; identical to SEQ ID NO: 15

SEQ ID NO: 17 - DNA, 2639 bp, Homo sapiens
actctcagcagctcaactgcccagcgtgaccagtggccacctctgcagtgtcttccacaacctggtcttgactcgtctgctgaacaaatcctctgacctcaggccggctgtgaacgtagttcctgagagatagcaaacatgcccaacagtgagcccgcatctctgctggagctgttcaacagcatcgccacacaaggggagctcgtaaggtccctcaaagcgggaaatgcgtcaaaggatgaaattgattctgcagtaaagatgttggtgtcattaaaaatgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatagctatgctgtggagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgctgcactccaccttcttcccagccctgcagggcgcccagaccaaaatgagtgccagcgaccccaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagtagcactcgttttacatatgcttataaaagaagtgatgtatcagtaatgtatcaataatcccagcccagtcaaagcaccgccacctgtaggcttctgtctcatggtaattactgggcctggcctctgtaagcctgtgtatgttatcaatactgtttcttcctgtgagttccattatttctatctcttatgggcaaagcattgtgggtaattggtgctggctaacattgcatggtcggatagagaagtccagctgtgagtctctccccaaagcagccccacagtggagcctttggctggaagtccatgggccaccctgttcttgtccatggaggactccgagggttccaagtatactcttaagacccactctgtttaaaaatatatattctatgtatgcgtatatggaattgaaatgtcattattgtaacctagaaagtgctttgaaatattgatgtggggaggtttattgagcacaagatgtatttcagcccatgccccctcccaaaaagaaattgataagtaaaagcttcgttatacatttgactaagaaatcacccagctttaaagctgcttttaacaatgaagattgaacagagttcagcaattttgattaaattaagacttgggggtgaaactttccagtttactgaactccagaccatgcatgtagtccactccagaaatcatgctcgcttcccttggcacaccagtgttctcctgccaaatgaccctagaccctctgtcctgcagagtcagggtggcttttcccctgactgtgtccgatgccaaggagtcctggcctccgcagatgcttcattttgacccttggctgcagtggaagtcagcacagagcagtgccctggctgtgtccctggacgggtggacttagctagggagaaagtcgaggcagcagccctcgaggccctcacagatgtctaggcaggcctcatttcatcacgcagcatgtgcaggcctggaagagcaaagccaaatctcagggaagtccttggttgatgtatctgggtctcctctggagcactctgccctcctgtcacccagtagagtaaataaacttccttggctcctg

SEQ ID NO: 18 - DNA, 2660 bp, Homo sapiens
gttccgtccggacggcgtttccacggacgcccaggagacagccggttgctgagccgggagcgctgactggcccggctgggcaggtcttgactcgtctgctgaacaaatcctctgacctcaggccggctgtgaacgtagttcctgagagatagcaaacatgcccaacagtgagcccgcatctctgctggagctgttcaacagcatcgccacacaaggggagctcgtaaggtccctcaaagcgggaaatgcgtcaaaggatgaaattgattctgcagtaaagatgttggtgtcattaaaaatgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatagctatgctgtggagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgctgcactccaccttcttcccagccctgcagggcgcccagaccaaaatgagtgccagcgaccccaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagtagcactcgttttacatatgcttataaaagaagtgatgtatcagtaatgtatcaataatcccagcccagtcaaagcaccgccacctgtaggcttctgtctcatggtaattactgggcctggcctctgtaagcctgtgtatgttatcaatactgtttcttcctgtgagttccattatttctatctcttatgggcaaagcattgtgggtaattggtgctggctaacattgcatggtcggatagagaagtccagctgtgagtctctccccaaagcagccccacagtggagcctttggctggaagtccatgggccaccctgttcttgtccatggaggactccgagggttccaagtatactcttaagacccactctgtttaaaaatatatattctatgtatgcgtatatggaattgaaatgtcattattgtaacctagaaagtgctttgaaatattgatgtggggaggtttattgagcacaagatgtatttcagcccatgccccctcccaaaaagaaattgataagtaaaagcttcgttatacatttgactaagaaatcacccagctttaaagctgcttttaacaatgaagattgaacagagttcagcaattttgattaaattaagacttgggggtgaaactttccagtttactgaactccagaccatgcatgtagtccactccagaaatcatgctcgcttcccttggcacaccagtgttctcctgccaaatgaccctagaccctctgtcctgcagagtcagggtggcttttcccctgactgtgtccgatgccaaggagtcctggcctccgcagatgcttcattttgacccttggctgcagtggaagtcagcacagagcagtgccctggctgtgtccctggacgggtggacttagctagggagaaagtcgaggcagcagccctcgaggccctcacagatgtctaggcaggcctcatttcatcacgcagcatgtgcaggcctggaagagcaaagccaaatctcagggaagtccttggttgatgtatctgggtctcctctggagcactctgccctcctgtcacccagtagagtaaataaacttccttggctcctgct

SEQ ID NO: 19 - DNA, 2488 bp, Homo sapiens
gttccgtccggacggcgtttccacggacgcccaggagacagccggttgctgagccgggagcgctgactggcccggctgggcagggatgaaattgattctgcagtaaagatgttggtgtcattaaaaatgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatagctatgctgtggagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgctgcactccaccttcttcccagccctgcagggcgcccagaccaaaatgagtgccagcgaccccaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagtagcactcgttttacatatgcttataaaagaagtgatgtatcagtaatgtatcaataatcccagcccagtcaaagcaccgccacctgtaggcttctgtctcatggtaattactgggcctggcctctgtaagcctgtgtatgttatcaatactgtttcttcctgtgagttccattatttctatctcttatgggcaaagcattgtgggtaattggtgctggctaacattgcatggtcggatagagaagtccagctgtgagtctctccccaaagcagccccacagtggagcctttggctggaagtccatgggccaccctgttcttgtccatggaggactccgagggttccaagtatactcttaagacccactctgtttaaaaatatatattctatgtatgcgtatatggaattgaaatgtcattattgtaacctagaaagtgctttgaaatattgatgtggggaggtttattgagcacaagatgtatttcagcccatgccccctcccaaaaagaaattgataagtaaaagcttcgttatacatttgactaagaaatcacccagctttaaagctgcttttaacaatgaagattgaacagagttcagcaattttgattaaattaagacttgggggtgaaactttccagtttactgaactccagaccatgcatgtagtccactccagaaatcatgctcgcttcccttggcacaccagtgttctcctgccaaatgaccctagaccctctgtcctgcagagtcagggtggcttttcccctgactgtgtccgatgccaaggagtcctggcctccgcagatgcttcattttgacccttggctgcagtggaagtcagcacagagcagtgccctggctgtgtccctggacgggtggacttagctagggagaaagtcgaggcagcagccctcgaggccctcacagatgtctaggcaggcctcatttcatcacgcagcatgtgcaggcctggaagagcaaagccaaatctcagggaagtccttggttgatgtatctgggtctcctctggagcactctgccctcctgtcacccagtagagtaaataaacttccttggctcctgct

SEQ ID NO: 20 - DNA, 2712 bp, Homo sapiens
tcgattctcaagagggtttcattggtctcaacctggccccccaggcaacccacccctgattggacagtctcatcaagaaggttggtcaagagctcaagtgtttctgagaatctgggtgatttataagaaacccttagctgaatgcagggtggggagaacgaaagacaaaagcatcttttttcagaagggaaactgaaagaaagaggggaagagtattaaagaccatttctggctgggcagggcactctcagcagctcaactgcccagcgtgaccagtggccacctctgcagtgtcttccacaacctgggatgaaattgattctgcagtaaagatgttggtgtcattaaaaatgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatagctatgctgtggagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgctgcactccaccttcttcccagccctgcagggcgcccagaccaaaatgagtgccagcgaccccaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagtagcactcgttttacatatgcttataaaagaagtgatgtatcagtaatgtatcaataatcccagcccagtcaaagcaccgccacctgtaggcttctgtctcatggtaattactgggcctggcctctgtaagcctgtgtatgttatcaatactgtttcttcctgtgagttccattatttctatctcttatgggcaaagcattgtgggtaattggtgctggctaacattgcatggtcggatagagaagtccagctgtgagtctctccccaaagcagccccacagtggagcctttggctggaagtccatgggccaccctgttcttgtccatggaggactccgagggttccaagtatactcttaagacccactctgtttaaaaatatatattctatgtatgcgtatatggaattgaaatgtcattattgtaacctagaaagtgctttgaaatattgatgtggggaggtttattgagcacaagatgtatttcagcccatgccccctcccaaaaagaaattgataagtaaaagcttcgttatacatttgactaagaaatcacccagctttaaagctgcttttaacaatgaagattgaacagagttcagcaattttgattaaattaagacttgggggtgaaactttccagtttactgaactccagaccatgcatgtagtccactccagaaatcatgctcgcttcccttggcacaccagtgttctcctgccaaatgaccctagaccctctgtcctgcagagtcagggtggcttttcccctgactgtgtccgatgccaaggagtcctggcctccgcagatgcttcattttgacccttggctgcagtggaagtcagcacagagcagtgccctggctgtgtccctggacgggtggacttagctagggagaaagtcgaggcagcagccctcgaggccctcacagatgtctaggcaggcctcatttcatcacgcagcatgtgcaggcctggaagagcaaagccaaatctcagggaagtccttggttgatgtatctgggtctcctctggagcactctgccctcctgtcacccagtagagtaaataaacttccttggctcctgct

SEQ ID NO: 21 - protein, 471 aa, Homo sapiens
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 22 - protein, 471 aa, Homo sapiens; identical to SEQ ID NO: 21

SEQ ID NO: 23 - protein, 430 aa, Homo sapiens
MLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 24 - protein, 430 aa, Homo sapiens; identical to SEQ ID NO: 23

SEQ ID NO: 25 - DNA, 6749 bp, Homo sapiens
gtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaagtatgtattcttcactactgagtttcttctttggaaaccatcactattgagagatgggaaaaacctgaatgtataaagcatttatttgtcaataaactgccttttgtaaggggttttcacataacatagaggagcttcccttttttgtttaagttttgtaacctttaatcctccatattctcatgtctgtcatcccaggggtgtcacaactgtaaaaatctacaatattagaaagcagctactacagatgtggaagagagaacacttgtataagacaatgctgtactgaagttatataacaaggtcccctgatacttatgcctgcattactttgagggatgctgaatgagaggaccatctccctgaaatatattaataattttcagataaaattatgaacataacataccttttctcaagtggaattagagttcatttcccatttaagtgattacactctatctcctaagagctgtgggaaccagttcatattgcagtggaaacattaactcttcgttgcactgttgagtgtcaaggctactgtggcaatgttttttgcaaagttctcagtaattttctctctgcaatcaaattagagttcatatttaatttgcagattctctcattcattgctccacactgctttatcaaatcaggtgaaagaaattaatgtagttttgcctattttacaaaatggtggttggttctttaaacaccatgttattactcttaataaatctacaaatatttattgagcatctatgatatgcttggtgctgttaggcactgtgtttttaaagcacaaactctcaaaagctgattccagcgtaatgttttatttttgtttttctgtgttttttatgacaaagtctgtctgatagacaaagcctggaaggtttcccccagggctttagaatttagaattagctggttttcatttcaaatgggaaattaatggagaaatgagataaaatcatctctgtgtaaatgcttgaacccacagaaagttttctaaatatggactaattgtgggtttgtgcatatttctgtgtctcctcttttcacttcacacccaccatgatataaaaagatgcatcagagagtgagcctgccttcaggacctgaaaggacatgactgaacatctagtagatctggtggaaattaatgttatacaaaagacacgtagactaagttgaccctgggagaggagttaatacattcctttgcaaagtgacatgtgccctttttttgacagtctggctttcatggtatgagatgaggactcacaggccttgggtaatttctgcttttgtatgaattttttcagatggttctgagctggttatgtccctggaggggaatttttcatttgccttagatgttcttcccatgagtaggtacatgagtgaagtttcttacacttttaaaattgcattggcaaattttcttttgctttatgaacccctgttttgtcattatgatttttcacttgttttaacagtttcagatgtcaccttggaatagcactgtttattttggcctttttatgattgctgatgataggtacagtctaaaggccaaaagttgccacttcattgtgataacattgttgcaacattcctaatatattagagaatgatgtaaaataagaatgttagagaacatagcacattctcattcagtttataaacaatatttgaaaatttccagtgagaattaaaaaaggactagtgatgatatagcaaacctggacctgaaatttgggggtggggggtggggggatatctgttttgcagagaaaatttaccagcctccagcaaaatctctgcatcttgtactcaatagcaaccattagtattgtcatttaaaaagaatgaatcaaaaattcatacgtaatggaatgaaaagaggaaaactttctattaaacactgtcaagaaagacccattgctatctatcacctatgaaattaaacctaaaaaaatttttttttattgtaaaagcgactgctcgttgatgtctggaagtgatccagactttgtttcactgtgatccaaaaagttagacattccttccatttctcaagatttggtaacaatttttttttaagtcagcatttcatataatacataaaattaagacaaattaacttactgatatgtggccaatatcaacccttctcacccaacccaaacatgttctggagatagaaggacttatcctcaattatattccagtaaatttatctccataatctgttttatcccctaatgtatgatatatctcaccaaatttttctagaattgaataaaagcagtttttgcttaacttgaatttccttccacaaatacaaccttcagctcttccctctctgtttcccttttgcccacattgcaaatagtcttcataatagaaccctcactgcaacttaaaagggtcccatttcttaactcagcatttcatataccaagaacacttgcaaagatttcttcataatgtgattttttaatttttcagttccagaagcatttcctacagttcgaacgttattttttctctcagattttcaacttgaagctttgatttgcactgaattaacttggataaaattttagtttctctcttcagctaacagatttggaagcaaattacagtatacttgaataattcaggtactacctgttgacctggaattttagtgattctgggaaagttttccagacaaagacaccaatagagtgaagggttttttccccaattactattttattgttttttaaaatgcctttttaaaacttgtgtgatgtggcaaaatagtcatatgttcaattaatttatattttattcattccttttcaattctggttattatgtaacctcaagtactttatctgttcttttaaagtattttataaaatgaacgttaactaaa

SEQ ID NO: 26 - DNA, 6685 bp, Homo sapiens
ggggccgggaaggggcctgagcagggaagggacacaggcgtcctgaaacaaagctttgggttgagggcagaggcgaggagccagagaagtgaactcctcctttccgtggccgctcttgtcatttttctcttttctcttttcaaatgggctatttgtgtgttaatttcatttggttcttgggaataacgactcaccgcgttgatttaaagaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaagtatgtattcttcactactgagtttcttctttggaaaccatcactattgagagatgggaaaaacctgaatgtataaagcatttatttgtcaataaactgccttttgtaaggggttttcacataacatagaggagcttcccttttttgtttaagttttgtaacctttaatcctccatattctcatgtctgtcatcccaggggtgtcacaactgtaaaaatctacaatattagaaagcagctactacagatgtggaagagagaacacttgtataagacaatgctgtactgaagttatataacaaggtcccctgatacttatgcctgcattactttgagggatgctgaatgagaggaccatctccctgaaatatattaataattttcagataaaattatgaacataacataccttttctcaagtggaattagagttcatttcccatttaagtgattacactctatctcctaagagctgtgggaaccagttcatattgcagtggaaacattaactcttcgttgcactgttgagtgtcaaggctactgtggcaatgttttttgcaaagttctcagtaattttctctctgcaatcaaattagagttcatatttaatttgcagattctctcattcattgctccacactgctttatcaaatcaggtgaaagaaattaatgtagttttgcctattttacaaaatggtggttggttctttaaacaccatgttattactcttaataaatctacaaatatttattgagcatctatgatatgcttggtgctgttaggcactgtgtttttaaagcacaaactctcaaaagctgattccagcgtaatgttttatttttgtttttctgtgttttttatgacaaagtctgtctgatagacaaagcctggaaggtttcccccagggctttagaatttagaattagctggttttcatttcaaatgggaaattaatggagaaatgagataaaatcatctctgtgtaaatgcttgaacccacagaaagttttctaaatatggactaattgtgggtttgtgcatatttctgtgtctcctcttttcacttcacacccaccatgatataaaaagatgcatcagagagtgagcctgccttcaggacctgaaaggacatgactgaacatctagtagatctggtggaaattaatgttatacaaaagacacgtagactaagttgaccctgggagaggagttaatacattcctttgcaaagtgacatgtgccctttttttgacagtctggctttcatggtatgagatgaggactcacaggccttgggtaatttctgcttttgtatgaattttttcagatggttctgagctggttatgtccctggaggggaatttttcatttgccttagatgttcttcccatgagtaggtacatgagtgaagtttcttacacttttaaaattgcattggcaaattttcttttgctttatgaacccctgttttgtcattatgatttttcacttgttttaacagtttcagatgtcaccttggaatagcactgtttattttggcctttttatgattgctgatgataggtacagtctaaaggccaaaagttgccacttcattgtgataacattgttgcaacattcctaatatattagagaatgatgtaaaataagaatgttagagaacatagcacattctcattcagtttataaacaatatttgaaaatttccagtgagaattaaaaaaggactagtgatgatatagcaaacctggacctgaaatttgggggtggggggtggggggatatctgttttgcagagaaaatttaccagcctccagcaaaatctctgcatcttgtactcaatagcaaccattagtattgtcatttaaaaagaatgaatcaaaaattcatacgtaatggaatgaaaagaggaaaactttctattaaacactgtcaagaaagacccattgctatctatcacctatgaaattaaacctaaaaaaatttttttttattgtaaaagcgactgctcgttgatgtctggaagtgatccagactttgtttcactgtgatccaaaaagttagacattccttccatttctcaagatttggtaacaatttttttttaagtcagcatttcatataatacataaaattaagacaaattaacttactgatatgtggccaatatcaacccttctcacccaacccaaacatgttctggagatagaaggacttatcctcaattatattccagtaaatttatctccataatctgttttatcccctaatgtatgatatatctcaccaaatttttctagaattgaataaaagcagtttttgcttaacttgaatttccttccacaaatacaaccttcagctcttccctctctgtttcccttttgcccacattgcaaatagtcttcataatagaaccctcactgcaacttaaaagggtcccatttcttaactcagcatttcatataccaagaacacttgcaaagatttcttcataatgtgattttttaatttttcagttccagaagcatttcctacagttcgaacgttattttttctctcagattttcaacttgaagctttgatttgcactgaattaacttggataaaattttagtttctctcttcagctaacagatttggaagcaaattacagtatacttgaataattcaggtactacctgttgacctggaattttagtgattctgggaaagttttccagacaaagacaccaatagagtgaagggttttttccccaattactattttattgttttttaaaatgcctttttaaaacttgtgtgatgtggcaaaatagtcatatgttcaattaatttatattttattcattccttttcaattctggttattatgtaacctcaagtactttatctgttcttttaaagtattttataaaatgaacgttaactaaa

SEQ ID NO: 27 - DNA, 6604 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacgaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaagtatgtattcttcactactgagtttcttctttggaaaccatcactattgagagatgggaaaaacctgaatgtataaagcatttatttgtcaataaactgccttttgtaaggggttttcacataacatagaggagcttcccttttttgtttaagttttgtaacctttaatcctccatattctcatgtctgtcatcccaggggtgtcacaactgtaaaaatctacaatattagaaagcagctactacagatgtggaagagagaacacttgtataagacaatgctgtactgaagttatataacaaggtcccctgatacttatgcctgcattactttgagggatgctgaatgagaggaccatctccctgaaatatattaataattttcagataaaattatgaacataacataccttttctcaagtggaattagagttcatttcccatttaagtgattacactctatctcctaagagctgtgggaaccagttcatattgcagtggaaacattaactcttcgttgcactgttgagtgtcaaggctactgtggcaatgttttttgcaaagttctcagtaattttctctctgcaatcaaattagagttcatatttaatttgcagattctctcattcattgctccacactgctttatcaaatcaggtgaaagaaattaatgtagttttgcctattttacaaaatggtggttggttctttaaacaccatgttattactcttaataaatctacaaatatttattgagcatctatgatatgcttggtgctgttaggcactgtgtttttaaagcacaaactctcaaaagctgattccagcgtaatgttttatttttgtttttctgtgttttttatgacaaagtctgtctgatagacaaagcctggaaggtttcccccagggctttagaatttagaattagctggttttcatttcaaatgggaaattaatggagaaatgagataaaatcatctctgtgtaaatgcttgaacccacagaaagttttctaaatatggactaattgtgggtttgtgcatatttctgtgtctcctcttttcacttcacacccaccatgatataaaaagatgcatcagagagtgagcctgccttcaggacctgaaaggacatgactgaacatctagtagatctggtggaaattaatgttatacaaaagacacgtagactaagttgaccctgggagaggagttaatacattcctttgcaaagtgacatgtgccctttttttgacagtctggctttcatggtatgagatgaggactcacaggccttgggtaatttctgcttttgtatgaattttttcagatggttctgagctggttatgtccctggaggggaatttttcatttgccttagatgttcttcccatgagtaggtacatgagtgaagtttcttacacttttaaaattgcattggcaaattttcttttgctttatgaacccctgttttgtcattatgatttttcacttgttttaacagtttcagatgtcaccttggaatagcactgtttattttggcctttttatgattgctgatgataggtacagtctaaaggccaaaagttgccacttcattgtgataacattgttgcaacattcctaatatattagagaatgatgtaaaataagaatgttagagaacatagcacattctcattcagtttataaacaatatttgaaaatttccagtgagaattaaaaaaggactagtgatgatatagcaaacctggacctgaaatttgggggtggggggtggggggatatctgttttgcagagaaaatttaccagcctccagcaaaatctctgcatcttgtactcaatagcaaccattagtattgtcatttaaaaagaatgaatcaaaaattcatacgtaatggaatgaaaagaggaaaactttctattaaacactgtcaagaaagacccattgctatctatcacctatgaaattaaacctaaaaaaatttttttttattgtaaaagcgactgctcgttgatgtctggaagtgatccagactttgtttcactgtgatccaaaaagttagacattccttccatttctcaagatttggtaacaatttttttttaagtcagcatttcatataatacataaaattaagacaaattaacttactgatatgtggccaatatcaacccttctcacccaacccaaacatgttctggagatagaaggacttatcctcaattatattccagtaaatttatctccataatctgttttatcccctaatgtatgatatatctcaccaaatttttctagaattgaataaaagcagtttttgcttaacttgaatttccttccacaaatacaaccttcagctcttccctctctgtttcccttttgcccacattgcaaatagtcttcataatagaaccctcactgcaacttaaaagggtcccatttcttaactcagcatttcatataccaagaacacttgcaaagatttcttcataatgtgattttttaatttttcagttccagaagcatttcctacagttcgaacgttattttttctctcagattttcaacttgaagctttgatttgcactgaattaacttggataaaattttagtttctctcttcagctaacagatttggaagcaaattacagtatacttgaataattcaggtactacctgttgacctggaattttagtgattctgggaaagttttccagacaaagacaccaatagagtgaagggttttttccccaattactattttattgttttttaaaatgcctttttaaaacttgtgtgatgtggcaaaatagtcatatgttcaattaatttatattttattcattccttttcaattctggttattatgtaacctcaagtactttatctgttcttttaaagtattttataaaatgaacgttaactaaa

SEQ ID NO: 28 - DNA, 6476 bp, Homo sapiens
gagggcagaggcgaggagccagagaagtgaactcctcctttccgtggccgctcttgtcatttttctcttttctcttttcaaatgggctatttgtgtgttaatttcatttggttcttgggaataacgactcaccgcgttgatttaaagaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaagtatgtattcttcactactgagtttcttctttggaaaccatcactattgagagatgggaaaaacctgaatgtataaagcatttatttgtcaataaactgccttttgtaaggggttttcacataacatagaggagcttcccttttttgtttaagttttgtaacctttaatcctccatattctcatgtctgtcatcccaggggtgtcacaactgtaaaaatctacaatattagaaagcagctactacagatgtggaagagagaacacttgtataagacaatgctgtactgaagttatataacaaggtcccctgatacttatgcctgcattactttgagggatgctgaatgagaggaccatctccctgaaatatattaataattttcagataaaattatgaacataacataccttttctcaagtggaattagagttcatttcccatttaagtgattacactctatctcctaagagctgtgggaaccagttcatattgcagtggaaacattaactcttcgttgcactgttgagtgtcaaggctactgtggcaatgttttttgcaaagttctcagtaattttctctctgcaatcaaattagagttcatatttaatttgcagattctctcattcattgctccacactgctttatcaaatcaggtgaaagaaattaatgtagttttgcctattttacaaaatggtggttggttctttaaacaccatgttattactcttaataaatctacaaatatttattgagcatctatgatatgcttggtgctgttaggcactgtgtttttaaagcacaaactctcaaaagctgattccagcgtaatgttttatttttgtttttctgtgttttttatgacaaagtctgtctgatagacaaagcctggaaggtttcccccagggctttagaatttagaattagctggttttcatttcaaatgggaaattaatggagaaatgagataaaatcatctctgtgtaaatgcttgaacccacagaaagttttctaaatatggactaattgtgggtttgtgcatatttctgtgtctcctcttttcacttcacacccaccatgatataaaaagatgcatcagagagtgagcctgccttcaggacctgaaaggacatgactgaacatctagtagatctggtggaaattaatgttatacaaaagacacgtagactaagttgaccctgggagaggagttaatacattcctttgcaaagtgacatgtgccctttttttgacagtctggctttcatggtatgagatgaggactcacaggccttgggtaatttctgcttttgtatgaattttttcagatggttctgagctggttatgtccctggaggggaatttttcatttgccttagatgttcttcccatgagtaggtacatgagtgaagtttcttacacttttaaaattgcattggcaaattttcttttgctttatgaacccctgttttgtcattatgatttttcacttgttttaacagtttcagatgtcaccttggaatagcactgtttattttggcctttttatgattgctgatgataggtacagtctaaaggccaaaagttgccacttcattgtgataacattgttgcaacattcctaatatattagagaatgatgtaaaataagaatgttagagaacatagcacattctcattcagtttataaacaatatttgaaaatttccagtgagaattaaaaaaggactagtgatgatatagcaaacctggacctgaaatttgggggtggggggtggggggatatctgttttgcagagaaaatttaccagcctccagcaaaatctctgcatcttgtactcaatagcaaccattagtattgtcatttaaaaagaatgaatcaaaaattcatacgtaatggaatgaaaagaggaaaactttctattaaacactgtcaagaaagacccattgctatctatcacctatgaaattaaacctaaaaaaatttttttttattgtaaaagcgactgctcgttgatgtctggaagtgatccagactttgtttcactgtgatccaaaaagttagacattccttccatttctcaagatttggtaacaatttttttttaagtcagcatttcatataatacataaaattaagacaaattaacttactgatatgtggccaatatcaacccttctcacccaacccaaacatgttctggagatagaaggacttatcctcaattatattccagtaaatttatctccataatctgttttatcccctaatgtatgatatatctcaccaaatttttctagaattgaataaaagcagtttttgcttaacttgaatttccttccacaaatacaaccttcagctcttccctctctgtttcccttttgcccacattgcaaatagtcttcataatagaaccctcactgcaacttaaaagggtcccatttcttaactcagcatttcatataccaagaacacttgcaaagatttcttcataatgtgattttttaatttttcagttccagaagcatttcctacagttcgaacgttattttttctctcagattttcaacttgaagctttgatttgcactgaattaacttggataaaattttagtttctctcttcagctaacagatttggaagcaaattacagtatacttgaataattcaggtactacctgttgacctggaattttagtgattctgggaaagttttccagacaaagacaccaatagagtgaagggttttttccccaattactattttattgttttttaaaatgcctttttaaaacttgtgtgatgtggcaaaatagtcatatgttcaattaatttatattttattcattccttttcaattctggttattatgtaacctcaagtactttatctgttcttttaaagtattttataaaatgaacgttaactaaa

SEQ ID NO: 29 - skipped

SEQ ID NO: 30 - protein, 1010 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 31 - protein, 982 aa, Homo sapiens [sequence omitted]

SEQ ID NO: 32 - protein, 961 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 33 - protein, 960 aa, Homo sapiens
MALAPGPDAHAHLPPLIELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 34 - protein, 938 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 35 - DNA, 4800 bp, Homo sapiens
agagcggcggccggtcccgcgcggagcccggcgcccctccagcccgagccaggacgccgccggccccggtcccggccccgggcacgcagcgagccagggatgtgagcggcgccccgcggcatggcagcctcaggggtgcccagaggatgcgacatcctcatcgtctacagcccggatgccgaggaatggtgccagtacctgcagaccctgttcctgtccagtcggcaggtccgcagccagaagatactgactcacaggctgggccccgaggcctccttctcggcagaggacctaagccttttcctcagcacccgctgtgtcgtggtgctgctgtccgcggagctggtgcagcacttccacaagcccgccttgctgcccctgctgcagagagctttccatcctccgcaccgcgtggtcaggctgctctgcggcgtgcgggacagcgaggagttcctagacttctttccagattgggcccattggcaggagctcacctgtgacgatgagccagagacctacgtggcagctgtgaaaaaagccatttccgaagattctggctgtgactcagtcactgacactgagcctgaggacgagaaggttgtttcctactcgaagcagcagaacctgccgacggtgacttcacctgggaacctgatggtggtgcagccggaccgcattcgctgtggggcagaaaccactgtctatgttattgtgagatgtaagctggatgacagggtggcgacagaagcagagttttctcctgaggattctccctctgtaaggatggaagccaaggtggagaatgagtacaccatttcagtgaaggctcccaacctttcatctgggaacgtttctctgaagatatattctggagacttagtggtgtgtgaaaccgttatcagctattatactgacatggaagaaattgggaatttattgtccaatgccgcgaatcctgtggaattcatgtgtcaggcctttaaaattgtgccctacaacacagagacccttgataaactgctaaccgaatccctgaagaacaatatccctgcaagcggactgcacctctttggaatcaaccagctggaagaagaagatatgatgacaaatcagagggatgaagagctgcccaccctgttgcattttgctgcgaagtatggactgaagaacctcactgccttgttgctcacctgcccaggagccctgcaggcgtacagcgtggccaacaagcatggccactaccccaacaccatcgctgagaaacacggcttcagggacctgcggcagttcatcgacgagtatgtggaaacggtggacatgctcaagagtcacattaaagaggaactgatgcacggggaggaggctgatgctgtgtacgagtccatggcccacctttccacagacctgcttatgaaatgctcgctcaaccccggctgtgacgaggatctctatgagtccatggctgcctttgtcccagctgccactgaagacctctatgttgaaatgcttcaggccagtacatctaacccaatccctggagatggtttctctcgggccactaaggactctatgatccgcaagtttttagaaggcaacagcatgggaatgaccaatctggagagagatcagtgccatcttggtcaggaagaagatgtttatcacacggtggatgacgatgaggccttttctgtggacctggccagcaggccccctgtcccagtgcccagaccagagaccactgctcctggtgctcaccagctgcctgacaacgaaccatacatttttaaagtttttgcagaaaaaagtcaagagcggcctgggaatttctacgtttcctcagagagcatcaggaaagggccgcccgtcagaccatggagggacaggccccagtcgagtatatatgacccttttgcgggaatgaaaacgccaggccagcggcagcttatcaccctccaggagcaggtgaagctgggcattgtcaacgtggatgaggctgtgctccacttcaaagagtggcagctcaaccagaagaaacgatcggagtcctttcgtttccagcaggaaaatcttaaacggctaagagacagcatcacccgaagacagagagagaagcaaaaatcaggaaagcagacagacttggagatcacggtcccaattcggcactcacagcacctgcctgcaaaagtggagtttggagtctatgagagtggccccaggaaaagtgtcattccccctaggacggagctgagacgaggagactggaaaacagacagcacctccagcacagcaagtagcacaagtaaccgctccagcacccggagcctcctcagtgtgagcagcgggatggaaggggacaacgaggataatgaagtccctgaggttaccagaagtcgcagtccaggccccccacaagtggatgggacacccaccatgtccctcgagagaccccccagggtgcctccgagagctgcctcacagaggcctccgaccagggagaccttccatcctcctccacctgttccacccagaggacgctgattccacctcctaaaacctgcctacttcaggactttaagactcacagtcttcagcctgttaatgatgtcttcatgttgagttttatagcatgactgttgaccttaagatccattctcattgctgataatgctgcagccctgctggtttgggcttgcctcgaagattttattaaggcacgaagaagtgaaaaactaagggcttcattcaccatcaccaagtatatcgaaccatatacttgtttgccaaaaggatgaagacttaatcgaaatacttacctctaatttgccatatcagaagcctaaaaagaatgatcataaatgtacttcaccagtgattttactgaaatgcacttatattagtctttatgtatttgctagttcagcctgatttctagaagaggttatagtgtgagacttgtagtattcaagtaagataagtgacctaattttaaaataattcttctacttttctgtatattcagcagggtatttaagtgctagggctggtcacacacaaccaactgaaaaagactagagggattagtacaaactcctcttatacagaaggcaaatctgaggttccacagaagtctggaaccaagactattcagttggttaaataaagaggttagtctagactgggcctgctcattctaggtcaccacattttccatctccaaatagccaggccctctctccctcaagaaatgcccagatgtagaaattcatcagtgcctattggtcttccagaattttccatcttccgtatctcccaggcatgagactaccaagtttgtttgttttctttccaatttgggaatttatacttcagtatggtttcaacgcagttatgtttccagagaacatctagaagtggctggaaaccagaagctggggattccagggaccccacttagtgctctatttcctttataggttttatttctggtcatagagagagaaggacctttgactttttcttcgttgaggcttctgaggaggaaaaacaaacctaaaatagaaatacagtcagcctttcaaatccatgggttctgtgtccgtggattcaaccaaccttggatcaaaaatatttgaaaaaaaatctacaaagtttcaaaaagcaaaacttgaatttgctgcatgccaagaagtatgttgaattcatgtaaatgaagtgatgtgtaggcattgtattagatattataagaaatctagaaatgatttaaagcatacaggaggatgtgcataggttatatgcaaatactatgctattttatatatgggacttgagcatttgtggattttgatactgggggatcctggaaccaatcccccatggataccaaagtacgactgtagttatctattttttacatacttattattaccaccatgctcagtaagtccatttttgcatggaatatggagccttaaaacatgtcatgaatttggagtccctggcacataaatctaccttcaaatcagaggtccttaatgatgcctaaacatacagtaaaattagaatcagaaatacttctttaaaaaatattcaaaatgtgtttgtttcccatgggattattctctatcccacacgaatgtaaaaaaatccacattaatgatccatttaagtatagttttattgggtccttttctaatgattaaaggttctttctcaatttcattcctcagtcctgcaagtaaggactcatactgaagagtactgaaacaaggacttcttgtcagaaacagcttctggaatcttgggttttgtttttgttttttgacaaaatacactattggccatgtccatcacgagagtgtttgtagtaattaattaccttgtacaggacctggcacttagtagcattcttcaaatgttccctcagtgatccttttactctccttgtcacttatttgggagaaataggggcacgtgagataagaagaagaataattttgatgttggtatgcttgccctgttacttatagacagtctttgtcataggcaaacttgaatttgatttaaaatagggctgggaaaaatattcaataactgtaagcccccttttggatgccaaacttagaattttgtacattctctgatgaacaagcatttagatcgtaacatggtaaagcctattaccagccaatgttgttagcatctttgtatgcacatcactgtttgtgcaatatatgaatattttgttgcattgtattcttatataaaaataagtagaaaacataaaattgaaattgctgatga

SEQ ID NO: 36 - protein, 805 aa, Homo sapiens
MAASGVPRGCDILIVYSPDAEEWCQYLQTLFLSSRQVRSQKILTHRLGPEASFSAEDLSLFLSTRCVVVLLSAELVQHFHKPALLPLLQRAFHPPHRVVRLLCGVRDSEEFLDFFPDWAHWQELTCDDEPETYVAAVKKAISEDSGCDSVTDTEPEDEKVVSYSKQQNLPTVTSPGNLMVVQPDRIRCGAETTVYVIVRCKLDDRVATEAEFSPEDSPSVRMEAKVENEYTISVKAPNLSSGNVSLKIYSGDLVVCETVISYYTDMEEIGNLLSNAANPVEFMCQAFKIVPYNTETLDKLLTESLKNNIPASGLHLFGINQLEEEDMMTNQRDEELPTLLHFAAKYGLKNLTALLLTCPGALQAYSVANKHGHYPNTIAEKHGFRDLRQFIDEYVETVDMLKSHIKEELMHGEEADAVYESMAHLSTDLLMKCSLNPGCDEDLYESMAAFVPAATEDLYVEMLQASTSNPIPGDGFSRATKDSMIRKFLEGNSMGMTNLERDQCHLGQEEDVYHTVDDDEAFSVDLASRPPVPVPRPETTAPGAHQLPDNEPYIFKVFAEKSQERPGNFYVSSESIRKGPPVRPWRDRPQSSIYDPFAGMKTPGQRQLITLQEQVKLGIVNVDEAVLHFKEWQLNQKKRSESFRFQQENLKRLRDSITRRQREKQKSGKQTDLEITVPIRHSQHLPAKVEFGVYESGPRKSVIPPRTELRRGDWKTDSTSSTASSTSNRSSTRSLLSVSSGMEGDNEDNEVPEVTRSRSPGPPQVDGTPTMSLERPPRVPPRAASQRPPTRETFHPPPPVPPRGR

SEQ ID NO: 37 - DNA, 1564 bp, Homo sapiens
gggctgtcctaaaaccagaagcttggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactgaaggatgggcctcagtctctaaggaaggcagagacctgggttgagcctcagaataaaagatcttcttccaagaaatgcaaacaggctgttcaccaccatctccagctgatcacagacaccagcaaagcaatgcactcctgaccaagtagattcttttaaaaattagagtgcattactttgaatcaaaaatttatttatatttcaagaataaagtactaagattgtgctcaatacacagaaaagtttcaaacctactaatccagcgacaatttgaatcggttttgtaggtagaggaataaaatgaaatactaaatctttctgta

SEQ ID NO: 38 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 39 - protein, 373 aa, Homo sapiens
MYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 40 - protein, 317 aa, Homo sapiens
MRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 41 - protein, 194 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILR

SEQ ID NO: 42 - DNA, 2411 bp, Homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 43 - DNA, 2515 bp, Homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 44 - DNA, 2587 bp, Homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 45 - DNA, 2467 bp, Homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 46 - DNA, 2804 bp, Homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 47 - protein, 165 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYENNPKGRSSKYGLLHCGNTEKDGKSPLTAHDARHTKAICL

SEQ ID NO: 48 - protein, 204 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEYIEEKKRVDICRETPNICPHSGENTEYDTIPHTNRTILKEDPANTVYSTVEIPKKMENPHSLLTMPDTPRLFAYENVI

SEQ ID NO: 49 - protein, 228 aa, Homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 50 - protein, 188 aa, Homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEEYIEEKKRVDICRETPNICPHSGENTEYDTIPHTNRTILKEDPANTVYSTVEIPKKMENPHSLLTMPDTPRLFAYENVI

SEQ ID NO: 51 - protein, 296 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEENNPKGRSSKYGLLHCGNTEKDGKSPLTAHDARHTKAICL

SEQ ID NO: 52 - DNA, 1387 bp, Homo sapiens
agagcagggcctgacatctccccagaacagacgtttgaacagagcaggcttctgaggtctccaaaatgcctgtcccagcctcctggccccatcctcctggtcctttcctgcttctgactctactgctgggacttacagaagtggcaggtgaggaggagctacagatgattcagcctgagaagctcctgttggtcacagttggaaagacagccactctgcactgcactgtgacctccctgcttcccgtgggacccgtcctgtggttcagaggagttggaccaggccgggaattaatctacaatcaaaaagaaggccacttccccagggtaacaacagtttcagacctcacaaagagaaacaacatggacttttccatccgcatcagtagcatcaccccagcagatgtcggcacatactactgtgtgaagtttcgaaaagggagccctgagaacgtggagtttaagtctggaccaggcactgagatggctttgggtgccaaaccctctgcccccgtggtattgggccctgcggcgaggaccacacctgagcatacagtgagtttcacctgtgagtcccatggcttctctcccagagacatcaccctgaaatggttcaaaaatgggaatgagctctcagacttccagaccaacgtggaccccacaggacagagtgtggcctacagcatccgcagcacagccagggtggtactggacccctgggacgttcgctctcaggtcatctgcgaggtggcccatgtcaccttgcagggggaccctcttcgtgggactgccaacttgtctgaggccatccgaggcccggcatcatcccttactgcgctgctcctcatagctgtcctcctgggccccatctacgtcccctggaagcagaagacctgactctccttccttcctcccctgccacgtgggaccctcatctctgctgcctccttcctttcctgagaggctcagcttgagagaatgagccagtgagaagcttctctagacttggctccaaacatctcccctcccaagacatctgcctgcccacaggctcctgttgctccttcacacagacctggatgccccagagcaaggtcttcattcatggtcctgagcaggtgccatgggattgggctctgggcactgacttaacggcacctccctagaaggcgagaaacatgccaaatctaaacacaccaggactcccatccatcgccttgagactgaccgtaaaccacagacgctctccaggttctcaagagttatcctgccttccagattcctgcctatcccaactccccagccttgttgaggttctctattgcctcttgaatacaaatgcactcccaaagtggttttaagaaaataaaaagattatccttccaaaa

SEQ ID NO: 53 - DNA, 1716 bp, Homo sapiens
agagcagggcctgacatctccccagaacagacgtttgaacagagcaggcttctgaggtctccaaaatgcctgtcccagcctcctggccccatcctcctggtcctttcctgcttctgactctactgctgggacttacagaagtggcaggtgaggaggagctacagatgattcagcctgagaagctcctgttggtcacagttggaaagacagccactctgcactgcactgtgacctccctgcttcccgtgggacccgtcctgtggttcagaggagttggaccaggccgggaattaatctacaatcaaaaagaaggccacttccccagggtaacaacagtttcagacctcacaaagagaaacaacatggacttttccatccgcatcagtagcatcaccccagcagatgtcggcacatactactgtgtgaagtttcgaaaagggagccctgagaacgtggagtttaagtctggaccaggcactgagatggctttgggtgccaaaccctctgcccccgtggtattgggccctgcggcgaggaccacacctgagcatacagtgagtttcacctgtgagtcccatggcttctctcccagagacatcaccctgaaatggttcaaaaatgggaatgagctctcagacttccagaccaacgtggaccccacaggacagagtgtggcctacagcatccgcagcacagccagggtggtactggacccctgggacgttcgctctcaggtcatctgcgaggtggcccatgtcaccttgcagggggaccctcttcgtgggactgccaacttgtctgaggccatccgagttccacccaccttggaggttactcaacagcccatgagggtggggaaccaggtaaacgtcacctgccaggtgaggaagttctacccccagagcctacagctgacctggtcggagaatggaaacgtgtgccagagagaaacagcctcgacccttacagagaacaaggatggtacctacaactggacaagctggttcctggtgaacatatctgaccaaagggatgatgtggtcctcacctgccaggtgaagcatgatgggcagctggcggtcagcaaacgccttgccctagaggtcacagtccaccagaaggaccagagctcagatgctacccctggcccggcatcatcccttactgcgctgctcctcatagctgtcctcctgggccccatctacgtcccctggaagcagaagacctgactctccttccttcctcccctgccacgtgggaccctcatctctgctgcctccttcctttcctgagaggctcagcttgagagaatgagccagtgagaagcttctctagacttggctccaaacatctcccctcccaagacatctgcctgcccacaggctcctgttgctccttcacacagacctggatgccccagagcaaggtcttcattcatggtcctgagcaggtgccatgggattgggctctgggcactgacttaacggcacctccctagaaggcgagaaacatgccaaatctaaacacaccaggactcccatccatcgccttgagactgaccgtaaaccacagacgctctccaggttctcaagagttatcctgccttccagattcctgcctatcccaactccccagccttgttgaggttctctattgcctcttgaatacaaatgcactcccaaagtggttttaagaaaataaaaagattatccttcc

SEQ ID NO: 54 - DNA, 1069 bp, Homo sapiens
agagcagggcctgacatctccccagaacagacgtttgaacagagcaggcttctgaggtctccaaaatgcctgtcccagcctcctggccccatcctcctggtcctttcctgcttctgactctactgctgggacttacagaagtggcaggtgaggaggagctacagatgattcagcctgagaagctcctgttggtcacagttggaaagacagccactctgcactgcactgtgacctccctgcttcccgtgggacccgtcctgtggttcagaggagttggaccaggccgggaattaatctacaatcaaaaagaaggccacttccccagggtaacaacagtttcagacctcacaaagagaaacaacatggacttttccatccgcatcagtagcatcaccccagcagatgtcggcacatactactgtgtgaagtttcgaaaagggagccctgagaacgtggagtttaagtctggaccaggcactgagatggctttgggtggcccggcatcatcccttactgcgctgctcctcatagctgtcctcctgggccccatctacgtcccctggaagcagaagacctgactctccttccttcctcccctgccacgtgggaccctcatctctgctgcctccttcctttcctgagaggctcagcttgagagaatgagccagtgagaagcttctctagacttggctccaaacatctcccctcccaagacatctgcctgcccacaggctcctgttgctccttcacacagacctggatgccccagagcaaggtcttcattcatggtcctgagcaggtgccatgggattgggctctgggcactgacttaacggcacctccctagaaggcgagaaacatgccaaatctaaacacaccaggactcccatccatcgccttgagactgaccgtaaaccacagacgctctccaggttctcaagagttatcctgccttccagattcctgcctatcccaactccccagccttgttgaggttctctattgcctcttgaatacaaatgcactcccaaagtggttttaagaaaataaaaagattatccttccaaaa

SEQ ID NO: 55-56 - skipped (2 entries)

SEQ ID NO: 57 - DNA, 2027 bp, Acyrthosiphon pisum
gatattgcattctgcgtaaaatgtaaaggtgtaataggtactatagtaataccctaatactgtaatccctaaagatttaagttataacttctaactcataaacatcgaagttctaccttgcaagtgtggtactgtggttttagcatccagttacagtacatgttaaggcttgcgagtatattttgttaattcattgaattaagaaacacattttatatttgcgaccatattgcatcagtgatcagtctaaacctcaaaaatgacatcaaaaactggcgctactggagtgtctgagggggagcaggtaaaatctaaaagccagttaaaaaaggaagaaaaacttgcagctaaagcaattaagaaggaacaattcaaaacaatgaataaaacaaatactaatgaccaggaatctggtgaagacatctctgttggaaagtacggagttttaaaaatgatccaaagttcaaaaagtgttgtatctgatcgagtatttgtggatgtaaaaaacattatgactattttagctgataatagtgtttggatacgtgggagacttcatactagtcgtgcaaaaggcaaacaatgttttatggttgtcagacagcagcaatggtctatccaagtgttagctgtagtatctgatactattagtaaacaatttgtaaagttttgttccaatattaacaaagaatctatagtagacattaaaggtaaagttacaagaacaccttctaaaattggttcgtgctcacaacaagatgttgaattacatattgaagaattgtgggttgttagtcagtctgctaatcaacttcctttacttattgaagatgcatctaggccagttaaaggggatgatgacgaggatcaaggtttaaatattaaagtcaatcaagatacaaggctagacaatagagtaattgatttacgtacccctgcaaatcaagctattttccgccttgaagctggtgtttgtaaaattttccgagatttgctcactgcaaaaggttttgttgaaattcatactccaaaaatgataaatgctgcaagtgaaggtggagcaaatgtttttacaatatcatatttcaaaggctctgcatatcttgctcaatcacctcagttgtataaacaaatggcaattgctggtgactttgatagagtatttacaattggtgcagtttttagagcagaagatagtaacacatatagacatttgactgaattcgttggcttagatttagaaatggcttttaaatatcactatcatgaagtggttgatgttattggttcactttttactaacatatttaaaggattacgtgacaattatcaatctgaaattaaaacaatttgcgaacaatatccagtagaaccatttaaattcttggatccacctttaaaacttgaatttccaacggctgttaatatgcttcgtgaggctggagttgaaatgggagatgaagatgatttatcaacaccaaatgaaaaattacttggacgtttggtcaaagcaaaatatgatacagatttctacattttggataagtttcctcttgccatacgacctttttacaccatgccagatcctgactcaaaagttgcttctaattcttatgatatgtttatgagaggtgaagaaattctatcgggtgctcagaggatacatgattttgattacttaatagaaagagccaaacatcatcaagttgatattgagaagattgcagcttatatagacgctttccggtatgggtgtccacctcatgctggtggtggaattggattagagagagttgttatgttgtacttgggattagacaacataagaaaagtatcaatgtttcctcgtgatcctaaaagacttactccatgattaaaattatactttataaaatatttattttattataactattttttaaataaagtatgttaataattaaatatgttatgaaattgtcatttaaattggtgaagcatttaataattaaatataaataatattttgaataaaaaaaaaaaaaaaa

SEQ ID NO: 58 - DNA, 3996 bp, Mus musculus
gtcgccagcgccgggcttggcggaaccactcatcccgccacaggtggctccgcatcaggcgtggccttgtgcggcgccccgggcctgagcgctgcgacggcgccttcgttttcagccgcccgcgaccgactcgccgaggctttactcgcttgccgccgtctttgtccgcacacagatgatttgagacatgtggatttgtgatatgaattcatttggaaagccactttggggctatgactgtgatttctgatgaaaccaaattggaataattttcatgatatttgtatatttatatctatatatattttaaaattttacatttgacttaaagtgccatgagaaaatttgcatattgcaaggtggttctagccacctccttggtttgggtactcttggatatgttcctgctgctttacttcagtgaatgcaacaaatgtgaagagaagcaggagagaggacttcctgctggtgacgttctagagctagtgcagaaacctcatgaaggtcctggagaaatggggaaaccagtcgtcattccgaaagaggatcaagaaaagatgaaagagatgtttaaaatcaatcagttcaacttaatggcaagtgagatgattgcactcaacagatccttaccagatgtgaggttagaggggtgtaaaacaaaggtgtatccagataaccttcctacaacaagtgtggtgattgttttccacaatgaggcatggagcacacttctgcgaacagtccatagtgtcattaatcgctcgccaagacatatgatagaagagattgttcttgtagatgatgccagtgaaagagactttttgaaaagacctctagagagttatgtgaaaaaactgaaagtgccggttcatgtaattcgaatggagcagcgctctgggttgatcagagctagattgaaaggagctgctgtgtccagaggccaagtgatcaccttcttagatgctcactgtgaatgcaccgcagggtggctggagcctctgttggcccggatcaaacatgacaggaggacagtggtgtgtcctatcatcgacgtgatcagtgatgacactttcgagtacatggcgggctctgacatgacctatggtggcttcaactggaagctcaattttcgttggtatcctgttccccagcgagaaatggacagaaggaaaggtgacaggactcttcctgtcagaacacctacaatggctggaggccttttttcaatagacagagattactttcaggaaattggaacatacgatgctggaatggatatttggggaggagaaaacctagaaatttcatttaggatttggcagtgtggaggaactttggaaatagtgacatgctcccatgttggtcatgtgtttcggaaagctacaccttacacatttccaggagggacagggcagattatcaataagaataacaggcggcttgcagaagtatggatggatgaattcaagaatttcttctatataatttctccaggtgttacaaaggtagattatggagatatatcatcaagacttggcctaagacgtaaactacaatgcaaacctttctcttggtacctagaaaatatttaccctgattctcaaattcctcgtcactatttctcattgggagagatacgaaatgtggaaacaaatcagtgtctagataacatggctagaaaagagaatgaaaaggttggaatctttaattgtcatggtatgggaggtaatcaggttttctcctacactgccaacaaagaaattagaacagacgacctttgtttggacgtttccaaacttaatggcccagtcaccatgctcaaatgccaccacctaaaaggcaaccagctctgggagtatgacccagtgaaattaaccctgcagcatgtgaacagcaatcagtgcctggataaagccacagaggaggacagccaggtgcccagcattagagactgcactggaagccggtcccagcagtggcttcttcgaaacgtcacccttccagaaatattctgagaccaaatttacaacaacaaagaaagaaggaaggattgactgggctacctcagcatacattctgccacattcttcagtagcaaaaaaggaaagtgcttccctcttctgtgggatgttaaggtttctcagccattaaaactcagactcctagcttttcacttgctgtgaaccggccttcctgtcccaggacgtgcgactgcaaagtagcgagactgtgcacactgatgtttacaaggctggaagagtcttcatcaagagcagcggcaaagccttcagatcccaaacaggcgcacgagcgctctcgggatagatgcactttctacagcgtgggtgcacgtcagccgtttctgctgaatgtgccttgtgaagaagagttttcctgattagaatcagtagctggaatattaaatattgatagaaaagtaagtggaaaagagctggaaccaggttcagaatcatgaaaacatttttacaacaataaaaattatgtcaaacagggtttaaaggaaattaaaaagaattatgagaagtacaatttgttatagtatagtatgagatttctatatagattttatacctcagtggggaaaaactgattccagtgatgttgatgttgttttcatctgtgatagtcatggatgcttctattttcctcggggtgctcagagtgagatagtgggtggggtgagctctctgaacatagtttgaatttctttttacaatattcttatttggtttgtgggctgttggaattgtaagtttaattgccattctttaaaaaaaaaaaaatggagatttactctgcctggtctcagttggaaagttttcgatctctctctgttgttcttgggtcaactggcgtacagactctgcctcgccctgcctccctccctccacacagtttcttaacatgctgtccacttaactctgctgagctgattgtgcttcatgcccaagactcacgctactgcactgcaggtttgtctccacagcaataacctctgacccctgcgagtcacttcacagagtgctgtttatctggagattgggagatcagacatttaatgggttttttgaaacccttgccactccacttagccgtgtgcttccctgtcggacgcacactcaggttcctcctcagttgtaatgttgatttgctgctttttcacaaaataaaaatggactgaaagtgtttaattggcattttttaatgagttagccaaagaagtgcaactgtgattccacatcacagtcctgcgtttcctccctaaattctattttgttatttggttttctttactctggtttttagtaacaggatcacgtattaaaatttgctgttaattaaagtttaccttttgattttacagaaatctcattgtgacatttttttccatagcagcccattggccaaaatgcataattctgcttcctgtctgtcaggtgatacttgatggtattcctctttttttgattatcaaagacttgcttcaggaagtgctgtttcttgtgcgtaagaatgttcctgaagtcacactcgtcagtgacaccatctgtcatcattcatccagtcgtcagcatcgaatatatggatgcttagggtaaggctgtcctccaggatgcagagactccccatgcaatctgaatgaaaccggtgttggctggaatactttttctcttctgtgagctatgttaattctgtctccaatgggcccaatgtttgaaaagtcagatgtctaataaattattttcatgctcagaatttcagatttttgtaccctaacaacatggttttattccttattgtaaaagtttaacagcgttgtgatttaaaaatgtgtctcacttttttaagtggggggatgtgagtgacttaattcatgggtacttttagaacctattagataatcttattgcctttatttttctaattaaaatattcctaaatactttgaaaataaaaaaaaaaaaaaaaaaaaaa

SEQ ID NO: 59 - DNA, 4193 bp, Mus musculus
agaggccgaggaggcggcgcggcggactgcggcccgagaggagcgcccgcgctgcggcccgcgggagcgcgccgccaccgcccgcgccggcctgaggagcgcctgccgccgcgcgggccgaggtgagtgcaccgcaggccggcgggctggtgacgccggagcggagcggagcggagcggggcgaggcgcaggcccgggcgcgcccgcctcctcggccctttgtggccgcgcggccggggcgcggggtccggcggcggggactcccggcccgcccggcgcgcggcgtctccctctgccgggcagccgcgcagagctgcgagttggcgtcctagtttgacattgagttagaaaccgtttttcttcagaagacggaagtttgttttattgacagagatgatttgagacatgtggatttgtgatatgaattcatttggaaagccactttggggctatgactgtgatttctgatgaaaccaaattggaataattttcatgatatttgtatatttatatctatatatattttaaaattttacatttgacttaaagtgccatgagaaaatttgcatattgcaaggtggttctagccacctccttggtttgggtactcttggatatgttcctgctgctttacttcagtgaatgcaacaaatgtgaagagaagcaggagagaggacttcctgctggtgacgttctagagctagtgcagaaacctcatgaaggtcctggagaaatggggaaaccagtcgtcattccgaaagaggatcaagaaaagatgaaagagatgtttaaaatcaatcagttcaacttaatggcaagtgagatgattgcactcaacagatccttaccagatgtgaggttagaggggtgtaaaacaaaggtgtatccagataaccttcctacaacaagtgtggtgattgttttccacaatgaggcatggagcacacttctgcgaacagtccatagtgtcattaatcgctcgccaagacatatgatagaagagattgttcttgtagatgatgccagtgaaagagactttttgaaaagacctctagagagttatgtgaaaaaactgaaagtgccggttcatgtaattcgaatggagcagcgctctgggttgatcagagctagattgaaaggagctgctgtgtccagaggccaagtgatcaccttcttagatgctcactgtgaatgcaccgcagggtggctggagcctctgttggcccggatcaaacatgacaggaggacagtggtgtgtcctatcatcgacgtgatcagtgatgacactttcgagtacatggcgggctctgacatgacctatggtggcttcaactggaagctcaattttcgttggtatcctgttccccagcgagaaatggacagaaggaaaggtgacaggactcttcctgtcagaacacctacaatggctggaggccttttttcaatagacagagattactttcaggaaattggaacatacgatgctggaatggatatttggggaggagaaaacctagaaatttcatttaggatttggcagtgtggaggaactttggaaatagtgacatgctcccatgttggtcatgtgtttcggaaagctacaccttacacatttccaggagggacagggcagattatcaataagaataacaggcggcttgcagaagtatggatggatgaattcaagaatttcttctatataatttctccaggtgttacaaaggtagattatggagatatatcatcaagacttggcctaagacgtaaactacaatgcaaacctttctcttggtacctagaaaatatttaccctgattctcaaattcctcgtcactatttctcattgggagagatacgaaatgtggaaacaaatcagtgtctagataacatggctagaaaagagaatgaaaaggttggaatctttaattgtcatggtatgggaggtaatcaggttttctcctacactgccaacaaagaaattagaacagacgacctttgtttggacgtttccaaacttaatggcccagtcaccatgctcaaatgccaccacctaaaaggcaaccagctctgggagtatgacccagtgaaattaaccctgcagcatgtgaacagcaatcagtgcctggataaagccacagaggaggacagccaggtgcccagcattagagactgcactggaagccggtcccagcagtggcttcttcgaaacgtcacccttccagaaatattctgagaccaaatttacaacaacaaagaaagaaggaaggattgactgggctacctcagcatacattctgccacattcttcagtagcaaaaaaggaaagtgcttccctcttctgtgggatgttaaggtttctcagccattaaaactcagactcctagcttttcacttgctgtgaaccggccttcctgtcccaggacgtgcgactgcaaagtagcgagactgtgcacactgatgtttacaaggctggaagagtcttcatcaagagcagcggcaaagccttcagatcccaaacaggcgcacgagcgctctcgggatagatgcactttctacagcgtgggtgcacgtcagccgtttctgctgaatgtgccttgtgaagaagagttttcctgattagaatcagtagctggaatattaaatattgatagaaaagtaagtggaaaagagctggaaccaggttcagaatcatgaaaacatttttacaacaataaaaattatgtcaaacagggtttaaaggaaattaaaaagaattatgagaagtacaatttgttatagtatagtatgagatttctatatagattttatacctcagtggggaaaaactgattccagtgatgttgatgttgttttcatctgtgatagtcatggatgcttctattttcctcggggtgctcagagtgagatagtgggtggggtgagctctctgaacatagtttgaatttctttttacaatattcttatttggtttgtgggctgttggaattgtaagtttaattgccattctttaaaaaaaaaaaaatggagatttactctgcctggtctcagttggaaagttttcgatctctctctgttgttcttgggtcaactggcgtacagactctgcctcgccctgcctccctccctccacacagtttcttaacatgctgtccacttaactctgctgagctgattgtgcttcatgcccaagactcacgctactgcactgcaggtttgtctccacagcaataacctctgacccctgcgagtcacttcacagagtgctgtttatctggagattgggagatcagacatttaatgggttttttgaaacccttgccactccacttagccgtgtgcttccctgtcggacgcacactcaggttcctcctcagttgtaatgttgatttgctgctttttcacaaaataaaaatggactgaaagtgtttaattggcattttttaatgagttagccaaagaagtgcaactgtgattccacatcacagtcctgcgtttcctccctaaattctattttgttatttggttttctttactctggtttttagtaacaggatcacgtattaaaatttgctgttaattaaagtttaccttttgattttacagaaatctcattgtgacatttttttccatagcagcccattggccaaaatgcataattctgcttcctgtctgtcaggtgatacttgatggtattcctctttttttgattatcaaagacttgcttcaggaagtgctgtttcttgtgcgtaagaatgttcctgaagtcacactcgtcagtgacaccatctgtcatcattcatccagtcgtcagcatcgaatatatggatgcttagggtaaggctgtcctccaggatgcagagactccccatgcaatctgaatgaaaccggtgttggctggaatactttttctcttctgtgagctatgttaattctgtctccaatgggcccaatgtttgaaaagtcagatgtctaataaattattttcatgctcagaatttcagatttttgtaccctaacaacatggttttattccttattgtaaaagtttaacagcgttgtgatttaaaaatgtgtctcacttttttaagtggggggatgtgagtgacttaattcatgggtacttttagaacctattagataatcttattgcctttatttttctaattaaaatattcctaaatactttgaaaata

SEQ ID NO: 60 - protein, 559 aa, Mus musculus
MRKFAYCKVVLATSLVWVLLDMFLLLYFSECNKCEEKQERGLPAGDVLELVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPRHMIEEIVLVDDASERDFLKRPLESYVKKLKVPVHVIRMEQRSGLIRARLKGAAVSRGQVITFLDAHCECTAGWLEPLLARIKHDRRTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRLGLRRKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCTGSRSQQWLLRNVTLPEIF

SEQ ID NO: 61 - protein, 559 aa, Mus musculus; identical to SEQ ID NO: 60

SEQ ID NO: 62 - protein, 559 aa, Mus musculus; identical to SEQ ID NO: 60

SEQ ID NO: 63 - DNA, 36 bp, Artificial sequence
  T7 RNA polymerase promoter sequence
scgattgaggccggtaatacgactcactataggggc

SEQ ID NO: 64 - DNA, 27 bp, Artificial sequence
  Single strand DNA oligonucleotide
ctgaaatattcgatgatcaattctcag

SEQ ID NO: 65 - DNA, 28 bp, Artificial sequence
  Single strand DNA oligonucleotide
tcattataaatgaaacagttccaaatcg

SEQ ID NO: 66 - DNA, 22 bp, Artificial sequence
  Single strand DNA oligonucleotide
agtgtgccaagtcgcttctcac

SEQ ID NO: 67 - DNA, 27 bp, Artificial sequence
  Single strand DNA oligonucleotide
gagacacagagaaacaaaggaaacttc

SEQ ID NO: 68 - DNA, 23 bp, Artificial sequence
  Single strand DNA oligonucleotide
  misc_feature (11)..(11): N is either C or T
agaggaaactnccctcctagatt

SEQ ID NO: 69 - DNA, 26 bp, Artificial sequence
  Single strand DNA oligonucleotide
cccagtttatctttcagtattatgag

SEQ ID NO: 70 - DNA, 25 bp, Artificial sequence
  Single strand DNA oligonucleotide
  misc_feature (14)..(14): N is either C or G
aaggagaagtcccnaatggctacaa

SEQ ID NO: 71 - DNA, 25 bp, Artificial sequence
  Single strand DNA oligonucleotide
agggtacaactgccaaaggagaggt

SEQ ID NO: 72 - DNA, 25 bp, Artificial sequence
  Single strand DNA oligonucleotide
ggcaaaggagaagtccctgatggtt

SEQ ID NO: 73 - DNA, 26 bp, Artificial sequence
  Single strand DNA oligonucleotide
ctgacaaagaagtccccaatggctac

SEQ ID NO: 74 - DNA, 25 bp, Artificial sequence
  Single strand DNA oligonucleotide
cactgacaaaggagaagtccccgat

SEQ ID NO: 75 - DNA, 24 bp, Artificial sequence
  Single strand DNA oligonucleotide
agacaaatcagggctgcccagtga

SEQ ID NO: 76 - DNA, 21 bp, Artificial sequence
  Single strand DNA oligonucleotide
gactcagggctgcccaacgat

SEQ ID NO: 77 - DNA, 24 bp, Artificial sequence
  Single strand DNA oligonucleotide
agaggttctgacttactcccagag

SEQ ID NO: 78 - DNA, 26 bp, Artificial sequence
  Single strand DNA oligonucleotide
gacttacttcaattatgaagcccaac

SEQ ID NO: 79 - DNA, 24 bp, Artificial sequence
  Single strand DNA oligonucleotide
ccagaatgaagctcaactagacaa

SEQ ID NO: 80 - DNA, 27 bp, Artificial sequence
  Single strand DNA oligonucleotide
gacttacttccagaatgaagctcaact

SEQ ID NO: 81 - DNA, 28 bp, Artificial sequence
  Single strand DNA oligonucleotide
gagcaaaaggaaacattcttgaacgatt

SEQ ID NO: 82 - DNA, 26 bp, Artificial sequence
  Single strand DNA oligonucleotide
  misc_feature (5)..(5): N is either A or T
ggctnatccattactcatatggtgtt

SEQ ID NO: 83 - DNA, 25 bp, Artificial sequence
  Single strand DNA oligonucleotide
gataaaggagaagtccccgatggct

SEQ ID NO: 84 - DNA, 25 bp, Artificial sequence
  Single strand DNA oligonucleotide
gattcacagttgcctaaggatcgat

SEQ ID NO: 85 - DNA, 23 bp, Artificial sequence
  Single strand DNA oligonucleotide
gattcagggatgcccgaggatcg

SEQ ID NO: 86 - DNA, 23 bp, Artificial sequence
  Single strand DNA oligonucleotide
gattcggggatgccgaaggatcg

SEQ ID NO: 87 - DNA, 25 bp, Artificial sequence
  Single strand DNA oligonucleotide
gcagagcgataaaggaagcatccct

SEQ ID NO: 88 - DNA, 25 bp, Artificial sequence
  Single strand DNA oligonucleotide
tccggtatgcccaacaatcgattct

SEQ ID NO: 89 - DNA, 27 bp, Artificial sequence
  Single strand DNA oligonucleotide
gattttaacaatgaagcagacacccct

SEQ ID NO: 90 - DNA, 26 bp, Artificial sequence
  Single strand DNA oligonucleotide
gatgaaacaggtatgcccaaggaaag

SEQ ID NO: 91 - DNA, 28 bp, Artificial sequence
  Single strand DNA oligonucleotide
tatcatagatgagtcaggaatgccaaag

SEQ ID NO: 92 - DNA, 27 bp, Artificial sequence
  Single strand DNA oligonucleotide
gactttcagaaaggagatatagctgaa

SEQ ID NO: 93 - DNA, 25 bp, Artificial sequence
  Single strand DNA oligonucleotide
caaggccacatacgagcaaggcgtc

SEQ ID NO: 94 - DNA, 28 bp, Artificial sequence
  Single strand DNA oligonucleotide
caaagatataaacaaaggagagatctct

SEQ ID NO: 95 - DNA, 26 bp, Artificial sequence
  Single strand DNA oligonucleotide
agagaagggagatctttcctctgagt

SEQ ID NO: 96 - DNA, 26 bp, Artificial sequence
  Single strand DNA oligonucleotide
gactgataagggagatgttcctgaag

SEQ ID NO: 97 - DNA, 28 bp, Artificial sequence
  Single strand DNA oligonucleotide
ggctgatctatttctcatatgatgttaa

SEQ ID NO: 98 - DNA, 27 bp, Artificial sequence
  Single strand DNA oligonucleotide
gccacatatgagagtggatttgtcatt

SEQ ID NO: 99 - DNA, 23 bp, Artificial sequence
  Single strand DNA oligonucleotide
ggtgccccagaatctctcagcct